(19) United States
(12) Patent Application Publication
(10) Pub. No.: US 2004/0033491 A1
(43) Pub. Date: Feb. 19, 2004

(54) Proteins and nucleic acids encoding same

(76) Inventors: John P. Alsobrook II, Madison, CT (US); David W. Anderson, Branford, CT (US); Catherine E. Burgess, Wethersfield, CT (US); Ferenc L. Boldog, North Haven, CT (US); Stacie J. Casman, North Haven, CT (US); Steven D. Colman, Guilford, CT (US); Shlomit R. Edinger, New Haven, CT (US); Karen Ellerman, Branford, CT (US); Valerie Gerlach, Branford, CT (US); Linda Gorman, Branford, CT (US); William M. Grosse, Branford, CT (US); Xiaojia Sasha Guo, Branford, CT (US); John L. Herrmann, Guilford, CT (US); Ramesh Kekuda, Danbury, CT (US); Denise M. Lepley, Branford, CT (US); Li Li, Branford, CT (US); John R. MacDougall, Hamden, CT (US); Isabelle Millet, Milford, CT (US); Carol E. A. Pena, New Haven, CT (US); John A. Peyman, New Haven, CT (US); Luca Rastelli, Guilford, CT (US); Daniel K. Rieger, Branford, CT (US); Richard A. Shimkets, Guilford, CT (US); Glennda Smithson, Guilford, CT (US); Kimberly A. Spytek, New Haven, CT (US); David J. Stone, Guilford, CT (US); Velizar T. Tchernev, Branford, CT (US); Corine A.M. Vernet, Branford, CT (US); Edward Z. Voss, Wallingford, CT (US); Bryan D. Zerhusen, Branford, CT (US); Haihong Zhong, Guilford, CT (US); Mei Zhong, Branford, CT (US)

Correspondence Address: Ivor R. Elrifi, MINTZ, LEVIN, COHN, FERRIS,, GLOVSKY and POPEO, P.C., One Financial Center, Boston, MA, 02111, US

(21) Appl. No.: 10/016,248

(22) Filed: Dec. 10, 2001

Related U.S. Application Data
(60) Provisional application No. 60/254,329, filed on Dec. 8, 2000.
(60) Provisional application No. 60/291,037, filed on May 15, 2001.
(60) Provisional application No. 60/255,648, filed on Dec. 14, 2000.
(60) Provisional application No. 60/297,173, filed on Jun. 8, 2001.
(60) Provisional application No. 60/309,258, filed on Jul. 31, 2001.
(60) Provisional application No. 60/326,393, filed on Oct. 1, 2001.
(60) Provisional application No. 60/315,639, filed on Aug. 29, 2001.

(51) Int. Cl. C12Q 1/68; C07H 21/04; C12N 9/00; C12P 21/02; C12N 5/06

(52) U.S. Cl. 435/6; 435/69.1; 435/183; 435/320.1; 435/325; 530/350; 536/23.2

(57) ABSTRACT

Disclosed herein are nucleic acid sequences that encode novel polypeptides. Also disclosed are polypeptides encoded by these nucleic acid sequences, and antibodies, which immunospecifically-bind to the polypeptide, as well as derivatives, variants, mutants, or fragments of the aforementioned polypeptide, polynucleotide, or antibody. The invention further discloses therapeutic, diagnostic and research methods for diagnosis, treatment, and prevention of disorders involving any one of these novel human nucleic acids and proteins.

### RELATED APPLICATIONS

This application claims priority from U.S. S. No. 60/254,329, filed Dec. 8, 2000; U.S. S. No. 60/291,037, filed May 15, 2001; U.S. S. No. 60/255,648, filed Dec. 14, 2000; U.S. S. No. 60/297,173, filed Jun. 8, 2001; U.S. S. No. 60/309,258, filed Jul. 31, 2001; U.S. S. No. 60/326,393, filed Oct. 1, 2001; U.S. S. No. 60/315,639, filed Aug. 29, 2001; each of which is incorporated by reference in its entirety.

### FIELD OF THE INVENTION

The invention generally relates to nucleic acids and polypeptides encoded thereby.

### BACKGROUND OF THE INVENTION

The invention generally relates to nucleic acids and polypeptides encoded therefrom. More specifically, the invention relates to nucleic acids encoding cytoplasmic, nuclear, membrane bound, and secreted polypeptides, as well as vectors, host cells, antibodies, and recombinant methods for producing these nucleic acids and polypeptides.

### SUMMARY OF THE INVENTION

The invention is based in part upon the discovery of nucleic acid sequences encoding novel polypeptides. The novel nucleic acids and polypeptides are referred to herein as NOVX, or NOV1, NOV2, NOV3, NOV4, NOV5, NOV6, NOV7, NOV8, NOV9, NOV10, and NOV11 nucleic acids and polypeptides. These nucleic acids and polypeptides, as well as derivatives, homologs, analogs and fragments thereof, will hereinafter be collectively designated as “NOVX” nucleic acid or polypeptide sequences.

In one aspect, the invention provides an isolated NOVX nucleic acid molecule encoding a NOVX polypeptide that includes a nucleic acid sequence that has identity to the nucleic acids disclosed in SEQ ID NOS:1, 3, 5, 7, 9, 11, 13, 15, 17, 19, 21, 23, 25, 27, 29, 31, 33, 35, 37, 39, 41 and 43. In some embodiments, the NOVX nucleic acid molecule will hybridize under stringent conditions to a nucleic acid sequence complementary to a nucleic acid molecule that includes a protein-coding sequence of a NOVX nucleic acid sequence. The invention also includes an isolated nucleic acid that encodes a NOVX polypeptide, or a fragment, homolog, analog or derivative thereof. For example, the nucleic acid can encode a polypeptide at least 80% identical to a polypeptide comprising the amino acid sequences of SEQ ID NOS:2, 4, 6, 8, 10, 12, 14, 16, 18, 20, 22, 24, 26, 28, 30, 32, 34, 26, 28, 40, 42 and 44. The nucleic acid can be, for example, a genomic DNA fragment or a cDNA molecule that includes the nucleic acid sequence of any of SEQ ID NOS:1, 3, 5, 7, 9, 11, 13, 15, 17, 19, 21, 23, 25, 27, 29, 31, 33, 35, 37, 39, 41 and 43.

Also included in the invention is an oligonucleotide, e.g., an oligonucleotide which includes at least 6 contiguous nucleotides of a NOVX nucleic acid (e.g., SEQ ID NOS:1, 3, 5, 7, 9, 11, 13, 15, 17, 19, 21, 23, 25, 27, 29, 31, 33, 35, 37, 39, 41 and 43) or a complement of said oligonucleotide.

Also included in the invention are substantially purified NOVX polypeptides (SEQ ID NOS:2, 4, 6, 8, 10, 12, 14, 16, 18, 20, 22, 24, 26, 28, 30, 32, 34, 26, 28, 40, 42 and 44). In certain embodiments, the NOVX polypeptides include an amino acid sequence that is substantially identical to the amino acid sequence of a human NOVX polypeptide.

The invention also features antibodies that immunoselectively bind to NOVX polypeptides, or fragments, homologs, analogs or derivatives thereof.

In another aspect, the invention includes pharmaceutical compositions that include therapeutically- or prophylactically-effective amounts of a therapeutic and a pharmaceutically-acceptable carrier. The therapeutic can be, e.g., a NOVX nucleic acid, a NOVX polypeptide, or an antibody specific for a NOVX polypeptide. In a further aspect, the invention includes, in one or more containers, a therapeutically- or prophylactically-effective amount of this pharmaceutical composition.

In a further aspect, the invention includes a method of producing a polypeptide by culturing a cell that includes a NOVX nucleic acid, under conditions allowing for expression of the NOVX polypeptide encoded by the DNA. If desired, the NOVX polypeptide can then be recovered.

In another aspect, the invention includes a method of detecting the presence of a NOVX polypeptide in a sample. In the method, a sample is contacted with a compound that selectively binds to the polypeptide under conditions allowing for formation of a complex between the polypeptide and the compound. The complex is detected, if present, thereby identifying the NOVX polypeptide within the sample.

The invention also includes methods to identify specific cell or tissue types based on their expression of a NOVX.

Also included in the invention is a method of detecting the presence of a NOVX nucleic acid molecule in a sample by contacting the sample with a NOVX nucleic acid probe or primer, and detecting whether the nucleic acid probe or primer bound to a NOVX nucleic acid molecule in the sample.

In a further aspect, the invention provides a method for modulating the activity of a NOVX polypeptide by contacting a cell sample that includes the NOVX polypeptide with a compound that binds to the NOVX polypeptide in an amount sufficient to modulate the activity of said polypeptide. The compound can be, e.g., a small molecule, such as a nucleic acid, peptide, polypeptide, peptidomimetic, carbohydrate, lipid or other organic (carbon containing) or inorganic molecule, as further described herein.

Also within the scope of the invention is the use of a therapeutic in the manufacture of a medicament for treating or preventing disorders or syndromes including, e.g., Alzheimer's disease, Neurodegenerative disease, Parkinson disease, type 3; Stroke, Parkinson's disease, Huntington's disease, Cerebral palsy, Epilepsy, Ataxia-telangiectasia, Behavioral disorders, Addiction, Anxiety, Pain, Neuroprotection, encephalopathy. pain, psychotic and neurological disorders, including anxiety, schizophrenia, manic depression, delirium, dementia, severe mental retardation, aneurysm, corticoneurogenic disease, gap-junction-related neuropathies and other pathological conditions of the nervous system, where dysfunctions of junctional communication are considered to play a casual role, demyelinating neuropathies (including Charcot-Marie-Tooth disease), Cardiovascular disease, Hemic and Lymphatic Diseases, acute heart failure, hypotension, hypertension, angina pectoris, myocardial infarction, ischemic heart disease, cardiomyopathy, atherosclerosis, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, hemophilia, hypercoagulation, idiopathic thrombocytopenic purpura, Erythrokeratodermia variabilis (EKV), atrioventricular (AV) conduction defects such as arrhythmia, and lens cataracts, bone disorders, Muscle Disorders, Alstrom syndrome; Orofacial cleft-2, Preeclampsia; Welander distal myopathy; Von Hippel-Lindau (VHL) syndrome, Tuberous sclerosis, hypercalceimia, Lesch-Nyhan syndrome, Multiple sclerosis, Cell adhesion, shape, interaction communication, cytokinesis disorders; myotonic dystrophy; muscular disorders and diseases; Angelman syndrome, Liddle's syndrome, Prader-Willi syndrome, Kallman syndrome, skin disorders, protease/protease inhibitor deficiency disorders, urinary retention, osteoporosis, Crohn's disease; multiple sclerosis; and Treatment of Albright Hereditary Ostoeodystrophy, ulcers, Dentatorubro-pallidoluysian atrophy (DRPLA) Hypophosphatemic rickets, autosomal dominant, Peutz-Heghers syndrome, fibromuscular dysplasia, congenital adrenal hyperplasia, endometriosis, cirrhosis, myasthenia gravis, psoriasis, actinic keratosis, excessive hair growth, allopecia, pigmentation disorders, cystitis, incontinence, renal artery stenosis, interstitial nephritis, glomerulonephritis, polycystic kidney disease, taste and scent detectability disorders, signal transduction pathway disorders, retinal diseases including those involving photoreception, deafness, keratinization disorders, oocyte maturation defects, Myotonia and Cancers including, colon and lung and breast cancer, Leukodystrophies, cancer (especially but not limited to prostate, and skin), Neoplasm; adenocarcinoma; lymphoma, uterus cancer, benign prostatic hypertrophy, enal cancer, multiple endocrine neoplasia type II, familial melanoma, ovarian cancer, adrenoleukodystrophy, Burkitt's lymphoma, Glucosidase I deficiency; severe infantile-onset Wolman disease and milder late onset cholesteryl ester storage disease (CESD), Diabetes, Pancretaitis, Obesity, digetive system disorders, anorexia, bulimia, gastrointestinal polyps, hyperthyroidism, hypothyroidism, endocrine dysfunctions, noninsulin-dependent diabetes mellitus (NIDDM1), immunological disorders and diseases, inflammatory and immune diseases, bacterial, fungal, protozoal and viral infections (particularly infections caused by HIV-1 or HIV-2), asthma, sepsis, graft versus host disease, transplantation, systemic lupus erythematosus, renal tubular acidosis or IgA nephropathy, MHCII and III diseases (immune diseases), hypogonadotropic hypogonadism, reproductive system disorders, infertility, and/or other pathologies and disorders of the like.

The therapeutic can be, e.g., a NOVX nucleic acid, a NOVX polypeptide, or a NOVX-specific antibody, or biologically-active derivatives or fragments thereof.

For example, the compositions of the present invention will have efficacy for treatment of patients suffering from the diseases and disorders disclosed above and/or other pathologies and disorders of the like. The polypeptides can be used as immunogens to produce antibodies specific for the invention, and as vaccines. They can also be used to screen for potential agonist and antagonist compounds. For example, a cDNA encoding NOVX may be useful in gene therapy, and NOVX may be useful when administered to a subject in need thereof. By way of non-limiting example, the compositions of the present invention will have efficacy for treatment of patients suffering from the diseases and disorders disclosed above and/or other pathologies and disorders of the like.

The invention further includes a method for screening for a modulator of disorders or syndromes including, e.g., the diseases and disorders disclosed above and/or other pathologies and disorders of the like. The method includes contacting a test compound with a NOVX polypeptide and determining if the test compound binds to said NOVX polypeptide. Binding of the test compound to the NOVX polypeptide indicates the test compound is a modulator of activity, or of latency or predisposition to the aforementioned disorders or syndromes.

Also within the scope of the invention is a method for screening for a modulator of activity, or of latency or predisposition to disorders or syndromes including, e.g., the diseases and disorders disclosed above and/or other pathologies and disorders of the like by administering a test compound to a test animal at increased risk for the aforementioned disorders or syndromes. The test animal expresses a recombinant polypeptide encoded by a NOVX nucleic acid. Expression or activity of NOVX polypeptide is then measured in the test animal, as is expression or activity of the protein in a control animal which recombinantly-expresses NOVX polypeptide and is not at increased risk for the disorder or syndrome. Next, the expression of NOVX polypeptide in both the test animal and the control animal is compared. A change in the activity of NOVX polypeptide in the test animal relative to the control animal indicates the test compound is a modulator of latency of the disorder or syndrome.

In yet another aspect, the invention includes a method for determining the presence of or predisposition to a disease associated with altered levels of a NOVX polypeptide, a NOVX nucleic acid, or both, in a subject (e.g., a human subject). The method includes measuring the amount of the NOVX polypeptide in a test sample from the subject and comparing the amount of the polypeptide in the test sample to the amount of the NOVX polypeptide present in a control sample. An alteration in the level of the NOVX polypeptide in the test sample as compared to the control sample indicates the presence of or predisposition to a disease in the subject. Preferably, the predisposition includes, e.g., the diseases and disorders disclosed above and/or other pathologies and disorders of the like. Also, the expression levels of the new polypeptides of the invention can be used in a method to screen for various cancers as well as to determine the stage of cancers.

In a further aspect, the invention includes a method of treating or preventing a pathological condition associated with a disorder in a mammal by administering to the subject a NOVX polypeptide, a NOVX nucleic acid, or a NOVX-specific antibody to a subject (e.g., a human subject), in an amount sufficient to alleviate or prevent the pathological condition. In preferred embodiments, the disorder, includes, e.g. the diseases and disorders disclosed above and/or other pathologies and disorders of the like.

In yet another aspect, the invention can be used in a method to identity the cellular receptors and downstream effectors of the invention by any one of a number of techniques commonly employed in the art. These include but are not limited to the two-hybrid system, affinity purification, co-precipitation with antibodies or other specific-interacting molecules.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The present invention provides novel nucleotides and polypeptides encoded thereby. Included in the invention are the novel nucleic acid sequences and their encoded polypeptides. The sequences are collectively referred to herein as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table A provides a summary of the NOVX nucleic acids and their encoded polypeptides.

TABLE A
Sequences and Corresponding SEQ ID Numbers

| | | SEQ ID | | |
|---|---|---|---|---|
| | | NO | | |
| NOVX | | (nucleic | SEQ ID NO | |
| Assignment | Internal Identification | acid) | (polypeptide) | Homology |
| 1a | 146642892/CG50377-01 | 1 | 2 | Cub and Sushi Domain- |
| | | | | Containing Protein |
| 1b | CG50377-02 | 3 | 4 | Cub and Sushi Domain- |
| | | | | Containing Protein |
| 2 | cg-118733234 | 5 | 6 | Myelin-like protein |
| 3 | cg-122561227 | 7 | 8 | von Willebrand Factor and |
| | | | | Kielin-like protein |
| 4a | SC70504370_A/CG59253-01 | 9 | 10 | Semaphorin-like protein |
| 4b | CG59253-02 | 11 | 12 | Semaphorin 6A1 (KIAA1479)- |
| | | | | like protein |
| 4c | CG59253-05 | 13 | 14 | Semaphorin-like protein |
| 4d | CG59253-06 | 15 | 16 | Semaphorin-like protein |
| 4e | CC59253-07 | 17 | 18 | Semaphorin-like protein |
| 4f | CG59253-08 | 19 | 20 | Semaphorin-like protein |
| 5a | CG50211-01 | 21 | 22 | serine/threonine protein |
| | | | | kinase-like protein |
| 5b | CG50211-02 | 23 | 24 | serine/threonine |
| | | | | protein kinase-like protein |
| 6a | CG50215-01 | 25 | 26 | TGF-beta binding protein |
| 6b | CG50215-03 | 27 | 28 | TGF-beta binding protein |
| 6c | CG50215-04 | 29 | 30 | TGF-beta binding protein |
| 6d | CG50215-05 | 31 | 32 | TGF-beta binding protein |
| 7 | GMAP000808_A_da1 | 33 | 34 | MAS PROTO-ONCOGENE-like |
| | | | | protein |
| 8 | AL163195_da2 | 35 | 36 | RIBONUCLEASE PANCREATIC |
| | | | | PRECURSOR-like protein |
| 9 | SC87421058_A | 37 | 38 | AMINOTRANSFERASE-like |
| | | | | protein |
| 10a | CG50235-01 | 39 | 40 | Tolloid-Like 2-like protein |
| 10b | CG50235-03 | 41 | 42 | Tolloid-Like 2-like protein |
| 11 | SC135004534_A | 43 | 44 | CYSTEINE SULFINIC ACID |
| | | | | DECARBOXYLASE-like protein |

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

NOV1 is homologous to a Cub and Sushi Domain-containing-like family of proteins. Thus, the NOV1 nucleic acids, polypeptides, antibodies and related compounds according to the invention will be useful in therapeutic and diagnostic applications implicated in, for example; cancer, obesity, inflammation, hypertension, neurological diseases, neuropsychiatric diseases, small stature, obesity, diabetes, hyperlipidemia and other diseases, disorders and conditions of the like.

NOV2 is homologous to the myelin-like family of proteins. Thus NOV2 nucleic acids, polypeptides, antibodies and related compounds according to the invention will be useful in therapeutic and diagnostic applications implicated in, for example; cancer, inflammation, neurological disorders, neuropsychiatric disorders, obesity, diabetes and other diseases, disorders and conditions of the like.

NOV3 is homologous to a family of von Willebrand Factor-like and Kielin-like proteins. Thus, the NOV3 nucleic acids and polypeptides, antibodies and related compounds according to the invention will be useful in therapeutic and diagnostic applications implicated in, for example: cancer, inflammation, neurological disorders, neuropsychiatric disorders, obesity, diabetes, bleeding disorders and other diseases, disorders and conditions of the like.

NOV4 is homologous to the semaphorin-like family of proteins. Thus, NOV4 nucleic acids, polypeptides, antibodies and related compounds according to the invention will be useful in therapeutic and diagnostic applications implicated in, for example: Parkinson's disease, psychotic and neurological disorders, Alzheimers disease, Lung and other cancers and other diseases, disorders and conditions of the like.

NOV5 is homologous to the serine/threonine kinase-like family of proteins. Thus NOV5 nucleic acids, polypeptides, antibodies and related compounds according to the invention will be useful in therapeutic and diagnostic applications implicated in systemic lupus erythematosus, autoimmune disease, asthma, emphysema, scleroderma, ARDS, fertility, endometriosis, hemophilia, hypercoagulation, idiopathic thrombocytopenic purpura, allergies, immunodeficiencies, transplantation, graft versus host disease (GVHD), lymphaedema, muscular dystrophy, Lesch-Nyhan syndrome, myasthenia gravis, psoriasis, actinic keratosis, tuberous sclerosis, acne, hair growth/loss, allopecia, pigmentation disorders, endocrine disorders, Von Hippel-Lindau (VHL) syndrome, Alzheimer's disease, stroke, hypercalceimia, Parkinson's disease, Huntington's disease, cerebral palsy, epilepsy, ataxia-telangiectasia, leukodystrophies, behavioral disorders, addiction, anxiety, pain, neuroprotection, tonsilitis and other diseases, disorders and conditions of the like.

NOV6 is homologous to the TGF-beta-like family of proteins. Thus NOV6 nucleic acids, polypeptides, antibodies and related compounds according to the invention will be useful in therapeutic and diagnostic applications implicated in, for example: atherosclerosis and fibrotic disease of the kidney, liver, and lung, cancer (e.g. epithelial, endothelial, and hematopoietic), hereditary hemorrhagic telangiectasia. and other diseases, disorders and conditions of the like.

NOV7 is homologous to members of the MAS proto-oncogene-like family of proteins. Thus, the NOV7 nucleic acids, polypeptides, antibodies and related compounds according to the invention will be useful in therapeutic and diagnostic applications implicated in, for example; Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, neuroprotection, neurological disorders and diseases involving developmental and other diseases, disorders and conditions of the like.

NOV8 is homologous to the ribonuclease pancreatic precursor-like family of proteins. Thus, NOV8 nucleic acids and polypeptides, antibodies and related compounds according to the invention will be useful in therapeutic and diagnostic applications implicated in, for example; anti-cancer and anti-tumor therapy, diabetes, Von Hippel-Lindau (VHL) syndrome, pancreatitis, obesity, hyperthyroidism and hypothyroidism and hancers including, but no limited to thyroid and pancreas, and other diseases, disorders and conditions of the like.

NOV9 is homologous to the aminotransferase-like family of proteins. Thus, NOV9 nucleic acids and polypeptides, antibodies and related compounds according to the invention will be useful in therapeutic and diagnostic applications implicated in, for example; liver toxicity and damage such as in cancer, cirrhosis, or troglitazone treatment for diabetes; brain and CNS disorders including cancer, Parkinson's, Alzheimer's, epilepsy, schizophrenia and other diseases, disorders and conditions of the like.

NOV10 is homologous to the tolloid-like-2-like family of proteins. Thus, NOV10 nucleic acids and polypeptides, antibodies and related compounds according to the invention will be useful in therapeutic and diagnostic applications implicated in, for example; fibrosis, scarring, keloids, surgical adhesion, wound and fracture healing, and other diseases, disorders and conditions of the like.

NOV11 is homologous to the cysteine sulfinic acid decarboxylase-like family of proteins. Thus, NOV11 nucleic acids and polypeptides, antibodies and related compounds according to the invention will be useful in therapeutic and diagnostic applications implicated in, for example; acute or chronic hyperosmotic plasma, Adrenoleukodystrophy, Congenital Adrenal Hyperplasia, Diabetes, Von Hippel-Lindau (VHL) syndrome, Pancreatitis, Obesity, Hyperparathyroidism, Hypoparathyroidism, Fertility, cancers such as those occurring in pancreas, bone, colon, brain, lung, breast, or prostate. Endometriosis, Xerostomia Scleroderma Hypercalceimia, Ulcers Von Hippel-Lindau (VHL) syndrome, Cirrhosis, Transplantation, Inflammatory bowel disease, Diverticular disease, Hirschsprung's disease, Crohn's Disease, Appendicitis Osteoporosis, Hypercalceimia, Arthritis, Ankylosing spondylitis, Scoliosis Arthritis, Tendinitis on Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Endocrine dysfunctions, Diabetes, obesity, Growth and reproductive disorders Multiple sclerosis, Leukodystrophies, Pain, Myasthenia gravis, Pain, Systemic lupus erythematosus, Autoimmune disease, Asthma, Emphysema, Scleroderma, allergy, ARDS, Psoriasis, Actinic keratosis, Tuberous sclerosis, Acne, Hair growth, allopecia, pigmentation disorders, Renal artery stenosis, Interstitial nephritis, Glomerulonephritis, Polycystic kidney disease, Systemic lupus erythematosus, Renal tubular acidosis, IgA nephropathy, Hypercalceimia, Lesch-Nyhan syndrome and other diseases, disorders and conditions of the like.

The NOVX nucleic acids and polypeptides can also be used to screen for molecules, which inhibit or enhance NOVX activity or function. Specifically, the nucleic acids and polypeptides according to the invention may be used as targets for the identification of small molecules that modulate or inhibit, e.g., neurogenesis, cell differentiation, cell proliferation, hematopoiesis, wound healing and angiogenesis.

Additional utilities for the NOVX nucleic acids and polypeptides according to the invention are disclosed herein.

NOV1

NOV1 includes two cub and sushi domain containing protein-like proteins disclosed below. The disclosed sequences have been named NOV1a and NOV1b.

NOV1a

A disclosed NOV1a nucleic acid of 10,136 nucleotides (also referred to as 146642892/CG50377-01) encoding a novel Cub and Sushi Domain-Containing Protein-like protein is shown in Table 1A. An open reading frame was identified beginning with an ATG initiation codon at nucleotides 1-3 and ending with a TGA codon at nucleotides 9313-9315. A putative untranslated region upstream from the initiation codon and downstream from the termination codon is underlined in Table 1A. The start and stop codons are in bold letters.

TABLE 1A
NOV1a nucleotide sequence.

(SEQ ID NO:1)

ATGGCGGGCGCCCCTCCCCCCGCCTTGCTGCTGCCTTGCAGTTTGATCTCAGACTGCTGTGCTAGCAATC
AGCGACACTCCGTGGGCGTAGGACCCTCCGAGCTAGTCAAGAAGCAAATTGAGTTGAAGTCTCGAGGTGT
GAAGCTGATGCCCAGCAAAGACAACAGCCAGAAGACGTCTGTGTTAACTCAGGTTGGTGTGTCCCAAGGA
CATAATATGTGTCCAGACCCTGGCATACCCGAAAGGGGCAAAAGACTAGGCTCGGATTTCAGGTTAGGAT
CCAGCGTCCAGTTCACCTGCAACGAGGGCTATGACCTGCAAGGGTCCAAGCGGATCACCTGTATGAAAGT
GAGCGACATGTTTGCGGCCTGGAGCGACCACAGGCCAGTCTGCCGAGCCCGCATGTGTGATGCCCACCTT
CGAGGCCCCTCGGGCATCATCACCTCCCCCAATTTCCCCATTCAGTATGACAACAATGCACACTGTGTGT
GGATCATCACAGCACTCAACCCCTCCAAGGTGATCAAGCTCGCCTTTGAGGAGTTTGATTTGGAGAGGGG
CTATGACACCCTGACGGTCGGTGATGGTGGTCAGGATGGGGACCAGAAGACAGTTCTCTACATGTCTCAA
AATGCCTGCAGTGACAGCCCTCACACCCCAGGCTCTCGCATCCCAGAGAGCATGTCTGGGGACATCTGGA
GGCAGAAATGGACTGTACTTGAGATCTGTCGTGACATTAGCAGTTCAGATGCAAGGTCAGGTTCAGTGAG
GAAGTCTCCAAAGACTTCTAATGCTGTGGAACTTGTTGCTCCTGGGACAGAGATCGAGCAGGGCAGTTGC
GGTGACCCTGGCATACCTGCATATGGCCGGAGGGAAGGCTCCCGGTTTCACCACGGTGACACACTCAAGT
TTGAGTGCCAGCCCGCCTTTGAGCTGGTGGGACAGAAGGCAATCACATGCCAAAAGAATAACCAATGGTC
GGCTAAGAAGCCAGGCTGCGTGTTCTCCTGCTTCTTCAACTTCACCAGCCCGTCTGGGGTTGTCCTGTCT
CCCAACTACCCAGAGGACTATGGCAACCACCTCCACTGTGTCTGGCTCATCCTGGCCAGGCCTGAGAGCC
GCATCCACCTGGCCTTCAACGACATTGACGTGGAGCCTCAGTTTGATTTCCTGGTCATCAAGGATGGGGC
CACCGCCGAGGCGCCCGTCCTGGGCACCTTCTCAGGAAACCAGCTTCCCTCCTCCATCACAAGCAGTGGC
CACGTGGCCCGTCTCGAGTTCCAGACTGACCACTCCACAGGGAAGAGGGGCTTCAACATCACTTTTACCA
CCTTCCGACACAACGAGTGCCCGGATCCTGGCGTTCCAGTAAATGGCAAACGGTTTGGGGACAGCCTCCA
GCTGGGCAGCTCCATCTCCTTCCTCTGTGATGAAGGCTTCCTTGGGACTCAGGGCTCAGAGACCATCACC
TGCGTCCTGAAGGAGGGCAGCGTGGTCTGGAACAGCGCTGTGCTGCGGTGTGAAGCTCCCTGTGGTGGTC
ACCTGACTTCGCCCAGCGGCACCATCCTCTCTCCGGGCTGGCCTGGCTTCTACAAGGATGCCTTGAGCTG
TGCCTCGGTGATTGAGGCCCAGCCACGCTACCCCATCAAAATCACCTTCGACAGATTCAAAACCGAGGTC
AACTATGACACCCTGGAAGTACGCGATGGGCGGACTTACTCAGCGCCCTTGATCGGGGTTTACCACGGGA
CCCAGGTTCCCCAGTTCCTCATCAGCACCAGCAACTACCTCTACCTCCTCTTCTCTACCGACAAGAGTCA
CTCGGACATCGGCTTCCAGCTCCGCTATGAGACTATAACACTGCAGTCAGACCACTGTCTGGATCCAGGA
ATCCCAGTAAATGGACAGCGTCATGGGAATGACTTCTACGTGGGCGCGCTGGTGACCTTCAGCTGTGACT
CGGGCTACACATTAAGTGACGGGGAGCCTCTGGAGTGTGAGCCCAACTTCCAGTGGAGCCGGGCCCTGCC
CAGTTGTGAAGCTCTCTGTGGTGGCTTCATTCAAGGCTCCAGTGGGACCATCTTGTCGCCAGGGTTCCCT
GACTTCTACCCCAACAACTTGAACTGCACCTGGATTATCGAAACATCTCATGGCAAGGGTGTGTTCTTCA
CTTTCCACACCTTCCACCTGGAAAGTCGCCATGACTACCTCCTCATCACTGACAACGGCAGCTTCACCCA
GCCCCTGAGGCAGCTAACTGGATCTCGGCTGCCAGCTCCCATCAGCGCTGGGCTCTATGCCAACTTCACT
GCCCAGGTCCGCTTCATCTCTGATTTCTCCATGTCATATGAAGGATTCAACATCACCTTCTCAGAGTACG
ACTTGGAGCCCTGTGAGGAGCCCGAGGTCCCAGCCTACACCATCCGGAAGGGCTTGCAGTTTGGCGTGGG
CGACACCTTGACCTTCTCCTGCTTCCCCGGGTACCGTCTGGAGGGCACCGCCCGCATCACGTGCCTGGGG
GGCAGACGGCGCCTGTGGAGCTCGCCTCTGCCAAGGTGTGTTGCTGAGTGTGCGAATTCAGTCACAGGCA
CTCAGGGTACTTTGCTGTCCCCCAACTTTCCTGTGAACTACAATAACAATCATGAATGCATCTACTCCAT
CCAGACCCAGCCAGGGAAGGGAATTCAGCTGAAAGCCAGGGCATTCGAACTCTCCGAAGGAGATGTCCTC
AAGGTTTATGATGGCAACAACAACTCCGCCCGTTTGCTGGGAGTTTTTAGCCATTCTGACATCATCGGGG
TGACTTTGAACAGCACATCCAGCAGTCTGTGGCTTGATTTCATCACTGATGCTGAAAACACCAGCAAGGG
CTTTGAACTGCACTTTTCCAGCTTTGAACTCATCAAATGTGAGGACCCAGGAACCCCCAAGTTTGGCTAC
AAGGTTCATGATGAAGGTCATTTTGCAGGGAGCTCCGTGTCCTTCAGCTGTGACCCTGGATACAGCCTGC
GGGGTAGTGAGGAGCTGCTGTGTCTGAGTGGAGAGCGCCGGACCTGGGACCGGCCTCTGCCCACCTGTGT
CGCCGAGTGTGGAGGGACAGTGAGAGGAGAGGTGTCGGGGCAGGTGCTGTCACCCGCGTATCCAGCTCCC
TATGAACACAATCTCAACTGCATCTGGACCATCGAAGCAGAGGCCGGCTGCACCATTGGGCTACACTTCC
TGGTGTTTGACACAGAGGAGGTTCACGACGTGCTGCGCATCTGGGATGGGCCTGTGGAGAGCGGGGTTCT
GCTGAAGGAGCTGAGTGGCCCGGCCCTGCCCAAGGACCTGCATAGCACCTTCAACTCGGTCGTCCTGCAG
TTCAGCACTGACTTCTTCACCAGCAAGCAGGGCTTTGCCATTCAATTTTCAGTGTCCACAGCAACGTCCT
GCAATGACCCTGGGATCCCGCAGAATGGGAGTCGGAGTGGTGACAGTTGGGAACCCGGCGACTCCACAGT
GTTCCAGTGTGACCCTGGCTACGCGCTGCAGGGAAGTGCAGAGATCAGCTGTGTGAAGATCGAGAACAGG
TTCTTCTGGCAGCCCAGCCCGCCAACATGCATCGCTCCCTGCGGGGGAGACCTGACAGGACCATCTGGAC
TCATCCTCTCACCAAATTACCCAGAACCCTACCCGCCAGGCAAGGACTGTGACTCGAAAGTGACCGTCTC
ACCAGACTACGTCATCGCCCTGGTATTTAACATCTTTAACCTGGAGCCTGGCTATGACTTCCTCCATATC
TACGACCGACGCGACTCTCTCAGCCCTCTCATAGGAAGCTTCTATCGCTCCCAGCTCCCAGGCCGCATTG
AAAGCAGCAGCAACAGCCTCTTCCTCGCCTTCCGCAGCGATGCATCTGTGAGCAATGCTGGCTTCGTCAT
TGACTATACAGAAAACCCGCGGGAGTCATGTTTTGATCCTGGTTCCATCAAGAACGGCACACGGGTGGGG
TCCGACCTGAAGCTGGGCTCCTCCGTCACCTACTACTGCCACGGGGGCTACGAAGTTGAGGGCACCTCGA
CCCTGAGCTGCATCCTGGGGCCTGATGGGAAGCCCGTGTGGAACAATCCCCGGCCAGTCTGCACAGCCCC
CTGTGGGCGACAGTATGTGGGTTCGGACGGAGTGGTCTTGTCCCCCAACTACCCCCAGAACTACACCAGT
GGACAGATCTGCTTGTATTTTGTTACTGTGCCCAAGGACTATGTGGTGTTTGGCCAGTTCGCCTTCTTTC
ACACGGCCCTCAACCACGTGGTGGAGGTTCACGACGGCCACAGCCAGCACTCGCGGCTCCTCAGCTCCCT
CTCGGGCTCCCATACAGGAGAATCACTGCCCTTGGCCACCTCCAATCAAGTTCTCATTAAGTTCAGCGCC
AAAGGCCTCGCACCAGCCAGAGGCTTCCACTTTGTCTACCAAGCGGTTCCTCGAACCAGCGCCACGCAGT
GCAGCTCTGTGCCGGAACCCCGCTATGGCAAGAGGCTGGGCAGTGACTTCTCGGTGGGGGCCATCGTCCG
CTTCGAATGCAACTCCGGCTATGCCCTCCAGGGGTCGCCAGAGATCGAGTGCCTCCCTGTGCCTGGCGCC
TTGGCCCAATGGAATGTCTCAGCGCCCACGTGTGTGGTGCCGTGTGGAGGCAACCTCACAGAGCGCAGGG
GCACCATCCTGTCCCCTGGCTTCCCAGAGCCGTACCTCAACAGCCTCAACTGTGTGTGGAAGATCGTGGT
CCCCGAAGGCGCTGGCATCCACATCCAAGTTGTCAGTTTTGTGACAGAGCAGAACTGGGACTCGCTGGAA
GTATTTGATGGTGCAGATAACACTGTAACCATGCTGGGGAGTTTCTCAGGAACAACCGTGCCTGCCCTTC
TGAACAGCACCTCCAACCAGCTCTACCTTCATTTCTACTCAGATATCAGCGTATCTCCAGCTGGCTTCCA
CTTCGAGTACAAAACGGTGGGCCTGAGCAGTTGTCCGGAACCTGCTGTGCCCAGTAACGGGGTGAAGACT
GGCGAGCGCTACTTGGTGAATGATGTGGTGTCTTTCCAGTGTGAGCCGGGATATGCCCTCCAGGGCCACG
CCCACATCTCCTGCATGCCCGGAACAGTGCGGCGATCGAACTACCCTCCTCCACTCTGTATTGCACAGTG
TGGGGCAACAGTGGAGGAGATGGACCGGGTGATCCTGAGCCCCGGCTTCCCAGGCAACTACCCCAGTAAC
ATGGACTGCTCCTGGAAAATAGCACTGCCCGTGGGCTTTGGAGCTCACATCCAGTTCCTGAACTTCTCCA
CCGAGCCCAACCACGACTACATAGAAATCCGGAATGGCCCCTATGAGACCAGCCGCATGATGGGAAGATT
CAGTGGAAGCGAGCTTCCAAGCTCCCTCCTCTCCACGTCCCACGAGACCACCGTGTATTTCCACAGCGAC
CACTCCCAGAATCGGCCAGGATTCAAGCTGGAGTATCACGCCTATGAACTTCAAGAGTGCCCAGACCCAG
AGCCCTTTGCCAATGGCATTGTGAGGGGAGCTGGCTACAACGTGGGACAATCAGTGACCTTCGAGTGCCT
CCCGGGGTATCAATTGACTGGCCACCCTCTCCTCACGTGTCAACATGGCACCAACCGGAACTGGGACCAC
CCCCTGCCCAAGTGTGAAGTCCCTTGTGGCGGGAACATCACTTCTTCCAACGGCACTGTGTACTCCCCGG
GGTTCCCTAGCCCGTACTCCAGCTCCCAGGACTGTGTCTGGCTGATCACCGTGCCCATTGGCCATGGCGT
CCGCCTCAACCTCAGCCTGCTGCAGACAGAGCCCTCTGGAGATTTCATCACCATCTGGGATGGGCCACAG
CAAACAGCACCACGGCTCGGCGTCTTCACCCGGAGCATGGCCAAGAAAACAGTGCAGAGTTCATCCAACC
AGGTCCTGCTCAAGTTCCACCGTGATGCAGCCACAGGGGGGATCTTCGCCATAGCTTTCTCCGCTTATCC
ACTCACCAAATGCCCTCCTCCCACCATCCTCCCCAACGCCGAAGTCGTCACAGAGAATGAAGAATTCAAT
ATAGGTGACATCGTACGCTACAGATGCCTCCCTGGCTTTACCTTAGTGGGGAATGAAATTCTGACCTGCA
AACTTGGAACCTACCTGCAGTTTGAAGGACCACCCCCGATATGTCAAGTGCACTGTCCAACAAATGAGCT
TCTGACAGACTCCACAGGCGTGATCCTGAGCCAGAGCTACCCTGGAAGCTATCCCCAGTTCCAGACCTGC
TCTTGGCTGGTGAGAGTGGAGCCCGACTATAACATCTCCCTCACAGTGGAGTACTTCCTCAGCGAGAAGC
AATATGATGAGTTTGAGATTTTTGATGGTCCATCAGGACAGAGTCCTCTGCTGAAAGCCCTCAGTGGGAA
TTACTCAGCTCCCCTGATTGTCACCAGCTCAAGCAACTCTGTGTACCTGCGTTCGTCATCTGATCACGCC
TACAATCGGAAGGGCTTCAAGATCCGCTATTCAGCCCCTTACTGCAGCCTGCCCAGGGCTCCACTCCATG
GCTTCATCCTAGGCCAGACCAGCACCCAGCCCGGGGGCTCCATCCACTTTGGCTGCAACGCCGGCTACCG
CCTGGTGGGACACAGCATGGCCATCTGTACCCGGCACCCCCAGGGCTACCACCTGTCGAGCGAAGCCATC
CCTCTCTGTCAAGCTCTTTCCTGTGGGCTTCCTGAGGCCCCCAAGAATGGAATGGTGTTTGGCAAGGAGT
ACACAGTGGGAACCAAGGCCGTGTACAGCTGCAGTGAAGGCTACCACCTCCAGGCAGGCGCTGAGGCCAC
TGCAGAGTGTCTGGACACAGGCCTATGGAGCAACCGCAATGTCCCACCACAGTGTGTCCCTGTGACTTGT
CCTGATGTCAGTAGCATCAGCGTGGAGCATGGCCGATGGAGGCTTATCTTTGAGACACAGTATCAGTTCC
AGGCCCAGCTGATGCTCATCTGTGACCCTGGCTACTACTATACTGGCCAAAGGGTCATCCGCTGTCAGGC
CAATGGCAAATGGAGCCTCGGGGACTCTACGCCCACCTGCCGAATCATCTCCTGTGGAGAGCTCCCGATT
CCCCCCAATGGCCACCGCATCGGAACACTGTCTGTCTACGGGCCAACAGCCATCTTCTCCTGCAATTCCG
GATACACACTGGTGGGCTCCAGGGTGCGTGAGTGCATGGCCAATGGGCTCTGGAGTGGCTCTGAAGTCCG
CTGCCTTGCTGGACACTGTGGGACTCCTGAGCCCATTGTCAACGGACACATCAATGGGGAGAACTACAGC
TACCGGGGCAGTGTGGTGTACCAATGCAATGCTGGCTTCCGCCTGATCGGCATGTCTGTGCGCATCTGCC
AGCAGGATCATCACTGGTCGGGCAAGACCCCTTTCTGTGTGCCAATTACCTGTGGACACCCAGGCAACCC
TGTCAACGGCCTCACTCAGGGTAACCAGTTTAACCTCAACGATGTCGTCAAGTTTGTTTGCAACCCTGGG
TATATGGCTGAGGGGGCTCCTAGGTCCCAATGCCTGGCCAGCGGGCAATGGAGTGACATGCTGCCCACCT
GCAGAATCATCAACTGTACAGATCCTGGACACCAAGAAAATAGTGTTCGTCAGGTCCACGCCAGCGGCCC
GCACAGGTTCAGCTTCGGCACCACTGTGTCTTACCCGTGCAACCACGGCTTCTACCTCCTGGGCACCCCA
GTGCTCAGCTGCCAGGGAGATGGCACATGGGACCGTCCCCGCCCCCAGTGTCTCTTGGTGTCCTGTGGCC
ATCCGGGCTCCCCGCCTCACTCCCAGATGTCTGGAGACACTTATACTGTGGGAGCAGTGGTGCGGTACAG
CTGCATCGGCAAGCGTACTCTGGTGGGAAACAGCACCCGCATGTGTGGGCTGGATGGACACTGGACTGGC
TCCCTCCCTCACTGCTCAGGAACCAGCGTGGGAGTTTGCCGTGACCCTGGGATCCCGGCTCATGGCATCC
GTTTGCGGGACAGCTTTGATCCACCCACTGTGATGCGCTTCAGCTGTGAAGCTGGCCACGTGCTCCGGGG
ATCGTCAGAGCGCACCTGTCAAGCCAATGGCTCGTGGAGCGGCTCGCAGCCTGAGTGTGGAGTGATCTCT
TGTGGGAACCCTGGGACTCCAAGTAATGCCCGAGTTGTGTTCAGTGATGGCCTGGTTTTCTCCACCTCTA
TCGTCTATGAGTGCCGGGAACGATACTACGCCACAGGCCTGCTCAGCCGTCACTGCTCGGTCAATGGTAC
CTGGACAGGCAGTGACCCTGAGTCCCTCGTCATAAACTGTGGTGACCCTGGGATTCCAGCCAATGGCCTT
CGGCTGGGCAATGACTTCAGGTACAACAAAACTGTGACATATCAGTGTGTCCCTGGCTATATGATGGAGT
CACATAGAGTATCTGTGCTGAGCTGCACCAAGGACCGGACATGGAATGGAACCAAGCCCGTCTGCAAAGC
TCTCATGTGCAAGCCACCTCCCCTCATCCCCAATGGGAAGGTGGTGGCGTCTGACTTCATGTGGGGCTCA
AGTGTGACTTATGCCTCCCTGGAGGGGTACCAGCTCTCCCTGCCCGCGGTGTTCACCTGTGAGCGAAATG
GGTCCTGGACCGGAGAGCTGCCTCAGTGTTTCCCTGTGTTCTGCGGGGATCCTGGTGTCCCGTCCCGTGG
GAGGAGAGAGGACCGACGCTTCTCCTACAGGTCATCTGTCTCCTTCTCCTGCCATCCCCCTCTGGTGCTG
GTGGGCTCTCCACGCAGGTTTTGCCAGTCAGATGGGACATGGAGTCGCACCCAGCCCAGCTGCATAGATC
CGACCCTGACCACGTGTGCGGACCCTGGTGTGCCACAGTTTGCGATACAGAACAATTCTCAGGGCTACCA
GGTTGGAAGCACAGTCCTCTTCCGTTGTCAAAAAGGCTACCTGCTTCAGGGCTCCACCACCAGGACCTGC
CTCCCAAACCTGACCTGGAGTGGAACCCCACCTGACTGTGTCCCCCACCACTGCAGGCAGCCAGAGACGC
CAACGCATGCCAACGTCGGGGCCCTGGATTTGCCCTCCATGGGCTACACGCTCATTACTCCTGCCAGGAG
GGCTTCTCCCTCAAGGGTGGCTCCGAGCACCGCACCTGCAAGGCGGATGGCAGCTGGACAGGCAACCCGC
CCATCTGCCTGGAGGTCCGGCCCAGTGGGAGACCCATCAACACTGCCCGGGAGCCACCGCTCACCCAAGC
CTTGATTCCTGGGGATGTTTTTGCCAAGAATTCCCTGTGGAAAGGGGCCTATGAATACCAGGGGAAGAAG
CAGCCAGCCATGCTCAGAGTGACTGGCTTCCAAGTTGCCAACAGCAAGGTCAATGCCACCATGATCGACC
ACAGTGGCGTGGAGCTGCACTTGGCTGGAACTTACAAGAAAGAAGATTTTCATCTCCTACTCCAGGTGTA
CCAGATTACAGGGCCTGTGGAGATCTTTATGAATAAGTTCAAAGATGATCACTGGGCTTTAGATGGCCAT
GTCTCGTCAGAGTCCTCCGGAGCCACCTTCATCTACCAAGGCTCTGTCAAGGGCCAAGGCTTTGGGCAGT
TCGGCTTTCAAAGACTGGACCTCAGGCTGCTGGAGTCAGACCCCGAGTCCATTGGCCGCCACTTTGCTTC
CAACAGCAGCTCAGTGGCAGCCGCGATCCTGGTGCCTTTCATCGCCCTCATTATTGCGGGCTTCGTGCTC
TATCTCTACAAGCACAGGAGAAGACCCAAAGTTCCTTTCAATGGCTATGCTGGCCACGAGAACACCAATG
TTCGGGCCACATTTGAGAACCCAATGTACGACCGCAACATCCACCCCACAGACATCATGGCCAGCGAGGC
GGAGTTCACAGTCAGCACAGTGTGCACAGCAGTATAGCCACCCGGCCTGGCCGCTTTTTTTGCTAGGTTG
AACTGGTACTCCAGCAGCCGCCGAAGCTGGACTGTACTGCTGCCATCTCAGCTCACTGCAACCTCCCTGC
CTGATTCCCCTGCCTCAGCCTGCCGAGTGCCTGCGATTGCAGGCGCGCACCGCCAC

In a search of public sequence databases, the NOV1a nucleic acid sequence, located on chromsome 1 257 of 259 bases (99%) identical to a gb:GENBANK-ID: AK022620|acc: AK022620.1 mRNA from Homo sapiens (Homo sapiens cDNA FLJ12558 fis, clone NT2RM4000787). Public nucleotide databases include all GenBank databases and the GeneSeq patent database.

In all BLAST alignments herein, the “E-value” or “Expect” value is a numeric indication of the probability that the aligned sequences could have achieved their similarity to the BLAST query sequence by chance alone, within the database that was searched. For example, the probability that the subject (“Sbjct”) retrieved from the NOV1 BLAST analysis, e.g., Homo sapiens cDNA FLJ12558 fis, matched the Query NOV1 sequence purely by chance is 1.1e -47. The Expect value (E) is a parameter that describes the number of hits one can “expect” to see just by chance when searching a database of a particular size. It decreases exponentially with the Score (S) that is assigned to a match between two sequences. Essentially, the E value describes the random background noise that exists for matches between sequences.

The Expect value is used as a convenient way to create a significance threshold for reporting results. The default value used for blasting is typically set to 0.0001. In BLAST 2.0, the Expect value is also used instead of the P value (probability) to report the significance of matches. For example, an E value of one assigned to a hit can be interpreted as meaning that in a database of the current size one might expect to see one match with a similar score simply by chance. An E value of zero means that one would not expect to see any matches with a similar score simply by chance. See, e.g., http://www.ncbi.nlm.nih.gov/Education/BLASTinfo/. Occasionally, a string of X's or N's will result from a BLAST search. This is a result of automatic filtering of the query for low-complexity sequence that is performed to prevent artifactual hits. The filter substitutes any low-complexity sequence that it finds with the letter “N” in nucleotide sequence (e.g., “NNNNNNNNNNNNN”) or the letter “X” in protein sequences (e.g., “XXXXXXXXX”). Low-complexity regions can result in high scores that reflect compositional bias rather than significant position-by-position alignment. (Wootton and Federhen, Methods Enzymol 266:554-571, 1996).

The disclosed NOV1a polypeptide (SEQ ID NO:2) encoded by SEQ ID NO:1 has 3104 amino acid residues and is presented in Table 1B using the one-letter amino acid code. Signal P, Psort and/or Hydropathy results predict that NOV1a has a signal peptide and is likely to be localized outside the cell with a certainty of 0.3700. In other embodiments, NOV1a may also be localized to the lysome (lumen) with a certainty of 0.1900, the microbody with a certainty or 0.1764, or in the endoplasmic reticulum (membrane) with a certainty of 0.1000. The most likely cleavage site for a NOV1a peptide is between amino acids 21 and 22, at: CCA-SN.

TABLE 1B
Encoded NOV1a protein sequence.

(SEQ ID NO:2)

MAGAPPPALLLPCSLISDCCASNQRHSVGVGPSELVKKQIELKSRGVKLMPSKDNSQKTSVLTQVGVSQG
HNMCPDPGIPERGKRLGSDFRLGSSVQFTCNEGYDLQGSKRITCMKVSDMFAAWSDHRPVCRARMCDAHL
RGPSGIITSPNFPIQYDNNAHCVWIITALNPSKVIKLAFEEFDLERGYDTLTVGDGGQDGDQKTVLYMSQ
NACSDSPHTPGSRIPESMSGDIWRQKWTVLEICRDISSSDARSGSVRKSPKTSNAVELVAPGTEIEQGSC
GDPGIPAYGRREGSRFHHGDTLKFECQPAFELVGQKAITCQKNNQWSAKKPGCVFSCFFNFTSPSGVVLS
PNYPEDYGNHLHCVWLILARPESRIHLAFNDIDVEPQFDFLVIKDGATAEAPVLGTFSGNQLPSSITSSG
HVARLEFQTDHSTGKRGFNITFTTFRHNECPDPGVPVNGKRFGDSLQLGSSISFLCDEGFLGTQGSETIT
CVLKEGSVVWNSAVLRCEAPCCGHLTSPSGTILSPGWPGFYKDALSCAWVIEAQPGYPIKITFDRFKTEV
NYDTLEVRDGRTYSAPLIGVYHGTQVPQFLISTSNYLYLLFSTDKSHSDIGFQLRYETITLQSDHCLDPG
IPVNGQRHGNDFYVGALVTFSCDSGYTLSDGEPLECEPNFQWSRALPSCEALCGGFIQGSSGTILSPGFP
DFYPNNLNCTWIIETSHGKGVFFTFHTFHLESGHDYLLITENGSFTQPLRQLTGSRLPAPISAGLYGNFT
AQVRFISDFSMSYEGFNITFSEYDLEPCEEPEVPAYSIRKGLQFGVGDTLTFSCFPGYRLEGTARITCLG
GRRRLWSSPLPRCVAECGNSVTGTQGTLLSPNFPVNYNNNHECIYSIQTQPGKGIQLKARAFELSEGDVL
KVYDGNNNSARLLGVFSHSEMMGVTLNSTSSSLWLDFITDAENTSKGFELHFSSFELIKCEDPGTPKFGY
KVHDEGHFAGSSVSFSCDPGYSLRGSEELLCLSGERRTWDRPLPTCVAECGGTVRGEVSGQVLSPGYPAP
YEHNLNCIWTIEAEAGCTIGLHFLVFDTEEVHDVLRTWDGPVESGVLLKELSGPALPKDLHSTFNSVVLQ
FSTDFFTSKQGFAIQFSVSTATSCNDPGIPQNGSRSGDSWEAGDSTVFQCDPGYALQGSAEISCVKIENR
FFWQPSPPTCIAPCGGDLTGPSGVILSPNYPEPYPPGKECDWKVTVSPDYVIALVFNIFNLEPGYDFLHI
YDGRDSLSPLIGSFYGSQLPGRIESSSNSLFLAFRSDASVSNAGFVIDYTENPRESCFDPGSIKNGTRVG
SDLKLGSSVTYYCHGGYEVEGTSTLSCILGPDGKPVWNNPRPVCTAPCGGQYVGSDGVVLSPNYPQNYTS
GQICLYFVTVPKDYVVFGQFAFFHTALNDVVEVHDGHSQHSRLLSSLSGSHTGESLPLATSNQVLIKFSA
KGLAPARGFHFVYQAVPRTSATQCSSVPEPRYGKRLGSDFSVGAIVRFECNSGYALQGSPEIECLPVPGA
LAQWNVSAPTCVVPCGGNLTERRGTILSPGFPEPYLNSLNCVWKIVVPEGAGIQIQVVSFVTEQNWDSLE
VFDGADNTVThLGSFSGTTVPALLNSTSNQLYLHFYSDISVSAAGFHLEYKTVGLSSCPEPAVPSNGVKT
GERYLVNDVVSFQCEPGYALQGHAHISCMPGTVRRWNYPPPLCIAQCGGTVEEMEGVILSPGFPGNYPSN
MDCSWKIALPVGFGAHIQFLNFSTEPNHDYIEIRNGPYETSRMMGRFSGSELPSSLLSTSHETTVYFHSD
HSQNRPGFKLEYQAYELQECPDPEPFANGIVRGAGYNVGQSVTFECLPGYQLTGHPVLTCQHGTNRNWDH
PLPKCEVPCGGNITSSNGTVYSPGFPSPYSSSQDCVWLITVPIGHGVRLNLSLLQTEPSGDFITIWDGPQ
QTAPRLGVFTRSMAKKTVQSSSNQVLLKFHRDAATGGIFAIAFSAYPLTKCPPPTILPNAEVVTENEEFN
IGDIVRYRCLPGFTLVGNEILTCKLGTYLQFEGPPPICEVHCPTNELLTDSTGVILSQSYPGSYPQFQTC
SWLVRVEPDYNISLTVEYFLSEKQYDEFEIFDGPSGQSPLLKALSGNYSAPLIVTSSSNSVYLRWSSDHA
YNRKCFKIRYSAPYCSLPRAPLUGFILGQTSTQPGGSIHFGCNAGYRLVGHSMAICTRHPQGYHLWSEAI
PLCQALSCGLPEAPKNGMVFGKEYTVGTKAVYSCSEGYHLQAGAEATAECLDTGLWSNRNVPPQCVPVTC
PDVSSISVEHGRWRLIFETQYQFQAQLMLICDPGYYYTGQRVIRCQANGKWSLGDSTPTCRIISCGELPI
PPNGHRIGTLSVYGATAIFSCNSGYTLVGSRVRECMANGLWSGSEVRCLAGHCGTPEPIVNGHINGENYS
YRGSVVYQCNAGFRLIGMSVRICQQDHHWSGKTPFCVPITCGHPGNPVNGLTQGNQFNLNDVVKFVCNPG
YMAEGAARSQCLASGQWSDMLPTCRIINCTDPGHQENSVRQVHASGPHRFSFGTTVSYRCNHGFYLLGTP
VLSCQGDGTWDRPRPQCLLVSCGHPGSPPHSQMSGDSYTVGAVVRYSCIGKRTLVGNSTRNCGLDGHWTG
SLPHCSGTSVGVCGDPGIPAHGIRLGDSFDPGTVMRPSCEAGHVLRGSSERTCQANGSWSGSQPECGVIS
CGNPGTPSNARVVFSDGLVFSSSIVYECRECYYATGLLSRECSVNGTWTGSDPECLVINCGDPGIPANGL
RLGNDFRYNKTVTYQCVPGYMMESHRVSVLSCTKDRThNGTKPVCKALMCKPPPLIPNCKVVGSDFMWGS
SVTYACLEGYQLSLPAVFTCEGNGSWTGELPQCPPVFCGDPGVPSRGRREDRGFSYRSSVSFSCHPPLVL
VGSPRRFCQSDGTWSGTQPSCIDPTLTTCADPGVPQFGIQNNSQGYQVGSTVLFRCQKGYLLQGSTTRTC
LPNLTWSGTPPDCVPHHCRQPETPTHANVGALDLPSMGYTLITPARRASPSRVAPSTAPARRMAAGQASR
PSAWRSGPVGDPSTLPGSHRSPKP

A search of sequence databases reveals that the NOV1a amino acid sequence has 145 of 489 amino acid residues (29%) identical to, and 216 of 489 amino acid residues (44%) similar to, the 2489 amino acid residue ptnr:SPTREMBL-ACC:Q16744 protein from Homo sapiens (Human) (COMPLEMENT RECEPTOR 1). Public amino acid databases include the GenBank databases, SwissProt, PDB and PIR.

NOV1 is expressed in at least the adrenal gland and the pituitary gland. This information was derived by determining the tissue sources of the sequences that were included in the invention including but not limited to SeqCalling sources, Public EST sources, Literature sources, and/or RACE sources.

NOV1b

A disclosed NOV1b nucleic acid of 8010 nucleotides (also referred to as CG50377-02) encoding a cub and sushi domain-containing protein-like protein is shown in Table 1C.

TABLE 1C
NOV1b nucleotide sequence.

| | |
|---|---|
| ATCGCGGGCGCCCCTCCCCCCGCCTTGCTGCTGCCTTGCAGTTTGATCTCAGACTGCTGT | (SEQ ID NO:3) |
| GCTAGCAATCAGCGACACTCCGTGGGCGTAGGACCCTCCGAGCTAGTCAAGAAGCAAATT | |
| GAGTTGAAGTCTCGAGGTGTGAAGCTGATGCCCAGCAAAGACAACAGCCAGAAGACGTCT | |
| GTGTTAACTCAGGTTGGTGTGTCCCAAGGACATAATATGTGTCCAGACCCTGGCATACCC | |
| GAAAGGGGCAAAAGACTAGGCTCGGATTTCAGGTTAGGATCCAGCGTCCAGTTCACCTGC | |
| AACGAGGGCTATGACCTGCAAGGGTCCAAGCGGATCACCTGTATGAAAGTGAGCGACATG | |
| TTTGCGGCCTCGAGCGACCACAGGCCAGTCTGCCGAGCCCGCATGTGTGATGCCCACCTT | |
| CGAGGCCCCTCGGGCATCATCACCTCCCCCAATTTCCCCATTCAGTATGACAACAATGCA | |
| CACTGTGTGTGGATCATCACAGCACTCAACCCCTCCAAGGTGATCAAGCTCGCCTTTGAG | |
| GAGTTTGATTTGGAGAGGCGCTATGACACCCTGACGGTCGGTGATGGTGGTCAGCATCGG | |
| GACCAGAAGACAGTTCTCTACATGTCTCAAAATGCCTGCAGTGACAGCCCTCACACCCCA | |
| GGCTCTCGCATCCCAGAGAGCATGTCTGGGGACATCTGGAGGCAGAAATGGACTGTACTT | |
| GAGATCTGTCGTGACATTAGCAGTTCAGATGCAAGGTCAGGTTCAGTGAGGAAGTCTCCA | |
| AAGACTTCTAATGCTGTGGAACTTGTTGCTCCTGGGACAGACATCGAGCAGGGCAGTTGC | |
| GGTGACCCTGGCATACCTGCATATGGCCGGAGGGAAGGCTCCCGGTTTCACCACGGTGAC | |
| ACACTCAAGTTTGAGTGCCAGCCCGCCTTTGAGCTGGTGGGACAGAAGGCAATCACATGC | |
| CAAAAGAATAACCAATGGTCGGCTAAGAAGCCAGGCTGCGTGTTCTCCTGCTTCTTCAAC | |
| TTCACCAGCCCGTCTGGGGTTGTCCTGTCTCCCAACTACCCAGAGGACTATGGCAACGAC | |
| CTCCACTGTGTCTGGCTCATCCTGGCCAGGCCTGAGAGCCGCATCCACCTGGCCTTCAAC | |
| GACATTGACGTGGAGCCTCAGTTTGATTTCCTGGTCATCAAGGATGGGCCCACCGCCGAG | |
| GCGCCCGTCCTCGGCACCTTCTCAGGAAACCAGCTTCCCTCCTCCATCACAAGCAGTCGC | |
| CACGTGGCCCGTCTCGAGTTCCAGACTGACCACTCCACAGGGAAGAGGGGCTTCAACATC | |
| ACTTTTACCACCTTCCGACACAACGAGTGCCCGGATCCTGCCGTTCCAGTAAATGGCAAA | |
| CGGTTTGGGGACAGCCTCCAGCTGGGCAGCTCCATCTCCTTCCTCTGTGATGAAGGCTTC | |
| CTTGGGACTCAGGGCTCAGAGACCATCACCTGCGTCCTGAAGGAGGGCAGCGTGGTCTGG | |
| AACAGCGCTGTGCTGCGGTGTGAAGCTCCCTGTGGTGGTCACCTGACTTCGCCCAGCGGC | |
| ACCATCCTCTCTCCGGGCTGGCCTGGCTTCTACAAGGATGCCTTGAGCTGTGCCTGGGTG | |
| ATTGAGGCCCAGCCAGGCTACCCCATCAAAATCACCTTCGACAGATTCAAAACCGAGGTC | |
| AACTATGACACCCTGGAAGTACGCGATGGCCGGACTTACTCAGCGCCCTTGATCGGGGTT | |
| TACCACGGGACCCAGGTTCCCCAGTTCCTCATCAGCACCAGCAACTACCTCTACCTCCTC | |
| TTCTCTACCGACAAGAGTCACTCGGACATCCGCTTCCAGCTCCGCTATGAGACTATAACA | |
| CTGCAGTCAGACCACTGTCTGGATCCACGAATCCCAGTAAATGGACAGCGTCATGGGAAT | |
| GACTTCTACGTGGGCGCGCTGGTGACCTTCAGCTGTGACTCGGGCTACACATTAAGTGAC | |
| GGGGAGCCTCTGGAGTGTGAGCCCAACTTCCAGTGGAGCCGCGCCCTGCCCAGTTGTGAA | |
| GCTCTCTGTGGTGGCTTCATTCAAGGCTCCAGTGGGACCATCTTGTCGCCAGCGTTCCCT | |
| GACTTCTACCCCAACAACTTGAACTGCACCTGGATTATCGAAACATCTCATGGCAAGGGT | |
| GTGTTCTTCACTTTCCACACCTTCCACCTGGAAAGTGGCCATGACTACCTCCTCATCACT | |
| GAGAACGGCAGCTTCACCCAGCCCCTGAGGCAGCTAACTGGATCTCGGCTGCCAGCTCCC | |
| ATCAGCGCTGGGCTCTATGGCAACTTCACTGCCCAGGTCCGCTTCATCTCTGATTTCTCC | |
| ATGTCATATGAAGGATTCAACATCACCTTCTCAGAGTACGACTTGGAGCCCTGTGAGGAG | |
| CCCGAGGTCCCAGCCTACACCATCCGGAAGGGCTTGCAGTTTGGCGTGGGCGACACCTTG | |
| ACCTTCTCCTGCTTCCCCGGGTACCGTCTGGAGGGCACCGCCCGCATCACGTGCCTGGGG | |
| GGCAGACGGCGCCTGTGGAGCTCGCCTCTGCCAAGGTGTGTTGCTGAGTGTCGGAATTCA | |
| GTCACAGGCACTCAGGGTACTTTGCTGTCCCCCAACTTTCCTGTGAACTACAATAACAAT | |
| CATGAATGCATCTACTCCATCCAGACCCAGCCAGGGAAGGGAATTCAGCTGAAAGCCAGG | |
| GCATTCGAACTCTCCGAACCAGATGTCCTCAAGGTTTATGATGGCAACAACAACTCCCCC | |
| CGTTTGCTGGGAGTTTTTAGCCATTCTGAGATGATGGGGGTGACTTTGAACAGCACATCC | |
| AGCAGTCTGTGGCTTGATTTCATCACTGATGCTGAAAACACCAGCAACGGCTTTGAACTG | |
| CACTTTTCCAGCTTTGAACTCATCAAATGTGAGGACCCAGGAACCCCCAAGTTTGGCTAC | |
| AAGGTTCATGATGAAGGTCATTTTGCAGGCACCTCCGTGTCCTTCAGCTGTGACCCTGGA | |
| TACAGCCTGCGGCGTAGTGAGGAGCTGCTGTGTCTGAGTGGAGAGCCCCGGACCTGGGAC | |
| CGGCCTCTGCCCACCTGTGTCGCCGAGTGTGGAGGGACAGTGAGAGGAGAGGTGTCGGGG | |
| CAGGTGCTGTCACCCGCGTATCCAGCTCCCTATGAACACAATCTCAACTGCATCTGGACC | |
| ATCGAAGCAGAGGCCGGCTGCACCATTGGGCTACACTTCCTGGTGTTTGACACAGAGGAG | |
| GTTCACGACGTGCTGCGCATCTGGGATGGGCCTGTGGAGAGCGGGGTTCTGCTGAAGGAG | |
| CTGAGTGGCCCGGCCCTGCCCAAGGACCTGCATAGCACCTTCAACTCCGTCGTCCTGCAG | |
| TTCAGCACTGACTTCTTCACCAGCAAGCAGGGCTTTGCCATTCAATTTTCAGTGTCCACA | |
| GCAACGTCCTGCAATGACCCTCGGATCCCGCAGAATGGGAGTCGGAGTGGTGACAGTTGG | |
| GAAGCCGGCGACTCCACAGTGTTCCAGTGTGACCCTGGCTACGCGCTGCAGGGAAGTGCA | |
| GAGATCAGCTGTGTGAAGATCGAGAACAGGTTCTTCTGGCAGCCCAGCCCGCCAACATGC | |
| ATCGCTCCCTGCGGGGGAGACCTGACAGGACCATCTGGAGTCATCCTCTCACCAAATTAC | |
| CCAGAACCCTACCCGCCAGGCAAGGAGTGTGACTGCAAAGTGACCGTCTCACCAGACTAC | |
| GTCATCGCCCTGGTATTTAACATCTTTAACCTGGAGCCTGGCTATGACTTCCTCCATATC | |
| TACGACGGACGGGACTCTCTCAGCCCTCTCATAGGAAGCTTCTATGGCTCCCAGCTCCCA | |
| GGCCGCATTGAAAGCAGCAGCAACAGCCTCTTCCTCGCCTTCCGCAGCGATGCATCTGTG | |
| AGCAATGCTGGCTTCGTCATTGACTATACAGAAAACCCGCGGGAGTCATGTTTTGATCCT | |
| GGTTCCATCAAGAACGGCACACCCGTGGGGTCCGACCTGAAGCTGGGCTCCTCCGTCACC | |
| TACTACTGCCACGGGGGCTACGAAGTTGAGGGCACCTCGACCCTGAGCTGCATCCTGGGG | |
| CCTGATGGGAAGCCCGTGTGGAACAATCCCCGGCCAGTCTGCACAGCCCCCTGTGGGGGA | |
| CAGTATGTGGGTTCGGACGGAGTGGTCTTGTCCCCCAACTACCCCCAGAACTACACCAGT | |
| GGACAGATCTGCTTGTATTTTGTTACTGTGCCCAAGGACTATGTGGTGTTTGGCCAGTTC | |
| GCCTTCTTTCACACGGCCCTCAACGACGTGGTGGAGGTTCACGACGGCCACAGCCAGCAC | |
| TCGCGGCTCCTCAGCTCCCTCTCGGGCTCCCATACAGGAGAATCACTGCCCTTGGCCACC | |
| TCCAATCAAGTTCTCATTAAGTTCAGCGCCAAAGGCCTCGCACCAGCCAGAGGCTTCCAC | |
| TTTGTCTACCAAGCCGTTCCTCGAACCAGCGCCACGCAGTGCAGCTCTGTGCCGGAACCC | |
| CGCTATGGCAAGAGGCTGGGCAGTGACTTCTCGGTGGGGGCCATCGTCCGCTTCGAATGC | |
| AACTCCGGCTATCCCCTGCAGGGGTCGCCAGAGATCGAGTGCCTCCCTGTGCCTGCGGCC | |
| TTGGCCCAATGGAATGTCTCAGCGCCCACGTGTGTGGTGCCGTGTGGAGGCAACCTCACA | |
| GAGCGCAGCCGCACCATCCTGTCCCCTGGCTTCCCAGAGCCGTACCTCAACAGCCTCAAC | |
| TGTGTGTGGAAGATCGTGGTCCCCGAAGGCGCTGGCATCCAGATCCAAGTTGTCACTTTT | |
| GTGACAGAGCAGAACTGGGACTCGCTGGAAGTATTTGATGGTGCAGATAACACTGTAACC | |
| ATGCTGGGGAGTTTCTCAGGAACAACCGTGCCTGCCCTTCTGAACAGCACCTCCAACCAG | |
| CTCTACCTTCATTTCTACTCAGATATCAGCGTATCTGCAGCTCGCTTCCACTTGGAGTAC | |
| AAAACGGTGGGCCTGAGCAGTTGTCCGGAACCTGCTGTGCCCAGTAACGGGGTCAACACT | |
| GGCGAGCGCTACTTGGTGAATGATGTGGTGTCTTTCCAGTGTGAGCCGGGATATGCCCTC | |
| CAGGGCCACGCCCACATCTCCTGCATGCCCGGAACAGTGCGGCGATGGAACTACCCTCCT | |
| CCACTCTGTATTGCACAGTGTGGGGGAACAGTGGAGGAGATGGAGGGGGTGATCCTGAGC | |
| CCCGGCTTCCCACGCAACTACCCCAGTAACATGGACTGCTCCTGGAAAATAGCACTGCCC | |
| GTGGGCTTTGGAGCTCACATCCAGTTCCTGAACTTCTCCACCGAGCCCAACCACGACTAC | |
| ATAGAAATCCGGAATGGCCCCTATGAGACCAGCCGCATGATGGGAAGATTCAGTGGAAGC | |
| GAGCTTCCAAGCTCCCTCCTCTCCACGTCCCACGAGACCACCGTGTATTTCCACAGCGAC | |
| CACTCCCAGAATCGGCCAGGATTCAAGCTGGAGTATCAGGCCTATGAACTTCAAGAGTGC | |
| CCAGACCCAGAGCCCTTTGCCAATCGCATTGTGAGGGGAGCTCGCTACAACGTGGGACAA | |
| TCAGTGACCTTCGAGTGCCTCCCGCGGTATCAATTGACTGGCCACCCTGTCCTCACCTGT | |
| CAACATGGCACCAACCGGAACTGGGACCACCCCCTGCCCAAGTGTGAAGTCCCTTGTGGC | |
| GGGAACATCACTTCTTCCAACCGCACTGTGTACTCCCCGGGGTTCCCTAGCCCGTACTCC | |
| AGCTCCCAGGACTGTGTCTGGCTGATCACCGTGCCCATTGGCCATGGCGTCCGCCTCAAC | |
| CTCAGCCTGCTGCAGACAGAGCCCTCTGGAGATTTCATCACCATCTGGGATGGGCCACAG | |
| CAAACAGCACCACGGCTCGGCGTCTTCACCCGGAGCATGGCCAAGAAAACAGTGCAGAGT | |
| TCATCCAACCAGGTCCTGCTCAAGTTCCACCGTGATGCAGCCACAGCGGGGATCTTCGCC | |
| ATAGCTTTCTCCGCTTATCCACTCACCAAATGCCCTCCTCCCACCATCCTCCCCAACGCC | |
| GAAGTCGTCACAGAGAATGAAGAATTCAATATAGGTGACATCGTACGCTACAGATGCCTC | |
| CCTCGCTTTACCTTAGTGGGGAATGAAATTCTGACCTGCAAACTTGGAACCTACCTGCAG | |
| TTTGAAGGACCACCCCCGATATGTGAAGTGCACTGTCCAACAAATGAGCTTCTGACAGAC | |
| TCCACAGGCGTGATCCTGAGCCAGAGCTACCCTGGAAGCTATCCCCAGTTCCAGACCTGC | |
| TCTTGGCTGGTGAGAGTGGACCCCCACTATAACATCTCCCTCACAGTGGAGTACTTCCTC | |
| AGCGAGAAGCAATATGATGAGTTJGAGATTTTTGATGGTCCATCAGGACAGAGTCCTCTG | |
| CTGAAAGCCCTCAGTGGGAATTACTCAGCTCCCCTGATTGTCACCAGCTCAAGCAACTCT | |
| CTGTACCTGCGTTCGTCATCTGATCACCCCTACAATCGGAAGGGCTTCAAGATCCGCTAT | |
| TCAGCCCCTTACTGCAGCCTGCCCAGGGCTCCACTCCATGGCTTCATCCTAGGCCAGACC | |
| AGCACCCAGCCCGGGGGCTCCATCCACTTTGGCTGCAACGCCGGCTACCGCCTGGTGGGA | |
| CACAGCATGGCCATCTGTACCCGGCACCCCCAGGGCTACCACCTGTGGAGCGAAGCCATC | |
| CCTCTCTGTCAAGCTCTTTCCTGTGCGCTTCCTGACGCCCCCAAGAATGGAATGGTGTTT | |
| CGCAAGGAGTACACAGTGGGAACCAAGCCCATGTACAGCTGCAGTGAAGGCTACCACCTC | |
| CAGGCACGCGCTGAGGCCACTGCAGAGTGTCTGGACACAGGCCTATGGAGCAACCGCAAT | |
| GTCCCACCACAGTGTGTCCGTGAGTCCTCCGGCAATGGAGGCGGGTCTGTCACTTGTCCT | |
| GATGTCAGTAGCATCAGCGTGGAGCATGGCCGATGGAGGCTTATCTTTGAGACACAGTAT | |
| CAGTTCCAGGCCCAGCTGATGCTCATCTGTGACCCTGGCTACTACTATACTGGCCAAAGG | |
| GTCATCCGCTGTCAGGCCAATGGCAAATGGAGCCTCGGGGACTCTACGCCCACCTGCCGA | |
| ATCATCTCCTGTGGAGACCTCCCGATTCCCCCCAATGGCCACCGCATCGGAACACTGTCT | |
| GTCTACGGGGCAACAGCCATCTTCTCCTGCAATTCCGGATACACACTGGTGGGCTCCAGG | |
| GTGCGTGAGTGCATCGCCAATCGGCTCTGGAGTGGCTCTGAAGTCCGCTGCCTTGCCACT | |
| CAGACCAAGCTCCACTCCATTTTCTATAAGCTCCTCTTCGATGTACTCTCTTCCCCATCC | |
| CTCACCAAAGCTGGACACTGTGGGACTCCTGAGCCCATTGTCAACGGACACATCAATGGG | |
| GAGAACTACAGCTACCCGGGCAGTGTGGTGTACCAATGCAATGCTGGCTTCCGCCTGATC | |
| GGCATGTCTGTGCGCATCTGCCAGCAGGATCATCACTGGTCGGGCAAGACCCCTTTCTGT | |
| GTGCATGTTAAGCAGCAGTTCCTGCTGCTGCTGCTGCTGTTGTGTGATGATGATGATGAT | |
| GAAGATGATGGTAGTGGTGCAATTACCTGTGGACACCCAGGCAACCCTGTCAACGGCCTC | |
| ACTCAGCGTAACCAGTTTAACCTCAACGATGTGGTCAAGTTTGTTTGCAACCCTGGGTAT | |
| ATGGCTGACGGGGCTGCTACGTCCCAATGCCTGGCCAGCGGGCAATGGAGTGACATGCTG | |
| CCCACCTGCAGAATCATCAACTGTACAGATCCTGGACACCAAGAAAATAGTGTTCGTCAG | |
| GTCCACGCCACCGGCCCGCACAGGTTCAGCTTCGGCACCACTGTGTCTTACCGGTGCAAC | |
| CACGGCTTCTACCTCCTGGGCACCCCAGTGCTCAGCTGCCAGGGAGATGGCACATGGGAC | |
| CGTCCCCGCCCCCAGTGTCTCTGTAAGTAG | |

The disclosed NOV1b polypeptide (SEQ ID NO:4) encoded by SEQ ID NO:3 has 2669 amino acid residues and is presented in Table 1D using the one-letter amino acid code.

TABLE 1D
Encoded NOV1b protein sequence.

| | |
|---|---|
| MAGAPPPALLLPCSLISDCCASNQRRSVGVGPSELVKKQIELKSRGVKLMPSKDNSQKTS | (SEQ ID NO:4) |
| VLTQVGVSQGHNMCPDPGIPERGKRLGSDFRLGSSVQFTCNEGYDLQGSKRITCMKVSDM | |
| FAAWSDHRPVCRARMCDAHLRGPSGIITSPNFPIQYDNNAHCVWIITALNPSKVIKLAFE | |
| EFDLERGYDTLTVGDGGQDGDQKTVLYMSQNACSDSPHTPGSRIPESMSGDIWRQKWTVL | |
| EICRDISSSDARSGSVRKSPKTSNAVELVAPGTEIEQGSCGDPGIPAYGRREGSRFHHGD | |
| TLKFECQPAFELVGQKAITCQKNNQWSAKKPGCVFSCFFNFTSPSGVVLSPNYPEDYGNH | |
| LHCVWLILARPESRIHLAFNDIDVEPQFDFLVIKDGATAEAPVLGTFSGNQLPSSITSSG | |
| HVARLEFQTDHSTGKRGFNITFTTFRHNECPDPGVPVNGKRFGDSLQLGSSISFLCDEGF | |
| LGTQGSETITCVLKEGSVVWNSAVLRCEAPCGGHLTSPSGTILSPGWPGFYKDALSCAWV | |
| IEAQPGYPIKITFDRFKTEVNYDTLEVRDGRTYSAPLIGVYHGTQVPQFLISTSNYLYLL | |
| FSTDKSHSDIGFQLRYETITLQSDHCLDPGIPVNGQRHGNDFYVGALVTFSCDSGYTLSD | |
| GEPLECEPNFQWSRALPSCEALCGGFIQGSSGTILSPGFPDFYPNNLNCTWIIETSHGKG | |
| VFFTFETFHLESGHDYLLITENGSFTQPLRQLTGSRLPAPISAGLYGNFTAQVRFISDFS | |
| MSYEGFNITFSEYDLEPCEEPEVPAYSIRKGLQFGVGDTLTBSCFPGYRLEGTARITCLG | |
| GRRRLWSSPLPRCVAECGNSVTGTQGTLLSPNFPVNYNNNHECIYSIQTQPGKGIQLKAR | |
| AFELSEGDVLKVYDGNNNSARLLGVFSHSEMMGVTLNSTSSSLWLDFITDAENTSKGFEL | |
| HFSSFELIKCEDPGTPKFGYKVHDEGHFAGSSVSFSCDPGYSLRGSEELLCLSGERRTWD | |
| RPLPTCVAECCGTVRGEVSGQVLSPGYPAPYEHNLNCIWTIEAEAGCTIGLHFLVFDTEE | |
| VHDVLRIWDGPVESGVLLKELSGPALPKDLHSTFNSVVLQFSTDFFTSKQGFAIQFSVST | |
| ATSCNDPGIPQNGSRSGDSWEAGDSTVFQCDPGYALQGSAEISCVKIENRFFWQPSPPTC | |
| IAPCGGDLTGPSGVILSPNYPEPYPPGKECDWKVTVSPDYVIALVFNIFNIEPGYDFLHI | |
| YDGRDSLSPLIGSFYGSQLPGRIESSSNSLFLAFRSDASVSNAGFVIDYTENPRESCFDP | |
| GSIKNGTRVGSDLKLGSSVTYYCHGGYEVEGTSTLSCILGPDGKPVWNNPRPVCTAPCGG | |
| QYVGSDGVVLSPNYPQNYTSGQICLYFVTVPKDYVVFGQFAFFHTALNDVVEVHDGHSQH | |
| SRLLSSLSGSHTGESLPLATSNQVLIKFSAKGLAPARGFHFVYQAVPRTSATQCSSVPEP | |
| RYGKRLGSDFSVGAIVRFECNSGYALQGSPEIECLPVPGALAQWNVSAPTCVVPCGGNLT | |
| ERRGTILSPGFPEFYLNSLNCVWKIVVPEGAGIQIQVVSFVTEQNWDSLEVFDGADNTVT | |
| MLGSFSGTTVPALLNSTSNQLYLHFYSDISVSAAGFHLEYKTVGLSSCPEPAVPSNGVKT | |
| GERYLVNDVVSFQCEPGYALQGHAHISCMPGTVRRWNYPPPLCIAQCCGTVEEMEGVILS | |
| PGFPGNYPSNMDCSWKIALPVGFGAHIQFLNFSTEPNHDYIEIRNGPYETSRMMGRFSGS | |
| ELPSSLLSTSHETTVYFHSDHSQNRPGFKLEYQAYELQECPDPEPFANGIVRQAGYNVGQ | |
| SVTFECLPGYQLTGHPVLTCQEGTNRNWDHPLPKCEVPCGGNITSSNGTVYSPGFPSPYS | |
| SSQDCVWLITVPIGHGVRLNLSLLQTEPSGDFITIWDGPQQTAPRLGVFTRSMAKKTVQS | |
| SSNQVLLKFHRDAATCGIFAIAFSAYPLTKCPPPTILPNAEVVTENEEFNIGDIVRYRCL | |
| PGFTLVGNEILTCKLGTYLQFEGPPPICEVHCPTNELLTDSTGVILSQSYPGSYPQFQTC | |
| SWLVRVEPDYNISLTVEYELSEKQYDEFEIFDGPSGQSPLLKALSGNYSAPLIVTSSSNS | |
| VYLRWSSDHAYNRKGFKIRYSAPYCSLPRAPLHGFILGQTSTQPCGSIHFGCNAGYRLVG | |
| HSMAICTRHPQGYULWSEAIPLCQALSCGLPEAPKNGMVFGKEYTVGTKANYSCSEGYHL | |
| QAGAEATAECLDTGLWSNRNVPPQCVRESSGNGGGSVTCPDVSSISVEHGRWRLIFETQY | |
| QFQAQLMLICDPGYYYTGQRVIRCQANGKWSLGDSTPTCRIISCCELPIPPNGERIGTLS | |
| VYGATAIFSCNSGYTLVGSRVRECMANGLWSGSEVRCLATQTKLHSIFYKLLFDVLSSPS | |
| LTKAGHCGTPEPIVNGHINGENYSYRGSVVYQCNAGFRLIGMSVRICQQDHHWSGKTPFC | |
| VHVKQQLLLLLLLLCDDDDDEDDGSGAITCGHPGNFVNGLTQGNQFNLNDVVKFVCNPGY | |
| MAEGAARSQCLASGQWSDMLPTCRIINCTDPGHQENSVRQVHASGPHRFSFGTTVSYRCN | |
| HGFYLLGTPVLSCQGDGTWDRPRPQCLCK | |

Homologies to either of the above NOV1 proteins will be shared by the other NOV1 protein insofar as they are homologous to each other as shown below. Any reference to NOV1 is assumed to refer to both of the NOV1 proteins in general, unless otherwise noted.

The disclosed NOV1a polypeptide has homology to the amino acid sequences shown in the BLASTP data listed in Table 1E.

TABLE 1E
BLAST results for NOV1a

| Gene Index/ | | Length | Identity | Positives | |
|---|---|---|---|---|---|
| Identifier | Protein/Organism | (aa) | (%) | (%) | Expect |
| gi\|16716457\|ref\|NP_ | CUB and Sushi | 3554 | 54 | 79 | 0.0 |
| 444401.1\| | multiple domains 1 | | | | |
| (NM_053171) | [Mus musculus] | | | | |
| >gi\|15100168\|ref\|NP_ | CUB and Sushi | 3508 | 31 | 45 | 0.0 |
| 150094.1\| | multiple domains 1 | | | | |
| (NM_033225) | [Homo sapiens] | | | | |
| gi\|14787181\|gb\|AAG5 | CUB and sushi multiple | 3389 | 31 | 46 | 0.0 |
| 2948.1\| (AY017307) | domains protein 1 short | | | | |
| | form [Homo sapiens] | | | | |
| gi\|16162671\|ref\|XP_ | hypothetical protein | 1043 | 70 | 84 | 0.0 |
| 053758.2\| | XP_053758 [Homo | | | | |
| (XM_053758) | sapiens] | | | | |
| gi\|15620839\|dbj\|BAB | KIAA1890 protein | 1048 | 70 | 84 | 0.0 |
| 67783.1\| (AB067477) | [Homo sapiens] | | | | |

The homology between these and other sequences is shown graphically in the ClustalW analysis shown in Table 1F. In the ClustalW alignment of the NOV1 proteins, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

[image]

[image]

[image]

[image]

[image]

[image]

[image]

[image]

[image]

The presence of identifiable domains in NOV1, as well as all other NOVX proteins, was determined by searches using software algorithms such as PROSITE, DOMAIN, Blocks, Pfam, ProDomain, and Prints, and then determining the Interpro number by crossing the domain match (or numbers) using the Interpro website (http:www.ebi.ac.uk/ interpro). DOMAIN results for NOV1 as disclosed in Table 1I, were collected from the Conserved Domain Database (CDD) with Reverse Position Specific BLAST analyses. This BLAST analysis software samples domains found in the Smart and Pfam collections. For Table 1I and all successive DOMAIN sequence alignments, fully conserved single residues are indicated by black shading or by the sign (|) and “strong” semi-conserved residues are indicated by grey shading or by the sign (+). The “strong” group of conserved amino acid residues may be any one of the following groups of amino acids: STA, NEQK, NHQK, NDEQ, QHRK, MILV, MILF, HY, FYW.

Table 1G lists the domain description from DOMAIN analysis results against NOV1a. This indicates that the NOV1a sequence has properties similar to those of other proteins known to contain this domain.

TABLE 1G
Domain Analysis of NOV1a

gnl|Pfam|pfam00431, CUB, CUB domain

| | |
|---|---|
| CD-Length = 110 residues, 100.0% aligned | Score = 120 bits (301), Expect = 1e−27 |

TABLE 1H
Domain Analysis of NOV1a

gnl|Pfam|pfam00084, sushi, Sushi domain (SCR repeat)

| | |
|---|---|
| CD-Length = 56 residues, 100.0% aligned | Score = 57.0 bits (136), Expect = 2e−08 |

CUB domains are important protein interaction domains that occur primarily in secreted protein, including a variety of biologically important growth factors. CUB domains, when coupled to EGF domains, are important for calcium binding. This protein may mediate cell-cell contact, growth, or other important cellular processes.

The Ca2+-dependent interaction between complement serine proteases C1r and C1s is mediated by their alpha regions, encompassing the major part of their N-terminal CUB-EGF-CUB (where EGF is epidermal growth factor) module array. In order to define the boundaries of the C1r domain(s) responsible for Ca2+ binding and Ca2+-dependent interaction with C1s and to assess the contribution of individual modules to these functions, the CUB, EGF, and CUB-EGF fragments were expressed in eucaryotic systems or synthesized chemically. Gel filtration studies, as well as measurements of intrinsic Tyr fluorescence, provided evidence that the CUB-EGF pair adopts a more compact conformation in the presence of Ca2+. Ca2+-dependent interaction of intact C1r with C1s was studied using surface plasmon resonance spectroscopy, yielding KD values of 10.9-29.7 nM. The C1r CUB-EGF pair bound immobilized C1s with a higher KD (1.5-1.8 microM), which decreased to 31.4 nM when CUB-EGF was used as the immobilized ligand and C1s was free. Half-maximal binding was obtained at comparable Ca2+ concentrations ranging from 5 microM with intact C1r to 10-16 microM for C1ralpha and CUB-EGF. The isolated CUB and EGF fragments or a CUB+EGF mixture did not bind C1s. These data demonstrate that the C1r CUB-EGF module pair (residues 1-175) is the minimal segment required for high affinity Ca2+ binding and Ca2+-dependent interaction with C1s and indicate that Ca2+ binding induces a more compact folding of the CUB-EGF pair. (See Thielens et al., J Biol Chem Apr. 2, 1999;274(14):9149-59) The disclosed NOV1 nucleic acid of the invention encoding a cub and sushi domain-containing protein-like protein includes the nucleic acid whose sequence is provided in Table 1A or 1C, or a fragment thereof. The invention also includes a mutant or variant nucleic acid any of whose bases may be changed from the corresponding base shown in Table 1A or 1C while still encoding a protein that maintains its a cub and sushi domain-containing protein-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 1% percent of the bases may be so changed.

The disclosed NOV1 protein of the invention includes the a cub and sushi domain-containing protein-like protein whose sequence is provided in Table 1B or 1D. The invention also includes a mutant or variant protein any of whose residues may be changed from the corresponding residue shown in Table 1B or 1D while still encoding a protein that maintains its a cub and sushi domain-containing protein-like activities and physiological functions, or a functional fragment thereof. In the mutant or variant protein, up to about 71% percent of the residues may be so changed.

The invention further encompasses antibodies and antibody fragments, such as F_{ab} or (F_{ab})₂, that bind immunospecifically to any of the proteins of the invention.

The above defined information for this invention suggests that this a cub and sushi domain-containing protein -like protein (NOV1) may function as a member of a “Calgizzarin family”. Therefore, the NOV1 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV1 nucleic acids and proteins of the invention are useful in potential therapeutic applications implicated in cancer including but not limited to various pathologies and disorders as indicated below. For example, a cDNA encoding the a cub and sushi domain-containing protein-like protein (NOV1) may be useful in gene therapy, and the a cub and sushi domain-containing protein-like protein (NOV1) may be useful when administered to a subject in need thereof. By way of nonlimiting example, the compositions of the present invention will have efficacy for treatment of patients suffering from cancer, obesity, inflammation, hypertension, neurological diseases, neuropsychiatric diseases, small stature, obesity, diabetes, hyperlipidemia and other diseases, disorders and conditions of the like. The NOV1 nucleic acid encoding the a cub and sushi domain-containing protein-like protein of the invention, or fragments thereof, may further be useful in diagnostic applications, wherein the presence or amount of the nucleic acid or the protein are to be assessed.

NOV1 nucleic acids and polypeptides are further useful in the generation of antibodies that bind immuno-specifically to the novel NOV1 substances for use in therapeutic or diagnostic methods. These antibodies may be generated according to methods known in the art, using prediction from hydrophobicity charts, as described in the “Anti-NOVX Antibodies” section below. The disclosed NOV1 proteins have multiple hydrophilic regions, each of which can be used as an immunogen. In one embodiment, a contemplated NOV1 epitope is from about amino acids 400 to 450. In other embodiments, a NOV1 epitope is from about amino acids 500 to 600, from about 1000-1100, from about 1500-1600 and 2500-2800. These novel proteins can be used in assay systems for functional analysis of various human disorders, which will help in understanding of pathology of the disease and development of new drug targets for various disorders.

NOV2

A disclosed NOV2 nucleic acid of 1464 nucleotides (also referred to as cg-118733234) encoding a novel myelin-like protein is shown in Table 2A. An open reading frame was identified beginning with an ATG initiation codon at nucleotides 334-336 and ending with a TGA codon at nucleotides 1071-1073.

TABLE 2A
NOV2 nucleotide sequence.

(SEQ ID NO:5)

CAAAACACAAAAAAGAATAAACAAAAGGTTATCCCCCTTGTCTGCCAACCCCCCTCCCCTCCCAAATTTT
CCCTCCTCTCTTTGACCTCTTATTAACCGTCCACCCTTCTTTCCCCTTTAGAATAGTGAACCCCAGTTAC
CACCACTGATTAGTCATAACCGGTATTACCCCTATCTACTCCACTGAAAGTTACCTGGACAAACAAGAAT
CATATCCCAACCGATATTTGTCTGTGACACATCAGGAACCACAAGCTGCACTTCATTAAAAAATTATTTG
CGTATCACGTGTGGCAAACATTCAAATTCTCCTTCAAACAGTTGGAAGAAAACATGTAATACATTCCAGA
GCAAAGATGAATCAAAAAGTATCTTTTTGCTCAGGAAAAGAATTTCTTCATTCAATTACAGCATAATTCA
TTGAAACGGGAAGTCATGAGTCTCTTATGAGACTTCCTGAACAGTTTATAAATACAACAAGAACATTTAT
TCAATAAATAAGTGGTTCCTAAAGTCTTTACTGATGATCTCCACGATTGTCCATCGCTATGGTCCAGGCC
AGCTCCACTTTCTCTGACAGGCTTTAGCTGCCAGTGGAATCGGATGTTTCCTGTCTTTAGGTGACTCTTC
TTGTGTCATACAGACTTTCATCAATATGTCTCTTCATAGTCTGAATCCAGGCACTCAGCGCAACGGACAC
AAGCCTCGCCATACACGCCTCTTCCTCCTCCTGATCAGTGTCATCGGAAACCTCAATAGATGACTTCTTA
TAGCCAGACCTGCTCCTCTTCTTCAGCCCAGCAGCCTTCCTCCCCATTCTCACCAGCAGCAGAGCAACCA
CCACGGCTGAGGGCACAAAGACAAGGATGGAAACAAGGGCCACAGAGGAAAGCATGGTCCCAAAACCCCT
TTCTGTGACTGTTAGCTCTGTCATGGGAATATTATGGTGCACATCTCGGGGATTCTTCACAGCACAGCTG
AATGTCCCATTGTCCTTTATGGTAGGGTTGCTTATACTTATAGATGCATCCCCTTTGTATACATTTCCAA
CCCAGGAAATCCGATCCCGAAATGTGCCTGCTGTGGTTGGGTACTGGAAAGACTGATAATGAAATATTGA
TACTGTGTGGCTGCTGCTGCGAGGGCGATATGTCCAGTCTATAGTAACCTTGTCAGTGACATCTGAAGTT
GACTTGAAAGTGCATTTCAACTTGATCTTTTCTCCAACATAACCTCGGACATGGGCATCTGCACGAATCT
CCAAGGAAAAGACGATATAAACACCCTGGAAGAACAGGACGCCCAGCAGAGGGAAGAGAGCGCAGCCACG
GCTTCCAGCTGCTCCTCTCTGCTGCATCCCGGCAGCTCTTCAGATGCTTGCACACCTTGTTTACAGCTCC
CGGTAACGACTACAGGTAACACCGGAAGTGACGTCAGACCAGGAGGCCGAGAGACAACTTAAAT

The disclosed NOV2 nucleic acid sequence, localized to chromsome 11, has has 175 of 283 bases (61%) identical to a gb:GENBANK-ID:AF030455|acc:AF030455.1 mRNA from Homo sapiens (Homo sapiens epithelial V-like antigen precursor (EVA) mRNA, complete cds).

A NOV2 polypeptide (SEQ ID NO:8) encoded by SEQ ID NO:7 has 246 amino acid residues and is presented using the one-letter code in Table 2B. Signal P, Psort and/or Hydropathy results predict that NOV2 contains a signal peptide with the most likely cleavage site between positions 31 and 32 (i.e. VFS-LE). A NOV2 polypeptide is likely to be localized to the endoplasmic reticulum (membrane) with a certainty of 0.6850. In other embodiments, NOV2 may also be localized to the plasma membrane with a certainty of 0.6400, the Golgi body with a certainty of 0.4600, or the endoplasmic reticulum (lumen) with a certainty of 0.1000.

TABLE 2B
Encoded NOV2 protein sequence.

(SEQ ID NO:6)

MQQRGAAGSRGCALFPLLGVLFFQGVYIVFSLEIRADAHVRGYVGEKIKLKCTFKSTSDVTDKLTIDWTY
RPPSSSHTVSIFHYQSFQYPTTAGTFRDRISWVGNVYKGDASISISNPTIKDNGTFSCAVKNPPDVHHNI
PMTELTVTERGFGTMLSSVALLSILVFVPSAVVVALLLVRMGRKAAGLKKRSRSGYKKSSIEVSDDTDQE
EEEACMARLVSVALSAWIQTMKRHIDESLYDTRRVT

The disclosed NOV2 amino acid sequence has 70 of 192 amino acid residues (36%) identical to, and 101 of 192 amino acid residues (52%) similar to, the 248 amino acid residue ptnr:SWISSNEW-ACC:P25189 protein from Homo sapiens (Human) (MYELIN P0 PROTEIN PRECURSOR).

NOV2 is expressed in at least pituitary gland and prostate. This information was derived by determining the tissue sources of the sequences that were included in the invention. SeqCalling sources: Adrenal Gland/Suprarenal gland, Amygdala, Bone, Bone Marrow, Brain, Colon, Coronary Artery, Dermis, Epidermis, Foreskin, Hair Follicles, Heart, Hippocampus, Hypothalamus, Kidney, Liver, Lung, Lymph node, Lymphoid tissue, Mammarygland/Breast, Oesophagus, Ovary, Pancreas, Parathyroid Gland, Peripheral Blood, Pineal Gland, Pituitary Gland, Placenta, Prostate, Retina, Salivary Glands, Small Intestine, Spleen, Stomach, Testis, Thalamus, Thymus, Tonsils, Trachea, UmbilicalVein, Uterus, Whole Organism.

NOV2 also has homology to the amino acid sequences shown in the BLASTP data listed in Table 2C.

TABLE 2C
BLAST results for NOV2

| Gene Index/ | Protein/ | Length | Identity | Positives | |
|---|---|---|---|---|---|
| Identifier | Organism | (aa) | (%) | (%) | Expect |
| gi\|14250688\|gb\|AAH | (protein for | 124 | 100 | 100 | 3e−54 |
| 08810.1\|AAH08810 | IMAGE: 3948909) | | | | |
| (BC008810) | [Homo | | | | |
| | sapiens] | | | | |
| gi\|127719\|sp\|P10522 | (MYELIN | 219 | 42 | 56 | 7e−21 |
| \|MYP0 BOVIN | PERIPHERAL | | | | |
| | PROTEIN) | | | | |
| | (MPP) | | | | |
| gi\|2119433\|pir\| \|P38053 | myelin protein | 251 | 35 | 52 | 4e−20 |
| | zero - human | | | | |
| gi\|4505243\|ref\|NP_ | (Charcot-Marie- | 258 | 35 | 52 | 5e−20 |
| 000521.1\| | Tooth | | | | |
| NM_000530) | neuropathy 1B); | | | | |
| | Myelin protein | | | | |
| | zero [Homo | | | | |
| | sapiens] | | | | |
| gi\|14724169\|ref\|XP_ | myelin protein | 248 | 35 | 52 | 5e−20 |
| 042459.1 | zero [Homo | | | | |
| (XM_042459) | sapiens] | | | | |

The homology of these sequences is shown graphically in the ClustalW analysis shown in Table 2D.

[image]

[image]

Tables 2E-F list the domain description from DOMAIN analysis results against NOV2. This indicates that the NOV2 sequence has properties similar to those of other proteins known to contain this domain.

TABLE 2E
Domain Analysis of NOV2

gnl|Smart|smart00406, IGv, Immunoglobulin V-Type
CD-Length = 80 residues, 98.8% aligned Score = 50.4 bits (119),
Expect = 1e−07

Myelin is an important insulating protein which protects nerve cells. Mutation of mylein proteins can cause a variety of neurological disorders. Pelizaeus-Merzbacher disease (PMD) and spastic paraplegia type 2 (SPG2) are X-linked developmental defects of myelin formation affecting the central nervous system (CNS). They differ clinically in the onset and severity of the motor disability but both are allelic to the proteolipid protein gene (PLP), which encodes the principal protein components of CNS myelin, PLP and its spliced isoform, DM20. 52 PMD and 28 SPG families without large PLP duplications or deletions were investigated by genomic PCR amplification and sequencing of the PLP gene. 29 and 4 abnormalities were discovered respectively. Patients with PLP mutations presented a large range of disease severity, with a continuum between severe forms of PMD, without motor development, to pure forms of SPG. Clinical severity was found to be correlated with the nature of the mutation, suggesting a distinct strategy for detection of PLP point mutations between severe PMD, mild PMD and SPG. Single amino-acid changes in highly conserved regions of the DM20 protein caused the most severe forms of PMD. Substitutions of less conserved amino acids, truncations, absence of the protein and PLP-specific mutations caused the milder forms of PMD and SPG. Therefore, the interactions and stability of the mutated proteins has a major effect on the severity of PLP-related diseases. (See Cailoux et al., Eur J Hum Genet November 2000;8(11):837-845).

A novel hereditary motor and sensory neuropathy (HMSN) phenotype, with partial steroid responsiveness, caused by a novel dominant mutation in the myelin protein zero (MPZ) gene has been discovered. Most MPZ mutations lead to the HMSN type I phenotype, with recent reports of Dejerine-Sottas, congenital hypomyelination, and HMSN II also ascribed to MPZ mutations. Differing phenotypes may reflect the effect of particular mutations on MPZ structure and adhesivity. Clinical, neurophysiological, neuropathological, and molecular genetic analyses of a family presenting with an unusual hereditary neuropathy were used. It was discovered that progressive disabling weakness, with positive sensory phenomena and areflexia, occurred in the proband with raised CSF protein and initial steroid responsiveness. Nerve biopsy in a less severely affected sibling disclosed a demyelinating process with disruption of compacted myelin. The younger generation were so far less severely affected, becoming symptomatic only after 30 years. All affected family members were heterozygous for a novel MPZ mutation (Ile99Thr), in a conserved residue. This broadens the range of familial neuropathy associated with MPZ mutations to include steroid responsive neuropathy, initially diagnosed as chronic inflammatory demyelinating polyneuropathy. (See Donaghy et al., J Neurol Neurosurg Psychiatry December 2000;69(6):799-805)

The disclosed NOV2 nucleic acid of the invention encoding a myelin-like protein includes the nucleic acid whose sequence is provided in Table 2A or a fragment thereof. The invention also includes a mutant or variant nucleic acid any of whose bases may be changed from the corresponding base shown in Table 2A while still encoding a protein that maintains its Myelin-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 39% percent of the bases may be so changed.

The disclosed NOV2 protein of the invention includes the Myelin-like protein whose sequence is provided in Table 2B. The invention also includes a mutant or variant protein any of whose residues may be changed from the corresponding residue shown in Table 2B while still encoding a protein that maintains its Myelin-like activities and physiological functions, or a functional fragment thereof. In the mutant or variant protein, up to about 64% percent of the residues may be so changed.

The NOV2 nucleic acids and proteins of the invention are useful in potential therapeutic applications implicated in neurological disorders, short stature, cancers, especially prostate cancer, metabolic disorders, inflammation and/or other pathologies and disorders. The NOV2 nucleic acid encoding myelin-like protein, and the myelin-like protein of the invention, or fragments thereof, may further be useful in diagnostic applications, wherein the presence or amount of the nucleic acid or the protein are to be assessed.

NOV2 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV2 protein has multiple hydrophilic regions, each of which can be used as an immunogen. In one embodiment, a contemplated NOV2 epitope is from about amino acids 5 to 35. In another embodiment, a NOV2 epitope is from about amino acids 145 to 180. In additional embodiments, NOV2 epitopes are from about amino acids 220 to 240. These novel proteins can be used in assay systems for functional analysis of various human disorders, which are useful in understanding of pathology of the disease and development of new drug targets for various disorders.

NOV3

A disclosed NOV3 nucleic acid of 5123 nucleotides (also referred to as CG122561227) encoding a novel vonWillebrand Factor (VWF)-like and kielin-like protein is shown in Table 3a. An open reading frame was identified beginning with a ATG initiation codon at nucleotides 4951-4948 and ending with a TGA codon at nucleotides 436-434.

TABLE 3A
NOV3 Nucleotide Sequence

(SEQ ID NO:7)

GCTTTTTACCATACCAGGGAGCCCACCTCAACATGACTGTGGAAGACCAAAGGATATACCTAGGTTCAGA
TTATAATAAATCACCCAGCACCACCTGAATGTATTATCCACAAAGATATAGCAATAATAAAGGTTATATA
TACATATATTTATCTTGGTAACCTGAGGGCTAAAAACGTGGAATACGATAATTCTTCTCAAGAGGTCCAT
AGGGCCGGCGGCCGCAGGTCACGAAGCCTCCAAGACAGGTACACAGGTTGCAGGGTTCTCGGGGGTCTGG
GAACTCCTGGTTACTCACGTACGACTCCCCCAGGTACTCACAGCCATCACAGTCAGGGCAGCAGTCGCCC
CTGGCAGGGAAGGGGCACAGTGCAGGGGCACATGCCTTGGGCTCGCAGCTCACGCTGCCCTCCCAGCAAA
GGCAGAGGTGGCAGGCACCAGTGGGCGATGGGAAGCGCTCCCCGCTGGCAAACTCCTTCCCCTGGTACAG
GCAGCCGTCGCAGGAGGCGCAGCAAGGCCCCTGGCGCGGGTGCGCGCAGGGCGCGGGCGGGCACGGCAGC
CGCTGGCACGACACGGAGCCGTCGAGGCAGAGGCAGCGGCGGCATGGATCGCCGGCCGGGGAGAAGTACT
CCTGGTGGCGGGCGTGGGCCGCGCCGCGCCGTGGGCAGCCGGCGGGGGCTGGGGCGGCTGCGGCGAGACA
GCGCATCAAAGCGGCCCAGGAACACGCCTCCGAGGGGACCCCCAGGGAGGAGCAGCCGAGGGGTGCGGCC
CCTACCTGGGCACTGCGGGCAGCACTCTCCCGGCAGCAGGACAGGCTCAGACAGCGACACACACGGCAGG
GGTCAGAGGGGTGGGGGAAGTCCGCTCCGCTGGGGTACTCTTTCCCGCCAAAGGCACAGCCGCTGCAGTC
GTTCGGGCAGCAGGTCCCAGGCAGCGGGTGGGCACAGGGGGCCCTGGGGCAGGGGCGAGGCTGGCAGTGG
GCATGGCCTTCCTCGCATCGGCACTCCTGGCAGGGGTCTCGGGGGTGGGAGAAGCTCTCGCCGTCCACAA
ACACCTCTTCCTCCAGGATGCAGTCTGGGGGGTAGGGGGGTGGGGGCCCTAGCGTGGTGGGGCCGGGGGC
AGTAGTTACTGGGCACCTGGGGCAACACTGGCCTGGTCCACTCTGGGCCCTGGCACAGGTCGTGGGAGGG
CAGTCAACCAAGGACCATGTCACAGTTCCATCCTGACAGTGGCAGCCATCGCAAGGGCTGTCTGCATCCG
TGAAGTTCTGCCCATTGGCATACACTTGGCTGTGGTAGGTGCAGCTGTCACAGCTGGGGCAGCAGGCACC
AGGGGGCTGGGTGGGGTGCTGGCAGGGGGCTGGGGGGCAGAGCACAGCCCCGCACTTGGGTACCCCATCT
TGACAGACGCAGGCGGTGCAGGGCCGACCATCAGGCTCCCACTGGACTCCCTCAGCAAACTCCTCTCCAT
CCAGCTCACAGGCTGGGCAGAGCTGGCGGCCAGAGGCAGGCAGGGCACAGGGGGTGACTCGGCACTCCTG
CTCCTCACAGGAGACCTCGCCAGCCTGGCAGGAGCAGCGGACACAGAGGCCCCGCTCTTGGAGTCTGAAG
GTCTCCTGGCTCTGATACTGGTGTCCCTGGTACTCACAGCCTGGATGTGACCCTCCCGGAGCCGCTGCTG
CTCGCGGTCCCGCCGCGGCGCTGCCGCCTGCCGCGCTGTGCAGCTCCAGCAGCCGCTGCAGAGCCGTCAC
CTGCTGCGCCGCGCTTAAGCCCGCAGGCGCCTTGGCGGTGCTGAGGGGCGCCGGCCACGCCGCCTTCAAG
GCCTCCTGCTCCCCCCGTGCGCGGCCCACTCCAGGGCCCTGCAGCCCGTGCGGATCGCGCTCCAGGCCTG
GGGGCCCCGGAGAGCCCCGAGGAGCCCCTGCTGCCCGGATACCTCGGCGCGCAGGGTCCCTCGCATCTCG
GCTCCGGCTCCGCGACCTGGGAGCCGCCGCCGGGCCGGGGGCTTCGGGCGGGTAAGCGCAGCCCACGCCC
CCTCCCGGGCGGCCCCCAGCTTTGCCACCGCCGGTGCCGACCTTTGTCGCTCGCCTTTCATCATGCTCTG
CGTCAGCGTGGTAGTCCTTCTCCGGAGGTTTGGGCTCTCCCTGCCCACAGGCTTTGGAGTCTGTGCTTTC
AGGGACCCGCAGGAGTCCCTCGCATGGCCTGAGGGGTCTCCGCTTTCTGATCCTGAGGGTCCTCCTCTCA
GACTCAGGGGTGTCCATCCTTGGTCGTCTTTGAAGTTCTGCTCTCTCCTGGCCCAGGTGCGGGTCCGAGC
CCAGCCCTTCAAGGGCATCTTGGGAAGGCAGGTTTTGCGAGGGCAGGTCCCCCGGCCCAGGGGTCCCGGG
AGTGAGCTTTCTTTTCTGGGTCTCAGGCTCTGCCTCACTCCTCTCTTCCCTCTGGGCCAGGTCCGGCTGC
TCAGGGTCCCCACCTGTGCTACCACCTGGCTGACAGCACTCGCTCCATGCTTGCTGGGCCTGGCCCCTCG
GACCGAAAAACTGGGCCTCGGGTGGCCAGAAGAGGCCCAGCTCCACACCAGTCAGCAGTTCCAAGTCCCA
CAGGAGCTGCTCCTCGGCCCGCCGGCTCCACTCCATCTGCAGCAGTACAGGGGGCAGCTCCAGCCCTGGG
GGATACCCGCCGCCCTCTTGCACGCAGTGGGCTGCCAGCTCCCCCAGCGGGATATGCTCATTGAAGCAGG
TGCGGGGACAGGGTGGGCCGCACTCATCAAACACGAAGCCACGCTCCAGGGGGCAGCCTACCACACACAG
CGTGGGGCCTCGCCAGGTAGGTGTCACTCCTGCCTGGCGACAGTGACTGGCGTAGGCTTCCAGGGCATCA
CACAGGCAGGCATCACCCGAGGAGCCAGGGCCACAGCCACACAGGTCATACACACAGGCGGCAAAGAAGG
GCTCCGGTGGCACCACAGCATGGCAGCGACTGAATGGGGAGGACTTCAGCACCCCACACCGGGCATTGGC
CTCACGCCTGGCACGGTAACCTGCTGCCCGGCACGCATCCACCTCTCGGCCTGCAGAACAGGGCCGGCCA
GGCCACAGCCCCTCTGAGACCTGCCAGCTATTCCCAAACGCAGCCTCCGAGGGCAGGAGCAGCCCCTCAG
GGCCCTGCAGATCGTCCTGGGCAAAGCCATTGAAGTTCCCACAGAGCCCACAAGTCCGGCCCTGGTAGGA
GCCAGGTACGCTCACCTCCACCTGGGACTGCCCATCCCACAGCACCTGGAGCCCGCGCTGGGCGTGCAGG
ATCACAGTGTGTCCTCGCAGCTCCACATACAGCAGCGGCTCCTGCACGAAGGGCAAGGCCACCGGGTGCC
CATCCACCGTGACTGCCCCGTCCTGCAGCAGCCGCACGGCCATGTCTCCCAGCAGCACCGCCACCTCCTG
GGTCCAGGCCACACTGCTCCGGCCCCGGTCATCATTGGTCACGTGCACACTGAAGTCCCCGCTGTGGCAG
TCCTTGGCCAGCACATAGCTGCAACTGCTCTGGAAGTGCAGCAGGCGGCCGTCGAAGGTGCGGTAATGGG
GGTCTCCGAAGGCCATGCAGGAAGCGGGCCGAGGCAGGCAGCGGGGGCAGCACCTGCCAGGACTCAGGGC
AGGGGCCTTGTCGGGGCCACACGAGAGCGGTGAGCAGCGCTCGCTCTGGCAACGCACGGTGCCCGCCATG
CAGGAGCAGCTGGTGCAGGTGTCCACAGTCCAGCGCTCTCCAGAGGCCACCTCACGGCCCTGGTGCACGC
AGGACTGGGTGGGAGCTTGGCATCGCTCACAGCAGCTGTCAGCCTCGGGCACCTTCGCCCAGCCATGGGG
GCAGGAGAGGGCCTGGCACTCCTCGAGGTGGCACTCCACATGGCCCCGATGGCAGGTGCACGCGATGCAC
GCATTGCTGGGGTCCCGCCAGCTCTCTCCATCTGCCACTCTCCGGCCCTCGGCCTCCACCACACATTCCC
GGCATACGGGGCAGCAGCTCCCAGGGGGAGTGTGGCCCTCTGAGAGGGGACAGCTGAGCTCAGGACAAGC
CTGGTGGATGCAGAGCCATGTCAGGTCCTGGCACTGGCACGTGTAGCAGGGGTCTGGTGGGGCTAGCTCA
GATCCCAGCAGGCCCTCTGAACAGTTACTCAAGGCCTCGGCACAGGTCGGACAGCAGTGCTGGGGCCCAG
GGGGCAGGAGCTGGCTCGGGGGGCAGCCCACCAGGCTGGGACACTGCCGCCGGTGACAGCGAAGGCTGGG
AGGCCCCTCAGGCTGTGGCTCGCAGATGCACACTTCACAGGGGTCTGCCCCAGGCTGGAAGCTCTCCCCA
GGCTCGTACTTCCGGCCCTCATGCTCACAGTCAGAGCATTGAGGACAGCAGTCATGGGGCCCTTGGCGGG
GCTGGGCGCAAGAGCTGATGCACTGGATGCGTGCACAGGTGACGACGCCCTCGTGACACACACAGGAGGA
GCAGGCACTGTCGGGGGGCACCCATCTACTGCCTTCGGGGTGCTCTTCCCCATGAGCCAGGCAGCCTGCT
CTCAAGACCAAGCTCCAGGCAGCAGGCACTGCCCAGTGCAGCCCGCACCGGCCACAGACCTGGCATGCAG
CAGCTGTGTCATAAATGCATGTGGACCAGGTGAATGGCTCCATCGCTAACCATGTGCCCAAGGTAACTCA
GTCTCTCCGGGCCTCAGTCTCCTCATCTGTTAAATGGGGATTTCCTCTGTCCTGCCTCCCTCCCAGAGCA
AGTCAAATGCTATGACAGTTCTGTGGTTCTGTAGAAACACCACCACACTGCGCAGGGTGTGGGACGAGGG
GGCAGTGGGCACGCTAAGAAAGGCTCAAGTTCAGAGCCAGATGGTCCAAGTGTCCATCTTGACTCTGCCA
CCTTCTCTACCTCTCTGTACCTCCACTTTCTCGTCTGTAAAATAGGAGGACTAAGAGTGCTTATCTCGTA
AGTTGTTGTGCTGATTAAATGAGATAATACACGTAAAGTGCTCAGGGCCTGGCACATGCTACCTGCTCAC
TGAATGTCAGGTATCTTGATGATGATGATGATGGTGGTGATGATGATGATGATGATGAATGGGGTGTGGT
TAGGAAGAGGGGC

The disclosed NOV3 nucleic acid sequence maps to chromosome 7 and has 1074 of 1729 bases (62%) identical to a gb:GENBANK-ID:AB026192|acc:AB026192.1 mRNA from Xenopus laevis (Xenopus laevis mRNA for Kielin, complete cds).

A disclosed NOV3 protein (SEQ ID NO:10) encoded by SEQ ID NO:9 has 1497 amino acid residues, and is presented using the one-letter code in Table 3B. Signal P, Psort and/or Hydropathy results predict that NOV3 does have a signal peptide, and is likely to be localized to the nucleus with a certainty of 0.6000. In other embodiments NOV3 is also likely to be localized to the mitochondrial matrix space with a certainty of 0.4270, to the mitochondrial inner membrane with a certainty of 0.1047, or to the mitochondrial inner membrane space with a certainty of 0.1047. The most likely cleavage site for NOV3 is between positions 43 and 44, (CLA-HG).

TABLE 3B
Encoded NOV3 protein seqnence.

(SEQ ID NO:8)

MEPFTWSTCIYDTAACQVCGRCGLHWAVPAAWSLVLRAGCLAHGEEHPEGSRWVPPDSACSSCVCHEGV
VTCARIQCISSCAQPRQGPHDCCPQCSDCEHEGRKYEPGESFQPGADPCEVCICEPQPEGPPSLRCHRRQ
CPSLVGCPPSQLLPPGPQHCCPTCAEALSNCSEGLLGSELAPPDPCYTCQCQDLTWLCIHQACPELSCPL
SERHTPPGSCCPVCRECVVEAEGRRVADGESWRDPSNACIACTCHRGHVECHLEECQALSCPHGWAKVPQ
ADSCCERCQAPTQSCVHQGREVASGERWTVDTCTSCSCMAGTVRCQSQRCSPLSCGPDKAPALSPGSCCP
RCLPRPASCMAFGDPHYRTFDGRLLHFQSSCSYVLAKDCHSGDFSVHVTNDDRQRSSVAWTQEVAVLLGD
MAVRLLQDGAVTVDGHPVALPFLQEPLLYVELRGHTVILHAQPGLQVLWDGQSQVEVSVPGSYQGRTCGL
CGNFNGFAQDDLQGPEGLLLPSEAAFGNSWQVSEGLWPGRPCSAGREVDPCRAAGYRARREANARCGVLK
SSPFSRCHAVVPPEPFFAACVYDLCACGPGSSADACLCDALEAYASHCRQAGVTPTWRGPTLCVVGCPLE
RGFVFDECGPPCPRTCFNQHIPLGELAAHCVQEGRGYPPGLELPPVLLQMEWSRRAQEQLLWDLELLTGV
ELGLFWPPQAQFFGPRGQAQQAWSQCCQPGGSTGGDPEQPDLAQREERSEAEPETQKRKLTPGTPGPGDL
PSQNLPSQDALEGLGSDPHLGQERAELQRPPRNDTPESERRTLRIRKRRPLSPSEGLLRVPESTDSKACG
QGEPKPPEKDYHADAEHDQRRATKVGTGGGKAGGRPGGGVGCAYPPEAPGPAAAPRSRSRSRDASDPARR
GIRAAGAPRGSPGPPGLERDPHGLQGPGVGPAPGEQEALKAAWPAPLSTAKAPAGLSAAQQVTALQRLLE
LHSAAGGSAAAGPRAAAAPGGSHPGCEYQGHQYQSQETFRLQERGLCVRCSCQAGEVSCEEQECPVTPCA
LPASGRQLCPACELDGEEFAEGVQWEPDGRPCTACVCQDGVPKCGAVLCPPAPCQHPTQPPGACCPSCDS
CTYHSQVYANGQNFTDADSPCEACHCQDGTVTCSLVDCPPTTCARPQSCPGQCCPRCPVTTAPRPTTLGP
PPPYPPDCILEEEVFVDGESFSHPRDPCQECRCQEGHAHCQPRPCPRAPCAHPLPGTCCPNDCSGCAFGG
KEYPSGADFPUPSDPCRLCRCLSLSCCRESAARSAQVGAAPLGCSSLGVPSEACSWAPLMRCLAAAAPAP
AGCPRPGAAHARHQEYFSPPGDPCRRCLCLDGSVSCQRLPCPPAPCAHPRQGPCCPSCDGCLYQGKEFAS
GERFPSPTAACHLCLCWEGSVSCEPKACAPALCPFPARGDCCPDCDGCEYLGESYLSNQEFPDPREPCNL
CTCLGGFVTCGRRPYGPLEKNYRIPRF

The disclosed NOV3 amino acid has 359 of 642 amino acid residues (55%) identical to, and 457 of 642 amino acid residues (71%) similar to, the 2327 amino acid residue ptnr:SPTREMBL-ACC:Q9IBG7 protein from Xenopus laevis (African clawed frog) (KIELIN).

The NOV3 sequence is predicted to be expressed in the Adrenal Gland/Suprarenal gland, Amygdala, Aorta, Bone, Bone Marrow, Brain, Cerebellum, Cervix, Chorionic Villus, Cochlea, Colon, Dermis, Epidermis, Foreskin, Hair Follicles, Heart, Hippocampus, Hypothalamus, Kidney, Liver, Lung, Lymph node, Lymphoid tissue, Mammary gland/Breast, Muscle, Myometrium, Ovary, Pancreas, Parotid Salivary glands, Pituitary Gland, Placenta, Prostate, Proximal Convoluted Tubule, Small Intestine, Spinal Chord, Spleen, Stomach, Substantia Nigra, Testis, Thymus, Thyroid, Tonsils, Umbilical Vein, Urinary Bladder, Uterus.

NOV3 also has homology to the amino acid sequences shown in the BLASTP data listed in Table 3C.

TABLE 3C
BLAST results for NOV3

| Gene Index/ | Protein/ | | | Positives | |
|---|---|---|---|---|---|
| Identifier | Organism | Length (aa) | Identity (%) | (%) | Expect |
| gi\|7768636\|dbj\|BAA9 | Kielin | 2327 | 55 | 70 | e−177 |
| 5483.1\| (AB026192) | [Xenopus | | | | |
| | laevis] | | | | |
| gi\|9864185\|gb\|AAG01 | Crossveinless 2 | 751 | 32 | 44 | 4e−69 |
| 337.1\|AF288223_1\| | [Drosophila | | | | |
| (AF288223) | melanogaster] | | | | |
| gi\|7291288\|gb\|AAF46 | CG15671 gene | 555 | 32 | 45 | 3e−55 |
| 719.1\| (AE003453) | product | | | | |
| | [Drosophila | | | | |
| | melanogaster] | | | | |
| gi\|12851935\|dbj\|BAB | Putative | 452 | 34 | 48 | 1e−51 |
| 29213.1\| (AK014221) | protein/mouse | | | | |
| gi\|12667418\|gb\|AAK0 | Sonadhesion | 2501 | 34 | 47 | 2e−39 |
| 1435.1\|AF332979_1\| | variant | | | | |
| (AF332979) | 5/human | | | | |

The homology of these sequences is shown graphically in the ClustalW analysis shown in Table 3D.

[image]

[image]

[image]

[image]

[image]

[image]

[image]

[image]

Table 3E lists the domain description from DOMAIN analysis results against NOV3. This indicates that the NOV3 sequence has properties similar to those of other proteins known to contain this domain.

TABLE 3E
Domain Analysis of NOV3

gnl|Smart|smart00216,
CD-Length = 162 residues, 99.4% aligned Score = 139
bits (351), Expect = 9e−34

Von Willebrand factor domains are present in a number of proteins important for growth and cell division. One such protein, Kielin, is important for early embryonic development, and may be an excellent target for cancer. The midline tissues are important inductive centers of early vertebrate embryos. By signal peptide selection screening, we isolated a secreted factor, Kielin, which contains multiple cys-rich repeats similar to those in chordin (Chd). Expression of Kielin starts at midgastrula stages in the notochord and is detected in the floor plate of neurula embryos. Kielin is induced in mesoderm and in ectoderm by nodal-related genes. Chd is sufficient to activate Kielin expression in mesoderm whereas Shh or HNF-3beta in addition to Chd is required for induction in ectoderm. Kielin has a distinct biological activity from that of Chd. Injection of Kielin mRNA causes dorsalization of ventral marginal zone explants and expansion of MyoD expression in neurula embryos. Unlike Chd, Kielin does not efficiently induce neural differentiation of animal cap ectoderm, suggesting that the activity of Kielin is not simply caused by BMP4 blockade. Kielin is a signaling molecule that mediates inductive activities of the embryonic midline. (See Matsui et al., Proc Natl Acad Sci U S A May 9, 2000;97(10):5291-6).

The disclosed NOV3 nucleic acid of the invention encoding a VWF-like and kielin-like protein includes the nucleic acid whose sequence is provided in Table 3A or a fragment thereof. The invention also includes a mutant or variant nucleic acid any of whose bases may be changed from the corresponding base shown in Table 3A while still encoding a protein that maintains its VWF-like and kielin-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 38 percent of the bases may be so changed.

The disclosed NOV3 protein of the invention includes the VWF-like and kielin-like protein whose sequence is provided in Table 3B. The invention also includes a mutant or variant protein any of whose residues may be changed from the corresponding residue shown in Table 3B while still encoding a protein that maintains its VWF-like and kielin-like activities and physiological functions, or a functional fragment thereof. In the mutant or variant protein, up to about 45 percent of the residues may be so changed.

The protein similarity information, expression pattern, and map location for the VWF-like and kielin-like protein and nucleic acid (NOV3) disclosed herein suggest that NOV3 may have important structural and/or physiological functions characteristic of the VWF-like and kielin-like kinase-like family. Therefore, the NOV3 nucleic acids and proteins of the invention are useful in potential diagnostic and therapeutic applications. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo.

The NOV3 nucleic acids and proteins of the invention are useful in potential diagnostic and therapeutic applications implicated in various diseases and disorders described below. For example, the compositions of the present invention will have efficacy for treatment of patients suffering from cancer, inflammation, neurological disorders, neuropsychiatric disorders, obesity, diabetes, bleeding disorders and/or other pathologies. The NOV3 nucleic acid, or fragments thereof, may farther be useful in diagnostic applications, wherein the presence or amount of the nucleic acid or the protein are to be assessed.

NOV3 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV3 protein have multiple hydrophilic regions, each of which can be used as an immunogen. In one embodiment, contemplated NOV3 epitope is from about amino acids 1 to 2. In another embodiment, a NOV3 epitope is from about amino acids 400 to 440. In additional embodiments, NOV3 epitopes are from about amino acids 900 to 950 and from about amino acids 1375 to 1425. This novel protein also has value in development of powerful assay system for functional analysis of various human disorders, which will help in understanding of pathology of the disease and development of new drug targets for various disorders.

NOV4

NOV4 includes six novel semaphorin-like proteins disclosed below. The disclosed sequences have been named NOV4a, NOV4b, NOV4c, NOV4d, NOV4e, and NOV4f.

NOV4a

A disclosed NOV4a nucleic acid of 1896 nucleotides (designated CuraGen Acc. No. SC70504370_A/CG59253-01) encoding a novel Sempahorin-like protein is shown in Table 4a. An open reading frame was identified beginning with an ATG initiation codon at nucleotides 46-48 and ending with a TAG codon at nucleotides 1474-1476.

TABLE 4A
NOV4a Nucleotide Sequence

(SEQ ID NO:9)

TGGCATTTCTGAGCAGGGGCCACCCTGACTTCACCTTGGCCCACCATGAGGGTCTTCCTGCTTTGTGCCT
ACATACTGCTGCTGATGGTTTCCCAGTTGAGGGCAGTCAGCTTTCCTGAAGATGATGAACCCCTTAATAC
TGTCGACTATCACTATTCAAGGCAATATCCGGTTTTTAGAGGACGCCCTTCAGGCAATGAATCGCAGCAC
AGGCTGGACTTTCAGCTGATGTTGAAAATTCGACACACACTTTATATTGCTGGCAGGGATCAAGTTTATA
CAGTAAACTTAAATGAAATGCCCAAAACAGAAGTAATACCCAACAAGAAACTGACATGGCGATCAAGACA
ACACGATCGAGAAAACTGTGCTATGAAAGGCAAGCATAAACATGAATGCCACAACTTTATCAAAGTATTT
GTTCCAAGAAACGATGAGATGGTTTTTGTTTGTGGTACCAATGCATTCAATCCCATGTGTAGATACTACA
GGTTGAGTACCTTAGAATATGATGGGGAAGAAATTAGTGCCCTGGCAAGATGCCCATTTGATGCCAGACA
AACCAATGTTGCCCTCTTTGCTGATGGGAAGCTGTATTCTGCCACAGTGGCTGACTTCTTGGCCAGCGAT
GCCGTTATTTATCGAACCATGGGTGATGGATCTGCCCTTCGCACAATAAAATATGATTCCAAATGGATAA
AAGAGCCACACTTTCTTCATGCCATAGAATATGGAAACTATCTCTATTTCTTCTTTCGAGAAATCGCTGT
CGAACATAATAATTTAGGCAACGCTGTGTATTCCCGCGTGGCCCGCATATGTAAAAACGACATGGGTGGT
TCCCAGCGGGTCCTGGAGAAACACTGGACTTCATTTCTAAAGGCTCGGCTGAACTGTTCTGTCCCTGGAG
ATTCGTTTTTCTACTTTGATGTTCTGCAGTCTATTACAGACATAATACAAATCAATGGCATCCCCACTGT
GGTCGGGGTGTTTACCACGCAGCTCAATAGCATCCCTGGTTCTGCTGTCTGTGCATTTAGCATGGATGAC
ATTGAAAAACTATTCAAAGGACGGTTTAAGGAACAGAAAACTCCAGATTCTGTTTGGACAGCAGTTCCCG
AAGACAAAGTGCCAAAGCCAAGGCCTCGCTGTTGTGCAAAACACGGCCTTGCCGAAGCTTATAAAACCTC
CATCGATTTCCCGGATGAAACTCTGTCATTCATCAAATCTCATCCCCTGATGGACTCTGCCGTTCCACCC
ATTGCCGATGAGCCCTGGTTCACAAAGACTCGGGTCAGGTACAGACTGACGGCCATCTCAGTGGACCATT
CACCCGGACCCTACCAGAACTACACACTCATCTTTGTTGGCTCTGAAGCTGGCATGGTACTTAAAGTTCT
GGCAAAGACCAGTCCTTTCTCTTTGAACGACAGCGTATTACTGGAAGAGATTGAAGCCTACAACCATGCA
AAGTAGGTATATGTTACGAGAACGCCCTTCAGCACTGCTCAAAAATTTTCGGCATGTATTTCATCTAGTC
ATGTCCTTTTGGTCCTCTAAATTAGCAGTGGTTTGGCATAATAGTGTTTTGTGTTTTTTTTCTCATTGAA
ATAAATCTTGGGTTTGTTTTTTTCCCGAGCCTGCTAGGGCGAGGGGGGTGAATGGTTGATGAGTTTAAAA
ATAATGCAGCCCTTGTTTTTCACCTGTAGAATATGAGAACATTTTAACAGCACCTCTCTTATCTTGCAGA
TATATTCCAAGATGCTACATGCAGCAGACAGCTGTGAGCTTGCATACACACACACACAAATATACATGCA
CATACATACACAGAATGCAGTACTAGTTAAGTATTTCCTTCCTATCTTTAATAAGTAAGAGAATATTTAG
ACCATT

A NOV4a nucleic acid is found in at least Brain (Hippocampus, Substantia Nigra), and Kidney. A NOV4a nucleic acid has 1588 of 1588 bases (100%) identical to a gb:GENBANK-ID:AK021660|acc:AK021660.1 mRNA from Homo sapiens (Homo sapiens cDNA FLJ11598 fis, clone HEMBA1003866, moderately similar to Mus musculus semaphorin VIa mRNA).

A NOV4a polypeptide (SEQ ID NO:16) encoded by SEQ ID NO:15 is 476 amino acid residues and is presented using the one letter code in Table 4B. Signal P, Psort and/or Hydropathy results predict that NOV4a has a signal peptide and is likely to be localized outside the cell with a certainty of 0.7380. In other embodiments, NOV4a may also be localized to the lysosome (lumen) with a certainty of 0.1900 or to the microbody with a certainty of 0.1875.

TABLE 4B
NOV4a protein sequence

(SEQ ID NO:10)

MRVFLLCAYILLLMVSQLRAVSFPEDDEPLNTVDYHYSRQYPVFRGRPSGNESQHRLDFQLMLKIRDTLY
IAGRDQVYTVNLNEMPKTEVIPNKKLTWRSRQQDRENCAMKGKEKDECHNFIKVFVPRNDEMVFVCGTNA
FNPMCRYYRLSTLEYDGEEISGLARCPFDARQTNVALFADGKLYSATVADFLASDAVIYRSMGDGSALRT
IKYDSKWIKEPHFLHAIEYGNYVYFFFREIAVEHNNLGKAVYSRVARICKNDMGGSQRVLEKHWTSFLKA
RLNCSVPGDSFFYFDVLQSITDIIQINGIPTVVGVFTTQLNSIPGSAVCAFSMDDIEKVFKGRFKEQKTP
DSVWTAVPEDKVPKPRPGCCAKHGLAEAYKTSIDFPDETLSFIKSHPLMDSAVPPIADEPWFTKTRVRYR
LTAISVDHSAGPYQNYTVIFVGSEAGMVLKVLAKTSPFSLNDSVLLEEIEAYNHAK

The full amino acid sequence of the protein of the invention was found to have 367 of 367 amino acid residues (100%) identical to, and 367 of 367 amino acid residues (100%) similar to, the 367 amino acid residue ptnr:TREMBLNEW-ACC:BAB13869 protein from Homo sapiens (Human) (CDNA FLJ11598 FIS, CLONE HEMBA1003866, MODERATELY SIMILAR TO MUS MUSCULUS SEMAPHORIN VIA MRNA).

NOV4b

A disclosed NOV4b nucleic acid of 3025 nucleotides (designated CuraGen Acc. No. CG59253-02) encoding a novel semaphorin-like protein is shown in Table 4C. An open reading frame was identified beginning with an ATG initiation codon at nucleotides 46-48 and ending with a TAG codon at nucleotides 3151-3153. Putative untranslated regions upstream of the initiation codon and downstream from the termination codon is underlined in Table 4C, and the start and stop codons are in bold letters.

TABLE 4C
NOV4b Nucleotide Sequence

| | |
|---|---|
| TGGCATTTCTGAGCAGGGGCCACCCTGACTTCACCTTGGCCCACCATGAGCGTCTTCCTG | (SEQ ID NO:11) |
| CTTTGTGCCTACATACTGCTGCTGATGGTTTCCCAGTTGAGGGCAGTCAGCTTTCCTGAA | |
| GATGATGAACCCCTTAATACTGTCGACTATCACTGTAAGTCGTCTAGGC~ATATCCGGTT | |
| TTTAGAGGACGCCCTTCAGGCAATGAATCGCAGCACAGGCTGGACTTTCACCTGATGTTG | |
| AAAATTCGAGACACACTTTATATTGCTGGCAGGGATCAAGTTTATACAGTAAACTTAAAT | |
| GAAATGCCCAAAACAGAAGTAATATGGCAACAGAAACTGACATGGCGATCAAGACAACAG | |
| GATCGAGAAAACTGTGCTATGAAAGGCAAGCATAAAGATGAATGCCACAACTTTATCAAA | |
| GTATTTGTTCCAAGAAACGATGAGATGGTTTTTGTTTGTGGTACCAATGCATTCAATCCC | |
| ATGTGTAGATACTACAGGGTAAGTACCTTAGAATATGATCGGGAAGAAATTAGTGGCCTG | |
| GCAAGATGCCCATTTGATGCCAGACAAACCAATGTTGCCCTCTTTGCTGATGGGAAGCTG | |
| TATTCTGCCACAGTGGCTGACTTCTTGGCCAGCGATGCCGTTATTTATCGAAGCATGGGT | |
| GATGGATCTGCCCTTCGCACAATAAAATATGATTCCAAATGCATAAAAGAGCCACACTTT | |
| CTTCATGCCATAGAATATGGAAACTATGTCTATTTCTTCTTTCGAGAAATCGCTGTCGAA | |
| CATAATAATTTAGGCAAGGCTGTGTATTCCCGCGTGGCCCGCATATGTAAAAACGACATG | |
| GGTGGTTCCCAGCGGGTCCTGGAGAAACACTGGACTTCATTTCTAAACGCTCGGCTGAAC | |
| TGTTCTGTCCCTGGAGATTCGTTTTTCTACTTTGATGTTCTCCAGTCTATTACAGACATA | |
| ATACAAATCAATGCCATCCCCACTGTGGTCGCGGTGTTTACCACCCAGCTCAATAGCATC | |
| CCTGGTTCTGCTGTCTGTGCATTTAGCATGGATGACATTGAAAAAGTATTCAAAGGACGG | |
| TTTAAGGAACAGAAAACTCCAGATTCTGTTTGGACAGCAGTTCCCGAAGACAAAGTGCCA | |
| AAGCCAAGGCCTGGCTGTTGTGCAAAACACGGCCTTGCCGAAGCTTATAAAACCTCCATC | |
| GATTTCCCGGATGAAACTCTGTCATTCATCAAATCTCATCCCCTGATGGACTCTGCCGTT | |
| CCACCCATTGCCGATGAGCCCTGGTTCACAAAGACTCGGGTCAGGTACAGACTGACCCCC | |
| ATCTCAGTGGACCATTCAGCCCGACCCTACCAGAACTACACAGTCATCTTTGTTGGCTCT | |
| GAAGCTGGCATGGTACTTAAAGTTCTGGCAAAGACCAGTCCTTTCTCTTTGAACGACAGC | |
| GTATTACTGGAAGAGATTGAAGCCTACAACCATGCAAAGTGCAGTGCTGAGAATGAGGAA | |
| GACAAAAAGGTCATCTCATTACAGTTGGATAAAGATCACCACGCTTTATATGTGGCGTTC | |
| TCTAGCTGCATTATCCGCATCCCCCTCAGTCGCTGTGAGCGTTATGGATCATGTAAAAAG | |
| TCTTGTATTCCATCTCGTGACCCGTATTGTGGCTGGTTAAGCCAGCCATCCTGTGGTACA | |
| GTGACCCCAAACCACAGTGCTGAAGGATATGAACAAGACACAGAATTCGGCAACACAGCT | |
| CATCTAGGGGACTGCCATGCATATCAACCATATGAAGGTCGTGTTGGCTCACTGAAAGCC | |
| ATTTGCTATTTATTATTATTTTTAAAAAGCACCTTATTCACATTGTCCCATGTGTCTATT | |
| TCAGGTGTACGATGGGAAGTCCAGTCTGGAGACTCCAACCAGATGGTCCACATGAATGTC | |
| CTCATCACCTGTGTCTTTGCTGCTTTTGTTTTGGGGGCATTCATTGCAGGTGTGGCAGTA | |
| TACTGCTATCGAGACATGTTTGTTCGGAAAAACAGAAAGATCCATAAAGATGCAGAGTCC | |
| GCCCAGTCATGCACAGACTCCAGTCGAAGTTTTGCCAAACTGAATGGTCTCTTTGACAGC | |
| CCTGTCAAGGAATACCAACAGAATATTGATTCTCCTAAACTGTATAGTAACCTGCTAACC | |
| AGTCGGAAAGAGCTACCACCCAATGGAGATACTAAATCCATGGTAATGGACCATCGAGGG | |
| CAACCTCCAGACTTGGCTGCTCTTCCTACTCCTGAGTCTACACCCGTGCTTCACCAGAAG | |
| ACCCTGCAGGCCATGAAGAGCCACTCAGAAAAGGCCCATGGCCATGGAGCTTCAAGGAAA | |
| GAAACCCCTCAGTTTTTTCCGTCTAGTCCGCCACCTCATTCCCCATTAAGTCATGCGCAT | |
| ATCCCCAGTGCCATTGTTCTTCCAAATGCTACCCATGACTACAACACGTCTTTCTCAAAC | |
| TCCAATGCTCACAAAGCTGAAAAGAAGCTTCAAAACATTGATCACCCTCTCACAAAGTCA | |
| TCCAGTAAGAGAGATCACCGGCGTTCTGTTGATTCCAGAAATACCCTCAATGATCTCCTG | |
| AAGCATCTGAATGACCCAAATAGTAACCCCAAAGCCATCATGGGAGACATCCAGATGGCA | |
| CACCAGAACTTAATGCTGGATCCCATGGGATCGATGTCTGAGGTCCCACCTAAAGTCCCT | |
| AACCGGGAGGCATCGCTATACTCCCCTCCTTCAACTCTCCCCAGAAATAGCCCAACCAAG | |
| CGAGTGGATGTCCCCACCACTCCTGWGGTCCCAATGACTTCTCTGGAAAGACAAACAGCT | |
| TATCACAAAAATTCCTCCCAGAGGCACTCTATATCTGCTATGCCTAAAAACTTAAACTCA | |
| CCAAATGGTGTTTTGTTATCCAGACAGCCTACTATGAACCGTCGAGGATATATGCCCACC | |
| CCCACTGGGGCGAAGGTGGACTATATTCAGGGAACACCAGTGAGTGTTCATCTGCAGCCT | |
| TCCCTCTCCAGACAGAGCAGCTACACCAGTAATGGCACTCTTCCTAGGACGGGACTAAAG | |
| AGGACGCCGTCCTTAAAACCTGACGTGCCACCAAAGCCTTCCTTTGTTCCTCAAACCCCA | |
| TCTGTCAGACCACTGAACAAATACACATACTAGGCCTCAAGTGTGCTATTCCCATGTGGC | |
| TTTATCCTGTCCGTCTTGTTGAGAG | |

The nucleic acid sequence of NOV4b maps to chromosome 15q21.1 and has 1134 of 1656 bases (68%) identical to a mouse semaphorin IV mRNA (Accession No. AF030430) (E=1.0e⁻¹³²).

A NOV4b polypeptide (SEQ ID NO:18) encoded by SEQ ID NO:17 is 1035 amino acid residues and is presented using the one letter code in Table 4D. Signal P, Psort and/or Hydropathy results predict that NOV4b has a signal peptide and is likely to be localized at the plasma membrane with a certainty of 0.4600. In other embodiments, NOV4b may also be localized to the endoplasmic reticulum (membrane or lumen) with a certainty of 0.1000, or outside the cell with a certainty of 0.1000. The most likely cleavage site is between positions 20 and 21 (LRA-VS).

TABLE 4D
NOV4b protein sequence

| | |
|---|---|
| MRVFLLCAYILLLNVSQLFAVSFPEDDEPLNTVDYHCKSSRQYPVFRGRPSGNESQHRLD | (SEQ ID NO:12) |
| FQLMLKIRDTLYIAGRDQVYTVNLNEMPKTEVIWQQKLTWRSRQQDRENCAMKGKHKDEC | |
| HNFIKVFVPRNDEMVFVCGTNAFNPMCRYYRVSTLEYDGEEISGLARCPFDARQTNVALF | |
| ADGKLYSATVADFLASDAVIYRSMGDGSALRTIKYDSKWIKEPHFLHAIEYGNYVYFFFR | |
| EIAVEHWNLGKAVYSRVARICKNDMGGSQRVLEKHWTSFLKARLNCSVPGDSFFYFDVLQ | |
| SITDIIQINGIPTVVGVFTTQLNSIPGSAVCAFSMDDIEKVFKGRFKEQKTPDSVWTAVP | |
| EDKVPKPRPGCCAKHGLAEAYKTSIDFPDETLSFIKSHPLMDSAVPPIADEPWPTKTRVR | |
| YRLTAISVDHSAGPYQNYTVIFVGSEAGMVLKVLAKTSPFSLNDSVLLEEIEAYNBAKCS | |
| AENEEDKKVISLQLDKDHHAIYVAFSSCTIRIPLSRCERYGSCKXSCIASRDPYCGWLSQ | |
| GSCGRVTPNHSAEGYEQDTEFGNTAHLGDCHAYEPYEGRVGSLKAICYLLLFLKSTLFTL | |
| SHVSISGVRWEVQSGESNQMVHNNVLITCVFAAFVLGAFIAGVAVYCYRDMFVRKNRKTH | |
| KDAESAQSCTDSSGSFAKLNGLFDSPVKEYQQNIDSPKLYSNLLTSRKELPPNGDTKSMV | |
| MDHRGQPPELAALPTPESTPVLHQKTLQAMKSHSEKAHGHGASRKETPQFFPSSPPPHSP | |
| LSHGHIPSAIVLPNATHDYNTSFSNSNAHKAEKKLQNIDEPLTKSSSKRDHRRSVDSRNT | |
| LNDLLKHLNDPNSNPKAIMGDIQMAHQNLMLDPMGSMSEVPPKVPNREASLYSPPSTLPR | |
| NSPTKRVDVPTTPGVPMTSLERQRGYHKNSSQRHSISAMPKNLNSPNGVLLSRQPSMNRG | |
| GYMPTPTGAKVDYIQGTFVSVHLQPSLSRQSSYTSNGTLPRTGLKRTPSLKPDVPPKPSF | |
| VPQTPSVRPLNKYTY | |

The full amino acid sequence of the protein of the invention was found to have 354 of 583 amino acid residues (60%) identical to, and 448 of 583 amino acid residues (76%) similar to, the 1030 amino acid residue ptnr:TREMBLNEW-ACC:Q9H2E6 semaphorein 6A1 protein from Homo sapiens (E=1.1e⁻²²²).

NOV4b is expressed in at least the following tissues: dipose, heart, pancreas, thyroid, liver, gall bladder, colon, brain, right cerebellum, left cerebellum, thalamus, hypothalamus, frontal lobe, parietal lobe, cerebral medulla/cerebral white matter, substantia nigra, hippocampus, spinal cord, peripheral nerves, mammary gland/breast, ovary, placenta, lung, kidney, skin, foreskin, and epidermis. Expression information was derived from the tissue sources of the sequences that were included in the derivation of the sequence of CuraGen Acc. No. CG59253-01.

NOV4c

A disclosed NOV4c nucleic acid of 2191 nucleotides (designated CuraGen Acc. No. CG59253-05) encoding a novel semaphorin-like protein is shown in Table 4E. An open reading frame was identified beginning with an ATG initiation codon at nucleotides 46-48 and ending with a TAG codon at nucleotides 2182-2184. Putative untranslated regions upstream of the initiation codon and downstream from the termination codon is underlined in Table 4E, and the start and stop codons are in bold letters.

TABLE 4E
NOV4c Nucleotide Sequence

| | |
|---|---|
| TGGCATTTCTGACCAGGGGCCACCCTGACTTCACCTTGGCCCACCATGAGGGTCTTCCTG | (SEQ ID NO:13) |
| CTTTGTGCCTACATACTGCTGCTGATGGTTTCCCAGTTGAGGGCAGTCAGCTTTCCTGAA | |
| GATGATGAACCCCTTAATACTGTCGACTATCACTGTAAGTCGTCTAGGCAATATCCGGTT | |
| TTTAGAGGACGCCCTTCAGGCAATGAATCGCAGCACAGGCTGGACTTTCAGCTGATGTTG | |
| AAAATTCGAGACACACTTTATATTGCTGGCAGGGATCAAGTTTATACAGTAAACTTAAAT | |
| GAAATGCCCAAAACAGAAGTAATATGGCAACAGAAACTGACATCGCGATCAAGACAACAG | |
| GATCGAGAAAACTGTGCTATGAAAGGCAAGCATAAAGATGAATGCCACAACTTTATCAAA | |
| GTATTTGTTCCAAGAAACGATGAGATGGTTTTTGTTTGTGGTACCAATGCATTCAATCCC | |
| ATGTGTAGATACTACAGGGTAAGTACCTTAGAATATGATGGGGAAGAAATTAGTGGCCTG | |
| GCAAGATGCCCATTTGATGCCAGACAAACCAATGTTGCCCTCTTTGCTGATGGGAAGCTG | |
| TATTCTGCCACAGTGGCTGACTTCTTGGCCAGCGATGCCGTTATTTATCGAAGCATGGGT | |
| GATGGATCTGCCCTTCGCACAATAAAATATGATTCCAAATGGATAAAAGAGCCACACTTT | |
| CTTCATGCCATACAATATGGAAACTATGTCTATTTCTTCTTTCGAGAAATCGCTGTCGAA | |
| CATAATAATTTAGGCAAGGCTGTGTATTCCCGCGTGGCCCGCATATGTAAAAACGACATG | |
| GGTGGTTCCCAGCGGGTCCTGGAGAAACACTGGACTTCATTTCTAAAGGCTCGGCTGPAC | |
| TGTTCTGTCCCTGGAGATTCGTTTTTCTACTTTGATGTTCTGCAGTCTATTACAGACATA | |
| ATACAAATCAATGGCATCCCCACTGTGGTCGGGGTGTTTACCACGCAGCTCAATAGCATC | |
| CCTGGTTCTGCTGTCTGTGCATTTAGCATGGATGACATTGAAAAAGTATTCAAAGGACGG | |
| TTTAAQGAACAGAAAACTCCAAATTCTGTTTGGACAGCAGTTCCCGAAGACAAAGTGCCA | |
| AAGCCAAGGCCTGGCTGTTGTGCAAAACACGGCCTTGCCGAAGCTTATAAAACCTCCATC | |
| GATTTCCCGGATGAAACTCTGTCATTCATCAAATCTCATCCCCTGATGGACTCTGCCGTT | |
| CCACCCATTGCCGATGAGCCCTGGTTCACAAAGACTCGGGTCAQGTACAGACTGACGGCC | |
| ATCTCAGTGGACCATTCAGCCGGACCCTACCAGAACTACACAGTCATCTTTGTTGGCTCT | |
| GAAGCTGGCATGGTACTTAAAGTTCTGGCAAAGACCAGTCCTTTCTCTTTGAACGACAGC | |
| GTATTACTGGAAGAGATTGAAGCCTACAACCATGCAAAGTGCAGTGCTGAGAATGAGGAA | |
| GACAAAAAGGTCATCTCATTACAGTTGGATAAAGATCACCACGCTTTATATCTCGCGTTC | |
| TCTAGCTGCATTATCCGCATCCCCCTCAGTCGCTGTGAGCGTTATGGATCATGTAAAAAG | |
| TCTTGTATTGCATCTCGTGACCCGTATTGTGGCTGGTTAAGCCAGGGATCCTGTGGTAGA | |
| GTGACCCCAGGGATGCTGCTGTTAACCGAAGACTTCTTTGCTTTCCATAACCACAGTGCT | |
| GAAGGATATGAACAAGACACAGAATTCGGCAACACAGCTCATCTAGGGGACTGCCATGAA | |
| ATTTTGCCTACTTCAACTACACCAGATTACAAAATATTTGGCGGTCCAACATCTCGTGTA | |
| CGATCGGAAGTCCAGTCTGGAGAGTCCAACCAGATGGTCCACATGAATGTCCTCATCACC | |
| TGTGTCTTTGCTGCTTTTGTTTTGGGGGCATTCATTGCAGGTGTGGCAGTATACTGCTAT | |
| CGAGACATGTTTGTTCGGAAAAACAGAAAGATCCATAAAGATGCAGAGTCCGCCCAGTCA | |
| TGCACAGACTCCAGTGGAAGTTTTGCCAAACTGAATGGTCTCTTTGACAGCCCTGTCAAG | |
| GAATACCAACAGAATATTGATTCTCCTAAACTGTATAGTAACCTGCTAACCAGTCGGAAA | |
| GAGCACGAATTCAGCGGCCGCTGAATTCTAG | |

The nucleic acid sequence of NOV4c maps to chromosome 15 and has 1161 of 1166 bases (99%) identical to a gb:GENBANK-ID:AK021660|acc:AK021660.1 mRNA from Homo sapiens (Homo sapiens cDNA FLJ11598 fis, clone HEMBA1003866, moderately similar to Mus musculus semaphorin VIa mRNA).

A NOV4c polypeptide (SEQ ID NO:18) encoded by SEQ ID NO:17 is 712 amino acid residues and is presented using the one letter code in Table 4D. Signal P, Psort and/or Hydropathy results predict that NOV4c has a signal peptide and is likely to be localized at the plasma membrane with a certainty of 0.4600. In other embodiments, NOV4c may also be localized to the microbody with a certainty of −0.1812, or to the endoplasmic reticulum (membrane or lumen) with a certainty of 0.1000. The most likely cleavage site is between positions 20 and 21 (LRA-VS).

TABLE 4F
NOV4c protein sequence

| | |
|---|---|
| MRVFLLCAYILLLMVSQLRAVSFPEDDEPLNTVDYHCKSSRQYPVFRGRPSCNESQHRLD | (SEQ ID NO:14) |
| FQLMLKIRDTLYIAGRDQVYTVNLNEMPKTEVIWQQKLTWRSRQQDRENCAMKGKHKDEC | |
| HNFIKVFVPRNDEMVFVCGTNAFNPMCRYYRVSTLEYDGEEISGLARCPFDARQTNVALF | |
| ADGKLYSATVADFLASDAVIYRSMGDGSALRTIKYDSKWIKEPHFLHAIEYGNYVYFFFR | |
| EIAVEHNNLGKAVYSRVARICKNDMGGSQRVLEKHWTSFLKARLNCSVPGDSFFYFDVLQ | |
| STTDIIQINGIPTVVGVFTTQLNSIPGSAVCAFSMDDIEKVFKGRFKEQKTPDSVWTAVP | |
| EDKVPKPRPGCCAKHGLAEAYKTSIDFPDETLSFIKSHPLMDSAVPPIADEPWFTKTRVR | |
| YRLTATSVDHSAGPYQNYTVIFVGSEAGMVLKVLAKTSPFSLNDSVLLEEIEAYNRAKCS | |
| AENEEDKKVISLQLDKDHHALYVAFSSCIIRIPLSRCERYGSCKKSCIASRDPYCGWLSQ | |
| GSCGRVTPGMLLLTEDFFAFHNHSAEGYEQDTEFGNTAHLGDCHEILPTSTTPDYKIFGG | |
| PTSGVRWEVQSGESNQMVHMNVLITCVFAAFVLGAFIACVAVYCYRDMFVRKNRKIHKDA | |
| ESAQSCTDSSGSEAKLNGLFDSPVKEYQQNIDSPKLYSNLLTSRKEHEFSCR | |

The full amino acid sequence of the protein of the invention was found to have 577 of 586 amino acid residues (98%) identical to, and 580 of 586 amino acid residues (98%) similar to, the 1022 amino acid residue ptnr:TREMBLNEW-ACC:BAA96003 protein from Homo sapiens (Human) (KIAA1479 PROTEIN).

NOV4c is expressed in at least the following tissues: whole embryo, mainly head and neck.

NOV4d

A disclosed NOV4d nucleic acid of 3196 nucleotides (designated CuraGen Acc. No. CG59253-06) encoding a novel semaphorin-like protein is shown in Table 4E. An open reading frame was identified beginning with an ATG initiation codon at nucleotides 46-48 and ending at nucleotides 3142-3144. Putative untranslated regions upstream of the initiation codon and downstream from the termination codon is underlined in Table 4E, and the start and stop codons are in bold letters.

TABLE 4G
NOV4d Nucleotide Sequence

| | |
|---|---|
| TGGCATTTCTGAGCAGGGGCCACCCTGACTTCACCTTGGCCCACCATGAGGGTCTTCCTG | (SEQ ID NO:15) |
| CTTTGTGCCTACATACTGCTGCTGATGGTTTCCCAGTTGAGGGCAGTCAGCTTTCCTGAA | |
| GATGATGAACCCCTTAATACTGTCGACTATCACTGTAAGTCGTCTAGGCAATATCCGGTT | |
| TTTAGAGGACGCCCTTCAGGCAATGAATCGCAGCACAGGCTGGACTTTCAGCTGATGTTG | |
| AAAATTCGAGACACACTTTATATTGCTGGCAGGGATCAAGTTTATACAGTAAACTTAAAT | |
| GAAATGCCCAAAACAGAAGTAATATGGCAACAGAAACTGACATGGCGATCAAGACAACAG | |
| GATCGACAAAACTGTGCTATGAAAGGCAAGCATAAAaATGAATGCCACAACTTTATCAAA | |
| GTATTTGTTCCAAGAAACGATGAGATGGTTTTTGTTTGTCGTACCAATGCATTCAATCCC | |
| ATGTGTAGATACTACAGGGTAAGTACCTTAGAATATGATGGGGAAGAAATTAGTGGCCTG | |
| GCAAGATGCCCATTTGATGCCAGACAAACCAATGTTGCCCTCTTTGCTGATGGGAAGCTG | |
| TATTCTGCCACAGTGGCTGACTTCTTGGCCAGCCATGCCGTTATTTATCGAAGCATGGGT | |
| GATGGATCTGCCCTTCGCACAATAAAATATGATTCCAAATGGATAAAAGAGCCACACTTT | |
| CTTCATGCCATAGAATATGGAAACTATGTCTATTTCTTCTTTCGACAAATCGCTGTCGAA | |
| CATAATAATTTAGGCAAGGCTGTGTATTCCCGCGTGGCCCGCATATGTAAAAACCACATG | |
| GGTGGTTCCCAGCGGGTCCTCGAGAAACACTGGACTTCATTTCTAAAGGCTCGGCTGAAC | |
| TGTTCTGTCCCTGGAGATTCGTTTTTCTACTTTGATGTTCTGCAGTCTATTACAGACATA | |
| ATACAAATCAATGGCATCCCCACTGTGGTCGGGGTGTTTACCACGCAGCTCAATAGCATC | |
| CCTCGTTCTGCTGTCTGTGCATTTAGCATGGATGACATTGAAAAAGTATTCAAAGGACGG | |
| TTTAAGGAACAGAAAACTCCAGATTCTGTTTGGACAGCAGTTCCCGAAGACAAAGTGCCA | |
| AAGCCAAGGCCTGGCTGTTGTGCAAAACACGGCCTTGCCGAAGCTTATAAAACCTCCATC | |
| GATTTCCCGGATGAAACTCTGTCATTCATCAAATCTCATCCCCTGATGGACTCTGCCGTT | |
| CCACCCATTGCCGATGAGCCCTGGTTCACAAAGACTCGGGTCAGGTACACACTGACGGCC | |
| ATCTCAGTGGACCATTCAGCCGGACCCTACCAGAACTACACAGTCATCTTTGTTGGCTCT | |
| GAAGCTGGCATGGTACTTAAAGTTCTGGCAAAGACCAGTCCTTTCTCTTTGAACGACAGC | |
| GTATTACTGGAAGAGATTGAAGCCTACAACCATGCAAAGTGCAGTGCTGAGAATGAGGAA | |
| GACAAAAAGGTCATCTCATTACAGTTGGATAAAGATCACCACGCTTTATATGTGGCGTTC | |
| TCTAGCTGCATTATCCGCATCCCCCTCAGTCGCTGTGAGCGTTATGGATCATGTAAAAAG | |
| TCTTGTATTGCATCTCGTGACCCGTATTGTCGCTGGTTAAGCCAGGGATCCTGTGGTAGA | |
| GTGACCCCAGGGATGCTGCTGTTAACCGAAGACTTCTTTGCTTTCCATAACCACAGTGCT | |
| GAACCATATGAACAAGACACAGAATTCGGCAACACAGCTCATCTAGGGGACTGCCATGAA | |
| ATTTTGCCTACTTCAACTACACCAGATTACAAAATATTTGGCGGTCCAACATCTGGTGTA | |
| CGATGGGAAGTCCAGTCTGGAGAGTCCAACCAGATGGTCCACATGAATGTCCTCATCACC | |
| TGTGTCTTTGCTGCTTTTGTTTTGGGGGCATTCATTGCAGGTGTCGCAGTATACTGCTAT | |
| CGAGACATGTTTGTTCGGAAAAACAGAAAGATCCATAAAGATGCAGAGTCCGCCCAGTCA | |
| TGCACAGACTCCAGTGGAAGTTTTGCCAAACTGAATGGTCTCTTTGACAGCCCTGTCAAG | |
| GAATACCAACAGAATATTGATTCTCCTAAACTGTATAGTAACCTGCTAACCAGTCGGAAA | |
| GAGCTACCACCCAATGGAGATTCTAAATCCATGGTAATGGACCATCGAGGGCAACCTCCA | |
| GAGTTGGCTGCTCTTCCTACTCCTGAGTCTACACCCGTGCTTCACCAGAAGACCCTGCAG | |
| GCCATGAAGAGCCACTCAGAAAAGGCCCATGGCCATGGAGCTTCAAGGAAAGAAACCCCT | |
| CAGTTTTTTCCGTCTAGTCCGCCACCTCATTCCCCATTAAGTCATGGGCATATCCCCAGT | |
| GCCATTGTTCTTCCAAATGCTACCCATGACTACAACACGTCTTTCTCAAACTCCAATGCT | |
| CACAAAGCTGAAAAGAAGCTTCAAAACATTGATCACCCTCTCACAAAGTCATCCAGTAAG | |
| AGAGATCACCGGCGTTCTGTTGATTCCAGAAATACCCTCAATGATCTCCTGAAGCATCTG | |
| AATGACCCAAATAGTAACCCCAAAGCCATCATGGGAGACATCCAGATGGCACACCAGAAC | |
| TTAATGCTGGATCCCATGGGATCGATGTCTGAGGTCCCACCTAAAGTCCCTAACCGGCAG | |
| GCATCGCTATACTCCCCTCCTTCAACTCTCCCCAGAAATAGCCCAACCAAGCGAGTGGAT | |
| GTCCCCACCACTCCTGGAGTCCCAATGACTTCTCTGGAAAGACAAAGAGGTTATCACAAA | |
| AATTCCTCCCAGAGGCACTCTATATCTGCTATGCCTAAAAACTTAAACTCACCAAATGGT | |
| GTTTTGTTATCCAGACAGCCTAGTATGAACCGTGGAGGATATATGCCCACCCCCACTGGG | |
| GCGAAGGTGGACTATATTCAGCGAACACCAGTGAGTGTTCATCTGCAGCCTTCCCTCTCC | |
| AGACAGAGCAGCTACACCAGTAATGGCACTCTTCCTAGGACCGOACTAAAGAGGACGCCG | |
| TCCTTAAAACCTGACGTGCCACCAAAGCCTTCCTTTGTTCCTCAAACCCCATCTGTCAGA | |
| CCACTGAACAAATACACATACTAGGCCTCAAGTGTGCTATTCCCATGTGGCTTTATCCTG | |
| TCCGTGTTGTTGAGAG | |

The nucleic acid sequence of NOV4d maps to chromosome 15 and has 1786 of 1798 bases (99%) identical to a gb:GENBANK-ID:AB040912|acc:AB040912.2 mRNA from Homo sapiens (Homo sapiens mRNA for KIAA1479 protein, partial cds).

A NOV4d polypeptide (SEQ ID NO:18) encoded by SEQ ID NO:17 is 1032 amino acid residues and is presented using the one letter code in Table 4D.

TABLE 4H
NOV4d protein sequence

| | |
|---|---|
| MRVFLLCAYILLLMVSQLPAVSFPEDDEPLNTVDYHCKSSRQYPVFRCRPSGNESQHRLD | (SEQ ID NO:16) |
| FQLMLIURDTLYIAGPDQVYTVNLNEMPKTEVIWQQKLTWRSRQQDRENCAMKGKHKDEC | |
| HNFIKVFVPRNDEMVFVCGTNAFNPMCRYYRVSTLEYDGEEISGLARCPFDARQTNVALF | |
| ADGKLYSATVADFLASDAVIYRSMGDGSALRTIKYDSKWIKEPHFLHAIEYGNYVYFFFR | |
| EIAVEHNNLGKAVYSRVARICKNDMGGSQRVLEKHWTSFLKARLNCSVPGDSFFYFDVLQ | |
| SITDIIQINGIPTVVGVFTTQLNSIPGSAVCAFSMDDIEKVFKGRFKEQKTPDSVWTAVP | |
| EDKVPKPRPGCCAKHGLAEAYKTSIDFPDETLSFIKSHPLMDSAVPPIADEPWFTKTRVR | |
| YPITAISVDHSAGPYQNYTVIFVGSEAGMVLKVLAKTSPFSLNDSVLLEEIEAYNHAKCS | |
| AENEEDKKVISLQLDKDHHALYVAFSSCIIRIPLSRCERYGSCKXSCIASRDPYCGWLSQ | |
| GSCGRVTPGMLLLTEDFFAFHNHSAEGYEQDTEFGNTAHLGDCHEILPTSTTPDYKIFGG | |
| PTSGVRWEVQSGESNQMVHMNVLITCVFAAFVLGAFIAGVAVYCYRDMFVRKNRKIHKDA | |
| ESAQSCTDSSGSFAKLNGLFDSPVKEYQQNIDSPKLYSNLLTSRKELPPNGDSKSMVMDH | |
| RGQPPELAALPTPESTPVLHQKTLQAMKSHSEKAHGHGASRKETPQFFPSSPPPHSPLSH | |
| GHIPSAIVLPNATHDYNTSFSNSNAHKAEKKLQNIDHPLTKSSSKRDHRRSVDSRNTLND | |
| LLKHLNDPNSNPKAIMGDIQMAHQNLMLDPMGSMSEVPPKVPNREASLYSPPSTLPRNSP | |
| TKRVDVPTTPGVPMTSLERQRGYHKNSSQRHSISAMPKNLNSPNGVLLSRQPSMNRGGYM | |
| PTPTGAKVDYIQGTPVSVHLQPSLSRQSSYTSNGTLPRTGLKRTPSLKPDVPPKPSFVPQ | |
| TPSVRPLNKYTY | |

The full amino acid sequence of the disclosed NOV4e protein was found to have 577 of 586 amino acid residues (98%) identical to, and 580 of 586 amino acid residues (98%) similar to, the 1022 amino acid residue ptnr:TREMBLNEW-ACC:BAA96003 protein from Homo sapiens (Human) (KIAA1479 PROTEIN).

NOV4e

A disclosed NOV4e nucleic acid of 2359 nucleotides (designated CuraGen Acc. No. CG59253-07) encoding a novel semaphorin-like protein is shown in Table 4E. An open reading frame was identified beginning with an ATG initiation codon at nucleotides 46-48 and ending at nucleotides 2350-2352. Putative untranslated regions upstream of the initiation codon and downstream from the termination codon is underlined in Table 4E, and the start and stop codons are in bold letters.

TABLE 41
NOV4e Nucleotide Sequence

| | |
|---|---|
| TGGCATTTCTGAGCAGGGGCCACCCTGACTTCACCTTGGCCCACCATGAGGGTCTTCCTG | (SEQ ID NO:17) |
| CTTTGTGCCTACATACTGCTGCTGATGGTTTCCCAGTTGAGGGCAGTCAGCTTTCCTGAA | |
| GATGATGAACCCCTTAATACTGTCGACTATCACTGTAAGTCGTCTAGGCAATATCCGGTT | |
| TTTAGAGGACGCCCTTCAGGCAATGAATCGCAGCACAGGCTGGACTTTCAGCTGATGTTG | |
| AAAATTCGAGACACACTTTATATTGCTGGCAGGGATCAAGTTTATACAGTAAACTTAAAT | |
| GAAATGCCCAAAACAGAAGTAATATGGCAACAGAAACTGACATGGCGATCAAGACAACAG | |
| GATCGAGAAAACTGTGCTATGAAAGGCAAGCATAAAGATGAATGCCACAACTTTATCAAA | |
| GTATTTGTTCCAAGAAACGATGAGATGGTTTTTGTTTGTGGTACCAATGCATTCAATCCC | |
| ATGTGTAGATACTACAGGGTAAGTACCTTAGAATATGATGGGGAAGAAATTAGTGGCCTG | |
| GCAAGATGCCCATTTGATGCCAGACAACCAATGTTGCCCTCTTTGCTGAAGTGGAAGCTG | |
| TATTCTGCCACAGTGGCTGACTTCTTGGCCAGCGATGCCGTTATTTATCGAAGCATGGGT | |
| GATGGATCTGCCCTTCGCACAATAAAAATATGATTCCAAATGGATAAAAGAGCCACATTT | |
| CTTCATGCCATAGAATATGGAAACTATGTCTATTTCTTCTTTCGAGAAATCGCTGTCGAA | |
| CATAATAATTTAGGCAAGGCTGTGTATTCCCGCGTGGCCCGCATATGTAAAAACGACATG | |
| GGTGGTTCCCAGCGGGTCCTGGAGAAACACTGGACTTCATTTCTAAAGGCTCGGCTGAAC | |
| TGTTCTGTCCCTGGAGATTCGTTTTTCTACTTTGATGTTCTGCAGTCTATTACAGACATA | |
| ATACAATCAATGGCATCCCCACTGTGGTCGGGGTGTTTACCACGCAGCTCAATAGCATGC | |
| CCTGGTTCTGCTGTCTGTGCATTTAGCATGGATGACATTGAAAAAGTATTCAAAGGACGG | |
| TTTAAGGAACAGAAAACTCCAGATTCTGTTTGGACAGCAGTTCCCGAAGACAAAGTGCCA | |
| AAGCCAAGGCCTGGCTGTTGTGCAAAACACGGCCTTGCCGAAGCTTATAAAACCTCCATC | |
| GATTTCCCGGATCAAACTCTGTCATTCATCAAATCTCATCCCCTGATGGACTCTGCCGTT | |
| CCACCCATTGCCGATGAGCCCTGGTTCACAAAGACTCGGGTCAGGTACAGACTGACGGCC | |
| ATCTCAGTGGACCATTCACCCGGACCCTACCAGAACTACAcAGTCATCTTTGTTGGCTCT | |
| GAAGCTGGCATGGTACTTAAGTTCTGGCAAAGACCAGTCCTTTCTCTTTTGAACGACAGC | |
| GTATTACTGGAAGAGATTGAAGCCTACAACCATGCAAAGTGCAGTGCTGAGAATGAGGAA | |
| GACAAAAAGGTCATCTCATTACAGTTGGATAAAGATCACCACGCTTTATATGTGGCGTTC | |
| TCTAGCTGCATTATCCGCATCCCCCTCAAGTCGCTGTGAGCGTTATGGATCATGTAAAAG | |
| TCTTGTATTGCATCTCGTGACCCGTATTGTGGCTGGTTAAGCCAGGGATCCTGTGGTAGA | |
| GTGACCCCAGGGATGCTGCTGTTAACCGAAGACTTCTTTGCTTTCCATAACCACAGTGCT | |
| GAAGGATATGAACAAGACACAGAATTCGGCAACACAGCTCATCTAGGGGACTGCCATGAA | |
| ATTTTGCCATACTTCAACTACACCAGATTACAAATATTTGGCGGTCCAACATCTGACATG | |
| GAGGTATCTTCATCTTCTGTTACCACAATGGCAAGTATCCCAGAAATCACACCTAAAGTG | |
| ATTGATACCTGGAGACCTAAACTGACAAGCTCTCGGAAATTTGTAGTTCAAGATGATCCA | |
| AACACTTCTGATTTTACTGATCCTTTATCGGGTATCCCAAAGGGTGTACCATGGGAAGTC | |
| CAGTCTGGAGAGTCCAACCAGATGGTCCACATGAATGTCCTCATCACCTGTGTCTTTGCT | |
| GCTTTTGTTTTGGGGGCATTCATTGCAGGTGTGGCAGTATACTGCTATCGAGACATGTTT | |
| GTTCGGAAAAACAGAAAGATCCATAAAGATGCAGAGTCCGCCCAGTCATGCACAGACTCC | |
| AGTGGAAGTTTTGCCAAACTGAATGGTCTCTTTGACAGCCCTGTCAAGGAATACCAACAG | |
| AATATTGATTCTCCTAAACTGTATAGTAACCTGCTAACCAGTCGGAAAGAGCACGAATTC | |
| AGCGGCCGCTGAATTCTAG | |

The nucleic acid sequence of NOV4e maps to chromosome 15.

A NOV4e polypeptide (SEQ ID NO:18) encoded by SEQ ID NO:17 is 768 amino acid residues and is presented using the one letter code in Table 4e.

TABLE 4J
NOV4e protein sequence

| | |
|---|---|
| MRVFLLCAYILLLMVSQLRAVSFPEDDEPLNTVDYHCKSSRQYPVFRGRPSGNESQHRLD | (SEQ ID NO:18) |
| FQLMLKIRDTLYIAGRDQVYTVNLNEMPKTEVIWQQKLTWRSRQQDRENCAMKGKHKDEC | |
| HNFIKVFVPRNDEMVFVCGTNAFNPMCRYYRVSTLEYDGEEISGLARCPFDARQTNVALF | |
| ADGKLYSATVADFLASDAVIYRSMGDGSALRTIKYDSKWIKEPHFLHAIEYGNYVYFFFR | |
| EIAVEHNNLGKAVYSRVARICKNDMGGSQRVLEKHWTSFLKARLNCSVPGDSFFYFDVLQ | |
| SITDIIQINGIPTVVGVFTTQLNSIPGSAVCAFSMDDIEKVFKCRFKEQKTPDSVWTAVP | |
| EDKVPKPRPGCCAKHGLAEAYKTSIDFPDETLSFIKSHPLMDSAVPPTADEPWFTKTRVR | |
| YRLTAISVDHSAGPYQNYTVIFVGSEAGMVLKVLAKTSPFSLNDSVLLEEIEAYNBAKCS | |
| AENEEDKKVISLQLDKDHHALYVAFSSCIIRIPLSRCERYGSCKKSCIASRDPYCGWLSQ | |
| GSCGRVTPGMLLLTEDFFAFHNHSAEGYEQDTEFGNTAHLGDCHEILPTSTTPDYKIFGG | |
| PTSDMEVSSSSVTTMASIPEITPKVIDTWRPKLTSSRKFVVQDDPNTSDFTDPLSGIPKG | |
| VRWEVQSGESNQMVHMNVLITCVFAAFVLGAFIAGVAVYCYRDMFVRKNRKIHKDAESAQ | |
| SCTDSSGSFAKLNGLFDSPVKEYQQNIDSPKLYSNLLTSRKEHEFSGR | |

NOV4f

A disclosed NOV4f nucleic acid of 3364 nucleotides (designated CuraGen Acc. No. CG59253-08) encoding a novel semaphorin-like protein is shown in Table 4f. An open reading frame was identified beginning with an ATG initiation codon at nucleotides 46-48 and ending at nucleotides 3310-3312. Putative untranslated regions upstream of the initiation codon and downstream from the termination codon is underlined in Table 4f, and the start and stop codons are in bold letters.

TABLE 4K
NOV4f Nucleotide Sequence

| | |
|---|---|
| TGGCATTTCTGAGCAGGGGCCACCCTGACTTCACCTTCGCCCACCATGAGGGTCTTCCTG | (SEQ ID NO:19) |
| CTTTGTGCCTACATACTGCTGCTGATGGTTTCCCAGTTGAGGGCAGTCAGCTTTCCTGAA | |
| GATGATGAACCCCTTAATACTGTCGACTATCACTGTAAGTCGTCTAGGCAATATCCGGTT | |
| TTTAGAGGACGCCCTTCAGGCAATGAATCGCAGCACAGGCTGGACTTTCAGCTGATGTTG | |
| AAAATTCGAGACACACTTTATATTGCTGGCAGGGATCAAGTTTATACAGTAAACTTAAAT | |
| GAAATCCCCAAAACAGAAGTAATATGGCAACAGAAACTGACATGGCGATCAAGACAACAG | |
| GATCGAQAAAACTGTGCTATCAAAGGCAAGCATAAAGATGAATGCCACAACTTTATCAAA | |
| GTATTTGTTCCAAGAAACGATGAGATGGTTTTTGTTTGTGGTACCAATGCATTCAATCCC | |
| ATGTGTAGATACTACAGGGTAAGTACCTTAGAATATGATGGGGAAGAAATTAGTGGCCTG | |
| GCAAGATGCCCATTTGATGCCAGACAAACCAATGTTGCCCTCTTTGCTGATGGGAACCTG | |
| TATTCTGCCACAGTGGCTGACTTCTTGGCCAGCGATGCCGTTATTTATCGAAGCATGCGT | |
| GATGGATCTGCCCTTCGCACAATAAAATATGATTCCAAATGGATAAAAGAGCCACACTTT | |
| CTTCATGCCATAGAATATGGAAACTATGTCTATTTCTTCTTTCGAGAAATCGCTGTCGAA | |
| CATAATAATTTAGGCAAGGCTGTGTATTCCCGCGTGGCCCGCATATGTAAAAACGACATQ | |
| CGTGGTTCCCAGCGGGTCCTGGAGAAACACTGGACTTCATTTCTAAAGGCTCGGCTGAAC | |
| TGTTCTGTCCCTGGAGATTCGTTTTTCTACTTTGATGTTCTGCAGTCTATTACAGACATA | |
| ATACAAATCAATGGCATCCCCACTGTGGTCGGGGTGTTTACCACGCAGCTCAATAGCATC | |
| CCTGGTTCTGCTGTCTGTGCATTTAGCATGGATGACATTGAAAAAGTATTCAAAGGACGG | |
| TTTAAGGAACAGAAAACTCCAGATTCTGTTTGCACAGCAGTTCCCGAAGACAAAGTGCCA | |
| AAGCCAAGGCCTGCCTGTTGTGCAAAACACGGCCTTGCCGAAGCTTATAAAACCTCCATC | |
| GATTTCCCGGATGAAACTCTGTCATTCATCAAATCTCATCCCCTGATCGACTCTCCCGTT | |
| CCACCCATTGCCGATQAGCCCTGGTTCACAAAGACTCGGGTCACGTACAGACTGACGGCC | |
| ATCTCAGTGGACCATTCAGCCGGACCCTACCAGAACTACACAGTCATCTTTGTTGGCTCT | |
| CAAGCTGGCATGGTACTTAAAGTTCTGGCAAAGACCAGTCCTTTCTCTTTGAACGACACC | |
| GTATTACTGGAAOAGATTGAAGCCTACAACCATGCAAAGTGCAGTGCTGAGAATGAGGAA | |
| GACAAAAAGGTCATCTCATTACAGTTGGATAAAGATCACCACGCTTTATATGTGGCGTTC | |
| TCTAGCTGCATTATCCGCATCCCCCTCAGTCGCTGTGAGCGTTATGGATCATGTAAAAAG | |
| TCTTGTATTGCATCTCGTGACCCGTATTGTGGCTCGTTAAGCCAGCGATCCTGTGGTAGA | |
| GTGACCCCACGGATGCTGCTGTTAACCGAAGACTTCTTTGCTTTCCATAACCACAGTQCT | |
| GAAGGATATGAACAAGACACAGAATTCCGCAACACACCTCATCTAGGGGACTGCCATGAA | |
| ATTTTGCCTACTTCAACTACACCAGATTACAAAATATTTGGCGGTCCAACATCTGACATG | |
| GAGGTATCTTCATCTTCTGTTACCACAATGGCAAGTATCCCAGAAATCACACCTAAAGTG | |
| ATTGATACCTGGAGACCTAAACTGACAAGCTCTCGGAAATTTGTAGTTCAAGATGATCCA | |
| AACACTTCTGATTTTACTGATCCTTTATCGGGTATCCCAAAGGGTGTACGATGGGAAGTC | |
| CAGTCTGGGGAGTCCAACCAGATGGTCCACATGAATGTCCTCATCACCTGTGTCTTTGCT | |
| GCTTTTGTTTTGGGGGCATTCATTGCAGGTGTGGCAGTATACTGCTATCGAGACATGTTT | |
| GTTCGGAAAAACAGAAAGATCCATAAAGATGCACAGTCCCCCCAGTCATGCACAGACTCC | |
| AGTGGAAGTTTTGCCAAACTGAATGGTCTCTTTGACAGCCCTGTCAAGGAATACCAACAG | |
| AATATTGATTCTCCTAAACTGTATAGTAACCTGCTAACCAGTCGGAAAGAGCTACCACCC | |
| AATGGAGATACTAAATCCATGGTAATGGACCATCGAGGGCAACCTCCAGAGTTGGCTGCT | |
| CTTCCTACTCCTGAGTCTACACCCGTGCTTCACCAGAAGACCCTGCAGGCCATGAAGAGC | |
| CACTCAGAAAAGGCCCATGGCCATCGAGCTTCAAGGAAAGAAACCCCTCAGTTTTTTCCG | |
| TCTAGTCCGCCACCTCATTCCCCATTAAGTCATGGGCATATCCCCAGTGCCATTGTTCTT | |
| CCAAATGCTACCCATGACTACAACACGTCTTTCTCAAACTCCAATGCTCACAAAGCTGAA | |
| AAGAAGCTTCAAAACATTGATCACCCTCTCACAAAGTCATCCAGTAAGAGAGATCACCGG | |
| CGTTCTGTTGATTCCAGAAATACCCTCAATGATCTCCTGAAGCATCTGAATGACCCAAAT | |
| AGTAACCCCAAAGCCATCATGGGAGACATCCAGATCGCACACCAGAACTTAATGCTGGAT | |
| CCCATGGGATCGATGTCTGAGGTCCCACCTAAAGTCCCTAACCGGGAGGCATCGCTATAC | |
| TCCCCTCCTTCAACTCTCCCCAGAAATAGCCCAACCAAGCGAGTGGATGTCCCCACCACT | |
| CCTCGAGTCCCAATGACTTCTCTGGAAAGACAAAGAGGTTATCACAAAAATTCCTCCCAG | |
| AGGCACTCTATATCTGCTATGCCTAAAAACTTAAACTCACCAAATGGTGTTTTGTTATCC | |
| AGACAGCCTAGTATGAACCGTGGAGGATATATGCCCACCCCCACTGGGGCGAAGGTGGAC | |
| TATATTCAGGGAACACCAGTGAGTGTTCATCTGCAGCCTTCCCTCTCCAGACAGAGCAGC | |
| TACACCAGTAATGGCACTCTTCCTAGGACGGGACTAAAGAGGACGCCGTCCTTAAAACCT | |
| GACGTGCCACCAAAGCCTTCCTTTGTTCCTCAAACCCCATCTGTCAGACCACTGAACAAA | |
| TACACATACTAGGCCTCAAGTGTGCTATTCCCATGTGGCTTTATCCTGTCCGTGTTGTTG | |
| AGAG | |

The nucleic acid sequence of NOV4f maps to chromosome 15.

A NOV4f polypeptide (SEQ ID NO:18) encoded by SEQ ID NO:17 is 768 amino acid residues and is presented using the one letter code in Table 4f.

TABLE 4L
NOV4f protein sequence

| | |
|---|---|
| MRVFLLCAYILLLMVSQLRAVSFPEDDEPLNTVDYHCKSSRQYFVFRGRPSGNESQHRLD | (SEQ ID NO:20) |
| FQLMLKIPDTLYIAGRDQVYTVNLNEMPKTEVIWQQKLTWRSRQQDRENCAMKGKHKDEC | |
| HNFIKVFVPRNDEMVFVCGTNAFNPMCRYYRVSTLEYDGEEISGLARCPFDARQTNVALF | |
| ADGKLYSATVADFLASDAVIYRSMGDGSALRTIKYDSKWIKEPHFLHAIEYGNYVYFFFR | |
| EIAVEHNNLGKAVYSRVARICKNDMCGSQRVLEKUWTSFLKARLNCSVPGDSFFYFDVLQ | |
| SITDIIQINGIPTVVGVFTTQLNSIPGSAVCAFSMDDIEKVFKGRFKEQKTPDSVWTAVP | |
| EDKVPKPRPGCCAKHGLAEAYKTSIDFPDETLSFIKSHPLMDSAVPPIADEPWFTKTRVR | |
| YRLTAISVDHSAGPYQNYTVIFVGSEAGMVLKVLAKTSPFSLNDSVLLEEIEAYNHAKCS | |
| AENEEDKKVISLQLDKDHHALYVAFSSCIIRIPLSRCERYGSCKKSCIASRDPYCGWLSQ | |
| GSCGRVTPGMLLLTEDFFAFHNHSAEGYEQDTEFGNTAHLGDCHEILPTSTTPDYKIFGG | |
| PTSDMEVSSSSVTTMASIPEITPKVIDTWRPKLTSSRKFVVQDDPNTSDFTDPLSGIPKG | |
| VRWEVQSGESNQMVHMNVLITCVFAAFVLGAFIAGVAVYCRRDMFVRKNRKIHKDAESAQ | |
| SCTDSSGSFAKLNGLFDSPVKEYQQNIDSPKLYSNLLTSRKELPPNGDTKSMVMDHRGQP | |
| PELAALPTPESTPVLHQKTLQAMKSUSEKAnGHGASRKETPQFFPSSPPPHSPLSHGHIP | |
| SAIVLPNATHDYNTSFSNSNAHKAEKKLQNIDHPLTKSSSKRDHRRSVDSRNTLNDLLKH | |
| LNDPNSNPKAIMGDIQMAHQNLMLDPMGSMSEVPPKVPNREASLYSPPSTLPRNSPTKRV | |
| DVPTTPGVPMTSLERQRGYEKNSSQRHSISAMPKNLNSPNGVLLSRQPSDNRGGYMPTPT | |
| GAKVDYIQGTPVSVHLQPSLSRQSSYTSNGTLPRTGLKRTPSLKPDVPPKPSPVPQTPSV | |
| PPLNKYTY | |

NOV4a also has homology to the amino acid sequences shown in the BLASTP data listed in Table 4M.

TABLE 4M
BLAST results for NOV4a

| Gene Index/ | | Length | Identity | Positives | |
|---|---|---|---|---|---|
| Identifier | Protein/ Organism | (aa) | (%) | (%) | Expect |
| gi\|14133251\|dbj\|BAA | KIAA1479 | 1022 | 100 | 100 | 0.0 |
| 96003.2\| (AB040912) | protein/human | | | | |
| gi\|14756857\|ref\|XP_ | hypothetical | 1011 | 100 | 100 | 0.0 |
| 016482.2\| | protein XP_016482 | | | | |
| (XM_016482) | [Homo sapiens] | | | | |
| gi\|13376457\|ref\|NP_ | hypothetical | 367 | 100 | 100 | 0.0 |
| 079242.1\| | protein FLJ11598 | | | | |
| (NM_024966) | [Homo sapiens] | | | | |
| gi\|11991660\|ref\|NP_ | Semaphorin | 1030 | 62 | 78 | e−180 |
| 066847.1\| | 6A1/Human | | | | |
| (NM_020796) | | | | | |
| gi\|9055334\|ref\|NP_ | Semaphorin 6A | 888 | 62 | 78 | e−179 |
| 061214.1\| | | | | | |

The homology of these sequences is shown graphically in the ClustalW analysis shown in Table 4N.

[image]

[image]

[image]

[image]

[image]

Tables 4O lists the domain description from DOMAIN analysis results against NOV4a. This indicates that the NOV4a sequence has properties similar to those of other proteins known to contain this domain.

TABLE 4N
Domain Analysis of NOV4a

gnl|Smart|smart00630, Sema, semaphorin domain
CD-Length = 430 residues, 96.0% aligned
Score = 436 bits (1122), Expect = 1e−123

The semaphorin/collapsin family of molecules plays a critical role in the guidance of growth cones during neuronal development. See semaphorin 3F (601124). They represent a family of conserved genes that encode nerve growth cone guidance signals. In the process of constructing a complete cosmid/P1 contig covering this region for the positional cloning of oncogenes, Sekido et al. (1996) identified 2 additional members of the human semaphorin family, semaphorin 3B, which they called semaphorin A(V), and semaphorin 3F, which they called semaphorin IV, in chromosome region 3p21.3. The 2 genes lie within approximately 70 kb of each other, to have widespread but distinct patterns of expression in nonneural tissues, and to have different patterns of expression in lung cancer. Human semaphorin A(V) has 86% amino acid homology with murine semaphorin A, whereas semaphorin IV is more closely related to murine semaphorin E, with 50% homology. The 2 semaphorin genes are flanked by 2 GTP-binding protein genes, GNAI2 (139360) and GNAT1 (139330). Sekido et al. (1996) stated that other human semaphorin gene sequences, for example, human semaphorin III (SEMA3A; 603961) and homologs of murine semaphorins B (SEMA4A) and C (SEMA4B), are not located on chromosome 3. Sekido et al. (1996) showed that human semaphorin A(V) is translated in vitro into a 90-kD protein that accumulates in the endoplasmic reticulum. Human semaphorin A(V) was expressed in only 1 out of 23 small cell lung cancers (SCLCs) and 7 out of 16 non-SCLCs, whereas semaphorin IV was expressed in 19 out of 23 SCLCs and 13 out of 16 non-SCLCs. Mutational analysis of semaphorin A(V) revealed mutations (germline in 1 case) in 3 of 40 lung cancers.

The semaphorins are a family of proteins that are involved in signaling. All the family members have a secretion signal, a 500-amino acid sema domain, and 16 conserved cysteine residues (Kolodkin et al., 1993). Sequence comparisons have grouped the secreted semaphorins into 3 general classes, all of which also have an immunoglobulin domain. The semaphorin III family, consisting of human semaphorin III (SEMA3A; 603961), chicken collapsin, and mouse semaphorins A, D, and E, all have a basic domain at the C terminus. Chicken collapsin contributes to path finding by axons during development by inhibiting extension of growth cones Luo et al. (1993) through an interaction with a collapsin response mediator protein of relative molecular mass 62K (CRMP-62) (Goshima et al., 1995), a putative homolog of an axonal guidance associated UNC-33 gene product (601168). Xiang et al. (1996) isolated a novel human semaphorin, which they termed semaphorin III/F, from a region of the 3p21.3 region involved in homozygous deletions in 2 small cell lung cancer (SCLC) cell lines. The gene was expressed as a 3.8-kb transcript in a variety of cell lines and tissues. It was detected as early as embryonic day 10 in mouse development. There was high expression in mammary gland, kidney, fetal brain, and lung and lower expression in heart and liver. Although there was reduced expression of the gene in several SCLC lines, no mutations were found. The new gene had characteristics of a secreted member of the semaphorin III family, with 52% identity with mouse semaphorin E and 49% identity with chicken collapsin/semaphorin D. Sekido et al. (1996) localized the SEMA3F and SEMA3B (601281) genes to 3p21.3.

The semaphorins comprise a large family of membrane-bound and secreted proteins, some of which have been shown to function in axon guidance. See semaphorin 3F (601124). Encinas et al. (1999) cloned a novel semaphorin, which they referred to as semaphorin W (SEMAW). Sequence analysis of the SEMAW gene indicated that SEMAW is a member of the class IV subgroup of transmembrane semaphorins. The mouse and rat forms of semaphorin W share 97% amino acid sequence identity, and each shows approximately 91% identity with the human form. The SEMAW gene contains 15 exons, up to 4 of which were absent in the human cDNAs sequenced by Encinas et al. (1999). Expression studies showed that SEMAW mRNA is expressed at high levels in postnatal brain and lung and, unlike many other semaphorins, at low levels in the developing embryo. Functional studies showed that semaphorin W can collapse retinal ganglion cell axons. By genetic mapping with human/hamster radiation hybrids, Encinas et al. (1999) mapped the human SEMAW gene to chromosome 2p13. By genetic mapping with mouse/hamster radiation hybrids, they mapped the mouse Semaw gene to chromosome 6; physical mapping placed the gene on BACs carrying microsatellite markers D6Mit70 and D6Mit189. This localization placed the mouse Semaw gene within the locus for motor neuron degeneration-2 of mouse, making it an attractive candidate for that disorder.

Neural networks that are very complicated but specific to each neuron are formed during development when growth cones make specific pathway choices and find their correct targets using a variety of guidance molecules in their surroundings. The semaphorins (SEMAs) are a family of transmembrane and secreted proteins that appear to function during growth cone guidance. These proteins contain a conserved sema domain of approximately 500 amino acids. Inagaki et al. (1995) cloned a novel mouse semaphorin gene, which they named semaphorin F (SemaF). In situ hybridization detected SemaF expression throughout the brain and spinal cord of E15.5, E16.5, and P1 mice. In the central nervous system, expression was very high in the primordia of the neocortex, hippocampus, thalamus, hypothalamus, tectum, pontine nuclei, spinal cord, and retina. High expression was also found in the primordia of various tissues, such as the olfactory epithelium, epithelium of the vomeronasal organ, enamel epithelium of teeth, anterior and intermediate lobes of the pituitary, epithelium of the inner ear, and sensory ganglia, including trigeminal and dorsal root ganglia. In addition, SemaF was expressed in the lung and kidney. In adult mice, SemaF expression was markedly decreased, with very low expression in several restricted regions of the brain, including the hippocampus. Inagaki et al. (1995) suggested that SemaF functions in forming the neural network during development.

The semaphorins are a family of proteins thought to be involved in axonal guidance. Most of the known semaphorins have a similar primary structure characterized by the semaphorin domain and a carboxy-terminal Ig motif. Here we report the cloning of two members (semF and G) of a novel class of membrane-bound semaphorins which contain seven carboxy-terminal thrombospondin repeats, a motif known to promote neurite outgrowth. SemF and G transcripts are expressed, together with semD and E, in specific regions of young mouse embryos, demarcating distinct compartments of the developing somites or the undifferentiated neuroepithelium. The identification of semF and G increases the number of vertebrate semaphorins to at least 20 and suggests that some semaphorins might act as positive axonal guidance cues.

The disclosed NOV4 nucleic acid of the invention encoding a Semaphorin-like protein includes the nucleic acid whose sequence is provided in Table 4A, or 4C or a fragment thereof. The invention also includes a mutant or variant nucleic acid any of whose bases may be changed from the corresponding base shown in Table 4A or 4C while still encoding a protein that maintains its Semaphorin-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 0 percent of the bases may be so changed.

The disclosed NOV4 protein of the invention includes the Semaphorin-like protein whose sequence is provided in Table 4B or 4D. The invention also includes a mutant or variant protein any of whose residues may be changed from the corresponding residue shown in Table 4B or 4D while still encoding a protein that maintains its Semaphorin-like activities and physiological functions, or a functional fragment thereof. In the mutant or variant protein, up to about 0 percent of the residues may be so changed.

The protein similarity information, expression pattern, and map location for the semaphorin-like protein and nucleic acid (NOV4) disclosed herein suggest that this NOV4 protein may have important structural and/or physiological functions characteristic of the Semaphorin family. Therefore, the NOV4 nucleic acids and proteins of the invention are useful in potential diagnostic and therapeutic applications. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo.

The NOV4 nucleic acids and proteins of the invention are useful in potential diagnostic and therapeutic applications implicated in various diseases and disorders described below. For example, the compositions of the present invention will have efficacy for treatment of patients suffering from Parkinson's disease, psychotic and neurological disorders, Alzheimers disease, Lung and other cancers and/or other pathologies. The NOV4 nucleic acids, or fragments thereof, may further be useful in diagnostic applications, wherein the presence or amount of the nucleic acid or the protein are to be assessed.

NOV4 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example, the disclosed NOV4a protein has multiple hydrophilic regions, each of which can be used as an immunogen. In one embodiment, a contemplated NOV4 epitope is from about amino acids 1 to 10. In another embodiment, a NOV4 epitope is from about amino acids 170 to 200. In additional embodiments, NOV4 epitopes are from about amino acids 270 to 325, and from about amino acids 425 to 460. These novel proteins can be used in assay systems for functional analysis of various human disorders, which will help in understanding of pathology of the disease and development of new drug targets for various disorders.

NOV5

NOV5 includes two novel serine/threonine kinase-like proteins disclosed below. The disclosed sequences have been named NOV5a and NOV5b.

NOV5a

A disclosed NOV5a nucleic acid of 2388 nucleotides (also referred to as CG50211-01) encoding a novel serine/threonine kinase-like protein is shown in Table 5A. An open reading frame was identified beginning with an ATG initiation codon at nucleotides 201-203 and ending with a TGA codon at nucleotides 2295-2297.

TABLE 5A
NOV5a Nucleotide Sequence

(SEQ ID NO:21)

TGCACGGGGCCACTAGGACCCTCGGCGTCCCTTCCCCTCCCCCGCCCTGCCCCCTCTCCCGCCGCGCGGA
CCCGGGCGTTCTCGCCGCCCAGCTTTTGAGCTCGCGTCCCCAGGCCGGCGGGGGGGGAGGGGAAGAGAGG
GGACCCTGGGACCCCCGCCCCCCCCACCCGGCCGCCCCTGCCCCCCGGGACCCGGAGAAGATGTCTTCGC
GGACGGTGCTGGCCCCGGGCAACGATCGGAACTCGGACACGCATGGCACCTTGGGCAGTGGCCGCTCCTC
GGACAAAGGCCCGTCCTGGTCCAGCCGCTCACTGGGTGCCCGTTGCCGGAACTCCATCGCCTCCTGTCCC
GAGGAGCAGCCCCACGTGGGCAACTACCGCCTGCTGAGGACCATTGGGAAGGGCAACTTTGCCAAAGTCA
AGCTGGCTCGGCACATCCTCACTGGTCGGGAGGTTGCCATCAAGATTATCGACAAAACCCAGCTGAATCC
CAGCAGCCTGCAGAAGCTGTTCCGAGAAGTCCGCATCATGAAGGGCCTAAACCACCCCAACATCGTGAAG
CTCTTTGAGGTGATTGAGACTGAGAAGACGCTGTACCTGGTGATGGAGTACGCAAGTGCTGGTGAGCCGC
CCACCCTCTCCGCCCTGCCCCTGTGCCACCTCCCCCTGCCGCTGCACCTGACCCTGACCCCGCTCGGCCT
CTGCCCTGCAGGAGAAGTGTTTGACTACCTCGTGTCGCATGGCCGCATGAAGGAGAAGGAAGCTCGAGCC
AAGTTCCGACAGATTGTTTCGGCTGTGCACTATTGTCACCAGAAAAATATTGTACACAGGGACCTGAAGG
CTGAGAACCTCTTGCTGGATGCCGAGGCCAACATCAAGATTGCTGACTTTGGCTTCAGCAACGAGTTCAC
ACTGGGATCGAAGCTGGACACGTTCTGCGGGAGCCCCCCATATGCCGCCCCGGAGCTGTTTCAGGGCAAG
AAGTACGACGGGCCGGAGGTGGACATCTGGAGCCTGGGAGTCATCCTGTACACCCTCGTCAGCGGCTCCC
TGCCCTTCGACGGGCACAACCTCAAGGAGCTGCGGGAGCGAGTACTCAGAGGGAAGTACCGGGTCCCTTT
CTACATGTCAACAGACTGTGAGAGCATCCTGCGGAGATTTTTGGTGCTGAACCCAGCTAAACGCTGTACT
CTCGAGCAAATCATGAAAGACAAATGGATCAACATCGGCTATGAGGGTGAGGAGTTGAAGCCATACACAG
AGCCCGAGGAGGACTTCGGGGACACCAAGAGAATTGAGGTGATGGTGGGTATGGGCTACACACGGGAAGA
AATCAAAGAGTCCTTGACCAGCCAGAAGTACAACGAAGTGACCGCCACCTACCTCCTGCTGGGCAGGAAG
CTGAGCCCGACGAGCACGGGGGAGGCGGAGCTGAAGGAGGAGCGGCTGCCAGGCCGGAAGGCGAGCTGCA
GCACCGCGGGGAGTGGGAGTCGAGGGCTGCCCCCCTCCAGCCCCATGGTCAGCAGCGCCCACAACCCCAA
CAAGGCAGAGATCCCAGAGCGGCGGAAGGACAGCACGCCGGTGAGTGACCAGGGCTGGGGGATGATGACC
CGCAGAAACACCTACGTTTGCACAGAACGCCCGGGGGCTGAGCGCCCGTCACTGTTGCCAAATGGGAAAG
AAAACCGGGTGCCCCCTGCCTCCCCCTCCAGTCACAGCCTGGCACCCCCATCAGGGGAGCGGAGCCGCCT
GGCACGTGGTTCCACCATCCGCAGCACCTTCCATGGTGGCCAGGTCCGGGACCGGCGGGCAGGGGGTGGG
GGTGGTGGGGGTGTGCAGAATGGGCCCCCTGCCTCTCCCACACTGGCCCATGAGGCTGCACCCCTGCCCG
CCGGGCGGCCCCGCCCCACCACCAACCTCTTCACCAAGCTGACCTCCAAACTGACCCGATCTCGCCTCAG
TTGCCATCTACCTTGGGATCAAACGGAAACCGCCCCCCGGCTGCTCCGATTCCCCTGGAGTGTGAAGCTG
ACCAGCTCGCGCCCTCCTGAGGCCCTGATGGCAGCTCTGCGCCAGGCCACAGCAGCCGCCCGCTGCCGCT
GCCGCCAGCCACAGCCGTTCCTGCTGGCCTGCCTGCACGGGGGTGCGGGCGGGCCCGAGCCCCTGTCCCA
CTTCGAAGTGGAGGTCTGCCAGCTGCCCCGGCCAGGCTTGCGGGGAGTTCTCTTCCGCCGTGTGGCGGGC
ACCGCCCTGGCCTTCCGCACCCTCGTCACCCGCATCTCCAACGACCTCGAGCTCTGAGCCACCACGGTCC
CAGGGCCCTTACTCTTCCTCTCCCTTGTCGCCTTCACTTCTACAGGAGGGGAAGGGGCCAGGGAGGGGAT
TCTCCCTT

The NOV5a nucleic acid was identified on chromosome 19 and has 592 of 842 bases (70%) identical to a gb:GENBANK-ID:RNMARK1|acc:Z83868.1 mRNA from Rattus norvegicus (R.norvegicus mRNA for serine/threonine kinase MARK1).

A disclosed NOV5a polypeptide (SEQ ID NO:20) encoded by SEQ ID NO:19 is 698 amino acid residues and is presented using the one-letter code in Table 5B. Signal P, Psort and/or Hydropathy results predict that NOV5a has no signal peptide and is likely to be localized in the cytoplasm with a certainty of 0.4500. In other embodiments, NOV5a may also be localized to the microbody with a certainty of 0.300, the mitochondrial matrix space with a certainty of 0.1000, or the lysosome (lumen) with a certainty of 0.1000.

TABLE 5B
Encoded NOV5a protein sequence

(SEQ ID NO:22)

MSSRTVLAPGNDRNSDTHGTLGSGRSSDKGPSWSSRSLGAPCPNSIASCPEEQPHVGNYRLLRTIGKGNF
AKVKLARRILTGREVAIKIIDKTQLNPSSLQKLFREVRIMKGLNHPNIVKLFEVIETGKTLYLVMEYASA
GEPPTLSALPLCHLPLPLHLTLTPLGLCPAGEVFDYLVSHGRMKEKEAPAKFRQIVSAVHYCHQKNIVHR
DLKAENLLLDAEANIKIADFCFSNEFTLGSKLDTFCGSPPYAAPELFQGKKYDGPEVDIWSLGVILYTLV
SGSLPFDGHNLKELRERVLRGKYRVPFYMSTDCESILRRPLVLNPAKRCTLEQIMKDKWINIGYEGEELK
PYTEPEEDFGDTKRIEVMVGMGYTREEIKESLTSQKYNEVTATYLLLGRKLSPTSTGEAELKEERLPGRK
ASCSTAGSGSRGLPPSSPMVSSAHNPNKAEIPERRKDSTPVSDQCWGMNTRPNTYVCTERPGAERPSLLP
NGKENRVPPASPSSHSLAPPSGERSRLARGSTIRSTFHGGQVRDRRAGGGGGGGVQNGPPASPTLAHEAA
PLPAGRPRPTTNLFTKLTSKTRSRLSCHLPWDQTGTAPRLLRFPWSVKLTSSRPPEALMAALRQRQATAG
RCRCRQPQPFLACLHGAGGPEPLSHFEVEVCQLPRPGLRGVLFRRVAGTALAFRTLVTRISGLEL

The disclosed NOV5a amino acid sequence have 237 of 401 amino acid residues (59%) identical to, and 279 of 401 amino acid residues (69%) similar to, the 729 amino acid residue ptnr:SPTREMBL-ACC:Q9JKE4 protein from Mus musculus (Mouse) (ELKL MOTIF KINASE 2 SHORT FORM).

NOV5a is expressed in at least: lung, placenta, ovary, liver, lymph, colon, testis, B-cell, muscle, skin, brain, tonsil. This information was derived by determining the tissue sources of the sequences that were included in the invention including but not limited to SeqCalling sources, Public EST sources, Literature sources, and/or RACE sources.

NOV5b

A disclosed NOV5b nucleic acid of 1549 nucleotides (also referred to as CG50211-02) encoding a novel serine/threonine kinase-like protein is shown in Table 5A. An open reading frame was identified beginning with an ATG initiation codon at nucleotides 23-25 and ending with a TGA at nucleotides 1547-1549.

TABLE 5C
NOV5b Nucleotide Sequence

| | |
|---|---|
| TCCCCCCCGGGACCCGGAGAAGATGTCTTCGCGGACGGTGCTGGCCCCCGGCAACGATCG | (SEQ ID NO:23) |
| GAACTCGGACACGCATGGCACCTTGGGCAGTGGCCGCTCCTCGGACAAAGGCCCGTCCTG | |
| GTCCAGCCGCTCACTGGGTGCCCGTTGCCGGAACTCCATCGCCTCCTGTCCCGAGGAGCA | |
| GCCCCACGTGGGCAACTACCGCCTGCTGAGGACCATTGCGAAGGGCAACTTTGCCAAAGT | |
| CAAGCTGGCTCGGCACATCCTCACTGGTCGGGAGGTTGCCATCAAGATTATCGACAAAAC | |
| CCAGCTGAATCCCAGCAGCCTGCAGAAGCTGTTCCGAGAAGTCCGCATCATGAAGGGCCT | |
| AAACCACCCCAACATCGTGAAGCTCTTTGAGGTGATTGAGACTGAGAAGACGCTGTACCT | |
| GGTGATGGAGTACGCAAGTGCTCGAGAAGTGTTTGACTACCTCGTGTCGCATGGCCGCAT | |
| GAAGGAGAAGGAAGCTCGAGCCAAGTTCCGACAGATTGTTTCGGCTGTGCACTATTGTCA | |
| CCAGAAAAATATTGTACACAGGGACCTGAAGGCTGAGAACCTCTTGCTGGATGCCGAGGC | |
| CAACATCAAGATTGCTGACTTTGGCTTCAGCAACGAGTTCACGCTGGGATCGAGGCTGGA | |
| CACGTTCTGCGGGAGCCCCCCATATGCCGCCCCGGAGCTGTTTCAGGGCAAGAAGTACGA | |
| CGGGCCGGAGGTGGACATCTGGAGCCTGGGAGTCATCCTGTACACCCTCGTCAGCGGCTC | |
| CCTGCCCTTCGACGGGCACAACCTCAAGGAGCTGCGGGAGCGAGTACTCAGACGGAAGTA | |
| CCGGGTCCCTTTCTACATGTCAACAGACTGTGAGAGCATCCTGCGGACATTTTTGGTGCT | |
| GAACCCAGCTAAACGCTGTACTCTCGAGCAAATCATGAAAGACAAATGGATCAACATCGG | |
| CTATGAGGGTGACGAGTTGAAGCCATACACAGAGCCCGAGGAGGACTTCGGGGACACCAA | |
| GAGAATTGAGGTGATGGTGGGTATGGGCTACACACGGGAAGAAATCAAAGAGTCCTTGAC | |
| CAGCCAGAAGTACAACGAAGTGACCGCCGGGCGGCCCCGCCCCACCACCAACCTCTTCAC | |
| CAAGCTGACCTCCAAACTGACCCGAAGGGTCCCAGACGAACCTGAGAGAATCGGCGGACC | |
| TGAGGTCACAAGTTGCCATCTACCTTGGGATCAAACGGAAACCGCCCCCCGGCTGCTCCG | |
| ATTCCCCTGGAGTGTGAAGCTGACCAGCTCGCGCCCTCCTGAGGCCCTGATGGCAGCTCT | |
| CCGCCAGGCCACAGCAGCCGCCCGCTGCCGCTGCCGCCAGCCACAGCCGTTCCTGCTGGC | |
| CTGCCTGCACGGGGGTGCGGGCGGGCCCGAGCCCCTGTCCCACTTCGAAGTGGAGGTCTG | |
| CCAGCTGCCCCGGCCAGGCTTGCGGGGAGTTCTCTTCCGCCGTGTGGCGGGCACCGCCCT | |
| GGCCTTCCGCACCCTCGTCACCCGCATCTCCAACGACCTCGAGCTCTGA | |

The NOV5b nucleic acid was identified on chromosome 19 and has 1107 of 1108 bases (99%) identical to a gb:GENBANK-ID:AB049127|acc:AB049127.1 mRNA from Homo sapiens (Homo sapiens MARKL1 mRNA for MAP/microtubule affinity-regulating kinase like 1, complete cds).

A disclosed NOV5b polypeptide (SEQ ID NO:20) encoded by SEQ ID NO:19 is 508 amino acid residues and is presented using the one-letter code in Table 5B. Signal P, Psort and/or Hydropathy results predict that NOV5b has no signal peptide and is likely to be localized in the cytoplasm with a certainty of 0.4500. In other embodiments, NOV5b may also be localized to the microbody with a certainty of 0.300, the mitochondrial matrix space with a certainty of 0.1000, or the lysosome (lumen) with a certainty of 0.1000.

TABLE 5D
Encoded NOV5b protein sequence

| | |
|---|---|
| MSSRTVLAPGNDRNSDTHGTLGSCRSSDKGPSWSSRSLGARCRNSIASCPEEQPHVGNYR | (SEQ ID NO:24) |
| LLRTIGKGNFAKVKLARHILTGREVAIKIIDKTQLNPSSLQKLFREVRIMKGLNHPNIVK | |
| LFEVIETEKTLYLVMEYASAGEVFDYLVSHGRMKEKEARAKFRQIVSAVEYCHQKNIVHR | |
| DLKAENLLLDAEANIKIADFGFSNEFTLCSKLDTFCGSPPYAAPELFQGKKYDGPEVDIW | |
| SLGVILYTLVSGSLPFDGHNLKELRERVLRGKYRVPFYMSTDCESILRRFLVLNPAKRCT | |
| LEQIMKDKWINIGYEGEELKPYTEPEEDFGDTKRIEVMVGMGYTREEIKESLTSQKYNEV | |
| TAGRPRPTTNLPTKLTSRLTRRVADEPERIGGPEVTSCHLPWDQTRTAPRLLRFPWSVKL | |
| TSSRPPEALMAALRQATAAARCRCRQPQPFLLACLHCGAGGPEPLSHFEVEVCQLPRPRL | |
| RGVLFRRVAGTALAFRTLVTRISNDLEE | |

The disclosed NOV5b amino acid sequence has 361 of 362 amino acid residues (99%) identical to, and 361 of 362 amino acid residues (99%) similar to, the 688 amino acid residue ptnr:SPTREMBL-ACC:Q9BYD8 protein from Homo sapiens (Human) (MAP/MICROTUBULE AFFINITY-REGULATING KINASE LIKE 1).

NOV5b is expressed in at least lung, placenta, ovary, liver, lymph, colon, testis, B-cell, muscle, skin, brain, tonsil. Expression information was derived from the tissue sources of the sequences that were included in the derivation of the sequence of CuraGen Acc. No. CG50211-02.

NOV5a also has homology to the amino acid sequences shown in the BLASTP data listed in Table 5E.

TABLE 5E
BLAST results for NOV5a

| Gene Index/ | | Length | Identity | Positives | |
|---|---|---|---|---|---|
| Identifier | Protein/Organism | (aa) | (%) | (%) | Expect |
| gi\|14763165\|ref\|XP_ | MAP/microtubule | 688 | 77 | [symbol] | 0.0 |
| 030962.1\| | affinity- | | | | |
| (XM_030962) | regulating kinase | | | | |
| | like 1 [Homo | | | | |
| | sapiens] | | | | |
| gi\|14017937\|dbj\|BAB_ | KIAA1860 | 689 | 77 | 78 | 0.0 |
| 47489.1\| (AB058763) | protein [Homo | | | | |
| | sapiens] | | | | |
| gi\|13899225\|ref\|NP_ | MAP/microtubule | 688 | 76 | 77 | 0.0 |
| 113605.1\| | affinity- | | | | |
| (NM_031417) | regulating kinase | | | | |
| | like 1 [Homo | | | | |
| | sapiens] | | | | |
| gi\|4505103\|ref\|NP_002367.1\| | MAP/microtubule | 713 | 59 | 70 | 0.0 |
| (NM_002376) | affinity- | | | | |
| | regulating kinase | | | | |
| | 3 [Homo sapiens] | | | | |

The homology of these sequences is shown graphically in the ClustalW analysis shown in Table 5F.

[image]

[image]

[image]

Tables 5G-I list the domain description from DOMAIN analysis results against NOV5a. This indicates that the NOV5a sequence has properties similar to those of other proteins known to contain this domain.

TABLE 5G
Domain Analysis of NOV5a

gnl|Smart|smart00220, S_TKc, Serine/Threonine protein kinases, catalytic
domain; Phosphotransferases. Serine or threonine-specific
kinase subfamily
CD-Length = 256 residues, 100.0% aligned
Score = 299 bits (765), Expect = 4e−82

TABLE 5H
Domain Analysis of NOV5a

gnl|Smart|smart00220, S_TKc, Serine/Threonine protein kinases, catalytic
domain; Phosphotransferases. Serine or threonine-specific
kinase subfamily
CD-Length = 256 residues, 100.0% aligned
Score = 299 bits (765), Expect = 4e−82

TABLE 5I
Domain Analysis of NOV5a

gnl|Smart|smart00219, TyrKc, Tyrosine kinase, catalytic domain;
Phosphotransferases. Tyrosine-specific kinase subfamily
CD-Length = 258 residues, 98.8% aligned
Score = 150 bits (378), Expect = 3e−37

Eukaryotic protein kinases (Hunter T. (1991) Protein kinase classification. Meth. Enzymol. 200: 3-37) are enzymes that belong to a very extensive family of proteins which share a conserved catalytic core common with both serine/threonine and tyrosine protein kinases. Protein phosphorylation is a fundamental process for the regulation of cellular functions. The coordinated action of both protein kinases and phosphatases controls the levels of phosphorylation and, hence, the activity of specific target proteins. One of the predominant roles of protein phosphorylation is in signal transduction, where extracellular signals are amplified and propagated by a cascade of protein phosphorylation and dephosphorylation events. Two of the best characterized signal transduction pathways involve the cAMP-dependent protein kinase and protein kinase C (PKC). Each pathway uses a different second-messenger molecule to activate the protein kinase, which, in turn, phosphorylates specific target molecules. Extensive comparisons of kinase sequences defined a common catalytic domain, ranging from 250 to 300 amino acids. This domain contains key amino acids conserved between kinases and are thought to play an essential role in catalysis. In the N-terminal extremity of the catalytic domain there is a glycine-rich stretch of residues in the vicinity of a lysine residue, which has been shown to be involved in ATP binding. In the central part of the catalytic domain there is a conserved aspartic acid residue which is important for the catalytic activity of the enzyme (Taylor S. S., Xuong N. -H., Knighton D. R., Zheng J., Ten Eyck L. F., Ashford V. A., Sowadski J. M. (1991) Crystal structure of the catalytic subunit of cyclic adenosine monophosphate-dependent protein kinase. Science 253: 407-414).

Protein kinases and phosphatases regulate cell-cycle progression, transcription, translation, protein sorting and cell adhesion events that are critical to the inflammatory process. Two of the best-characterized immunosuppressants, cyclosporin and rapamycin, are also effective anti-inflammatory drugs. They act directly on protein phosphorylation and, as such, validate the concept that small-molecule modulators of phosphorylation cascades possess anti-inflammatory properties (Bhagwat S S, Manning A M, Hoekstra M F, Lewis A. Gene-regulating protein kinases as important anti-inflammatory targets. Drug Discov Today. October 1999;4(10):472-479).

Some examples of the role of serine/threonine protein kinases that are important in cell proliferation and disease include AKT, RAF1 and PIM1. Dudek et al. (Dudek, H.; Datta, S. R.; Franke, T. F.; Birnbaum, M. J.; Yao, R.; Cooper, G. M.; Segal, R. A.; Kaplan, D. R.; Greenberg, M. E.: Regulation of neuronal survival by the serine-threonine protein kinase Akt. Science 275: 661-663, 1997) demonstrated that AKT is important for the survival of cerebellar neurons. Thus, the ‘orphan’ kinase moved center stage as a crucial regulator of life and death decisions emanating from the cell membrane. Holland et al. (Holland, E. C.; Celestino, J.; Dai, C.; Schaefer, L.; Sawaya, R. E.; Fuller, G. N.: Combined activation of Ras and Akt in neural progenitors induces glioblastoma formation in mice. Nature Genet. 25: 55-57, 2000.) transferred, in a tissue-specific manner, genes encoding activated forms of Ras and Akt to astrocytes and neural progenitors in mice. These authors found that although neither activated Ras nor Akt alone was sufficient to induce glioblastoma multiforme (GBM) formation, the combination of activated Ras and Akt induced high-grade gliomas with the histologic features of human GBMs. These tumors appeared to arise after gene transfer to neural progenitors, but not after transfer to differentiated astrocytes. Increased activity of Ras is found in many human GBMs and Akt activity is increased in most of these tumors, implying that combined activation of these 2 pathways accurately models the biology of this disease (Holland, E. C.; Celestino, J.; Dai, C.; Schaefer, L.; Sawaya, R. E.; Fuller, G. N.: Combined activation of Ras and Akt in neural progenitors induces glioblastoma formation in mice. Nature Genet. 25: 55-57, 2000.).

Another disease that involves yet another serine/threonine kinase is Peutz-Jeghers syndrome (PJS), an autosomal dominant disorder characterized by melanocytic macules of the lips, buccal mucosa, and digits, multiple gastrointestinal hamartomatous polyps, and an increased risk of various neoplasms. Jenne et al. (Jenne, D. E.; Reimann, H.; Nezu, J.; Friedel, W.; Loff, S.; Jeschke, R.; Muller, O.; Back, W.; Zimmer, M.: Peutz-Jeghers syndrome is caused by mutations in a novel serine threonine kinase. Nature Genet. 18: 38-43, 1998.) identified and characterized the serine/threonine kinase STK11 and identified mutations in PJS patients. All 5 germline mutations were predicted to disrupt the function of the kinase domain. They concluded that germline mutations in STK11, probably in conjunction with acquired genetic defects of the second allele in somatic cells according to the Knudson model, caused the manifestations of PJS. These authors commented that PJS was the first cancer susceptibility syndrome identified that is due to inactivating mutations in a protein kinase and found mutations in the STK11 gene in 11 of 12 unrelated families with PJS. Ten of the 11 were truncating mutations. All were heterozygous in the germline. Su et al. found that of 53 PJS patients with cancer reported to that time, 6 (11%) were diagnosed with pancreatic adenocarcinoma. Su et al. (Su, J. -Y.; Erikson, E.; Maller, J. L.: Cloning and characterization of a novel serine/threonine protein kinase expressed in early Xenopus embryos. J. Biol. Chem. 271: 14430-14437, 1996) presented evidence that the STK11 gene plays a role in the development of both sporadic and familial (PJS) pancreatic and biliary cancers. They found that in sporadic cancers, the STK11 gene was somatically mutated in 5% of pancreatic cancers and in at least 6% of biliary cancers examined. In the patient with pancreatic cancer associated with PJS, there was inheritance of a mutated copy of the STK11 gene and somatic loss of the remaining wildtype allele.

The disclosed NOV5 nucleic acid of the invention encoding a Serin/threonine kinase -like protein includes the nucleic acid whose sequence is provided in Table 5A or a fragment thereof. The invention also includes a mutant or variant nucleic acid any of whose bases may be changed from the corresponding base shown in Table 5A while still encoding a protein that maintains its Serin/threonine kinase -like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 1 percent of the bases may be so changed.

The disclosed NOV5a protein of the invention includes the Serin/threonine kinase -like protein whose sequence is provided in Table 5B. The invention also includes a mutant or variant protein any of whose residues may be changed from the corresponding residue shown in Table 5B while still encoding a protein that maintains its Serin/threonine kinase-like activities and physiological functions, or a functional fragment thereof. In the mutant or variant protein, up to about 1 percent of the residues may be so changed.

The NOV5 nucleic acids and proteins of the invention are useful in potential therapeutic applications implicated in various diseases, disorders and conditions. The NOV5 nucleic acid, or fragments thereof, may further be useful in diagnostic applications, wherein the presence or amount of the nucleic acid or the protein are to be assessed.

NOV5 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV5a protein have multiple hydrophilic regions, each of which can be used as an immunogen. In one embodiment, a contemplated NOV5a epitope is from about amino acids 120 to 160. In other embodiments, NOV5a epitope is from about amino acids 260 to 280, from about amino acids 310 to 330 and from about amino acids 660 to 690. This novel protein also has value in development of powerful assay system for functional analysis of various human disorders, which will help in understanding of pathology of the disease and development of new drug targets for various disorders.

NOV6

NOV6 includes four novel TGF-beta binding protein-like proteins disclosed below. The disclosed sequences have been named NOV6a, NOV6b, NOV6c and NOV6d..

NOV6a

A disclosed NOV6a nucleic acid of 4818 nucleotides (also referred to as CG50215-01) encoding a novel TGF-beta binding protein-like protein is shown in Table 6A. An open reading frame was identified beginning with an ATG initiation codon at nucleotides 137-139 and ending with a TGA codon at nucleotides 4544-4546.

TABLE 6A
NOV6a Nucleotide Sequence

(SEQ ID NO:25)

CGGGCGGCGTGCGGCTGCTCTGGGTGTCGCTATTGGTGCTGCTGGCGCAGCTAGGGGCCGCAGCCTGGACTGGGCCGGCT
CGGAGAGCGTCTCCGCGTGCGCTTCACCCCGGTCGTGTGCGGCCTGCGCTGCGTCCATGGGCCGACCGGCTCCCGCTGTA
CCCCGACCTGCGCGCCCCGCQAACGCCACCAGCGTGGACAGCGGCGCTCCCGGCGGGGCGGCCCCGGGGGGACCCGGGCT
CCGCGCCTTCCTGTGTCCCTTGATCTGTCACAATGGCGGTGTGTGCGTGAAGCCTGACCGCTGCCTCTGTCCCCCGGACT
TCGCTGGCAAGTTCTGCCAGTTGCACTCCTCGGGCGCCCGGCCCCCGGCCCCGGCTATACCAGGCCTCACCCGCTCCGTG
TACACTATGCCACTGGCCAACCACCGCGACGACGAGCACGGCGTGGCATCTATGGTGAGCGTCCACGTGGAGCACCCGCA
GGAGGCGTCGGTGGTGGTGCACCAGGTGGAGCGTGTGTCTGGCCCTTGGGAGGAGGCGGACGCTGAGGCGGTGGCGCGGG
CGGAAGCGGCGGCGCGGGCGGAGGCGGCAGCGCCCTACACGGTGTTGGCACAGAGCGCGCCGCGGGAGGACGGCTACTCA
GATGCCTCGGGCTTCGGTTACTGCTTTCGGGAGCTGCGCGGAGGCGAATGCGCGTCCCCGCTGCCCGGGCTCCGGACGCA
GGAGGTCTGCTGCCGAGGGGCCGGCTTGGCCTGGGGCGTTCACGACTGTCAGCTGTGCTCCGAGCGCCTGGGGAACTCCG
AAAGAGTGAGCGCCCCAGATGGACCTTGTCCAACCGGCTTTGAAAGAGTTAATGGGTCCTGCGAAGATGTGGATGAGTGC
GCGACTGGCGGGCGCTGCCAGCACGGCGAGTGTGCAAACACGCGCGGCGGGTACACGTGTGTGTGCCCCGACGGCTTTCT
GCTCGACTCGTCCCGCAGCAGCTGCATCTCCCAACACGTGATCTCAGAGGCCAAAGGGCCCTGCTTCCGCGTGCTCCGCG
ACGGCGGCTGTTCGCTGCCCATTCTGCGGAACATCACTAAACAGATCTGCTGCTGCAGCCGCGTAGGCAAGGCCTGGGGC
CGGGGCTGCCAGCTCTGCCCACCCTTCGGCTCAGAGGGTTTCCGGGAGATCTGCCCGGCTGGTCCTGGTTACCACTACTC
GGCCTCCGACCTCCGCTACAACACCAGACCCCTGGGCCAGGAGCCACCCCGAGTGTCACTCAGCCAGCCTCGTACCCTGC
CAGCCACCTCTCGGCCATCTGCAGGCTTTCTGCCCACCCATCGCCTGGAGCCCCGGCCTGAACCCCGGCCCGATCCCCGG
CCCGGCCCTGAGTTTCCCTTGCCCAGCATCCCTGCCTGGACTGGTCCTGAGATTCCTGAATCAGGTCCTTCCTCCGGCAT
GTGTCAGCGCAACCCCCAGGTCTGCGGCCCAGGACGCTGCATTTCCCGGCCCAGCGGCTACACCTGCGCTTGCGACTCTG
GCTTCCGGCTCAGCCCCCAGGGCACCCGATGCATTGATGTGGACGAATGTCGCCGCGTGCCCCCGCCCTGTGCTCCCGGG
CGCTGCGAGAACTCACCAGGCAGCTTCCGCTGCGTGTGCGGCCCGGGCTTCCGAGCCGGCCCACGGGCTGCGGAATGCCT
GGATGTGGACGAGTGCCACCGCGTGCCGCCGCCGTGTGACCTCGGGCGCTGCGAGAACACGCCAGGCAGCTTCCTGTGCG
TGTGCCCCGCCGGGTACCAGGCTGCACCGCACGGAGCCAGCTGCCAGGATGTGGATGAATGCACCCAGAGCCCAGGCCTG
TGTGGCCGAGGGGCCTGCAAGAACCTGCCTGGCTCTTTCCGCTGTGTTTGCCCGGCTGGCTTCCGGGGCTCGGCGTGTGA
AGAGGATGTGGATGAGTGTGCCCAGGAGCCGCCGCCCTGTGGGCCCGGCCGCTGTGACAACACGGCAGGCTGGTTTCACT
GTGCCTGCCCTGCTGGCTTCCGCTCCCGAGGGCCCGGGGCCCCCTGCCAAGATGTGGATGAGTGTGCCCGAAGCCCCCCA
CCCTGCACCTACGGCCGGTGTGAGAACACAGAAGGCAGCTTCCAGTGTGTCTGCCCCATGGGCTTCCAACCCAACGCTGC
TGGCTCCGAGTGCGAGGATGTGGATGAGTGTGAGAACCACCTCGCATGCCCTGGGCAGGAGTGTGTGAACTCGCCCGGCT
CCTTCCAGTGCAGGGCCTGTCCTTCTGGCCACCACCTGCACCGTGGCAGATGCACTGATGTGGACGAATGCAGTTCGGGT
GCCCCTCCCTGTGGTCCCCACGGCCACTGCACTAACACCGAAGGCTCCTTCCGCTGCAGCTGCGCGCCAGGCTACCGGGC
GCCGTCGGGTCGGCCCGGGCCCTGCGCAGACGTGAACGAGTGCCTGGAGGGCGATTTCTGCTTCCCTCACGGCGAGTGCC
TCAACACTGACGGCTCCTTTGCCTGTACTTGTGCCCCTGGCTACCGACCCGGACCCCGCGGAGCCTCTTGCCTCGACGTT
GACGAGTGCAGCGAGGAGGACCTTTGCCAGAGCGGCATCTGTACCAACACCGACGGCTCCTTCGAGCGCATCTGTCCTCC
GGGACACCGCGCTGGCCCGGACCTCGCCTCCTGCCTCGACGTGGACGAATGTCGCGAGCGAGGCCCAGCCCTGTGCGGGT
CGCAGCGCTGTGAGAACTCTCCCGGCTCCTACCGCTGTGTCCGGGACTGCGATCCTGGGTACCACGCGGGCCCCGAGGGC
ACCTGTGACGATGTGGATGAGTGCCAAGAATATGGTCCCGAGATTTGTGGAGCCCAGCGTTGTGAGAACACCCCTGGCTC
CTACCGCTGCACACCAGCCTGTGACCCTGGCTATCAGCCCACGCCAGGGGGCGGATGCCAGGATGTGAACGAGTGTGAAA
CACTACAGGGTGTATGTGGAGCTGCCCTGTGTGAAAATGTCGAAGGCTCCTTCCTCTGTGTCTGCCCCAACAGCCCGGAA
GAGTTTGACCCCATGACTGGACGCTGTGTTCCCCCACGAACTTCTGCTGGCACGTTCCCAGGCTCGCAGCCCCAGGCACC
TGCTAGCCCCGTTCTGCCCGCCAGGCCACCTCCGCCACCCCTGCCCCGCCGACCCAGCACACCTAGGCAGGGCCCTGTGG
GGAGTGGGCGCCGGGAGTGCTACTTTGACACAGCGGCCCCGGATGCATGTGACAACATCCTGGCTCGGAATGTGACATGG
CAGGAGTGCTGCTGTACTGTGGGTGAGGGCTGGGGCAGCGGCTGCCGCATCCAGCAGTGCCCGGGCACCGAGACAGCTGA
GTACCAGTCATTGTGCCCTCACGGCCGGGGCTACCTGGCGCCCAGTGGAGACCTGAGCCTCCGGAGAGACGTGGACGAAT
GTCAGCTCTTCCGAGACCAGGTGTGCAAGAGTGGCGTGTGTGTGAACACGGCCCCGGGCTACTCATGCTATTGCAGCAAC
GGCTACTACTACCACACACAGCGGCTGGAGTGCATCGACAATGACGAGTGCGCCGATGAGGAACCGGCCTGTGAGGGCGG
CCGCTGTGTCAACACTGTGGGCTCTTATCACTGTACCTGCGAGCCCCCACTGGTGCTGGATGGCTCGCAGCGCCGCTGCG
TCTCCAACGAGAGCCAGAGCCTCGATGACAATCTGGGAGTCTGCTGGCAGGAAGTGGGGGCTGACCTCGTGTGCAGCCAC
CCTCGGCTGGACTGTCAGGCCACCTACACAGAGTGCTGCTGCCTGTATGGAGAGGCCTGGGGCATGGACTGCGCCCTCTG
CCCTGCGCAGGACTCAGATGACTTCGAGGCCCTGTGCAATGTGCTACGCCCCCCCCGCATATAGCCCCCGCGACCAGGTG
GCTTTGGACTCCCCTACGAGTACGGCCCAGACTTAGGTCCACCTTACCAGGGCCTCCCATATGGGCCTGAGTTGTACCCA
CCACCTGCGCTACCCTACGACCCCTACCCACCGCCACCTGGGCCCTTCGCCCGCCGGGAGGCTCCTTATGGGGCACCCCG
CTTCGACATGCCAGACTTTGAGGACGATGGTGGCCCCTATGGCGAATCTGAGGCTGGTGCGCCACCTGGCCCGGGCACCC
GCTGGCCCTATCGGTCCCGGGACACCCGCCGCTCCTTCCCAGAGCCCGAGGAGCCTCCTGAAGGTGGAAGCTATGCTGGT
TCCCTGGCTGAGCCCTACGAGGAGCTGGAGGCCGAGGAGTGCGGGATCCTGGACGGCTGCACCAACGGCCGCTGCGTGCG
CGTCCCCGAAGGCTTCACCTGCCGTTGCTTCGACGGCTACCGCCTGGACATGACCCGCATGGCCTGCGTTGACATCAACG
AGTGTGATGAGGCCGAGGCTGCCTCCCCGCTGTGCGTCAACGCGCGTTGCCTCAACACGGATGGCTCCTTCCGCTGCATC
TGCCGCCCAGGATTTGCACCCACGCACCAGCCACACCACTGTGCGCCCGCACGACCCCGGGCCTGAGCCCTGGCACCCGA
TGGCCACCCACCCGCGCCCGCCACTCGGGGCCCCTGCCCCGCATCCTGCAGCCCGCTTAGTCTGATGACGAGGAAGCCCG
CCAGAAAGTCCAGAAGAAGGAACGACGGACGCAAAGCGGCGCCGCCTACCATGCCTCCCCCCCCCACCACCACCCCCCCC
AACTGTGGTCGTCCCCGCCCGGCCCACCCCGCCCCCATTTCTCCCCCCTTCTTTCAATAAAAATTTCAATCATAAAAAAC
CACCTATAAAAAAAAAA

The disclosed NOV6a nucleic acid sequence, which is mapped to chromosome 19q13.1-13.2, has has 2989 of 3024 bases (98%) identical to a gb:GENBANK-ID:AF051344|acc:AF051344.1 mRNA from Homo sapiens (Homo sapiens latent transforming growth factor-beta binding protein 4S mRNA, complete cds).

A disclosed NOV6a polypeptide (SEQ ID NO:22) encoded by SEQ ID NO:21 is 1469 amino acid residues and is presented using the one-letter amino acid code in Table 6B. Signal P, Psort and/or Hydropathy results predict that NOV6a contains no signal peptide and is likely to be localized in the cytoplasm with a certainty of 0.6500. In other embodiments, NOV6a is also likely to be localized to the mitochondrial matrix space with a certainty of 0.1000, or the lysosome (lumen) with a certainty of 0.1000.

TABLE 6B
Encoded NOV6a protein sequence.

(SEQ ID NO:26)

MGRPAPAVPRPARPATPPAWTAALPAGRPRCDPGFRAFLCPLICHNGGVCVKPDRCLCPPDFAGCFCQLH
SSGARPPAPAIPGLTRSVYTMPLANHPDDEHGVASMVSVHVEHPQEASVVVHQVERVSGPWEEADAEAVA
RAEAAARAEAAAPYTVLAQSAPREDGYSDASGFGYCFRELRGGECASPLPGLRTQEVCCRGAGLAWGVHD
CQLCSERLGNSERVSAPDGPCPTGBERVNGSCEDVDECATGGRCQHGECANTRGGYTCVCPDGFLLDSSR
SSCISQHVISEAKGPCFRVLRDGGCSLPILRNITKQICCCSRVGKAWGRGCQLCPPFGSEGFREICPAGP
GYHYSASDLRYNTRPLGQEPPRVSLSQPRTLPATSRPSAGFLPTHRLEPRPEPRPDPRPGPEFPLPSIPA
WTGPEIPESGPSSGMCQRNPQVCGPGRCISRPSGYTCACDSGFRLSPQGTRCIDVDECRRVPPPCAPGRC
ENSPGSFRCVCGPGFRAGPRAAECLDVDECHRVPPPCDLGRCENTPGSFLCVCPAGYQAAPEGASCQDVD
ECTQSPGLCGRGACKNLPGSFRCVCPAGFRGSACEEDVDECAQEPPPCGPGRCDNTAGSFHCACPAGFRS
RGPGAPCQDVDECRSPPPCTYGRCGNTEGSFQCVCPMGGQPNAAGSECEDVDECENGILACPGQECVNSP
GSFQCRACPSGHHLHRGRCTDVDECSSGAPPCGPHGHCTNTEGSFRCSCAPGYRAPSGRPGPCADVNECL
EGDFCFPHGECLNTDGSFACTCAPGYRPGPRGASCLDVDECSEEDLCQSGICTNTDGSFERICPPGRRAG
PDLASCLDVDECRERGPACGSQRCENSPGSYRCVRDCDPGYflAGPEGTCDDVDECQEYGPEICGAQRCE
NTPGSYRCTPACDPGYQPTPGGGCQDVNECETLQGVCQAALCENVEGSFLCVCPNSPEEFDPMTGRCVPP
RTSAGTFPGSQPQAPASPVLPARPPPPPLPRRPSTPRQGPVGSGRRECYFDTAAPDACDNILARNVTWQE
CCCTVGEGWGSGCRIQQCPGTETAEYQSLCPHGRGYLAPSGDLSLRRDVDECQLFRDQVCKSGVCVNTAP
GYSCYCSNGYYYHTQRLECIDNDECADEEPACEGGRCVNTVGSYHCTCEPPLVLDGSQRRCVSNESQSLD
DNLGVCWQEVGADLVCSHPRLDCQATYTECCCLYGEAWGMDCALCPAQDSDDFEALCNVLRPPAYSPPRP
GGFGLPYEYGPDLGPPYQGLPYGPELYPPPALPYDPYPPPPGPFARREAPYGAPRFDMPDFEDDGGPYGS
SEAPAPPGPGTRWPYRSRDTRRSFPEPEEPPEGGSYAGSLAEPYEELEAEECGILDGCTNGRCVRVPEGE
TCRCFDGYRLDMTRMACVDINECDEAEAASPLCVNARCLNTDGSFRCICRPGFAPTHQPHHCAPARPRA

The disclosed NOV6a amino acid sequence has 950 of 968 amino acid residues (98%) identical to, and 956 of 968 amino acid residues (98%) similar to, the 1511 amino acid residue ptnr:SPTREMBL-ACC:O75412 protein from Homo sapiens (Human) (LATENT TRANSFORMING GROWTH FACTOR-BETA BINDING PROTEIN 4S).

NOV6a is expressed in Adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus, Bone, Cervix, Lung, and Ovary.

NOV6b

A disclosed NOV6b nucleic acid of 4812 nucleotides (also referred to as CG50215-03) encoding a novel TGF-beta binding protein-like protein is shown in Table 6A. An open reading frame was identified beginning with an ATG initiation codon at nucleotides 137-139 and ending with a TGA codon at nucleotides 4538-4540. A putative untranslated region upstream from the initiation codon and downstream from the termination codon is underlined in Table 6A, and the start and stop codons are in bold letters.

TABLE 6C
NOV6b Nucleotide Sequence

| | |
|---|---|
| CGGGCGGCGTGCGGCTGCTCTGGGTGTCGCTATTGGTGCTGCTGGCGCAGCTAGGGGCCG | (SEQ ID NO:27) |
| CAGCCTGGACTGGGCCGGCTCGGAGAGCGTCTCCGCGTGCGCTTCACCCCGGTCGTGTGC | |
| GGCCTGCGCTGCGTCCATGGGCCGACCGGCTCCCGCTGTACCCCCGACCTGCGCGCCCCGC | |
| AACGCCACCAGCGTGGACAGCGGCGCTCCCGGCGGGGCGGCCCCGGGGGGACCCGGGCTT | |
| CCGCGCCTTCCTGTGTCCCTTGATCTGTCACAATGGCGGTGTGTGCGTGAAGCCTGACCG | |
| CTGCCTCTGTCCCCCGGACTTCGCTGGCAAGTTCTGCCAGTTGCACTCCTCGGGCGCCCG | |
| GCCCCCGGCCCCGGCTATACCAGGCCTCACCCGCTCCGTGTACACTATGCCACTGGCCAA | |
| CCACCGCGACGACGAGCACGGCGTGGCATCTATGGTGAGCGTCCACGTGGAGCACCCGCA | |
| GGAGGCGTCGGTGGTGGTGCACCAGGTGGAGCGTGTGTCTGGCCCTTGGGAGGAGGCGGA | |
| CGCTGAGGCGGTGGCGCGGGCGGAAGCGGCGGCGCGGGCGGAGGCGGCAGCGCCCTACAC | |
| GGTGTTGGCACAGAGCGCGCCGCGGGAGGACGGCTACTCAGATGCCTCGGGCTTCGGTTA | |
| CTGCTTTCGGGAGCTGCGCGGAGGCGAATGCGCGTCCCCGCTGCCCGGGCTCCGGACGCA | |
| GGAGGTCTGCTGCCGAGGGGCCGGCTTGGCCTGGGGCGTTCACGACTGTCAGCTGTGCTC | |
| CGAGCGCCTGGGGAACTCCGAAAGAGTGAGCGCCCCAGATGGACCTTGTCCAACCGGCTT | |
| TGAAAGAGTTAATGGGTCCTGCGAAGATGTGGATGAGTGCGCGACTGGCGGGCGCTGCCA | |
| GCACGGCGAGTGTGCAAACACGCGCGGCGGGTACACGTGTGTGTGCCCCGACGGCTTTCT | |
| GCTCGACTCGTCCCGCAGCAGCTGCATCTCCCAACACGTGATCTCAGAGGCCAAAGGGCC | |
| CTGCTTCCGCGTGCTCCGCGACGGCGGCTGTTCGCTGCCCATTCTGCGGAACATCACTAA | |
| ACAGATCTGCTGCTGCAGCCGCGTAGGCAAGGCCTGGGGCCGGGGCTGCCAGCTCTGCCC | |
| ACCCTTCGGCTCAGAGGGTTTCCGGGAGATCTGCCCGGCTGGTCCTGGTTACCACTACTC | |
| GGCCTCCGACCTCCGCTACAACACCAGACCCCTGGGCCAGGAGCCACCCCGAGTGTCACT | |
| CAGCCAGCCTCGTACCCTGCCAGCCACCTCTCGGCCATCTGCAGGCTTTCTGCCCACCCA | |
| TCGCCTGGAGCCCCGGCCTGAACCCCGGCCCGATCCCCGGCCCGGCCCTGAGTTTCCCTT | |
| GCCCAGCATCCCTGCCTGGACTGGTCCTGAGATTCCTGAATCAGGTCCTTCCTCCGGCAT | |
| GTGTCAGCGCAACCCCCAGGTCTGCGGCCCAGGACGCTGCATTTCCCGGCCCAGCGGCTA | |
| CACCTGCGCTTGCGACTCTGGCTTCCGGCTCAGCCCCCAGGGCACCCGATGCATTGATGT | |
| GGACGAATGTCGCCGCGTGCCCCCGCCCTGTGCTCCCGGGCGCTGCGAGAACTCACCAGG | |
| CAGCTTCCGCTGCGTGTGCGGCCCGGGCTTCCGAGCCGGCCCACGGGCTGCGGAATGCCT | |
| GGATGTGGACGAGTGCCACCGCGTGCCGCCGCCGTGTGACCTCGGGCGCTGCGAGAACAC | |
| GCCAGGCAGCTTCCTGTGCGTGTGCCCCGCCGGGTACCAGGCTGCACCGCACGGAGCCAG | |
| CTGCCAGGATGTGGATGAATGCACCCAGAGCCCAGGCCTGTGTGGCCGAGGGGCCTGCAA | |
| GAACCTGCCTGGCTCTTTCCGCTGTGTTTGCCCGGCTGGCTTCCGGGGCTCGGCGTGTGA | |
| AGAGGATGTGGATGAGTGTGCCCAGGAGCCGCCGCCCTGTGGGCCCGGCCGCTGTGACAA | |
| CACGGCAGGCTCCTTTCACTGTGCCTGCCCTGCTGGCTTCCGCTCCCGAGGGCCCGGGGC | |
| CCCCTGCCAAGATGTGGATGAGTGTGCCCGAAGCCCCCCACCCTGCACCTACGGCCGGTG | |
| TGAGAACACAGAAGGCAGCTTCCAGTGTGTCTGCCCCATGGGCTTCCAACCCAACGCTGC | |
| TGGCTCCGAGTGCGAGGATGTGGATGAGTGTGAGAACCACCTCGCATGCCCTGGGCAGGA | |
| GTGTGTGAACTCGCCCGGCTCCTTCCAGTGCAGGGCCTGTCCTTCTGGCCACCACCTGCA | |
| CCGTGGCAGATGCACTGATGTGGACGAATGCAGTTCGGGTGCCCCTCCCTGTGGTCCCCA | |
| CGGCCACTGCACTAACACCGAAGGCTCCTTCCGCTGCAGCTGCGCGCCAGGCTACCGGGC | |
| GCCGTCGGGTCGGCCCGGGCCCTGCGCAGACGTGAACGAGTGCCTGGAGGGCGATTTCTG | |
| CTTCCCTCACGGCGAGTGCCTCAACACTGACGGCTCCTTTGCCTGTACTTGTGCCCCTGG | |
| CTACCGACCCGGACCCCGCGGAGCCTCTTGCCTCGACGTTTGACGAGTGCAGCGAGGAGA | |
| CCTTTGCCAGAGCGGCATCTGTACCAACACCGACGGCTCCTTCGAGTGCATCTGTCCTCC | |
| GGGACACCGCGCTGGCCCGGACCTCGCCTCCTGCCTCGACGTGGACGAATGTCGCGAGCG | |
| AGGCCCAGCCCTGTGCGGGTCGCAGCGCTGTGAGAACTCTCCCGGCTCCTACCGCTGTGT | |
| CCGGGACTGCGATCCTGGGTACCACGCGGGCCCCGAGGGCACCTGTGACGATGTGGACGA | |
| ATGCCGGAACCGGTCCTTCTGCGGTGCCCACGCCGTGTGCCAGAACCTGCCCGGCTCCTT | |
| CCAGTGCCTCTGTGACCAGGGTTACGAGGGGGCACGGGATGGGCGTCACTGCGTGGATGT | |
| GAACGAGTGTGAAACACTACAGGGTGTATGTGGAGCTGCCCTGTGTGAAAATGTCGAAGG | |
| CTCCTTCCTCTGTGTCTGCCCCAACAGCCCGGAAGAGTTTGACCCCATGACTGGACGCTG | |
| TGTTCCCCCACGAACTTCTGCTGGCACGTTCCCAGGCTCGCAGCCCCAGGCACCTGCTAG | |
| CCCCGTTCTGCCCGCCAGGCCACCTCCGCCACCCCTGCCCCGCCGACCCAGCACACCTAG | |
| GCAGGGCCCTGTGGGGAGTGGGCGCCGGGAGTGCTACTTTGACACAGCGGCCCCGGATGC | |
| ATGTGACAACATCCTGGCTCGGAATGTGACATGGCAGGAGTGCTGCTGTACTGTGGGTGA | |
| GGGCTGGGGCAGCGGCTGCCGCATCCAGCAGTGCCCGGGCACCGAGACAGCTGAGTACCA | |
| GTCATTGTGCCCTCACGGCCGGGGCTACCTGGCGCCCAGTGGAGACCTGAGCCTCCGGAG | |
| AGACGTGGACGAATGTCAGCTCTTCCGAGACCAGGTGTGCAAGAGTGGCGTGTGTGTGAA | |
| CACGGCCCCGGGCTACTCATGCTATTGCAGCAACGGCTACTACTACCACACACAGCGGCT | |
| GGAGTGCATCGACAATGACGAGTGCGCCGATGAGGAACCGGCCTGTGAGGGCGGCCGCTG | |
| TGTCAACACTGTGGGCTCTTATCACTGTACCTGCGAGCCCCCACTGGTGCTGGATGGCTC | |
| GCAGCGCCGCTGCGTCTCCAACGAGAGCCAGAGCCTCGATGACAATCTGGGAGTGTGCTG | |
| GCAGGAAGTGGGGGCTGACCTCGTGTGCAGCCACCCTCGGCTGGACCGTCAGGCCACCTA | |
| CACAGAGTGCTGCTGCCTGTATGGAGAGGCCTGGGGCATGGACTGCGCCCTCTGCCCTGC | |
| GCAGGACTCAGATGACTTCGAGGCCCTGTGCAATGTGCTACGCCCCCCCGCATATAGCCC | |
| CCCGCGACCAGGTGGCTTTGGACTCCCCTACGAGTACGGCCCAGACTTAGGTCCACCTTA | |
| CCAGGGCCTCCCATATGGGCCTGAGTTGTACCCACCACCTGCGCTACCCTACGACCCCTA | |
| CCCACCGCCACCTGGGCCCTTCGCCCGCCGGGAGGCTCCTTATGGGGCACCCCGCTTCGA | |
| CATGCCAGACTTTGAGGACGATGGTGGCCCCTATGGCGAATCTGAGGCTCCTGCGCCACC | |
| TGGCCCGGGCACCCGCTGGCCCTATCGGTCCCGGGACACCCGCCGCTCCTTCCCAGAGCC | |
| CGAGGAGCCTCCTGAAGGTGGAAGCTATGCTGGTTCCCTGGCTGAGCCCTACGAGGAGCT | |
| GGAGGCCGAGGAGTGCGGGATCCTGGACGGCTGCACCAACGGCCGCTGCGTGCGCGTCCC | |
| CGAAGGCTTCACCTGCCGTTGCTTCGACGGCTACCGCCTGGACATGACCCGCATGGCCTG | |
| CGTTGACATCAACGAGTGTGATGAGGCCGAGGCTGCCTCCCCGCTGTGCGTCACAACGCG | |
| TTGCCTCAACACGGATGGCTCCTTCCGCTGCATCTGCCGCCCAGGATTTGCACCCACGCA | |
| CCAGCCACACCACTGTGCGCCCGCACGACCCCGGGCCTGAGCCCTGGCACCCGATGGCCA | |
| CCCACCCGCGCCCGCCACTCGGGGCCCCTGCCCCGCATCCTGCAGCCCGCTTAGTCTGAT | |
| GACGAGGAAGCCCGCCAGAAAGTCCAGAAGAAGGAACGACGGACGCAAAGCGGCGCCGCC | |
| TACCATGCCTCCCCCCCCCACCACCACCCCCCCCAACTGTGGTCGTCCCCGCCCGGCCCA | |
| CCCCGCCCCCATTTCTCCCCCCTTCTTTCAATAAAAATTTCAATCATAAAAAACCACCTA | |
| TAAAAAAAAAAA | |

PCR cloning of a NOV6b nucleic acid is disclosed in Example 4.

The disclosed NOV6b nucleic acid sequence, which maps to chromosome 19 has 2940 of 3024 bases (97%) identical to a gb:GENBANK-ID:AF051344|acc:AF051344.1 mRNA from Homo sapiens (Homo sapiens latent transforming growth factor-beta binding protein 4S mRNA, complete cds).

A disclosed NOV6b polypeptide (SEQ ID NO:22) encoded by SEQ ID NO:21 is 1467 amino acid residues and is presented using the one-letter amino acid code in Table 6B. Signal P, Psort and/or Hydropathy results predict that NOV6b contains no signal peptide and is likely to be localized in the cytoplasm with a certainty of 0.6500. In other embodiments, NOV6b is also likely to be localized to the mitochondrial matrix space with a certainty of 0.1000, or the lysosome (lumen) with a certainty of 0.1000.

TABLE 6D
Encoded NOV6b protein sequence.

| | |
|---|---|
| MGRPAPAVPRPARPATPPAWTAALPAGRPRGDPGFRAFLCFLICHNGGVCVKPDRCLCPP | (SEQ ID NO:28) |
| DFAGKFCQLHSSGARPPAPAIPGLTRSVYTMPLAHHRDDEHGVASMVSVHVEHPQEASVV | |
| VHQVERVSGPWEEANAEAVARAEAAARAEAAAPYTVLAQSAPREDGYSDASGFGYCFREL | |
| RGGECASPLPGLRTQEVCCROAGLAWGVHDCQLCSERLGNSERVSAPDGPCPTGFERVNG | |
| SCEDVDECATGGRCQHGECANTRGGYTCVCPDGFLLDSSRSSCISQHVISEAKGPCFRVL | |
| RDGGCSLPILRNITKQICCCSRVGKAWGRGCQLCPPFGSEGFREICPAGPGYHYSASDLR | |
| YNTRPLGQEPPRVSLSQPRTLPATSRPSAGFLPTHRLEPRPEPRPDPRPGPEFPLPSIPA | |
| WTGPEIPESGPSSGMCQRNPQVCGPGRCISRPSGYTCACDSGFRLSPQGTRCIDVDECRR | |
| VPPPCAPGRCENSFGSFRCVCGPGFRAGPRAAECLDVDECHRVPPPCDLGRCENTPGSFL | |
| CVCPAGYQAAPHGASCQDVDECTQSPGLCGRGACKNLPGSFRCVCPAGFRGSACEEDVDE | |
| CAQEPPPCGPGRCDNTAGSFHCACPAGFRSRGPGAPCQDVDECARSPPPCTYGRCENTEG | |
| SPQCVCPMGFQPNAAGSECEDVDECENHLACPGQECVNSPGSFQCRACPSGHHLHRGRCT | |
| DVDECSSGAPPCGPHGHCTNTEGSGRCSCAPGYRAPSGRPGPCADVNECLECDFCFPHGE | |
| CLNTDGSFACTCAPGYRPGPRGASCLDVDECSEEDLCQSGICTNTDGSFECICPPGHRAG | |
| PDLASCLDVDECRERGPALCGSQRCENSPGSYRCVRDCDPGYHAGPEGTCDDVDECRNRS | |
| FCGAHAVCQNLPGSFQCLCDQGYEGARDGRHCVDVNECETLQGVCGAALCENVGEGSFLG | |
| CPNSPEEFDPNTGRCVPPRTSAGTFPGSQPQAPASPVLPARPPPPPLPRRPSTPRQGPVG | |
| SGRRECYFDTAAPDACDNILARNVTWQECCCTVGEGWGSGCRIQQCPGTETAEYQSLCPH | |
| GRGYLAPSGDLSLRRDVDECQLFRDQVCKSGVCVNTAPGYSCYCSNGYYYGTQRLECIDN | |
| DECADEEPACEGGRCVNTVGSYHCTCEPPLX1LDGSQRRCVSNESQSLDDNLGVCWQEVG | |
| DLVCSHPRLDRQATYTECCCLYGEAWGMDCALCPAQDSDDFEALCNVLRPPAYSPPRPGG | |
| FGLPYEYGPDLGPPYQGLPYGPELYPPPALPPGPGPYPPPPGPFGRGPYGAPRFDMPDFE | |
| DDGGPYGESEAPAPPGPGTRWPYRSRDTRRSFPEPEEPPEGGSYAGSLAEPYEELEAEEC | |
| GILDGCTNGRCVRVPEGFTCRCFDGYRLDMTRMACVDINECDEAEAASPLCVGARCLNTD | |
| GSFRCICRPGFAPTHQPHHCAPA2PRA | |

The disclosed NOV6b amino acid sequence has 927 of 968 amino acid residues (95%) identical to, and 938 of 968 amino acid residues (96%) similar to, the 1511 amino acid residue ptnr:SPTREMBL-ACC:O75412 protein from Homo sapiens (Human) (LATENT TRANSFORMING GROWTH FACTOR-BETA BINDING PROTEIN 4S).

NOV6b is expressed in heart, lung. Expression information was derived from the tissue sources of the sequences that were included in the derivation of the sequence of CuraGen Acc. No. CG50215-03. NOV6b is predicted to be expressed in the following tissues because of the expression pattern of (GENBANK-ID: gb:GENBANK-ID:AF051344|acc:AF051344.1) a closely related Homo sapiens latent transforming growth factor-beta binding protein 4S mRNA: heart, lung, aorta, uterus, and small intestine.

NOV6c

A disclosed NOV6c nucleic acid of 4479 nucleotides (also referred to as CG50215-04) encoding a novel TGF-beta binding protein-like protein is shown in Table 6A. An open reading frame was identified beginning with an ATG initiation codon at nucleotides 137-139 and ending at a TGA at nucleotides 4205-4207. A putative untranslated region upstream from the initiation codon and downstream from the termination codon is underlined in Table 6A, and the start and stop codons are in bold letters.

TABLE 6E
NOV6c Nucleotide Sequence

| | |
|---|---|
| CGGGCGGCGTGCGGCTGCTCTGGGTGTCGCTATTGGTGCTGCTGGCGCAGCTAGGGGCCG | (SEQ ID NO:29) |
| CAGCCTGGACTGGGCCGGCTCGGAGAGCGTCTCCGCGTGCGCTTCACCCCGGTCGTGTGC | |
| GGCCTGCGCTGCGTCCATGGGCCGACCGGCTCCCGCTGTACCCCGACCTGCGCGCCCCGC | |
| AACGCCACCAGCGTGGACAGCGGCGCTCCCGGCGGGGCGGCCCCGGGGGGACCCGGGCTT | |
| CCGCGCCTTCCTGTGTCCCTTGATCTGTCACAATGGCGGTGTGTGCGTGAAGCCTGACCG | |
| CTGCCTCTGTCCCCCGGACTTCGCTGGCAAGTTCTGCCAGTTGCACTCCTCGGGCGCCCG | |
| GCCCCCGGCCCCGGCTATACCAGGCCTCACCCGCTCCGTGTACACTATGCCACTGGCCAA | |
| CCACCGCGACGACGAGCACGGCGTGGCATCTATGGTGAGCGTCCACGTGGAGCACCCGCA | |
| GGAGGCGTCGGTGGTGGTGCACCAGGTGGAGCGTGTGTCTGGCCCTTGGGAGGAGGCGGA | |
| CGCTGAGGCGGTGGCGCGGGCGGAAGCGGCGGCGCGGGCGGAGGCGGCAGCGCCCTACAC | |
| GGTGTTGGCACAGAGCGCGCCGCGGGAGGACGGCTACTCAGATGCCTCGGGCTTCGGTTA | |
| CTGCTTTCGGGAGCTGCGCGGAGGCGAATGCGCGTCCCCGCTGCCCGGGCTCCGGACGCA | |
| GGAGGTCTGCTGCCGAGGGGCCGGCTTGGCCTGGGGCGTTCACGACTGTCAGCTGTGCTC | |
| CGAGCGCCTGGGGAACTCCGAAAGAGTGAGCGCCCCAGATGGACCTTGTCCAACCAACTT | |
| TGAAAGAGTTAATGGGTCCTGCGAAGATGTGGATGAGTGCGCGACTGGCGGGCGCTGCCA | |
| GCACGGCGAGTGTGCAAACACGCGCGGCGGGTACACGTGTGTGTGCCCCGACGGCTTTCT | |
| GCTCGACTCGTCCCGCAGCAGCTGCATCTCCCAACACGTGATCTCAGAGGCCAAAGGGCC | |
| CTGCTTCCGCGTGCTCCGCGACGGCGGCTGTTCGCTGCCCATTCTGCGGAACATCACTAA | |
| ACAGATCTGCTGCTGCAGCCGCGTAGGCAAGGCCTGGGGCCGGGGCTGCCAGCTCTGCCC | |
| ACCCTTCGGCTCAGAGGGTTTCCGGGAGATCTGCCCGGCTGGTCCTGGTTACCACTACTC | |
| GGCCTCCGACCTCCGCTACAACACCAGACCCCTGGGCCAGGAGCCACCCCGAGTGTCACT | |
| CAGCCAGCCTCGTACCCTGCCAGCCACCTCTCGGCCATCTGCAGGCTTTCTGCCCACCCA | |
| TCGCCTGGAGCCCCGGCCTGAACCCCGGCCCGATCCCCGGCCCGGCCCTGAGTTTCCCTT | |
| GCCCAGCATCCCTGCCTGGACTGGTCCTGAGATTCCTGAATCAGGTCCTTCCTCCGGCAT | |
| GTGTCAGCGCAACCCCCAGGTCTGCGGCCCAGGACGCTGCATTTCCCGGCCCAGCGGCTA | |
| CACCTGCGCTTGCGACTCTGGCTTCCGGCTCAGCCCCCAGGGCACCCGATGCATTGATGT | |
| GGACGAATGTCGCCGCGTGCCCCCGCCCTGTGCTCCCGGGCGCTGCGAGAACTCACCAGG | |
| CAGCTTCCGCTGCGTGTGCGGCCCGGGCTTCCGAGCCGGCCCACGGGCTGCGGAATGCCT | |
| GGATGTGGACGAGTGCCACCGCGTGCCGCCGCCGTGTGACCTCGGGCGCTGCGAGAACAC | |
| GCCAGGCAGCTTCCTGTGCGTGTGCCCCGCCGGGTACCAGGCTGCACCGCACGGAGCCAG | |
| CTGCCAGGATGTGGATGAATGCACCCAGAGCCCAGGCCTGTGTGGCCGAGGGGCCTGCAA | |
| GAACCTGCCTGGCTCTTTCCGCTGTGTTTGCCCGGCTGGCTTCCGGGGCTCGGCGTGTGA | |
| AGAGGATGTGGATGAGTGTGCCCAGGAGCCGCCGCCCTGTGGGCCCGGCCGCTGTGACAA | |
| CACGGCAGGCTCCTTTCACTGTGCCTGCCCTGCTGGCTTCCGCTCCCGAGGGCCCGGGGC | |
| CCCCTGCCAAGATGTGGATGAGTGTGCCCGAAGCCCCCCACCCTGCACCTACGGCCGGTG | |
| TGAGAACACAGAAGGCAGCTTCCAGTGTGTCTGCCCCATGGGCTTCCAACCCAACGCTGC | |
| TGGCTCCGAGTGCGAGGATGTGGATGAGTGTGAGAACCACCTCGCATGCCCTGGGCAGGA | |
| GTGTGTGAACTCGCCCGGCTCCTTCCAGTGCAGGGCCTGTCCTTCTGGCCACCACCTGCA | |
| CCGTGGCAGATGCACTGATGTGGACGAATGCAGTTCGGGTGCCCCTCCCTGTGGTCCCCA | |
| CGGCCACTGCACTAACACCGAAGGCTCCTTCCGCTGCAGCTGCGCGCCAGGCTACCGGGC | |
| GCCGTCGGGTCGGCCCGGGCCCTGCGCAGACGTGAACGAGTGCCTGGAGGGCGATTTCTG | |
| CTTCCCTCACGGCGAGTGCCTCAACACTGACGGCTCCTTTGCCTGTACTTGTGCCCCTGG | |
| CTACCGACCCGGACCCCGCGGAGCCTCTTGCCTCGACGTTGACGAGTGCAGCGAGGAGGA | |
| CCTTTGCCAGAGCGGCATCTGTACCAACACCGACGGCTCCTTCGAGCGCATCTGTCCTCC | |
| GGGACACCGCGCTGGCCCGGACCTCGCCTCCTGCCTCGACGTGGACGAATGTCGCGAGCG | |
| AGGCCCAGCCCTGTGCGGGTCGCAGCGCTGTGAGAACTCTCCCGGCTCCTACCGCTGTGT | |
| CCGGGACTGCGATCCTGGGTACCACGCGGGCCCCGAGGGCACCTGTGACGATGTGGATGA | |
| GTGCCAAGAATATGGTCCCGAGATTTGTGGAGCCCAGCGTTGTGAGAACACCCCTGGCTC | |
| CTACCGCTGCACACCAGCCTGTGACCCTGGCTATCAGCCCACGCCAGGGGGCGGATGCCA | |
| GGATGTGAACGAGTGTGAAACACTACAGGGTGTATGTGGAGCTGCCCTGTGTGAAAATGT | |
| CGAAGGCTCCTTCCTCTGTGTCTGCCCCAACAGCCCGGAAGAGTTTGACCCCATGACTGG | |
| ACGCTGTGTTCCCCCACGAACTTCTGCTGACGTGGACGAATGTCAGCTCTTCCGAGACCA | |
| GGTGTGCAAGAGTGGCGTGTGTGTGAACACGGCCCCGGGCTACTCATGCTATTGCAGCAA | |
| CGGCTACTACTACCACACACAGCGGCTGGAGTGCATCGATAATGACGAGTGCGCCGATGA | |
| GGAACCGGCCTGTGAGGGCGGCCGCTGTGTCAACACTGTGGGCTCTTATCACTGTACCTG | |
| CGAGCCCCCACTGGTGCTGGATGGCTCGCAGCGCCGCTGCGTCTCCAACGAGAGCCAGAG | |
| CCTCGATGACAATCTGGGAGTGTGCTGGCAGGAAGTGGGGGCTGACCTCGTGTGCAGCCA | |
| CCCTCGGCTGGACCGTCAGGCCACCTACACAGAGTGCTGCTGCCTGTATGGAGAGGCCTG | |
| GGGCATGGACTGCGCCCTCTGCCCTGCGCAGGACTCAGATGACTTCGAGGCCCTGTGCAA | |
| TGTGCTACGCCCCCCCGCATATAGCCCCCCGCGACCAGGTGGCTTTGGACTCCCCTACGA | |
| GTACGGCCCAGACTTAGGTCCACCTTACCAGGGCCTCCCATATGGGCCTGAGTTGTACCC | |
| ACCACCTGCGCTACCCTACGACCCCTACCCACCGCCACCTGGGCCCTTCGCCCGCCGGGA | |
| GGCTCCTTATGGGGCACCCCGCTTCGACATGCCAGACTTTGAGGACGATGGTGGCCCCTA | |
| TGGCGAATCTGAGGCTCCTGCGCCACCTGGCCCGGGCACCCGCTGGCCCTATCGGTCCCG | |
| GGACACCCGCCGCTCCTTCCCAGAGCCCGAGGAGCCTCCTGAAGGTGGAAGCTATGCTGG | |
| TTCCCTGGCTGAGAAATACGAGGAGCTGGAGGCCGAGGAGTGCGGGATCCTGGACGGCTG | |
| CACCAACGGCCGCTGCGTGCGCGTCCCCGAAGGCTTCACCTGCCGTTGCTTCGACGGCTA | |
| CCGCCTGGACATGACCCGCATGGCCTGCGTTGACATCAACGAGTGTGATGAGGCCGAGGC | |
| TGCCTCCCCGCTGTGCGTCAACGCGCGTTGCCTCAACACGGATGGCTCCTTCCGCTGCAT | |
| CTGCCGCCCAGGATTTGCACCCACGCACCAGCCACACCACTGTGCGCCCGCACGACCCCG | |
| GGCCTGAGCCCTGGCACCCGATGGCCACCCACCCGCGCCCGCCACTCGGGGCCCCTGCCC | |
| CGCATCCTGCAGCCCGCTTAGTCTGATGACGAGGAAGCCCGCCAGAAAGTCCAGAAGAAG | |
| GAACGACGGACGCAAAGCGGCGCCGCCTACCATGCCTCCCCCCCCCACCACCACCCCCCC | |
| CAACTGTGGTCGTCCCCGCCCGGCCCACCCCGCCCCCATTTCTCCCCCCTTCTTTCAATA | |
| AAAATTTCAATCATAAAAAACCACCTATAAAAAAAAAAA | |

The disclosed NOV6c nucleic acid sequence, which maps to chromosome 19 has 2940 of 3024 bases (97%) identical to a gb:GENBANK-ID:AF051344|acc:AF051344.1 mRNA from Homo sapiens (Homo sapiens latent transforming growth factor-beta binding protein 4S mRNA, complete cds).

A disclosed NOV6c polypeptide (SEQ ID NO:22) encoded by SEQ ID NO:21 is 1356 amino acid residues and is presented using the one-letter amino acid code in Table 6B. Signal P, Psort and/or Hydropathy results predict that NOV6c contains no signal peptide and is likely to be localized in the cytoplasm with a certainty of 0.6500. In other embodiments, NOV6c is also likely to be localized to the mitochondrial matrix space with a certainty of 0.1000, or the lysosome (lumen) with a certainty of 0.1000.

TABLE 6F
Encoded NOV6c protein sequence.

| | |
|---|---|
| MGRPAPAVPRPARPATPPAWTAALPAGRPRGDPGFRAFLCPLICHNGGVCVKPDRCLCPP | (SEQ ID NO:30) |
| DFAGKFCQLHSSGARPPAPAIPGLTRSVYTMPLANHRDDEHGVASMVSVHVEHPQEASVV | |
| VHQVERVSGPWEEADAEAVARAEAAARAEAAAPYTVLAQSAPREDGYSDASGFGYCFREL | |
| RGGECASPLPGLRTQEVCCRGAGLAWGVHDCQLCSERLGNSERVSAPDGPCPTGFERVNG | |
| SCEDVDECATGGRCQHGECANTRGGYTCVCPDGFLLDSSRSSCISQHVISEAKGPCFRVL | |
| RDGGCSLPILRNITKQICCCSRVGKAWGRGCQLCPPFGSEGFREICPAGPGYHYSASDLR | |
| YNTRPLGQEPPRVSLSQPRTLPATSRPSAGFLPTHRLEPRPEPRPDPRPGPEFPLPSIPA | |
| WTGPEIPESGPSSGMCQRNPQVCGPGRCISRPSGYTCACDSGFRLSPQGTRCIDVDECRR | |
| VPPPCAPGRCENSPGSFRCVCGPGFRAGPRAAECLDVDECHRVPPPCDLGRCENTPGSFL | |
| CVCPAGYQAAPHGASCQDVDECTQSPGLCGRGACKNLPGSFRCVCPAGFRGSACEEDVDE | |
| CAQEPPPCGPGRCDNTAGSFHCACPAGFRSRGPGAPCQDVDECARSPPPCTYGRCENTEG | |
| SFQCVCPMGFQPNAAGSECEDVDECENHLACPGQECVNSPGSFQCRACPSGHHLHRGRCT | |
| DVDECSSGAPPCGPHGHCTNTEGSFRCSCAPGYRAPSGRPGPCADVNECLEGDFCFPHGE | |
| CLNTDGSFACTCAPGYRPGPRGASCLDVDECSEEDLCQSGICTNTDGSFERICPPGHRAG | |
| PDLASCLDVDECRERGPALCGSQRCENSPGSYRCVRDCDPGYHAGPEGTCDDVDECQEYG | |
| PEICGAQRCENTPGSYRCTPACDPGYQPTPGGGCQDVNECETLQGVCGAALCENVEGSFL | |
| CVCPNSPEEFDPMTGRCVPPRTSADVDECQLFRDQVCKSGVCVNTAPGYSCYCSNGYYYH | |
| TQRLECIDNDECADEEPACEGGRCVNTVGSYHCTCEPPLVLDGSQRRCVSNESQSLDDNL | |
| GVCWQEVGADLVCSHPRLDRQATYTECCCLYGEAWGMDCALCPAQDSDDFEALCNVLRPP | |
| AYSPPRPGGFGLPYEYGPDLGPPYQGLPYGPELYPPPALPYDPYPPPPGPFARREAPYGA | |
| PRFDMPDFEDDGGPYGESEAPAPPGPGTRWPYRSRDTRRSFPEPEEPPEGGSYAGSLAEP | |
| YEELEAEECGTLDGCTNGRCVRVPEGFTCRCFDGYRLDMTRMACVDINECDEAEAASPLC | |
| VNARCLNTDGSFRCICRPGFAPTHQPHHCAPARPRA | |

The disclosed NOV6c amino acid sequence has 2989 of 3024 bases (98%) identical to a gb:GENBANK-ID:AF051344|acc:AF051344.1 mRNA from Homo sapiens (Homo sapiens latent transforming growth factor-beta binding protein 4S mRNA, complete cds).

NOV6c is expressed in brain. Expression information was derived from the tissue sources of the sequences that were included in the derivation of the sequence of CuraGen Acc. No. CG50215-04. The sequence is predicted to be expressed in the following tissues because of the expression pattern of (GENBANK-ID: gb:GENBANK-ID:AF051344|acc:AF051344.1) a closely related Homo sapiens latent transforming growth factor-beta binding protein 4S mRNA, complete cds homolog in species Homo sapiens: heart, lung, aorta, uterus and small intestine.

NOV6d

A disclosed NOV6d nucleic acid of 4473 nucleotides (also referred to as CG50215-05) encoding a novel TGF-beta binding protein-like protein is shown in Table 6A. An open reading frame was identified beginning with an ATG initiation codon at nucleotides 137-139 and ending at a TGA at nucleotides 4199-4201. A putative untranslated region upstream from the initiation codon and downstream from the termination codon is underlined in Table 6A, and the start and stop codons are in bold letters.

TABLE6G
NOV6d Nucleotide Sequence

| | |
|---|---|
| CGGGCGGCGTGCGGCTGCTCTGGGTGTCGCTATTGGTGCTGCTGGCGCAGCTAGGGGCCG | (SEQ ID NO:31) |
| CAGCCTGGACTGGGCCGGCTCGGAGAGCGTCTCCGCGTGCGCTTCACCCCCGGTCGTGTGC | |
| GGCCTGCGCTGCGTCCATGGGCCGACCGGCTCCCGCTGTACCCCGACCTGCGCGCCCCGC | |
| CCGCGCCTTCCTGTGTCCCTTGATCTGTCACAATGGCGGTGTGTGCGTGGGCCTGACCG | |
| CTGCCTCTGTCCCCCGGACTTCGCTGGCAAGTTCTGCCAGTTGCACTCCTCGGCGCCCG | |
| GCCCCCGGCCCCGGCTATACCAGGCCTCACCCGCTCCGTGTACACTATGCCACTGCCCG | |
| CCACCGCGACGACGAGCACGGCGTGGCATCTATGGTGAGCGTCCACGTGGAGCACCCGCA | |
| GGAGGCGTCGGTGGTGGTGCACCAGGTGGAGCGTGTGTCTGGCCCTTGGGAGGAGCGGA | |
| CGCTGAGGCGGTGGCGCGGGCGGAAGCGGCGGCGCGGGCGGACGCACAGCGCCCTACAC | |
| GGTGTTGGCACAGAGCGCGCCGCGGGAGGACGGCTACTCAGATGCCTCGGGCTTGCTTA | |
| CTGCTTTCGGGAGCTGCGCGGAGGCGAATGCGCGTCCCCGCTGCCCGGGCTCCGGACGCA | |
| GGAGGTCTGCTGCCGAGGGGCCGGCTTGGCCTGGGGCGTTCACGACTGTCAGCTGTGCTC | |
| CGAGCGCCTGGGGAACTCCGAAAGAGTGAGCGCCCCAGATGGACCTTGTCCACCGGCTT | |
| TGAAAGAGTTAATGGGTCCTGCGAAGATGTGGATGAGTGCGCGACTGGCGGGCGCTGCCA | |
| GCACGGCGAGTGTGCAAACACGCGCGGCGGGTACACGTGTGTGTGCCCCGACGGCTTTCT | |
| GCTCGACTCGTCCCGCAGCAGCTGCATCTCCCAACACGTGATCTCAGAGGCCAAAGGGCC | |
| CTGCTTCCGCGTGCTCCGCGACGGCCCTGTTCGCTGCCCATTCTGCGGAACATCACTAA | |
| ACAGATCTGCTGCTGCAGCCGCGTAGGCAAGGCCTGGGGCCGGGGCTGCCAGCTCTGCCC | |
| ACCCTTCGGCTCAGAGGGTTTCCGGGAGATCTGCCCGGCTGGTCCTGGTTACCACTACTC | |
| GGCCTCCGACCTCCGCTACAACACCAGACCCCTGGGCCAGGAGCCACCCCGAGTGTCACT | |
| CAGCCAGCCTCGTACCCTGCCAGCCACCTCTCGGCCATCTGCAGGCTTTCTGCCCACCCA | |
| TCGCCTGGAGCCCCGGCCTGAACCCCGGCCCGATCCCCGGCCCGGCCCTGAGTTTCCCTT | |
| GCCCAGCATCCCTGCCTGGACTGGTCCTGAGATTCCTGAATCAGGTCCTTCCTCCGGCAT | |
| GTGTCAGCGCAACCCCCAGGTCTGCGGCCCAGGACGCTGCATTTCCCGGCCCAGCGGCTA | |
| CACCTGCGCTTGCGACTCTGGCTTCCGGCTCAGCCCCCAGGGCACCCGATGCATTGATGT | |
| GGACGAATGTCGCCGCGTGCCCCCGCCCTGTGCTCCCGGGCGCTGCGAGAACTCACCAGG | |
| CAGCTTCCGCTGCGTGTGCGGCCCGGGCTTCCGAGCCGGCCCACGGGCTGCGGAATGCCT | |
| GGATGTGGACGAGTGCCACCGCGTGCCGCCGCCGTGTGACCTCGGGCGCTGCGAGAACAC | |
| GCCAGGCAGCTTCCTGTGCGTGTGCCCCGCCGGGTACCAGGCTGCACCGCACGGAGCCAG | |
| CTGCCAGGATGTGGATGAATGCACCCAGAGCCCAGGCCTGTGTGGCCGAGGGGCCTGCAA | |
| GAACCTGCCTGGCTCTTTCCGCTGTGTTTGCCCGGCTGGCTTCCGGGGCTCGGCGTGTGA | |
| AGAGGATGTGGATGAGTGTGCCCAGGAGCCGCCGCCCTGTGGGCCCGGCCGCTGTGACAA | |
| CACGGCAGGCTCCTTTCACTGTGCCTGCCCTGCTGGCTTCCGCTCCCGAGGGCCCGGGGC | |
| CCCCTGCCAAGATGTGGATGAGTGTGCCCGAAGCCCCCCACCCTGCACCTACGGCCGGTG | |
| TGAGAACACAGAAGGCAGCTTCCAGTGTGTCTGCCCCATGGGCTTCCAACCCAACGCTGC | |
| TGGCTCCGAGTGCGAGGATGTGGATGAGTGTGAGAACCACCTCGCATGCCCTGGGCAGGA | |
| GTGTGTGAACTCGCCCGGCTCCTTCCAGTGCAGGGCCTGTCCTTCTGGCCACCACCTGCA | |
| CCGTGGCAGATGCACTGATGTGGACGAATGCAGTTCGGGTGCCCCTCCCTGTGGTCCCCA | |
| CGGCCACTGCACTAACACCGAAGGCTCCTTCCGCTGCAGCTGCGCGCCAGGCTACCGGGC | |
| GCCGTCGGGTCGGCCCGGGCCCTGCGCAGACGTGAACGAGTGCCTGGAGGGCGATTTCTG | |
| CTTCCCTCACGGCGAGTGCCTCAACACTGACGGCTCCTTTGCCTGTACTTGTGCCCCTGG | |
| CTACCGACCCGGACCCCGCGGAGCCTCTTGCCTCGACGTTGACGAGTGCAGCGAGGAGGA | |
| CCTTTGCCAGAGCGGCATCTGTACCAACACCGACGGCTCCTTCGAGTGCATCTGTCCTCC | |
| GGGACACCGCGCTGGCCCGGACCTCGCCTCCTGCCTCGACGTGGACGAATGTCGCGAGCG | |
| AGGCCCAGCCCTGTGCGGGTCGCAGCGCTGTGAGAACTCTCCCGGCTCCTACCGCTGTGT | |
| CCGGGACTGCGATCCTGGGTACCACGCGGGCCCCGAGGGCACCTGTGACGATGTGGACGA | |
| ATGCCGGAACCGGTCCTTCTGCGGTGCCCACGCCGTGTGCCAGAACCTGCCCGGCTCCTT | |
| CCAGTGCCTCTGTGACCAGGGTTACGAGGGGGCACGGGATGGGCGTCACTGCGTGGATGT | |
| GAACGAGTGTGAAACACTACAGGGTGTATGTGGAGCTGCCCTGTGTGAAAATGTCGAACG | |
| CTCCTTCCTCTGTGTCTGCCCCAACAGCCCGGAAGAGTTTGACCCCATGACTGGACGCTG | |
| TGTTCCCCCACGAACTTCTGCTGACGTGGACGAATGTCAGCTCTTCCGAGACCAGGTGTG | |
| CAAGAGTGGCGTGTGTGTGAACACGGCCCCGGGCTACTCATGCTATTGCAGCAACGGCTA | |
| CTACTACCACACACAGCGGCTGGAGTGCATCGATAATGACGAGTGCGCCGATGAGGAACC | |
| GGCCTGTGAGGGCGGCCGCTGTGTCAACACTGTGGGCTCTTATCACTGTACCTGCGAGCC | |
| CCCACTGGTGCTGGATGGCTCGCAGCGCCGCTGCGTCTCCAACGAGAGCCAGAGCCTCGA | |
| TGACAATCTGGGAGTGTGCTGGCAGGAAGTGGGGGCTGACCTCGTGTGCAGCCACCCTCG | |
| GCTGGACCGTCAGGCCACCTACACAGAGTGCTGCTGCCTGTATGGAGAGGCCTGGGGCAT | |
| GGACTGCGCCCTCTGCCCTGCGCAGGACTCAGATGACTTCGAGGCCCTGTGCAATGTGCT | |
| ACGCCCCCCCGCATATAGCCCCCCGCGACCAGGTGGCTTTGGACTCCCCTACGAGTACGG | |
| CCCAGACTTAGGTCCACCTTACCAGGGCCTCCCATATGGGCCTGAGTTGTACCCACCACC | |
| TGCGCTACCCTACGACCCCTACCCACCGCCACCTGGGCCCTTCGCCCGCCGGGAGGCTCC | |
| TTATGGGGCACCCCGCTTCGACATGCCAGACTTTGAGGACGATGGTGGCCCCTATGGCGA | |
| ATCTGAGGCTCCTGCGCCACCTGGCCCGGGCACCCGCTGGCCCTATCGGTCCCGGGACAC | |
| CCGCCGCTCCTTCCCAGAGCCCGAGCAGCCTCCTGAAGGTGGAAGCTATGCTGGTTCCCT | |
| GGCTGAGCCCTACGAGGAGCTGGAGGCCGAGGAGTGCGGGATCCTGGACGGCTGCACCAA | |
| CGGCCGCTGCGTGCGCGTCCCCGAAGGCTTCACCTGCCGTTGCTTCGACGGCTACCGCCT | |
| GGACATGACCCGCATGGCCTGCGTTGACATCAACGAGTGTGATGAGGCCGAGGCTGCCTC | |
| CCCGCTGTGCGTCAACGCGCGTTCCCTCAACACGGATGGCTCCTTCCGCTGCATCTGCCG | |
| CCCAGGATTTGCACCCACGCACCAGCCACACCACTGTGCGCCCGCACGACCCCGGGCCTG | |
| AGCCCTGGCACCCGATGGCCACCCACCCGCGCCCGCCACTCGGGGCCCCTGCCCCGCATC | |
| CTGCAGCCCGCTTAGTCTGATGACGAGGAAGCCCGCCAGAAAGTCCAGAAGAAGGAACGA | |
| CGGACGCAAAGCGGCGCCGCCTACCATGCCTCCCCCCCCCACCACCACCCCCCCCAACTG | |
| TGGTCGTCCCCGCCCGGCCCACCCCGCCCCCATTTCTCCCCCCTTCTTTCAATAAAAATT | |
| TCAATCATAAAAACCACCTATAAAAAAAAAAA | |

The disclosed NOV6d nucleic acid sequence, which maps to chromosome 19 has 2940 of 3024 bases (97%) identical to a gb:GENBANK-ID:AF051344|acc:AF051344.1 mRNA from Homo sapiens (Homo sapiens latent transforming growth factor-beta binding protein 4S mRNA, complete cds).

A disclosed NOV6d polypeptide (SEQ ID NO:22) encoded by SEQ ID NO:21 is 1354 amino acid residues and is presented using the one-letter amino acid code in Table 6B. Signal P, Psort and/or Hydropathy results predict that NOV6d contains no signal peptide and is likely to be localized in the cytoplasm with a certainty of 0.6500. In other embodiments, NOV6d is also likely to be localized to the mitochondrial matrix space with a certainty of 0.1000, or the lysosome (lumen) with a certainty of 0.1000.

TABLE 6H
Encoded NOV6d protein sequence.

| | |
|---|---|
| MGRPAPAVPRPARPATPPAWTAALPAGRPRGDPGFRAFLCPLICIINGGVCVKPDRCLCPP | (SEQ ID NO:32) |
| DFAGKFCQLHSSGARPPAPAIPPGLTRSVYTMPLANHRDDEHGVASMVSVHVEHPQEASVV | |
| VHQVERVSGPWEEADAEAEVARAEAAARAEAAAPYTVLAQSAPREDGYSDASGFGYCFREL | |
| RGGECASPLPGLRTQEVCCRGAGLAWGVHDCQLCSERLGNSERVSAPDGPCPTGFERVNG | |
| SCEDVDECATGGRCQHGECANTRGGYTCVCPDGFLLDSSRSSCISQHVISEARGPCFRVL | |
| RDGGCSLPILRNITKQICCCSRVGKAWGRGCQLCPPFGSEGFRETCPAGPGYHYSASDLR | |
| YNTRPLGQEPPRVSLSQPRTLPATSRPSAGFLPTHRLEPRPEPRPDPRPGPEFPLPSIPA | |
| WTGPEIPESGPSSGMCQRNPQVCGPGRCISRPSGYTCACDSGFRLSPQGTRCIDVDECRR | |
| VPPPCAPGRCENSPGSFRCVCGPGFRAGPRAAECLDVDECHRVPPPCDLGRCENTPGSFL | |
| CVCPAGYQAAPHGASCQDVDECTQSPGLCGRGACKNLPGSFRCVCPAGFRGSACEEDVDE | |
| CAQEPPPCGPGRCDNTAGSFHCACPAGFRSRGPGAPCQDVDECAISPPPCTYGRCENTEG | |
| SFQCVCPMGFQPNAAGSECEDVDECENHLACPGQECVNSPGSFQCRACPSGHHLHRGRCT | |
| DVDECSSGAPPCGPHGHCTNTEGSFRCSCAPGYRAPSGRPGPCADVNECLEGDFCFPHGE | |
| CLNTDGSFACTCAPGYRPGPRGASCLDVDECSEEDLCQSGICTNTDGSFECICPPGHRAG | |
| PDLASCLDVDECRERGPALCGSQRCENSPGSYRCVRDCDPGYHAGPEGTCDDVDECRNRS | |
| FCGAHAVCQNLPGSFQCLCDQGYEGARDGRHCVDVNECETLQGVCGAALCENVEGSFLCV | |
| CPNSPEEFDPMTGRCVPPRTSADVDECQLFRDQVCBZSCVCVNTAPGYSCYCSNGYYYHTQ | |
| RLECIDNDECADEEPACECGRCVNTVGSYHCTCEPPLVLDGSQRRCVSNESQSLDDNLGV | |
| CWQEVGADLVCSHPRLDRQATYTECCCLYGEAWGMDCALCPAQDSDDFEALCNVLRPPAY | |
| SPPRPGGFGLPYEYGPDLGPPYQGLPYGPELYPPPALPYDPYPPPPGPFARREAPYGAPR | |
| FDMPDFEDDGGPYGESEAPAPPGPGTRWPYRSRDTRRSFPEPEEPPEGGSYAGSLAEPYE | |
| ELEAEECGILDGCTNGRCVRVPECFTCRCFDGYRLDMTRMACVDINECDEAEAASPLCVN | |
| ARCLNTDGSFRCICRPGFAPTUQPHHCAPARPRA | |

The disclosed NOV6d amino acid sequence has 2940 of 3024 bases (97%) identical to a gb:GENBANK-ID:AF051344|acc:AF051344.1 mRNA from Homo sapiens (Homo sapiens latent transforming growth factor-beta binding protein 4S mRNA, complete cds).

NOV6d is expressed in Adrenal gland, bone marrow, brain, kidney, liver, lung, heart, mammary gland, pancreas, pituitary gland, placenta, prostate, salivary gland, skeletal muscle, small intestine, spinal cord, spleen, stomach, testis, thyroid, trachea, uterus, bone, cervix, and ovary. Expression information was derived from the tissue sources of the sequences that were included in the derivation of the sequence of CuraGen Acc. No. CG50215-05. The sequence is predicted to be expressed in the following tissues because of the expression pattern of (GENBANK-ID: gb:GENBANK-ID:AF051344|acc:AF051344.1) a closely related Homo sapiens latent transforming growth factor-beta binding protein 4S mRNA: heart.

NOV6 also has homology to the amino acid sequences shown in the BLASTP data listed in Table 6I.

TABLE 6I
BLAST results for NOV6

| Gene Index/ | | Length | Identity | Positives | |
|---|---|---|---|---|---|
| Identifier | Protein/ Organism | (aa) | (%) | (%) | Expect |
| gi\|3227808\|gb\|AAC39879.1\| | latent | 1511 | 97 | 97 | 0.0 |
| (AF051344) | transforming | | | | |
| | growth factor- | | | | |
| | beta binding | | | | |
| | protein 4S [Homo | | | | |
| | sapiens] | | | | |
| gi\|4505037\|ref\|NP_003564.1\| | latent | 1587 | 92 | 92 | 0.0 |
| (NM_003573) | transforming | | | | |
| | growth factor | | | | |
| | beta binding | | | | |
| | protein 4 [Homo | | | | |
| | sapiens] | | | | |
| gi\|14787032\|ref\|XP_ | latent | 888 | 97 | 97 | 0.0 |
| 047374.1\| | transforming | | | | |
| (XM_047374) | growth factor | | | | |
| | beta binding | | | | |
| | protein 4 [Homo | | | | |
| | sapiens] | | | | |
| gi\|3327814\|gb\|AAC39882.1\| | latent | 669 | 91 | 91 | 0.0 |
| (AF054502) | transforming | | | | |
| | growth factor- | | | | |
| | beta binding | | | | |
| | protein 4 [Homo | | | | |
| | sapiens] | | | | |
| gi\|14787036\|ref\|XP_ | hypothetical | 775 | 99 | 99 | 0.0 |
| 008868.4\| | protein XP_008868 | | | | |
| (XM_008868) | [Homo sapiens] | | | | |

The homology of these sequences is shown graphically in the ClustalW analysis shown in Table 6J.

[image]

[image]

[image]

[image]

[image]

[image]

In human tissues, normal homeostasis requires intricately balanced interactions between cells and the network of secreted proteins known as the extracellular matrix. These cooperative interactions involve numerous cytokines acting through specific cell-surface receptors. When the balance between the cells and the extracellular matrix is perturbed, disease can result. This is clearly evident in the interactions mediated by the cytokine transforming growth factor (beta) (TGF-(beta)).

TGF-(beta) is a member of a family of dimeric polypeptide growth factors that includes bone morphogenic proteins and activins. All of these growth factors share a cluster of conserved cysteine residues that form a common cysteine knot structure held together by intramolecular disulfide bonds. Virtually every cell in the body, including epithelial, endothelial, hematopoietic, neuronal, and connective-tissue cells, produces TGF-(beta) and has receptors for it. TGF-(beta) regulates the proliferation and differentiation of cells, embryonic development, wound healing, and angiogenesis. The essential role of the TGF-(beta) signaling pathway in these processes has been demonstrated by targeted deletion of the genes encoding members of this pathway in mice.

The biological activity of the transforming growth factor-beta's (TGF-beta) is tightly controlled by their persistance in the extracellular compartment as latent complexes. Each of the three mammalian isoform genes encodes a product that is cleaved intracellularly to form two polypeptides, each of which dimerizes. Mature TGF-beta, a 24 kD homodimer, is noncovalently associated with the 80 kD latency-associated peptide (LAP). LAP is a fundamental component of TGF-beta that is required for its efficient secretion, prevents it from binding to ubiquitous cell surface receptors, and maintains its availability in a large extracellular reservoir that is readily accessed by activation. This latent TGF-beta complex (LTGF-beta) is secreted by all cells and is abundant both in circulating forms and bound to the extracellular matrix. Activation describes the collective events leading to the release of TGF-beta. Despite the importance of TGF-beta regulation of growth and differentiation in physiological and malignant tissue processes, remarkably little is known about the mechanisms of activation in situ. Recent studies of irradiated mammary gland reveal certain features of TGF-beta 1 activation that may shed light on its regulation and potential roles in the normal and neoplastic mammary gland.

Transforming growth factor (TGF)-betas are secreted in large latent complexes consisting of TGF-beta, its N-terminal latency-associated peptide (LAP) propeptide, and latent TGF-beta binding protein (LTBP). LTBPs are required for secretion and subsequent deposition of TGF-beta into the extracellular matrix. TGF-beta1 associates with the 3(rd) 8-Cys repeat of LTBP-1 by LAP. All LTBPs, as well as fibrillins, contain multiple 8-Cys repeats. 8-Cys repeat has been found to interact with TGF-beta1*LAP by direct cysteine bridging. LTBP-1 and LTBP-3 bind efficiently all TGF-beta isoforms, LTBP-4 has a much weaker binding capacity, whereas LTBP-2 as well as fibrillins −1 and −2 are negative. A short, specific TGF-beta binding motif has been identified in the TGF-beta binding 8-Cys repeats. Deletion of this motif in the 3(rd) 8-Cys repeat of LTBP-1 results in loss of TGF-beta*LAP binding ability, while its inclusion in non-TGF-beta binding 3(rd) 8-Cys repeat of LTBP-2 results in TGF-beta binding. Molecular modeling of the 8-Cys repeats has revealed a hydrophobic interaction surface and lack of three stabilizing hydrogen bonds introduced by the TGF-beta binding motif necessary for the formation of the TGF-beta*LAP - 8-Cys repeat complex inside the cells.

LTBP-4 gene has been localized to chromosomal position 19q13.1-19q13.2. The major LTBP-4 mRNA form is about 5.1 kilobase pairs in size and is predominantly expressed in the heart, aorta, uterus, and small intestine. Immunoblotting analysis has indicated that LTBP-4 was secreted from cultured human lung fibroblasts both in a free form and in a disulfide bound complex with a TGF-beta. LAP-like protein. The matrix-associated LTBP-4 was susceptible to proteolytic release with plasmin. LTBP-4 is a member of the growing LTBP-fibrillin family of proteins and offers an alternative means for the secretion and targeted matrix deposition of TGF-betas or related proteins.

LTBP4 consists of 20 EG modules, 17 of them with a consensus sequence for calcium binding, 4 TB modules with 8 cysteines and several proline-rich regions. Northern blots demonstrated a single 5 kb mRNA which is highly expressed in heart but also present in skeletal muscle, pancreas, placenta and lung. The modular structure predicts that LTBP-4 should be a microfibrillar protein which probably also binds TGF-beta.

Increases or decreases in the production of TGF-(beta) have been linked to several disease states, including atherosclerosis and fibrotic disease of the kidney, liver, and lung, as well as in development. Mice lacking TGF-(beta)2 have cardiac, lung, craniofacial, and urogenital defects, and mice lacking TGF-(beta)3 have cleft palates. Polymorphisms in the gene for TGF-(beta)3 have been linked to the development of cleft palate in humans. Mutations in the genes for TGF-(beta), its receptors, or intracellular signaling molecules associated with TGF-(beta) are also important in the pathogenesis of diseases, particularly cancer and hereditary hemorrhagic telangiectasia.

The disclosed NOV6 nucleic acid of the invention encoding a TGF-beta binding protein-like protein includes the nucleic acid whose sequence is provided in Table 6A or a fragment thereof. The invention also includes a mutant or variant nucleic acid any of whose bases may be changed from the corresponding base shown in Table 6A while still encoding a protein that maintains its TGF-beta binding protein-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 3 percent of the bases may be so changed.

The disclosed NOV6 protein of the invention includes the TGF-beta binding protein-like protein whose sequence is provided in Table 6B. The invention also includes a mutant or variant protein any of whose residues may be changed from the corresponding residue shown in Table 6B while still encoding a protein that maintains its TGF-beta binding protein-like activities and physiological functions, or a functional fragment thereof. In the mutant or variant protein, up to about 15 percent of the residues may be so changed.

The above defined information for this invention suggests that these TGF-beta binding protein-like proteins (NOV6) may function as a member of a “TGF-beta binding protein family”. Therefore, the NOV6 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The nucleic acids and proteins of NOV6 are useful in from atherosclerosis and fibrotic disease of the kidney, liver, and lung, and cancer (e.g. cancer of epithelial, endothelial, and hematopoietic cells), hereditary hemorrhagic telangiectasia., and/or other pathologies and disorders. The novel NOV6 nucleic acid encoding NOV6 protein,, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

NOV6 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV6a protein have multiple hydrophilic regions, each of which can be used as an immunogen. In one embodiment, contemplated NOV6 epitope is from about amino acids 1 to 50. In other embodiments, NOV6 epitope is from about amino acids 220 to 300, from about amino acids 900 to 950, or from about amino acids 1150 to 1200. This novel protein also has value in development of powerful assay system for functional analysis of various human disorders, which will help in understanding of pathology of the disease and development of new drug targets for various disorders.

NOV7

A disclosed NOV7 nucleic acid of 973 nucleotides (also referred to as GMAP00808_A_da1) encoding a novel MAS proto-oncogene-like protein is shown in Table 7A. An open reading frame was identified beginning with an ATG initiation codon at nucleotides 3-5 and ending with a TGA codon at nucleotides 966-968.

TABLE 7A
NOV7 Nucleotide Sequence

(SEQ ID NO:33)

GGATGAACCAGACTTTGAATAGCAGTGCGACCGTGGAGTCAGCCCTAAACTATTCCAGAGGGAGCACAGT
GCACACGGCCTACCTGGTGCTGAGCTCCCTGGCCATGTTCACCTGCCTGTGCGCGATGGCAGGCAACAGC
ATGGTGATCTGGCTGCTAGGCTTTCGAATGCACAGOAACCCCTTCTGCATCTATATCCTCAACCTGGCGG
CAGCCGACCTCCTCTTCCTCTTCAGCATGGCTTCCACGCTCAGCCTGCAAACCCAGCCCCTGGTCAATAC
CACTGACAAGGTCCACGAGCTGATGAAGAGACTGATGTACTTTGCCTACACAGTGGGCCTGAGCCTGCTG
ACGGCCATCAGCACCCAGCGCTGTCTCTCTGTCCTCTTCCCTATCTGGTTCAAGTGTCACCCGCCCACGC
ACCTGTCAGCCTGGGTGTGTGGCCTGCTGTGGACGCTCTGTCTCCTGATGAACGGGTTGACCTCTTCCTT
CTGCAOCAAGTTCTTGAAATTCAATGAAGATCCGTGCTTCAGGGTGAACATGGTCCAGGCCGCCCTCATC
ATGGGGGTCTTAACCCCAGTGATGACTCTGTCCAGCCTGACCCTCTTTGTCTGGGTGCGGAGGAGCTCCC
AGCAGTGGCGGCGGCAGCCCACACGGCTGTTCGTGGTGGTCCTGGCCTCTGTCCTGGTGTTCCTCGTCTC
TTCCCTCCCTCTGAGCATCTACTGGTTTGTCCTCTACTGGTTGAGCCCGCCGCCCGAGATGCAGGTCCTG
TGCTTCAGCTTGTCACGCCTCTCCTCGTCCGTAAGCAGCAGCGCCAACCCCGTCATCTACTTCCTGGTGG
GCAGCCGGAGGAGCCACAGGCTGCCCACCAGGTCCCTGGGGACTGTGCTCCAACAGGCGCTTCGCGAGGA
GCCCGAGCTGGAAGGTGGGGAGACGCCCACCGTGGGCACCAATGACATGGGGGCTTGAGAGCC

The disclosed NOV7 nucleic acid sequence, localized to chromosome 11, has 413 of 676 bases (61%) identical to a gb:GENBANK-ID:HUMMAS|acc:M13150.1 mRNA from Homo sapiens (Human mas proto-oncogene mRNA, complete cds).

A disclosed NOV7 polypeptide (SEQ ID NO:24) encoded by SEQ ID NO:23 is 321 amino acid residues and is presented using the one-letter amino acid code in Table 7B. Signal P, Psort and/or Hydropathy results predict that NOV7 has a signal peptide and is likely to be localized at the plasma membrane with a certainty of 0.6000. In other embodiments, NOV7 is also likely to be localized to the golgi body with a certainty of 0.4000, to the enoplasmic reticulum (membrane) with a certainty of 0.3000, or the microbody with a certainty of 0.3000. The most likely cleavage site for a NOV7 peptide is between amino acids 44 and 45, at: MAG-NS.

TABLE 7B
Encoded NOV7 protein sequence.

(SEQ ID NO:34)

MNQTLNSSGTVESALNYSRGSTVHTAYLVLSSLAIFTCLCGMAGNSMVIWLLGPRMHRNPFCIYILNLAA
ADLLFLFSMASTLSLETQPLVNTTDKTHELMKRLMYFAYTVGLSLLTAISTQRCLSVLFPIWFKCHRPRH
LSAWVCGLLWTLCLLMJGLTSSFCSKFLKFNEDRCFRVDMVQAALIMGVLTPVMTLSSLTLFVWVRRSSQ
QWRRQPTRLFVVVLASVLVFLICSLPLSIYWEVLYWLSPPPEMQVLCFSLSRLSSSVSSSANPVIYFLVG
SRRSHRLPTRSLGTVLQQALREEPELBGGETPTVGTNEMGA

The disclosed NOV7 amino acid sequence has 114 of 318 amino acid residues (35%) identical to, and 185 of 318 amino acid residues (58%) similar to, the 324 amino acid residue ptnr:SWISSPROT-ACC:P12526 protein from Rattus norvegicus (Rat) (MAS PROTO-ONCOGENE).

NOV7 also has homology to the amino acid sequence shown in the BLASTP data listed in Table 7C.

TABLE 7C
BLAST results for NOV7

| Gene Index/ | | Length | | | |
|---|---|---|---|---|---|
| Identifier | Protein/ Organism | (aa) | Identity (%) | Positives (%) | Expect |
| gi\|15546023\|gb\|AAK | RF-amide G | 322 | 40 | 58 | 8e−43 |
| 91787.1\| (AY042191) | protein-coupled | | | | |
| | receptor [Mus | | | | |
| | musculus] | | | | |
| gi\|13507682\|ref\|NP_ | G protein-coupled | 321 | 36 | 56 | 1e−40 |
| 109651.1\| (NM_030726) | receptor 90; G- | | | | |
| | protein coupled | | | | |
| | receptor GPR90 | | | | |
| | [Mus musculus] | | | | |
| gi\|16876455\|ref\|NP_ | G protein-coupled | 322 | 41 | 58 | 2e−40 |
| 473373.1\| (NM_054032) | receptor MRGX4 | | | | |
| | [Homo sapiens] | | | | |
| gi\|15546054\|gb\|AAK | MrgD G protein- | 321 | 58 | 72 | 3e−83 |
| 91800.1\| (AY042209) | coupled receptor | | | | |
| | [Mus musculus] | | | | |
| gi\|15546062\|gb \|AAK | MrgX1 G | 322 | 40 | 58 | 8e−43 |
| 91804.1\| (AY042213) | protein-coupled | | | | |
| | receptor [Homo | | | | |
| | sapiens] | | | | |

The homology of these sequences is shown graphically in the ClustalW analysis shown in Table 7D.

[image]

[image]

Table 7E lists the domain description from DOMAIN analysis results against NOV7. This indicates that the NOV7 sequence has properties similar to those of other proteins known to contain this domain.

TABLE 7E
Domain Analysis of NOV7

gnl|Pfam|pfam00001, 7tm_1, 7 transmembrane receptor (rhodopsin
family).
CD-Length = 254 residues, Score = 38.9 bits (89), Expect = 5e−04

The human mas oncogene was originally detected by its ability to transform NIH 3T3 cells. We previously showed that the protein encoded by this gene is unique among cellular oncogene products in that it has seven hydrophobic potential transmembrane domains and shares strong sequence similarity with a family of hormone-receptor proteins (Young D, et.al.; Proc Natl Acad Sci U S A July 1988;85(14):5339-42). We have now cloned the rat homolog of the mas oncogene, determined its DNA sequence, and examined its expression in various rat tissues. A comparison of the predicted sequences of the rat and human mas proteins shows that they are highly conserved, except in their hydrophilic amino-terminal domains. Our examination of the expression of mas, determined by RNA-protection studies, indicates that high levels of mas RNA transcripts are present in the hippocampus and cerebral cortex of the brain, but not in other neural regions or in other tissues. This pattern of expression and the similarity of mas protein to known receptor proteins suggest that mas encodes a receptor that is involved in the normal neurophysiology and/or development of specific neural tissues.

The human mas oncogene, which renders transfected NIH/3T3 cells tumorigenic, was identified as a subtype of angiotensin receptor by transient expression in Xenopus oocytes and stable expression in the mammalian neuronal cell line, NG115-401L (Hanley M R, et.al.; Ciba Found Symp 1990;150:23-38; discussion 38-46). The mas receptor preferentially recognizes angiotensin III, and is expressed at high levels in brain. The mas/angiotensin receptor functions through the breakdown of inositol lipids and can drive DNA synthesis, unlike another inositol-linked peptide receptor, that for bradykinin. Comparative analysis of several early biochemical events elicited by either angiotensin or bradykinin stimulation of mas-transfected cells has not indicated a specific difference correlated with mitogenic activity. In particular, the inositol lipid kinase, phosphatidylinositol-3-kinase, thought to be involved in the mitogenic mechanism of platelet-derived growth factor receptors, is unaffected by activation of mas. These results have shown that a proto-oncogene encodes a neural peptide receptor, indicating that peptide receptors may be involved in differentiation and proliferation processes, as are other identified proto-oncogenes.

The class of receptors coupled to GTP-binding proteins share a conserved structural motif which is described as a ‘seven-transmembrane segment’ following the prediction that these hydrophobic segments form membrane-spanning alpha-helices (Jackson T R, et.al.; Nature Sep. 29, 1988;335(6189):437-40). Identified examples include the mammalian opsins, alpha 1-, alpha 2-, beta 1- and beta 2-adrenergic receptors, the muscarinic receptor family, the 5-HT1C-receptor, and the substance-K receptor. In addition, two mammalian genes have been identified that code for predicted gene products with sequence similarity to these receptors, but whose ligand specificity is unknown namely, G21 and the mas oncogene. The mas oncogene shows the greatest sequence similarity to the substance-K receptor, and on this basis it was predicted that it would encode a peptide receptor with mitogenic activity which would act through the inositol lipid signalling pathways. The mas oncogene product was transiently expressed in Xenopus oocytes, and stably expressed in a transfected mammalian cell line. The results demonstrate that the mas gene product is a functional angiotensin receptor.

The disclosed NOV7 nucleic acid of the invention encoding a MAS proto-oncogene Precursor-like protein includes the nucleic acid whose sequence is provided in Table 7A or a fragment thereof. The invention also includes a mutant or variant nucleic acid any of whose bases may be changed from the corresponding base shown in Table 7A while still encoding a protein that maintains its MAS proto-oncogene Precursor-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 39 percent of the bases may be so changed.

The disclosed NOV7 protein of the invention includes the MAS proto-oncogene Precursor-like protein whose sequence is provided in Table 7B. The invention also includes a mutant or variant protein any of whose residues may be changed from the corresponding residue shown in Table 7B while still encoding a protein that maintains its MAS proto-oncogene Precursor-like activities and physiological functions, or a functional fragment thereof. In the mutant or variant protein, up to about 65 percent of the residues may be so changed.

The protein similarity information, expression pattern, and map location for the MAS proto-oncogene Precursor-like protein and nucleic acid (NOV7) disclosed herein suggest that NOV7 may have important structural and/or physiological functions characteristic of the MAS proto-oncogene Precursor-like family. Therefore, the NOV7 nucleic acids and proteins of the invention are useful in potential diagnostic and therapeutic applications. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo.

The NOV7 nucleic acids and proteins of the invention are useful in potential diagnostic and therapeutic applications implicated in various diseases and disorders described below and/or other pathologies. For example, the compositions of the present invention will have efficacy for treatment of patients suffering from hypogonadotropic hypogonadism, Kallman syndrome, bacterial/viral infection, immunological and inflammatory diseases and disorders, and/or other pathologies/disorders. The NOV7 nucleic acid, or fragments thereof, may further be useful in diagnostic applications, wherein the presence or amount of the nucleic acid or the protein are to be assessed.

NOV7 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV7 protein have multiple hydrophilic regions, each of which can be used as an immunogen. In one embodiment, contemplated NOV7 epitope is from about amino acids 20 to 80. In other embodiments, contemplated NOV7 epitopes are from amino acids 105 to 125, from amino acids 140 to 160, from amino acids 175 to 200, or from amino acids 215 to 275. This novel protein also has value in development of powerful assay system for functional analysis of various human disorders, which will help in understanding of pathology of the disease and development of new drug targets for various disorders.

NOV8

A disclosed NOV8 nucleic acid of 671 nucleotides (also referred to as AL163195_da2) encoding a novel ribonuclease pancreatic precursor-like protein is shown in Table 8A. An open reading frame was identified beginning with at nucleotides 3-5 and ending with a TAA codon at nucleotides 465-467.

TABLE 8A
NOV8 nucleotide sequence.

(SEQ ID NO:35)

ATGCGAAGTCACTCTTACCTCTGATGATAATAATGGCATGATAATTTTCTTCTGCTTCTQTTCTGGAAAA
TGAGGTGAATGATGAGCAGTGATGTCCAAACTTTAGACACTTGCATGTGGACTACCCTCAGAATGACGTT
CCCGTTCCTGCAAGGTACTGCAACCACATGATCATACACAAGAGTTATCAGGGAACCTGACACACTTGTA
AAAAGGAGCATGTCTTCATCCATGAGAGGCCTCGAAAAATCAATGGTATTTGCATTTCTCCCAAGAAGGT
TGCTTGCCAAAACCTTTCGGCCATTTTCTGCTTTCAGAGTGAGACAAAGTTCAAAATGACAGTCTGTCAG
CTCATTGAAGGCACAAGATACCCTGCCTGCAGGTACCACTATTCCCCCACAGAGGGGTTTGTTCTTGTCA
CTTGTGATGACTTGAGGCCAGATAGTTTCCACTTGGCTATGTTAAATAAACTGATCAGCTCCCGAGTCTG
AGATCTCTTCTCTCAATGGCATTGGAGCTGGCTGTGCCTGAGGCAGACCTGGACCGTGGACATGGGGCAA
TGCTTGAACGGAAGGGGAAGCCACTGGTAATTAATTTATCCTTCCTGTATTGCTGGGTTGGGATTGTTT
TATTCTGCTTCAATAAATAATCTTTACTGAATTAAAAAAA

The NOV8 nucleic acid sequence is located on chromsome 14.

The disclosed NOV8 polypeptide (SEQ ID NO:26) encoded by SEQ ID NO:25 has 154 amino acid residues and is presented in Table 8B using the one-letter amino acid code. Signal P, Psort and/or Hydropathy results predict that NOV8 has a signal peptide and is likely to be localized at the plasma membrane with a certainty of 0.6800. In other embodiments, NOV8 may also be localized to the endoplasmic reticulum (membrane) with a certainty of 0.6400, the golgi body with a certainty of 0.3700, or the endoplasmic reticulum (lumen) with a certainty of 0.1000. The most likely cleavage site for NOV8 is between positions 27 and 28, VND-EA.

TABLE 8B
Encoded NOV8 protein sequence.

(SEQ ID NO:36)

AKSLLPLMIIMVIIFLVLLFWENEVNDEAVMSTLEHLHVDYPQNDVPVPARYCNHMIIQRVIREPDHTCIK
KENVFIHERPRKINGICISPKKVACQNLSAIFCFQSETKFKMTVCQLIEGTRYPACRYHYSPTEGFVLVT
CDDLRPDSFLGYVK

A search of sequence databases reveals that the NOV8 amino acid sequence has 43 of 141 amino acid residues (30%) identical to, and 75 of 141 amino acid residues (53%) similar to, the 156 amino acid residue ptnr:SWISSPROT-ACC:P07998 protein from Homo sapiens (Human) (RIBONUCLEASE PANCREATIC PRECURSOR (EC 3.1.27.5) (RNASE 1) (RNASE A) (RNASE UPI-1) (RIB-1)).

NOV8 is found in at least lung, testis, and B-cell. This information was derived by determining the tissue sources of the sequences that were included in the invention including but not limited to SeqCalling sources, Public EST sources, Literature sources, and/or RACE sources.

NOV8 also has homology to the amino acid sequence shown in the BLASTP data listed in Table 8C.

TABLE 8C
BLAST results for NOV8

| Gene Index/ | | Length | Identity | Positives | |
|---|---|---|---|---|---|
| Identifier | Protein/Organism | (aa) | (%) | (%) | Expect |
| gi\|13399882\|pdb\|1DZA\|A | Chain A, 3-D | 129 | 30 | 50 | 5e−09 |
| | Structure Of A | | | | |
| | Hp-Rnase | | | | |
| gi\|12853968\|dbj\|BAB2 | Putative | 208 | 34 | 54 | 6e−09 |
| 9898.1\| (AK015573) | protein/mouse | | | | |
| gi\|133226\|sp\|P19644\| | RIBONUCLEASE | 128 | 34 | 55 | 6e−09 |
| RNP_PREEN | PANCREATIC (RNASE | | | | |
| | 1) (RNASE A) | | | | |
| gi\|464659\|sp\|P80287\| | RIBONUCLEASE | 119 | 27 | 49 | 1e−08 |
| RNP_IGUIG | PANCREATIC (RNASE | | | | |
| | 1) (RNASE A) | | | | |
| gi\|13124491\|sp\|Q9QYX3\| | RIBONUCLEASE | 149 | 28 | 50 | 3e−09 |
| RNP_MUSPA | PANCREATIC | | | | |
| | PRECURSOR (RNASE | | | | |
| | 1) (RNASE A) | | | | |

The homology of these sequences is shown graphically in the ClustalW analysis shown in Table 8D.

[image]

Table 8E lists the domain description from DOMAIN analysis results against NOV8. This indicates that the NOV8 sequence has properties similar to those of other proteins known to contain this domain.

TABLE 8E
Domain Analysis of NOV8

gnl|Smart|smart00092, RNAse_Pc, Pancreatic ribonuclease
CD-Length = 123 residues, 80.5% aligned Score = 66.6 bits (161),
Expect = 1e−12

Enzymic properties of members of the ribonuclease A superfamily, like the activity on RNA, the preference for either cytosine or racil in the primary binding site B1, the preference for the other side of the cleaved phosphodiester bond, the B2 site, and features of the two noncatalytic phosphate-binding sites P0 and P2 are discussed in several articles in this multi-author review, and are summarized in this closing article (See Beintema J J, et.al.; Cell Mol Life Sci August 1998;54(8):825-32). A special feature of members of the ribonucleases 1 family is their destabilizing action on double-stranded nucleic acid structures. A feature of the ribonuclease A superfamily is the frequent occurrence of gene duplications, both in ancestral vertebrate lineages and in recently evolved taxa. Three different bovine ribonucleases 1 have been identified in pancreas, semen and brain, respectively, which are the result of two gene duplications in an ancestral ruminant. Similar gene duplications have been identified in other ribonuclease families in several mammalian and other vertebrate taxa. The ribonuclease family, of which the human members have been assigned numbers 2, 3 and 6, underwent a still mysterious pattern of gene duplications and functional expression as proteins with ribonuclease activity and other physiological properties.

Pancreatic ribonuclease (EC 3.1.27.5 ) is one of the digestive enzymes secreted in abundance by the pancreas. Elliott et al. (Cytogenet. Cell Genet. 42: 110-112, 1986) mapped the mouse gene to chromosome 14 by Southern blot analysis of genomic DNA from recombinant inbred strains of mice, using a probe isolated from a pancreatic cDNA library with the rat cDNA. A polymorphic BamHI site was used to demonstrate complete concordance of the Rib-1 locus with Tcra and Np-2, encoding the alpha subunit of the T-cell receptor (186880) and nucleoside phosphorylase (164050), respectively. The assignment to mouse 14 and the close linkage to the other 2 loci was confirmed by study of one of Snell's congenic strains: the 3 loci went together. Elliott et al. (1986) predicted that the homologous human gene RIB1 is on chromosome 14.

Human pancreatic RNase is monomeric and is devoid of any biologic activity other than its RNA degrading ability. Piccoli et al. (Proc. Nat. Acad. Sci. 96: 7768-7773,1999) engineered the monomeric form into a dimeric protein with cytotoxic action on mouse and human tumor cells, but lacking any appreciable toxicity on human and mouse normal cells. The dimeric variant of human pancreatic RNase selectively sensitized cells derived from a human thyroid tumor to apoptotic death. Because of its selectivity for tumor cells, and because of its human origin, this protein was thought to represent an attractive tool for anticancer therapy.

The disclosed NOV8 nucleic acid of the invention encoding a Ribonuclease pancreatic precursor-like protein includes the nucleic acid whose sequence is provided in Table 8A, or a fragment thereof. The invention also includes a mutant or variant nucleic acid any of whose bases may be changed from the corresponding base shown in Table 8A while still encoding a protein that maintains its Ribonuclease pancreatic precursor-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 100% percent of the bases may be so changed.

The disclosed NOV8 protein of the invention includes the Ribonuclease pancreatic precursor-like protein whose sequence is provided in Table 8B. The invention also includes a mutant or variant protein any of whose residues may be changed from the corresponding residue shown in Table 2 while still encoding a protein that maintains its Ribonuclease pancreatic precursor-like activities and physiological functions, or a functional fragment thereof. In the mutant or variant protein, up to about 70% percent of the residues may be so changed.

The invention further encompasses antibodies and antibody fragments, such as F_{ab} or (F_{ab})₂, that bind immunospecifically to any of the proteins of the invention.

The above defined information for this invention suggests that this Ribonuclease pancreatic precursor-like protein (NOV8) may function as a member of a “Ribonuclease pancreatic precursor family”. Therefore, the NOV8 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV8 nucleic acids and proteins of the invention are useful in potential therapeutic applications implicated in cancer including but not limited to Inflamation, Autoimmune disorders, Aging and Cancer. For example, a cDNA encoding the Ribonuclease pancreatic precursor-like protein (NOV8) may be useful in gene therapy, and the Ribonuclease pancreatic precursor-like protein (NOV8) may be useful when administered to a subject in need thereof. By way of nonlimiting example, the compositions of the present invention will have efficacy for treatment of patients suffering from Diabetes, Von Hippel-Lindau (VHL) syndrome, Pancreatitis, Obesity, Hyperthyroidism and Hypothyroidism and Cancers including, but no limited to Thyroid and Pancreas, and other such conditions. The NOV8 nucleic acid encoding Ribonuclease pancreatic precursor-like protein, and the ribonuclease pancreatic precursor-like protein of the invention, or fragments thereof, may further be useful in diagnostic applications, wherein the presence or amount of the nucleic acid or the protein are to be assessed.

NOV8 nucleic acids and polypeptides are further useful in the generation of antibodies that bind immuno-specifically to the novel NOV8 substances for use in therapeutic or diagnostic methods. These antibodies may be generated according to methods known in the art, using prediction from hydrophobicity charts, as described in the “Anti-NOVX Antibodies” section below. The disclosed NOV8 protein has multiple hydrophilic regions, each of which can be used as an immunogen. In one embodiment, a contemplated NOV8 epitope is from about amino acids 5 to 25. In another embodiment, a NOV8 epitope is from about amino acids 90 to 100. These novel proteins can be used in assay systems for functional analysis of various human disorders, which will help in understanding of pathology of the disease and development of new drug targets for various disorders.

NOV9

A disclosed NOV9 nucleic acid of 1476 nucleotides (also referred to as SC87421058_A) encoding a novel Aminotransferase-like protein is shown in Table 9A. An open reading frame was identified beginning with an ATG initiation codon at nucleotides 26-28 and ending with a TAA codon at nucleotides 1379-1381. The start and stop codons are in bold letters.

TABLE 9A
NOV9 nucleotide sequence.

(SEQ ID NO:37)

CAGGTGCAAACCAGCCCCAGGCTCCATGGCTTCAAGAAGGTCGAAGTTCAAGGGAAGCACCAAGGCTCCC
TTGTGGGTCTGGAAATCTGCATTGGTAAATGCTTTAGGCTTTTTTACTTCTTCATGCAAAGTTTTCTTTG
CATCGGATCCCATCAAAATAGTGAGAGCCCAGAGGCAGTACATGTTTGATGAGAACGGTGAACAGTACTT
GGACTGCATCAACAATGTTGCCGTGGGACACTGTCACCCAGGAGTGGTCAAAGCTGCCCTGAAACAGATG
GAACTGCTAAATACAAATTCTCGATTCCTCCACGACAACATTGTTGAGTATGCCAGACGCCTTTCAGCAA
CTCTGCCGGAGAAACTCTCTGTTTGTTATTTTACAAATTCAGGGTCCGAAGCCAACGACTTAGCCTTACG
CCTGGCTCGGCAGTTCAGAGGCCACCAGGATGTGATCACTCTTGACGCTTACCATGGTCACCTATCATCC
TTAATTGAGATTAGCCCATATAAGTTTCAGAAAGGAAAAGATGTCAAAAAAGAATTTGTACATGTGGCAC
CAACTCCAGATACTTACAGAGGAAAATATAGAGAAGACCATGCAGACTCAGCCAGTGCTTATGCAGATGA
AGTGAAGAAAATCATTGAAGATGCTCATAACAGTGGAAGGAAGGTTGCTGCCTTTATTGCTGAATCCATG
CAGAGTTGTGGCGGACAAATAATTCCTCCAGCAGGCTACTTCCAGAAAGTGGCACAGTATGTACACGGTG
CAGGGGGTGTGTTTATACCTGATGAAGTTCAAGTACGCTTTGGCAGAGTTGGGAAACATTTCTGGAGCTT
CCAGATGTATGGTGAAGACTTTGTTCCAGACATCGTCACAATGGGAAAACCGATGGGCAACGGCCACCCG
GTCCCATGTGTGGTAACAACCAAAGAAATTGCAGAAGCCTTCAGCAGCTCTGGGATGGAATATTTTAATA
CGTATGGAGGAAATCCAGTATCTTGTGCTGTTGGTTTGGCTGTCCTGGATATAATTGAAAATGAAGACCT
TCAAGGAAATGCCAAGAGAGTAGGGAATTATCTCACTGAGTTACTGAAAAAACAGAAGGCTAAACACACT
TTGATAGGAGATATTAGCGCCATTGGCCTTTTTATTGAATTAATTTCAGTGAAGGACCATCTGAAAAGGA
CCCCTGATATGTATTTAGCTTTGGGGACAATTTTGGTTCTGGAAAAAGAAAAACGAGTGCTTCTCACTGC
CGATGGACCTCATAGAAATGTACTTAAAATAAAACCACCTATGTGCTTCACTGAAGAAGATGCAAAGTTC
ATGGTGGACCAACTTGATAGGATTCTAACAGGTGGGTCCATGGATCTTTAAGATGTCTTCTTGTTCCCTC
TCCCAAACCCACCCCTCAAACCCTGGTCTAGTCATAATGAGCATATGCATCTTGTTATTCATGATGGAAG
TGAGGC

The disclosed NOV9 nucleic acid sequence, localized to chromosome 4, has 342 of 540 bases (63%) identical to a gb:GENBANK-ID:AK023470|acc:AK023470.1 mRNA from Homo sapiens (Homo sapiens cDNA FLJ13408 fis, clone PLACE1001672, weakly similar to PROBABLE AMINOTRANSFERASE T01B11.2 (EC 2.6.1.-).

The disclosed NOV9 polypeptide (SEQ ID NO:28) encoded by SEQ ID NO:27 has 451 amino acid residues and is presented in Table 9B using the one-letter amino acid code. Signal P, Psort and/or Hydropathy results predict that NOV9 has a signal peptide and is likely to be localized in the mitochondrial matrix space with a certainty of 0.5365. In other embodiments, NOV9 may also be localized to the nucleus with a certainty of 0.3600, the microbody with a certainty of 0.2667, or the mitochondrial inner membrane with a certainty of 0.2612. The most likely cleavage site for NOV9 is between positions 34 and 35, SSC-KV.

TABLE 9B
Encoded NOV9 protein sequence.

(SEQ ID NO:38)

MASRRSKFKGSTKAPLNVWKSALVNALGFFTSSCKVFFASDPIKIVRAQRQYMFDENGEQYLDCNNVAV
GHCHPGVVKAALKQMELLNTNSRFLHDNIVEYAKRLSATLPEKLSVCYFTNSGSEANDLALRARQFRGH
QDVITLDAYHGHSSLIEISPYKFQKGKDVKVKKFVVAPTPDTYRGKYREDHADSASAYADEVKKIIEDA
HNSGRKVAAIAESMQSCGGQIIPPAGYFQKVAEYVEGAGGVFIADEVQVGFGRVGKIFWSFQMYGEDFV
PDIVTMGKPMGNGHPVACVVTTKEIAEAFSSSGMEYFNTYGGNPVSCAVGLAVLDLENEDLQGNAKRVG
NYLTELLKKQKAKHTLIGDIRGIGLFIGIDLVKDHLKRTPDMYLALGTILVLEKEKRVLLSADGPHRNVL
KIKPPMCFTEEDAKFMVDQLDRILTGGSMDL

A search of sequence databases reveals that the NOV9 amino acid sequence has 197 of 340 amino acid residues (57%) identical to, and 256 of 340 amino acid residues (75%) similar to, the 474 amino acid residue ptnr:SPTREMBL-ACC:Q9VU95 protein from Drosophila melanogaster (Fruit fly) (CG8745 PROTEIN).

NOV9 is expressed in the brain and the hypothalamus.

The disclosed NOV9 polypeptide has homology to the amino acid sequences shown in the BLASTP data listed in Table 9C.

TABLE 9C
BLAST results for NOV9

| Gene Index/ | | | Identity | Positives | |
|---|---|---|---|---|---|
| Identifier | Protein/Organism | Length (aa) | (%) | (%) | Expect |
| gi\|13775190\|ref\|NP | alanine- | 493 | 95 | 95 | 0.0 |
| 112569.1\| | glyoxylate | | | | |
| (NM_031279) | aminotransferase | | | | |
| | 2-like 1 | | | | |
| | [Homosapiens] | | | | |
| gi\|12836724\|dbj\|BAB | Putative | 499 | 85 | 91 | 0.0 |
| 23784.1\| (AK005060) | protein/mouse | | | | |
| gi\|14734126\|ref\|XP | alanine- | 426 | 96 | 96 | 0.0 |
| 034659.1\| | glyoxylate | | | | |
| (XM_034659) | aminotransferase | | | | |
| | 2-like 1 [Homo | | | | |
| | sapiens] | | | | |
| gi\|12850870\|dbj\|BAB | Putative | 473 | 65 | 80 | e−164 |
| 28878.1\| (AK013489) | protein/mouse | | | | |
| gi\|16768880 gb\|AAL2 | LD09584p | 494 | 58 | 74 | e−138 |
| 8659.1\| (AY061111) | [Drosophila | | | | |
| | melanogaster] | | | | |

The homology between these and other sequences is shown graphically in the ClustalW analysis shown in Table 9D. In the ClustalW alignment of the NOV9 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

[image]

Table 9E lists the domain description from DOMAIN analysis results against NOV9. This indicates that the NOV9 sequence has properties similar to those of other proteins known to contain this domain.

TABLE 9E
Domain Analysis of NOV9

gnl|Pfam|pfam00202, aminotran_3, Aminotransferase class-III
CD-Length = 406 residues, 96.6% aligned
Score = 266 bits (681), Expect = 1e−72

A disclosed NOV9 nucleic acid encodes for a novel member of the Transferase superfamily of enzymes. Specifically, the sequence encodes a amino-transferase-like protein. Amino-transferase enzyes play crucial roles in liver metabolism. Serum amnino-transferase concentrations have been used as an accurate diagnostic measure in cases of liver toxicity and damage such as in liver cancer, cirrhosis due to alcohol abuse, or troglitazone treatment for diabetes. For this reason the enzyes of the amino-transferase superfamily are potentially useful as diagnostic indicators. The protein described here is known to be expressed in brain tissue, which may indicate a role in brain and CNS disorders. The amino-transferase-like protein (NOV9; SC87421058_A) described here could be used in diagnostic tools to detect liver damage due to cirrhosis, cancer, or chemical toxicity; or to detect or treat certain brain and CNS pathologies.

Acute hormonal regulation of liver carbohydrate metabolism mainly involves changes in the cytosolic levels of cAMP and Ca2+. Epinephirine, acting through beta 2-adrenergic receptors, and glucagon activate adenylate cyclase in the liver plasma membrane through a mechanism involving a guanine nucleotide-binding protein that is stimulatory to the enzyme. The resulting accumulation of cAMP leads to activation of cAMP-dependent protein kinase, which, in turn, phosphorylates many intracellular enzymes involved in the regulation of glycogen metabolism, gluconeogenesis, and glycolysis. These are (1) phosphorylase b kinase, which is activated and, in turn, phosphorylates and activates phosphorylase, the rate-limiting enzyme for glycogen breakdown; (2) glycogen synthase, which is inactivated and is rate-controlling for glycogen synthesis; (3) pyruvate kinase, which is inactivated and is an important regulatory enzyme for glycolysis; and (4) the 6-phosphofructo-2-kinase/fructose 2,6-bisphosphatase bifunctional enzyme, phosphorylation of which leads to decreased formation of fructose 2,6-P2, which is an activator of 6-phosphofructo-1-kinase and an inhibitor of fructose 1,6-bisphosphatase, both of which are important regulatory enzymes for glycolysis and gluconeogenesis. In addition to rapid effects of glucagon and beta-adrenergic agonists to increase hepatic glucose output by stimulating glycogenolysis and gluconeogenesis and inhibiting glycogen synthesis and glycolysis, these agents produce longer-term stimulatory effects on gluconeogenesis through altered synthesis of certain enzymes of gluconeogenesis/glycolysis and amino acid metabolism. For example, P-enolpyruvate carboxykinase is induced through an effect at the level of transcription mediated by cAMP-dependent protein kinase. Tyrosine amino-transferase, serine dehydratase, tryptophan oxygenase, and glucokinase are also regulated by cAMP, in part at the level of specific messenger RNA synthesis. The sympathetic nervous system and its neurohumoral agonists epinephrine and norepinephrine also rapidly alter hepatic glycogen metabolism and gluconeogenesis acting through alpha 1-adrenergic receptors. The primary response to these agonists is the phosphodiesterase-mediated breakdown of the plasma membrane polyphosphoinositide phosphatidylinositol 4,5-P2 to inositol 1,4,5-P3 and 1,2-diacylglycerol. This involves a guanine nucleotide-binding protein that is different from those involved in the regulation of adenylate cyclase. Inositol 1,4,5-P3 acts as an intracellular messenger for Ca2+ mobilization by releasing Ca2+ from the endoplasmic reticulum.

The disclosed NOV9 nucleic acid of the invention encoding a Aminotransferase-like protein includes the nucleic acid whose sequence is provided in Table 9A, or a fragment thereof. The invention also includes a mutant or variant nucleic acid any of whose bases may be changed from the corresponding base shown in Table 9A while still encoding a protein that maintains its Aminotransferase-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 37 percent of the bases may be so changed.

The disclosed NOV9 protein of the invention includes the Aminotransferase-like protein whose sequence is provided in Table 9B. The invention also includes a mutant or variant protein any of whose residues may be changed from the corresponding residue shown in Table 2 while still encoding a protein that maintains its Aminotransferase-like activities and physiological functions, or a functional fragment thereof. In the mutant or variant protein, up to about 43 percent of the residues may be so changed.

The invention further encompasses antibodies and antibody fragments, such as F_{ab} or (F_{ab})₂, that bind immunospecifically to any of the proteins of the invention.

The above defined information for this invention suggests that this Aminotransferase-like protein (NOV9) may function as a member of a “Aminotransferase family”. Therefore, the NOV9 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV9 nucleic acids and proteins of the invention are useful in potential therapeutic applications implicated in liver toxicity and damage such as in cancer, cirrhosis, or troglitazone treatment for diabetes; brain and CNS disorders including cancer, Parkinson's, Alzheimer's, epilepsy, schizophrenia and other diseases, disorders and conditions of the like. For example, a cDNA encoding the Aminotransferase-like protein (NOV9) may be useful in gene therapy, and the Aminotransferase-like protein (NOV9) may be useful when administered to a subject in need thereof. By way of nonlimiting example, the compositions of the present invention will have efficacy for treatment of patients suffering from bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation. Dentatorubro-pallidoluysian atrophy (DRPLA) Hypophosphatemic rickets, autosomal dominant (2) Acrocallosal syndrome and dyskinesias, such as Huntington's disease or Gilles de la Tourette syndrome, and/or other pathologies or conditions. The NOV9 nucleic acid encoding Aminotransferase-like protein, and the Aminotransferase-like protein of the invention, or fragments thereof, may further be useful in diagnostic applications, wherein the presence or amount of the nucleic acid or the protein are to be assessed.

NOV9 nucleic acids and polypeptides are further useful in the generation of antibodies that bind immuno-specifically to the novel NOV9 substances for use in therapeutic or diagnostic methods. These antibodies may be generated according to methods known in the art, using prediction from hydrophobicity charts, as described in the “Anti-NOVX Antibodies” section below. The disclosed NOV9 protein has multiple hydrophilic regions, each of which can be used as an immunogen. In one embodiment, a contemplated NOV9 epitope is from about amino acids 10 to 40. In another embodiment, a NOV9 epitope is from about amino acids 60 to 75. In alterative embosiments, a NOV9 epitope is from about amino acids 210 to 250, from about amino acids 310 to 340, and from about amino acids 360 to 390. These novel proteins can be used in assay systems for functional analysis of various human disorders, which will help in understanding of pathology of the disease and development of new drug targets for various disorders.

NOV10

NOV10 includes two tolloid-like 2-like proteins disclosed below. The disclosed sequences have been named NOV10a and NOV10b.

NOV10a

A disclosed NOV10A nucleic acid of 3350 nucleotides (also referred to as CG50235-01) encoding a novel Tolloid-like 2-like protein is shown in Table 10A. An open reading frame was identified beginning with an ATG initiation codon at nucleotides 365-367 and ending with a TAG codon at nucleotides 3341-3343. The start and stop codons are in bold letters.

TABLE 10A
NOV10A nucleotide sequence.

(SEQ ID NO:39)

CGCCCATTGGCTCCTCAGCCAAGCACGTACACCAAATGTCTGAACCTGCGGTTCCTCTCGTACTGAGCAGGATTACCATG
GCAACAACACATCATCAGTAGGGTAAAACTAACCTGTCTCACGACGGTCTAAACCCAGGCAGCCTCGGCCGCCGGGCAAG
TAGCTCCGAGCGGCTGCTTCCCGGTTGCCTCGAAGAAGACAGGGGGCGCCGCGCTCCGCTTGCTCCGCGCCTGAGCCATG
CCCAGCGCCCTGTGTAACCACCGGTCCCGCCGGAGCCGACCGACCAAGTGCAGTGCGCCGTCTTTCGGCCGAGCTAAGCT
TTCGTGCACGCAACTCCCTCTGCCCCAGCCGGCCCCGCGCCACCATGCCCCGGGCGACTGCACTTGGGGCCCTGGTGTCA
CTGCTGCTGCTGCTGCCGCTGCCTCGCGGCGCCGGGGACTCGGGGAGCGCCCGGACGCCACCGCAGACTACTCAGAGCT
GGACGGCGAGGAGGGCACGGAGCAGCAGCTGGAGCATTACCACGACCCTTGCAAAGCCGCTGTCTTTTGGGGAGACATTG
CCTTAGATGAAGATGACTTGAAGCTGTTTCACATTGACAAAGCCAGAGACTGGACCAAGCAGACAGTGGGGGCAACAGGA
CACAGCACAGGTGGGCTTGAAGAGCAGGCATCTGAGAGCAGCCCAGACACCACAGCCATGGACACTGGCACCAAGGAAGC
TGGAAAGGATGGCCGGGAGAATACCACACTCCTGCACAGCCCTGGGAGCCTTGCATGCCGCAAGACCTTCTCTCCCC
GGGTCCGAAGAGCCACAACCTCAAGGACAGAGAGGATARGGCCTGGAGGAGTCATCCCCTACGTCATTGGAGGGAACTTC
ACTGGGAGCCAGAGGGCCATTTTTAAGCAGGCCATGAGACACTGGGAGAAGCACACCTGTGTGACCTTCATAGAAAGGAC
GGATGAGGAAAGCTTTATTGTATTCAGTTACAGAACCTGTGGCTGTTGCTCCTATGTTGGCCGCCGAAAGGAGGCCACAC
AGGCCATATCCATTGGAAGAACTGTGACAAGTTTGGCATTGTGGCTCACGAGCTGGGCCATGTGGTTGGGTTTTGGCAT
GAACACACCCGGCCAGACAGAGACCAACAATGTCACCATCATCAGGGAAAACATCCAGCCAGGTCAGGAGTATAATTTCTT
AAAAATGGAAGCTGGGGAAGTGAGCTCTCTGGGAGAGACATACGACTTTGACAGCATCATGCACTACGCCCGGAACACCT
TCTCAAGAGGAGTTTTCTTAGACACCATCCTTCCCCGTCAAGATGACATGGCGTCAGAACAGCCATTGACCAGCGCGTG
CGGCTCAGTCAGGGAGAATAGCTCAAGCCCGGAGCTGTACAAATGCCCAGCGTGTGGGGAGACCCTGCAGGACACCC
GGGAAACTTTTCTGCACCTGGTTTCCCAAATGGGTACCCATCTTACTCCCACTGCGTCTGGAGGATCTCGGTCACCCCAG
GGGAAAAGATCGTATTAAACTTCACATCCATCGATTTGTTTAAAAGCCCACTGTGCTCGTATGATTACGTGGAGGTCCGG
GATGGTTACTGGAGAAAAGCCCCCCTTTTGGGCACGTTTTGTGGCGATAAGATCCCCCAGCCCCTCGTCTCCCCGCACAG
CCGGCTCTGGGTGGAGTTCCGAGCAGAGCACATCTTGGGCAAGGGCTTCTTTGCAGCGTACGAAGCTACCTCCGGGG
GAGACATGAACAAAGATGCCGGTCAGATTCAATCTCCCAACTATCCGGATGACTACAGACCTTCCCAAGGATGTGTCTGG
AGGATTACGGTTTCAGAGGGGTTTCACGTGGGACTTACCTTCCAAGCTTTTGAGATTGAAAGGCACGACAGCTGTGCATA
TGACTACCTGGAAGTCCGGGATGGCCCCACGGAAGAGAGTGCCCTGATCGGCCACTTTTGTGGCTATGAGAAGCCACAGG
ATGTGAATCGAGCTCCAACAACGGACTAATCGATGAAGTTTGTGTCCGATGGCTCTATCAATAAAGCGGGCTTTGCAGCCT
TTTTTCAAGGAAGTGGATGAGTGTTCCTGGCCAGATCACGGCGGGTGCGACATCGCTGTCTGACACGCTGGGCAGCTA
CAAGTGTGCCTGTGACCCTGGCTACGAGCTGGCCGCCCATAAGAAATGTGTGAAGTGGCCTGTCGCAGATTTCATTACCA
AGCTGAATGGAACCATCACCAGCCCTGGGTGGCCGAAGGAGACGTATCCCACACAACTGTGTCTGAGGTGGTGGCC
CCCACTCAGTACCCGACATCTCCCTTCAGTTTGAAGTGTTTCAACTGGAAGGAAGTCACGTCTGTAAGTACGACTTTGTAGA
GGTGCGCAGCGCCTGTCCCCCGACGCGAGCTGCACGAGGTTCTGCGGCTCTGAGACGCCGAAAGTAATCACCTCGC
AGAGCAACAACCATGCGCGTGGAGTTCAAGTCCGACAACACCGTCTCCAAGCGCGGCTTCAGGGCCCACTTCTTCTCAGAT
AAGGACGAGTGTGCCAAGGACAACGGCGGGTGTCAGCATGAGTGCGTCAACACCTTCGGGAGCTACCTGTGCAGGTGCAG
AACGCCTACTGGCTCCACGAGAATGGGCATGACTAAGCAAAGAGGCTGGCTGTGCACACAAGATCAGCAATGTGGAGGGGA
CCCTGGCGAGCCCCAACTGGCCTGACAAATACCCCAGCCGGAGGGAGTGTACCTGGAACATCTCATTCGACATGGCACCAC
AGAGTGAACTCACCTTTAATGAGTTTGAGATCGAGCAGCACCAGGAATGTGCCTATGACCACCTGGAAATGTATGACGG
GCCGGACAGCCTGGCCCCCATTCTCGGCCGTTTCTGCAGCAGCAAGAAACCAGACCCCACGGTAGCTTCCGGCAGCAAGT
GCGGGGGCAGCCTGAAGGCTGAAGTGCAGACCAAAGAGCTCTATTCCCACGCCCAGTTTGGGGACAAACACTACCCGAGC
GAGGCCCGCTGTGACTGGGTGATCGTGGCAGAGCACGGCTACGGCGTGGAGCTGACATTCCGGACCTTTGAGGTTGAGGA
GAGGCCCACTGCGCTACGACTAATGGAAGCCTACGACGGCTACGACAGCTCAGCGCCCAGGCTCAAGACCGCTTCTGTG
GCTCTCTGGCCATTAGAAGAAATCTACTCTGCAGGTGATTCCCTGATGATTCGATTCCGCACAGATGACACCAATCAAG
AAAGGCTTTCATGCCCGATACACCAGCACCAAGTTCCAGGATGGCCTGCACATGAAGAAATAGTGCTGAT

In a search of public sequence databases, the NOV10A nucleic acid sequence, which maps to chromosome 10, has 2955 of 2957 bases (99%) identical to a gb:GENBANK-ID:AF059516|acc:AF059516.1 mRNA from Homo sapiens (Homo sapiens tolloid-like 2 protein (TLL2) mRNA, complete cds).

The disclosed NOV10A polypeptide (SEQ ID NO:30) encoded by SEQ ID NO:29 has 992 amino acid residues and is presented in Table 10B using the one-letter amino acid code. Signal P, Psort and/or Hydropathy results predict that NOV10A has a signal peptide and is likely to be localized extracellularly with a certainty of 0.7523. In other embodiments, NOV10A may also be localized to the microbody (peroxisome) with acertainty of 0.2280, the lysosome (lumen) with a certainty of 0.1900, or in the endoplasmic reticulum (membrane) with a certainty of 0.1000.

TABLE 10B
Encoded NOV10A protein sequence.

(SEQ ID NO:40)

MPRATALGALVSLLLLLPLPRGAGGLGERPDATADYSELDGEEGTEQQLEHYHDPCKAAVFWGDIADED
DLKLFHIDKARDWTKQTVGATGHSTGGLEEQASESSPDTTAMDTGTKEAGGKDGRENTTLLHSPGTHAAA
KTFSPRVRRATTSRTERIWPGGVTPYVICGNFTGSQRAIFKQANRHWEKHTCVTFIERTDESSFIVFSYR
TCGCCSYVGRRGGQPQAISTGKNCDKFGIVAHELGHVXTGFWHEHTRPDRDQHVTIRENIQPGQEYNFLK
MFGEVSSLGETYDFDSIMTTYARNTFSRGVFLDTILPRQDDNGVRPTIGQRVRLSQGDIAQARKLYKCPA
CGETLQDTTGNFSAPGFPNGYPSYSHCVWRISVTPGEKIVLNFTSNDLFKSRLCWYDYVEVRDGYWRKAP
LLGRFCGDKIPEPLVSTDSRLWVEFRSSSNILGKGFFAAYEATCGGDRMKDAGQIQSPNYPDDYRPSKEC
VWRITVSEGFHVGLTFQAFEIERHDSCAYDYLEVRDGPTEESALIGHFCGYEKPEDVKSSSNRLWMKFVS
DGSINKAGFAANFFKEVDECSWPDHGGCEHRCVNTLGSYKCACDPGYELAADKKMCEVACGGFITKLNGT
ITSPGWPKEYPTNKNCVWQVVAPTQYRISLQFSVFELEGNDVCKYDFVEVRSGLSPDAKLHGRFCGSETP
EVITSQSNNMRVEFKSDNTVSKRGFRFFSDKLECAKLNGGCQHECVNTFGSYLCRCRNGYWLHENGHD
CKEAGCAIKISSVEGTLASPNWPDKYPSRRECTWNISSTAGHRVKLTFNEFEIEQHQECAYDHLEMYDGP
DSLAPILGRFCGSKKPDPTVASGSKCCGGRLKAEVQTKELYSHAQFGDNNYPSEATCDWVIVAEDGYGVEL
TFRTFEVEEEADCGYDYMEAYDGYDSSAPRLGRFCGSGPLEEIYSAGDSLMIRFRTDDTINKKGFHARYT
STKFQDGLHMKK

A search of sequence databases reveals that the NOV10A amino acid sequence has 868 of 879 amino acid residues (98%) identical to, and 868 of 879 amino acid residues (98%) similar to, the 1015 amino acid residue ptnr:SPTREMBL-ACC:Q9Y6L7 protein from Homo sapiens (Human) (TOLLOID-LIKE 2 PROTEIN).

NOV10A is expressed in at least the colon, lung, parotid salivary glands and whole organism.

NOV10b

A disclosed NOV10B nucleic acid of 3146 nucleotides (also referred to as CG50235-03) encoding a novel Tolloid-like 2-like protein is shown in Table 10A. An open reading frame was identified beginning with an ATG initiation codon at nucleotides 227-229 and ending with a TAG codon at nucleotides 3137-3139. The start and stop codons are in bold letters.

TABLE 10C
NOV10B nucleotide sequence.

| | |
|---|---|
| GCAGCCTCGGCCGCCGGGCAGTAGCTCCGAGCGGCTGCTTCCCGGTTGCCTCGACGAAG | (SEQ ID NO:41) |
| ACAGGGGCGCCGCGCTCCGCTTCCTCCGCGCCTGAGCCATGCCCAGCAGCCCTGTGTAA | |
| CCACCGAGTCCCGGCCGGAGCCGACCGACCCAGTGTCGCCGTCTTTCGGCCCAGCTGAG | |
| CTTTCGTGCACGCAACTCCCTCTGCCCCAGCCGGCCCCGCCCCACCATGCCCCGGGCGAC | |
| TGACTTGGGGCCCTGGTGTCACTGTGCTGCTGCTGCTGCCGCTGCCTCGCGGCGCCGGGGG | |
| ACTCGGGGAGCGCCCGGACGCCACCGCAGACTACTCAGAGCTCGGACGGCGAGGAGGGCAC | |
| GGAGCAGCAGCTGGAGCATTTACCACGACCCTTTGAAAGCCGCTGTCTTTTGGGGAGACAT | |
| TGCCTTAGATGAAGATGACTTGAAGCTGTTTCACATTGACAAAGCCAGAGACTGAGACCAA | |
| GCAGACAGTGGGGGCAACAGGACACAGCACAGGTGGGCTTGAAGAGCAGGCATCTCGAGAG | |
| CAGCCCAGACACCACAGCCATGGACACTGGCACCAAGGAAGCTTGGAAAGGGGAGCCAGAG | |
| GGCCATTTTTAAGCAGGCCATGAGACACTGGGAGAAGCACACCTGTGTGACCTTCAATAGA | |
| AAGGACGGATGAGGAAGCTTTATTGTATTCAGTTACAGATTACCTGTAACTGTTGCTCCTA | |
| TGTTGGCGCCGAGGAGGAGGCCCACAGGCCATATCACCATTGGGAGTTCTGTAATTGGGTT | |
| TGGCATTGTGGCTCACGAGCTGGGCCATGTGGTTGGGTTTTGGCATGAACACACCCCGGCC | |
| AGACAGAGACCAACATGTCACCATCATCAGGGAAAACATCCAGCCAGGTTCAGGAGTATAA | |
| TTTCTTAAAATGGAAGCTGGGGCAAAGTGAGCTCTCTGGGAGAGACATACGACTTTGACAG | |
| CATCATGCACTACGCCCGGAACACCTTCTCAAGAGGAGTTTTTTTAGACACCAATCCTTCC | |
| CCGTCGATGACATGGCGTCAGGCCAACCATTGGACCCCAGCGCGTGCCGCTTCAGTCAGGG | |
| AGACATAGCTGCAAGCCCGGAAGCTGTACAAATGCCCAGGTCCTACTTGTCCTTTTGTTAG | |
| CCAGAAAACATCAATCTGCTTGCTACACTTCTCACCAACCTGTTCCGAGGGGCTTTGGCTG | |
| GCAAAGGGCGTGTGGGAACCACCCTGCAGGACACAACGGGAAACTTTTCTGCACCTGGTTT | |
| CCCAAATGGGTACCCATCTTACTCCCACTGCGTCTGGAGGATCTCGGTCACCCCCAGGGGA | |
| AAAGATCGTATTAAACTTCACATCCATGGATTTGTTTAAAAGCCGACTGTGCTGGTATTGA | |
| TTACGTGGAGGTCCGGGATGGTTACTGGAGAAAAAGCCCCCCTTTTGGGCAGGTTTTGTGG | |
| CGATAAGATCCCGGAGCCCCTCGTCTCCACGGACAGCCGGCTCTGGGTCGGAGTTCCGCAG | |
| CAGCACCACATCTTGGGCAGGGACTTCTTTGCAGCGTACGAAGAGCTACCTGCGGGGGAGA | |
| CATGAACAAAGATGCCCGTCAGATTCATCTCCCAACTATCCGGATGACTACAGAGACCTTC | |
| CAAGGAATGTGTCTGGAGGATTACGGTTTCAGAGGGGTTTCACGTGGGACTTACCTTCCCA | |
| AGCTTTTGAGATTGAGGCACGACAGCTGTGCATATGACTACCTGGAAGTCCGTGGGGATGG | |
| CCCCACGGGAGAGTGCCCTGATCGGCAGCCACTTTTGTGGCTATGAGAAGCCGGAGGATGT | |
| GAATCGAGCTCCAACAGACTGTGGATCAAGTTTGTGTCCGGCATGGCTCTATCAATAAAGC | |
| GGGCTTTGCAGCCAATTTTTTCAAGGAGGTGGATGAGTGTTCCTTGGCCAGATCACGGCGG | |
| GTGCGAGCATCGCTGTGTGAACACGCTGGGCAGCTACAAGTGTTGCCTGTTCCCTGAGCTA | |
| CGAGCTGGCCGCCGATAAGAAGATGTGTGAAGTGGCCTGTGGCGGTTTCATTACCAAGCT | |
| GAATGGAACCATCACCAGCCCTGGGTGGCCGAAGGAGTATCCCACAAACAAAAACTGTGT | |
| CTGGCAGGTGGTGGCCCCCACTCAGTACCGGATCTCCCTTCAGTTTGCAGTGTTTGAACT | |
| GGAAGGCAATGACGTCTGTAAGTACGACTTTGTAGAGGTGCGCACCAACCTGTCCCCCGA | |
| CGCCAGCTGCACGGCAGGTTCTGCGGCTCTGAGACGCCGGAGGTTTATTGACCTCGCAGAG | |
| CAACAACATGCGCGTCGAGTTCAAGTCCGACACACCGTCTCCAAAGCGCGGCTTCAGGGC | |
| CCACTTCTTCTCAGATAAGGACGAGTGTGCCAAGGACCGGCGGGTGTCAGGCATTGAGTG | |
| CGTCAACACCTTCGGGAGCTACCTGTGCAGGTGCAGACGGCTACTGGCTCCGAGAA | |
| TGGGCATGACTGCAAAGAGGCTGGCTGTGCACACAAGATCAGCAGTGTGGAGACCCT | |
| GGCGAGCCCCAACTGGCCTGACAATACCCCAGCCGAGGGAGTGTACCTGGAACATCTC | |
| TTCGACTGCAGGCCACAGAGTGAAACTCACCTTTAATGAGTTTGAGATCGAGCAGCACCA | |
| GGAATGTGCCTATGACCACCTGGAATGTATGACCGCCCCCGATTCCCTGGCCCCATTCT | |
| GGCCGTTTCTGCGGTAGCAGAAAGCCAGACCCCACGGTGGCTTCCGGCAGCAAGTGCGG | |
| GGGCAGGCTGAAGGCTGAAGTGCAGACCAAAGAGCTCTATTCCCACGCCTGTTTGGGGA | |
| CAACAACTACCCGAGCGAGGCCCGCTGTGACTGGGTGATCGTGGAGAGGACGGCTACGG | |
| CGTGGAGCTGACATTCCGGACCTTTGAGGTTGAGGAGGAGGCCGACTGCTCTACGACTA | |
| CATGGAAGCCTACGACGGCTACGACAGCTCAGCGCCCAGGCTCGCCGCTTCTGTGGCTC | |
| TGGGCCATTAGAAGAATCTACTCTGCAGGTGATTCCCTGATGATTCGATTCCGCACAGA | |
| TGACACCATCAACAGAAGGCTTTCATGCCCCATACACCAAGCACAAGTTCCAGGGATGC | |
| CCTGCACATGAAGAAATAGTGCTGAT | |

In a search of public sequence databases, the NOV10B nucleic acid sequence, which maps to chromosome 10, has 1882 of 1884 bases (99%) identical to a gb:GENBANK-ID:AK026106|acc:AK026106.1 mRNA from Homo sapiens (Homo sapiens cDNA: FLJ22453 fis, clone HRC09679, highly similar to AF059516 Homo sapiens tolloid-like 2 protein (TLL2) mRNA).

The disclosed NOV10B polypeptide (SEQ ID NO:30) encoded by SEQ ID NO:29 has 970 amino acid residues and is presented in Table 10B using the one-letter amino acid code. Signal P, Psort and/or Hydropathy results predict that NOV10B has a signal peptide and is likely to be localized extracellularly with a certainty of 0.7523. In other embodiments, NOV10B may also be localized to the microbody (peroxisome) with acertainty of 0.2291, the lysosome (lumen) with a certainty of 0.1900, or in the endoplasmic reticulum (membrane) with a certainty of 0.1000. The most likely cleavage site of the disclosed NOV10b polypeptide is between positions 25 and 26 (AAG-LG).

TABLE 10D
Encoded NOV10B protein sequence.

| | |
|---|---|
| MPRATALGALVSLLLLLPLPRGAGGLGERPDATALYSELDGEEGTEQQLEHYHDPCKAAV | (SEQ ID NO:42) |
| FWGDIALDEDDLKLFHIDKAJDWTKQTVGATGHSTGGLEEQASESSPDTTAMDTGTKEAG | |
| KGSQRAIFKQARHWEKHTCVTFIERTDEESFIVFSYRTCGCCSYVGRRGGGQPQAISIGK | |
| NCDKFGIVAHELGHVVGFWHEHTRPDRDQHVTIIRENIQPGQEYNFLKMEAGEVSSLGET | |
| YDFDSIDMHYARNTFSRGVFLDTILPRQDDNGXRPTIGQRVRLSQGDIAQARIYKCPGPT | |
| CAFVSQKTSICLLHFSPTCSEGFGWQRACGETLQDTTGNFSAPGFPNGYPSYSHCVWRIS | |
| VTPGEKIVLNFTSPHDLFKSRLCWYDYVELGYWRKAAPLLGRFCGDKIPEPLVSTDSRLW | |
| VEFRSSSNILGKGFFAAYEATCGGDMNKDAGQIQSPNYPDDTRPSKECVWRITVSEGFHV | |
| GLTFQAFEIERIHDSCAYDYLEVRDGPTEESALIGHFCGYEFPDVKSSSNRLWMKFVSDG | |
| SINKAGFAANFFKEVDECSWPDHCGCEHRCVNTLGSYKCACDPGYELAADKKMCEVACGG | |
| FITKLNGTITSPGWPKEYPTNKNCVWQVVAPTQYRISLQFEVFELEGNDVCKYDFVEVRS | |
| GLSPDAKLHGRFCGSETPEVITSQSNNMRVEFKSDNTVSKRGFRAHFFSDKDECAKDNGG | |
| CQHECVNTFGSYLCRCRNGYWLHENGHDCKEAGCAHKISSVEGTLASPNWPDKYPSRREC | |
| TWNISSTAGHRVLTFNEFEFIEQHQECAYDHLEMYDGPDSLAPILGRFCGSKKPDPTVAS | |
| GSKCGGRLKAEVQTKELYSHAQFGDNNYPSEARCDWVIVAEDGYGVELTFRTFEVEEEAD | |
| CGYDYMEAYDGYDSSAPRLGRFCGSGPLEEIYSAGDSLMIRFRTDDTINKKGFHARYTST | |
| KFQDALHMKK | |

A search of sequence databases reveals that the NOV10B amino acid sequence has 519 of 530 amino acid residues (97%) identical to, and 519 of 530 amino acid residues (97%) similar to, the 1015 amino acid residue ptnr:SPTREMBL-ACC:Q9Y6L7 protein from Homo sapiens (Human) (TOLLOID-LIKE 2 PROTEIN).

NOV10B is expressed in at least the Parotid Salivary glands, Colon, Spinal Chord, and Lung.

The disclosed NOV10A polypeptide has homology to the amino acid sequences shown in the BLASTP data listed in Table 10E.

TABLE 10E
BLAST results for NOV10A

| Gene Index/ | | | Identity | Positives | |
|---|---|---|---|---|---|
| Identifier | Protein/Organism | Length (aa) | (%) | (%) | Expect |
| gi\|6678363\|ref\|NP_ | tolloid-like [Mus | 1013 | 70 | 81 | 0.0 |
| 033416.1 | musculus] | | | | |
| (NM_009390) | | | | | |
| gi\|6755807\|ref\|NP_ | tolloid-like 2 | 1012 | 87 | 90 | 0.0 |
| 036034.1\| | [Mus musculus] | | | | |
| (NM_011904) | Length = | | | | |
| | 1012 | | | | |
| gi\|6912724\|ref\|NP_ | tolloid-like 2; | 1015 | 97 | 97 | 0.0 |
| 036597.1\| | KIAA0932 protein | | | | |
| (NM_012465) | [Homo sapiens] | | | | |
| gi\|5902808\|ref\|NP_ | bone | 823 | 72 | 81 | 0.0 |
| 006119.1\| | morphogenetic | | | | |
| (NM_006128) | protein 1, | | | | |
| | isoform 2, | | | | |
| | precursor; PCP | | | | |
| | [Homo sapiens] | | | | |
| gi\|2695979\|emb\|CAA7 | xolloid [Xenopus | 1019 | 75 | 85 | 0.0 |
| C854.1\| (Y09661) | laevis] | | | | |

The homology between these and other sequences is shown graphically in the ClustalW analysis shown in Table 10F. In the ClustalW alignment of the NOV10A protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

[image]

Tables 10G-10I lists the domain description from DOMAIN analysis results against NOV10A. This indicates that the NOV10A sequence has properties similar to those of other proteins known to contain this domain.

TABLE 10G
Domain Analysis of NOV10A

gnl|Pfam|pfam01400, Astacin, Astacin (Peptidase family M12A)
CD-Length = 189 residues, 100.0% aligned
Score = 280 bits (715), Expect 4e−76

TABLE 10H
Domain Analysis of NOV10A

gnl|Pfam|pfam00431, CUB, CUB domain
CD-Length = 110 residues, 100.0% aligned
Score = 159 bits (403), Expect = 5e−40

TABLE 10I
Domain Analysis of NOV10A

gnl|Smart|smart00235, ZnMc, Zinc-dependent metalloprotease;
Neutral zinc metallopeptidases
CD-Length = 143 residues, 99.3% aligned
Score = 130 bits (328), Expect = 3e−31

Vertebrate bone morphogenetic protein 1 (BMP-1) and Drosophila Tolloid (TLD) are prototypes of a family of metalloproteases with important roles in various developmental events. BMP-1 affects morphogenesis, at least partly, via biosynthetic processing of fibrillar collagens, while TLD affects dorsal-ventral patterning by releasing TGFbeta-like ligands from latent complexes with the secreted protein Short Gastrulation (SOG). In a screen for additional mammalian members of this family of developmental proteases, Scott et al. (Dev Biol 1999;213:283-300) identified novel family member mammalian Tolloid-like 2 (mTLL-2) and compare enzymatic activities and expression domains of all four known mammalian BMP-1/TLD-like proteases [BMP-1, mammalian Tolloid (mTLD), mammalian Tolloid-like 1 (mTLL-1), and mTLL-2].

Despite high sequence similarities, distinct differences are shown in ability to process fibrillar collagen precursors and to cleave Chordin, the vertebrate orthologue of SOG. As previously demonstrated for BMP-1 and mTLD, mTLL-1 is shown to specifically process procollagen C-propeptides at the physiologically relevant site, while mTLL-2 is shown to lack this activity. BMP-1 and mTLL-1 are shown to cleave Chordin, at sites similar to procollagen C-propeptide cleavage sites, and to counteract dorsalizing effects of Chordin upon overexpression in Xenopus embryos. Proteases mTLD and mTLL-2 do not cleave Chordin. Differences in enzymatic activities and expression domains of the four proteases suggest BMP-1 as the major Chordin antagonist in early mammalian embryogenesis and in pre- and postnatal skeletogenesis.

Lysyl oxidase catalyzes the final enzymatic step required for collagen and elastin cross-linking in extracellular matrix biosynthesis. Pro-lysyl oxidase is processed by procollagen C-proteinase activity, which also removes the C-propeptides of procollagens I-III. The Bmp1 gene encodes two procollagen C-proteinases: bone morphogenetic protein 1 (BMP-1) and mammalian Tolloid (mTLD). Mammalian Tolloid-like (mTLL)-1 and -2 are two genetically distinct BMP-1-related proteinases, and mTLL-1 has been shown to have procollagen C-proteinase activity. Uzel et al. (2001) directly compared pro-lysyl oxidase processing by these four related proteinases. In vitro assays with purified recombinant enzymes show that all four proteinases productively cleave pro-lysyl oxidase at the correct physiological site but that BMP-1 is 3-, 15-, and 20-fold more efficient than mTLL-1, mTLL-2, and mTLD, respectively. To more directly assess the roles of BMP-1 and mTLL-1 in lysyl oxidase activation by connective tissue cells, fibroblasts cultured from Bmp1-null, Tll1-null, and Bmp1/Tll1 double null mouse embryos, thus lacking BMP-1/mTLD, mTLL-1, or all three enzymes, respectively, were assayed for lysyl oxidase enzyme activity and for accumulation of pro-lysyl oxidase and mature approximately 30-kDa lysyl oxidase. Wild type cells or cells singly null for Bmp1 or Tll1 all produced both pro-lysyl oxidase and processed lysyl oxidase at similar levels, indicating apparently normal levels of processing, consistent with enzyme activity data. In contrast, double null Bmp1/Tll1 cells produced predominantly unprocessed 50-kDa pro-lysyl oxidase and had lysyl oxidase enzyme activity diminished by 70% compared with wild type, Bmp1-null, and Tll1-null cells. Thus, the combination of BMP-1/mTLD and mTLL-1 is shown to be responsible for the majority of processing leading to activation of lysyl oxidase by murine embryonic fibroblasts, whereas in vitro studies identify pro-lysyl oxidase as the first known substrate for mTLL-2. (See Uzel et al. J Biol Chem Jun. 22, 2001; 276(25):22537-22543).

The disclosed NOV10A nucleic acid of the invention encoding a Tolloid-like 2-like protein includes the nucleic acid whose sequence is provided in Table 10A or a fragment thereof. The invention also includes a mutant or variant nucleic acid any of whose bases may be changed from the corresponding base shown in Table 10A while still encoding a protein that maintains its Tolloid-like 2-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 1 percent of the bases may be so changed.

The disclosed NOV10A protein of the invention includes the Tolloid-like 2-like protein whose sequence is provided in Table 10B. The invention also includes a mutant or variant protein any of whose residues may be changed from the corresponding residue shown in Table 10B while still encoding a protein that maintains its Tolloid-like 2-like activities and physiological functions, or a functional fragment thereof. In the mutant or variant protein, up to about 3 percent of the residues may be so changed.

The invention further encompasses antibodies and antibody fragments, such as F_{ab} or (F_{ab})₂, that bind immunospecifically to any of the proteins of the invention.

The above defined information for this invention suggests that this Tolloid-like 2-like protein (NOV10A) may function as a member of a “Tolloid-like 2-family”. Therefore, the NOV10A nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV10A nucleic acids and proteins of the invention are useful in potential therapeutic applications implicated in cancer including but not limited to various pathologies and disorders as indicated below. For example, a cDNA encoding the Tolloid-like 2-like protein (NOV10A) may be useful in gene therapy, and the Tolloid-like 2-like protein (NOV10A) may be useful when administered to a subject in need thereof. By way of nonlimiting example, the compositions of the present invention will have efficacy for treatment of patients suffering from: xerostomia, multiple sclerosis, leukodystrophies, pain, neuroprotection, systemic lupus erythematosus, autoimmune disease, asthma, emphysema, scleroderma, allergy, ARDS, cancer, trauma, regeneration (in vitro and in vivo), viral/bacterial/parasitic infections, as well as other diseases, disorders and conditions.

The NOV10A nucleic acid encoding the Tolloid-like 2-like protein of the invention, or fragments thereof, may further be useful in diagnostic applications, wherein the presence or amount of the nucleic acid or the protein are to be assessed.

NOV10A nucleic acids and polypeptides are further useful in the generation of antibodies that bind immuno-specifically to the novel NOV10A substances for use in therapeutic or diagnostic methods. These antibodies may be generated according to methods known in the art, using prediction from hydrophobicity charts, as described in the “Anti-NOVX Antibodies” section below. The disclosed NOV10A protein has multiple hydrophilic regions, each of which can be used as an immunogen. In one embodiment, a contemplated NOV10A epitope is from about amino acids 1 to 30. In another embodiment, a NOV10A epitope is from about amino acids 300 to 330. These novel proteins can be used in assay systems for functional analysis of various human disorders, which will help in understanding of pathology of the disease and development of new drug targets for various disorders.

NOV11

A disclosed NOV11 nucleic acid of 1604 nucleotides (also referred to as SV135004534_A) encoding a novel Cysteine sulfinic acid decarboxylase-like protein is shown in Table 11C. An open reading frame was identified beginning with an ATG initiation codon at nucleotides 61-63 and ending with a TAG codon at nucleotides 1543-1545.

TABLE 11A
NOV11 nucleotide sequence.

(SEQ ID NO:43)

TAGATTATCTCTCAAACACAATTTGTTTGCTTGCTTCCAGCAGATATTGATCAACAAGAGATGATTCCAA
GTAAGAAGGGGGTTGTCCTGAATCGTGATGCAAAAGCTGGAGAAAAATTTGTTGAAGAGGCCTGTAGGCT
AATAATGGAAGAGGTGGTTTTGAAAGCTACAGATGTCAATGAGAAGGTATGTGAATGGAGGCCTCCTGAA
CAACTGAAACAGCTTCTTGATTTGGAGATGAGAGACTCAGCCCAGCCACCCCATAAACTATTGGAACTCT
GTCGGGATGTCATACACTACAGTGTCAAAACACACCACCCAAGATTTTTCAACCAATTGTATGCTGGACT
TGATTATTACTCCTTGGTGGCCCGATTTATGACCGAAGCATTGAATCCAAGTAGTTATACGTATGAGGTG
TCCCCAGTGTTTCTGTTAGTCGAAGAAGCGGTTCTGAAGAAAATGATTGAATTTATTCGCTGGAAAGAAG
GGGATGGAATATTTAACCCAGGTGGCTCAGTGTCCAATATGTATGCAATGAATTTAGCTAGATACAAATA
TTGTCCTGATATTAAGGAAAAGGGGCTGTCTGGTTCGCCAAGATTAATCCTTTTCACATCTGCAGAGTGT
CATTACTCTATGAAGAACGCAGCCTCTTTTCTTGGGATTCGCACTGAGAATGTTTGCTTTGTGGAAACAG
ATAGAGGTAAAATGATACCTGAGGAACTGGAGAAGCAAGTCTGGCAACCCAGAAAAGAGGGGGCAGCACC
GTTTCTTGTCTGTGCCACTTCTGGTACAACTGTGTTGCGAGCTTTTGACCCTCTGGATGAAATAGCAGAC
ATCTGCGAGAGGCACAGCCTCTCGCTTCATGTACATGCTTCTTGAGGTGGCTCAGCTTTGATATCGAGGA
AGCACCGCAGCTTCTGCATGGCATCCACAGGGCTTGACTCTGTGGCCTGGAACCCACACAAGATGCTGAT
GGCTGGGATCCAGTGCTGTCCTCTCCTTGTGAAAGACAAATCTGACTTAGAAAAGAGATGCCAAGAGTTT
GTGCCTGCCTATCTCTGGCAGGAAGACATATTTTATAATGTTGCTTTTCAGAAAAATGGTACAAAATTTA
CCCATGAAACTCAGGTGGGAAGGAATTGCAGAAGCCTGTGGTTCACCTCGAAAGCCAGGGGTGGTGAGGG
GTTCGCGTGGTTCAGGTGCCCCATGCTAGGTGATAGGAGGTACCTAGTACATGAAATCAAGAAAAGAGAA
GGATTCAAGTTACTGATCGAACCTGAATATGCCAATATTTGCTTTTGGTACATTCCACCGAGCCTCAGAG
AGATGGAAGAAGGACCCGAGTTCTCGGCAAAACTTACACAGGTGGCCCCAGCCATTAAGGAGAGGATGAT
GAAGAAGGGAAGCTTGATGCTGGGCTACCAGCCGCACTTTACAAAGGTCAACTTCTTCCGCCAGGTGGTG
ATCAGCCCTCAAGTGAGCCGGGAGGACATGGACTTCCTCCTGGATGAGATAGACTTACTGGGTAAAGACA
TGTAGCTGTGGCTTTGGTCCCCCAGAGGCATAGATCCTATCCTGGGAGAGTTTAGATCCAGAAC

In a search of public sequence databases, the NOV11 nucleic acid sequence, located on chromosome 3 has 985 of 1512 bases (65%) identical to a gb:GENBANK-ID:AF116547|acc:AF116547.1 mRNA from Homo sapiens (Homo sapiens cysteine sulfinic acid decarboxylase-related protein 3 (CSAD) mRNA, complete cds).

The disclosed NOV11 polypeptide (SEQ ID NO:32) encoded by SEQ ID NO:31 has 494 amino acid residues and is presented in Table 11D using the one-letter amino acid code. Signal P, Psort and/or Hydropathy results predict that NOV11 has no signal peptide and is likely to be localized in the nucleus with a certainty of 0.6000. In other embodiments, NOV11 may also be localized to the microbody (peroxisome) with acertainty of 0.5720, the mitochondrial matrix space with a certainty of 0.1000, or in the lysosome (lumen) with a certainty of 0.1000.

TABLE 11B
Encoded NOV11 protein sequence.

(SEQ ID NO:44)

MIPSKKGVVLNGDAKAGEKFVEEACRLIMEEVVLKATDVNEKVCEWRPPEQLKQLLDLEMRDSGEPPHKL
LELCRDVIHYSVKTDHPRFFNQLYAGLDYYSLVARFMTEALNPSSYTYEVSPVFLLVEEAVLKKMIEFIG
WKEGDGIFNPGGSVSNMYAMNLARYKYCPDIKEKGLSGSPRLILFTSAECHYSMKKAASFLGIGTENVCF
VETDRGKMIPEELEKQVWQARKEGAAPFLVCATSGTTVLGAFDPLDEIADICERHSLWLHVDASWGGSAL
MSRKHRKLLHGIHRADSVAWNPHKMLMAGIQCCALLVKLKSDLEKRCQEFVPAYLWQEDKFYNVAFQKNG
TKFTHETQVGRNCRSLWFTWKARGGEGLGWLRCPMLGDGRYLVDEIKKREGFKLLMEPEYANICFWYIPP
SLREMEEGPEFWAKLTQVAPAIKERMMKKGSLMLGYQPHFTKVNFFRQVVISPQVSREDMDFLLDEIDLL
GKDM

A search of sequence databases reveals that the NOV11 amino acid sequence has 290 of 494 amino acid residues (58%) identical to, and 376 of 494 amino acid residues (76%) similar to, the 493 amino acid residue ptnr:SWISSPROT-ACC:Q64611 protein from Rattus norvegicus (Rat) (CYSTEINE SULFINIC ACID DECARBOXYLASE (EC 4.1.1.29) (SULFINOALANINE DECARBOXYLASE) (CYSTEINE-SULFINATE DECARBOXYLASE)).

The disclosed NOV11 polypeptide has homology to the amino acid sequences shown in the BLASTP data listed in Table 11C.

TABLE 11C
BLAST results for NOV11

| Gene Index/ | | Length | Identity | Positives | |
|---|---|---|---|---|---|
| Identifier | Protein/ Organism | (aa) | (%) | (%) | Expect |
| gi\|11120696\|ref\|NP | cysteine- | 493 | 58 | 75 | e−175 |
| 068518.1\| | sulfinate | | | | |
| (NM_021750 | decarboxylase | | | | |
| | [Rattus norvegicus] | | | | |
| gi\|12836642\|dbj\|BAB23747.1\| | Putative | 493 | 58 | 75 | e−171 |
| (AK005015) | protein/mouse | | | | |
| gi\|14757624\|ref\|XP | hypothetical protein | 493 | 57 | 75 | e−168 |
| 029712.1\| | XP_029712 [Homo | | | | |
| (XM_029712) | sapiens] | | | | |
| gi\|6685337\|sp\|Q9Y600\| | CYSTEINE SULFINIC | 493 | 57 | 74 | e−168 |
| CSD HUMAN | ACID | | | | |
| | DECARBOXYLASE | | | | |
| | (SULFINOALANINE | | | | |
| | DECARBOXYLASE) | | | | |
| | (CYSTEINE- | | | | |
| | SULFINATE | | | | |
| | DECARBOXYLASE) | | | | |
| gi\|4894562\|gb\|AAD32546.1\| | cysteine sulfinic | 493 | 57 | 75 | e−167 |
| AF116548.1 | acid | | | | |
| (AF116548) | decarboxylase- | | | | |
| | related protein 4 | | | | |
| | [Homo sapiens] | | | | |

The homology between these and other sequences is shown graphically in the ClustalW analysis shown in Table 11D. In the ClustalW alignment of the NOV11 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

Tables 1E-1F lists the domain description from DOMAIN analysis results against NOV11. This indicates that the NOV11 sequence has properties similar to those of other proteins known to contain this domain.

TABLE 11E
Domain Analysis of NOV11

gnl|Pfam|pfam00282, pyridoxal_deC, Pyridoxal-dependent
decarboxylase conserved domain.
CD-Length = 372 residues, 99.7% aligned Score = 279 bits (714),
Expect = 2e−76

TABLE 11F
Domain Analysis of NOV11

gnl|Pfam|pfam00266, aminotran_5, Aminotransferase class-V
CD-Length = 354 residues Score = 42.7 bits (99), Expect = 5e−05

Cysteine sulfinic acid decarboxylase (CSAD), the rate-limiting enzyme in taurine biosynthesis, was found to be activated under conditions that favor protein phosphorylation and inactivated under conditions favoring protein dephosphorylation. Direct incorporation of 32P into purified CSAD has been demonstrated with [gamma 32P]ATP and PKC, but not PKA. In addition, the 32P labeling of CSAD was inhibited by PKC inhibitors suggesting that PKC is responsible for phosphorylation of CSAD in the brain. Okadaic acid had no effect on CSAD activity at 10 microM suggesting that protein phosphatase-2C (PrP-2C) might be involved in the dephosphorylation of CSAD. Furthermore, it was found that either glutamate- or high K(+)-induced depolarization increased CSAD activity as well as 32P-incorporation into CSAD in neuronal cultures, supporting the notion that the CSAD activity is endogenously regulated by protein phosphorylation in the brain. A model to link neuronal excitation, phosphorylation of CSAD and increase in taurine biosynthesis is proposed.

Met metabolism occurs primarily by activation of Met to AdoMet and further metabolism of AdoMet by either the transmethylation-transsulfuration pathway or the polyamine biosynthetic pathway. The catabolism of the methyl group and sulfur atom of Met ultimately appears to be dependent upon the transmethylation-transsulfuration pathway because the MTA formed as the co-product of polyamine synthesis is efficiently recycled to Met. On the other hand, the fate of the four-carbon chain of Met appears to depend upon the initial fate of the Met molecule. During transsulfuration, the carbon chain is released as alpha-ketobutyrate, which is further metabolized to CO2. In the polyamine pathway, the carboxyl carbon of Met is lost in the formation of dAdoMet, whereas the other three carbons are ultimately excreted as polyamine derivatives and degradation products. The role of the transamination pathway of Met metabolism is not firmly established. Cys (which may be formed from the sulfur of Met and the carbons of serine via the transsulfuration pathway) appears to be converted to taurine and CO2 primarily by the cysteinesulfinate pathway, and to sulfate and pyruvate primarily by desulfuration pathways in which a reduced form of sulfur with a relatively long biological half-life appears to be an intermediate. With the exception of the nitrogen of Met that is incorporated into polyamines, the nitrogen of Met or Cys is incorporated into urea after it is released as ammonium [in the reactions catalyzed by cystathionase with either cystathionine (from Met) or cystine (from Cys) as substrate] or it is transferred to a keto acid (in Cys or Met transamination). Many areas of sulfur-containing amino acid metabolism need further study. The magnitude of AdoMet flux through the polyamine pathway in the intact animal as well as details about the reactions involved in this pathway remain to be determined. Both the pathways and the possible physiological role of alternate (AdoMet-independent) Met metabolism, including the transamination pathway, must be elucidated. Despite the growing interest in taurine, investigation of Cys metabolism has been a relatively inactive area during the past two decades. Apparent discrepancies in the reported data on Cys metabolism need to be resolved. Future work should consider the role of extrahepatic tissues in amino acid metabolism as well as species differences in the relative roles of various pathways in the metabolism of Met and Cys.

Both immunocytochemical and electrophysiological methods have been employed to determine whether the localization of the taurine synthetic enzyme, cysteine sulfinic acid decarboxylase, (CSAD) and the postsynaptic action of taurine in the CA1 region of rat hippocampus are consistent with the hypothesis that taurine may be used as a neurotransmitter by some hippocampal neurons. At the light microscopic level, CSAD-immunoreactivity (CSAD-IR) was found in the pyramidal basket cells, and around pyramidal cells in stratum pyramidale and stratum radiatum. At the electron microscopic level, CSAD-IR was seen most often in the soma and the dendrites and was rather infrequent in the axon or the nerve terminals. Electrophysiological observations on the in vitro hippocampal slice demonstrated that pyramidal neurons respond to artificially applied taurine with inhibition that depended in large part upon an increased chloride conductance. Although electrophysiological observations are consistent with a neurotransmitter role for taurine, results from immunocytochemical studies suggest a minor role for taurine as a neurotransmitter. In fact, immunocytochemical observations suggested that taurine may be used as a neurotransmitter only by a small number of pyramidal basket interneurons, the vast majority of CSAD-positive neurons may use taurine for other functions.

The effect of 3-acetylpyridine (3-AP) administration on the biosynthesis of taurine in the rat brain has been studied. Treatment with 3-AP induced a significant decrease in the cerebellar contents of taurine and its metabolic precursors, cysteine sulfinic acid (CSA) and cysteic acid (CA), as well as a selective degeneration of climbing fibers in the molecular layer of the cerebellum. It was found that the activity of cerebral cysteine dioxygenase, the enzyme catalyzing the formation of CSA from cysteine, consisted of two systems with low and high Km values. The 3-AP-induced attenuation of cysteine dioxygenase activity with a low Km value was noted only in the cerebellum, while that with a high Km value was detected not only in the cerebellum but also in other brain areas such as the medulla oblongata, striatum and cerebral cortex. In contrast, no alteration in the activity of cysteine sulfinic acid decarboxylase (CSD) was observed in any brain areas examined following the administration of 3-AP. Furthermore, it was found that essentially no cystamine as well as a very low activity of cysteamine dioxygenase is present in the brain. The present results suggest that taurine in the brain is synthesized from cysteine, mainly by the CSA and CA pathways, and the observed decline of cerebellar taurine in 3-AP-treated rats may be due to an attenuation of the biosynthesis, possibly at the step of cysteine dioxygenase. A possible regulatory role of cysteine dioxygenase with a low Km value in the biosynthesis of cerebral taurine is also suggested.

The activity of cysteinesulfinic acid decarboxylase (CSAD, EC 4.1.1.29) in extracts of liver of seven mammals varied greatly, whereas in extracts of brain from the same species, the variation was less marked. CSAD activity was readily measured in extracts of spinal cord from the same species, except those from rhesus monkey and man. The most noteworthy observation was the complete absence of CSAD activity in extracts of optic nerves and of sciatic nerves from all seven mammals. This suggests that taurine biosynthesis does not occur within axons and that intraaxonal taurine is supplied by axonal transport from the cell body.

Taurine, cysteinesulfinic acid decarboxylase (CSAD), glutamate, gamma-aminobutyric acid (GABA), and glutamic acid decarboxylase (GAD) were measured in subcellular fractions prepared from occipital lobe of fetal and neonatal rhesus monkeys. In addition, the distribution of [35S]taurine in subcellular fractions was determined after administration to the fetus via the mother, to the neonate via administration to the mother prior to birth, and directly to the neonate at various times after birth. CSAD, glutamate, GABA, and GAD all were found to be low or unmeasurable in early fetal life and to increase during late fetal and early neonatal life to reach values found in the mother. Taurine was present in large amounts in early fetal life and decreased slowly during neonatal life, arriving at amounts found in the mother not until after 150 days of age. Significant amounts of taurine, CSAD, GABA, and GAD were associated with nerve ending components with some indication that the proportion of brain taurine found in these organelles increases during development. All subcellular pools of taurine were rapidly labeled by exogenously administered [35S]taurine. The subcellular distribution of all the components measured was compatible with the neurotransmitter or putative neurotransmitter functions of glutamate, GABA, and taurine. The large amount of these three amino acids exceeds that required for such function. The excess of glutamate and GABA may be used as a source of energy. The function of the excess of taurine is still not clear, although circumstantial evidence favors an important role in the development and maturation of the CNS.

The disclosed NOV11 nucleic acid of the invention encoding a Cysteine sulfinic acid decarboxylase -like protein includes the nucleic acid whose sequence is provided in Table 11A or a fragment thereof. The invention also includes a mutant or variant nucleic acid any of whose bases may be changed from the corresponding base shown in Table 11A while still encoding a protein that maintains its Cysteine sulfinic acid decarboxylase-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 35 percent of the bases may be so changed.

The disclosed NOV11 protein of the invention includes the Cysteine sulfinic acid decarboxylase-like protein whose sequence is provided in Table 11B. The invention also includes a mutant or variant protein any of whose residues may be changed from the corresponding residue shown in Table 11B while still encoding a protein that maintains its Cysteine sulfinic acid decarboxylase -like activities and physiological functions, or a functional fragment thereof. In the mutant or variant protein, up to about 42 percent of the residues may be so changed.

The invention further encompasses antibodies and antibody fragments, such as F_{ab} or (F_{ab})₂, that bind immunospecifically to any of the proteins of the invention.

The above defined information for this invention suggests that this Cysteine sulfinic acid decarboxylase-like protein (NOV11) may function as a member of a “Cysteine sulfinic acid decarboxylase family”. Therefore, the NOV11 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV11 nucleic acids and proteins of the invention are useful in potential therapeutic applications implicated in cancer including but not limited to various pathologies and disorders as indicated below. For example, a cDNA encoding the Cysteine sulfinic acid decarboxylase-like protein (NOV11) may be useful in gene therapy, and the Cysteine sulfinic acid decarboxylase -like protein (NOV11) may be useful when administered to a subject in need thereof. By way of nonlimiting example, the compositions of the present invention will have efficacy for treatment of patients suffering from Adrenoleukodystrophy, Congenital Adrenal Hyperplasia, Diabetes, Von Hippel-Lindau (VHL) syndrome, Pancreatitis, Obesity, Hyperparathyroidism, Hypoparathyroidism, Fertility, cancers such as those occurring in pancreas, bone, colon, brain, lung, breast, or prostate. Endometriosis, Xerostomia Scleroderma Hypercalceimia, Ulcers Von Hippel-Lindau (VHL) syndrome, Cirrhosis, Transplantation, Inflammatory bowel disease, Diverticular disease, Hirschsprung's disease, Crohn's Disease, Appendicitis Osteoporosis, Hypercalceimia, Arthritis, Ankylosing spondylitis, Scoliosis Arthritis, Tendinitis on Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Endocrine dysfunctions, Diabetes, obesity, Growth and reproductive disorders Multiple sclerosis, Leukodystrophies, Pain, Myasthenia gravis, Pain, Systemic lupus erythematosus, Autoimmune disease, Asthma, Emphysema, Scleroderma, allergy, ARDS, Psoriasis, Actinic keratosis, Tuberous sclerosis, Acne, Hair growth, allopecia, pigmentation disorders, Renal artery stenosis, Interstitial nephritis, Glomerulonephritis, Polycystic kidney disease, Systemic lupus erythematosus, Renal tubular acidosis, IgA nephropathy, Hypercalceimia, Lesch-Nyhan syndrome and other diseases, disorders and conditions of the like. The NOV11 nucleic acid encoding the Cysteine sulfinic acid decarboxylase-like protein of the invention, or fragments thereof, may further be useful in diagnostic applications, wherein the presence or amount of the nucleic acid or the protein are to be assessed.

NOV11 nucleic acids and polypeptides are further useful in the generation of antibodies that bind immuno-specifically to the novel NOV11 substances for use in therapeutic or diagnostic methods. These antibodies may be generated according to methods known in the art, using prediction from hydrophobicity charts, as described in the “Anti-NOVX Antibodies” section below. The disclosed NOV11 protein has multiple hydrophilic regions, each of which can be used as an immunogen. In one embodiment, a contemplated NOV11 epitope is from about amino acids 25 to 50. In another embodiment, a NOV11 epitope is from about amino acids 100 to 140. In additional embodiments, a NOV11 epitope is from about amino acids 140 to 170, from about amino acids 235 to 260, and from about amino acids 300 to 320. These novel proteins can be used in assay systems for functional analysis of various human disorders, which will help in understanding of pathology of the disease and development of new drug targets for various disorders.

NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNAs) and fragments for use as PCR primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

An NOVX nucleic acid can encode a mature NOVX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product, encoded by the corresponding gene. Alternatively, it may be defined as the polypeptide, precursor or proprotein encoded by an ORF described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

The term “probes”, as utilized herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as utilized herein, is one, which is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence SEQ ID NOS:1, 3, 5, 7, 9, 11, 13, 15, 17, 19, 21, 23, 25, 27, 29, 31, 33, 35, 37, 39, 41 and 43, or a complement of this aforementioned nucleotide sequence, can be isolated using standard molecular biology techniques and the sequence information provided herein. Using all or a portion of the nucleic acid sequence of SEQ ID NOS:1, 3, 5, 7, 9, 11, 13, 15, 17, 19, 21, 23, 25, 27, 29, 31, 33, 35, 37, 39, 41 and 43 as a hybridization probe, NOVX molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides SEQ ID NOS:1, 3, 5, 7, 9, 11, 13, 15, 17, 19, 21, 23, 25, 27, 29, 31, 33, 35, 37, 39, 41 and 43, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NOS:1, 3, 5, 7, 9, 11, 13, 15, 17, 19, 21, 23, 25, 27, 29, 31, 33, 35, 37, 39, 41 and 43, or a portion of this nucleotide sequence (e.g., a fragment that can be used as a probe or primer or a fragment encoding a biologically-active portion of an NOVX polypeptide). A nucleic acid molecule that is complementary to the nucleotide sequence shown SEQ ID NOS:1, 3, 5, 7, 9, 11, 13, 15, 17, 19, 21, 23, 25, 27, 29, 31, 33, 35, 37, 39, 41 or 43 is one that is sufficiently complementary to the nucleotide sequence shown SEQ ID NOS:1, 3, 5, 7, 9, 11, 13, 15, 17, 19, 21, 23, 25, 27, 29, 31, 33, 35, 37, 39, 41 or 43 that it can hydrogen bond with little or no mismatches to the nucleotide sequence shown SEQ ID NOS:1, 3, 5, 7, 9, 11, 13, 15, 17, 19, 21, 23, 25, 27, 29, 31, 33, 35, 37, 39, 41 and 43, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for an NOVX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NOS:1, 3, 5, 7, 9, 11, 13, 15, 17, 19, 21, 23, 25, 27, 29, 31, 33, 35, 37, 39, 41 and 43, as well as a polypeptide possessing NOVX biological activity. Various biological activities of the NOVX proteins are described below.

An NOVX polypeptide is encoded by the open reading frame (“ORF”) of an NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bonafide cellular protein, a minimum size requirement is often set, e.g., a stretch of DNA that would encode a protein of 50 amino acids or more.

The nucleotide sequences determined from the cloning of the human NOVX genes allows for the generation of probes and primers designed for use in identifying and/or cloning NOVX homologues in other cell types, e.g. from other tissues, as well as NOVX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence SEQ ID NOS:1, 3, 5, 7, 9, 11, 13, 15, 17, 19, 21, 23, 25, 27, 29, 31, 33, 35, 37, 39, 41 or 43; or an anti-sense strand nucleotide sequence of SEQ ID NOS:1, 3, 5, 7, 9, 11, 13, 15, 17, 19, 21, 23, 25, 27, 29, 31, 33, 35, 37, 39, 41 or 43; or of a naturally occurring mutant of SEQ ID NOS:1, 3, 5, 7, 9, 11, 13, 15, 17, 19, 21, 23, 25, 27, 29, 31, 33, 35, 37, 39, 41 and 43.

Probes based on the human NOVX nucleotide sequences can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissues which mis-express an NOVX protein, such as by measuring a level of an NOVX-encoding nucleic acid in a sample of cells from a subject e.g., detecting NOVX mRNA levels or determining whether a genomic NOVX gene has been mutated or deleted.

“A polypeptide having a biologically-active portion of an NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion SEQ ID NOS:1, 3, 5, 7, 9, 11, 13, 15, 17, 19, 21, 23, 25, 27, 29, 31, 33, 35, 37, 39, 41 or 43, that encodes a polypeptide having an NOVX biological activity (the biological activities of the NOVX proteins are described below), expressing the encoded portion of NOVX protein (e.g., by recombinant expression in vitro) and assessing the activity of the encoded portion of NOVX.

NOVX Nucleic Acid and Polypeptide Variants

The invention further encompasses nucleic acid molecules that differ from the nucleotide sequences shown in SEQ ID NOS:1, 3, 5, 7, 9, 11, 13, 15, 17, 19, 21, 23, 25, 27, 29, 31, 33, 35, 37, 39, 41 and 43 due to degeneracy of the genetic code and thus encode the same NOVX proteins as that encoded by the nucleotide sequences shown in SEQ ID NOS:1, 3, 5, 7, 9, 11, 13, 15, 17, 19, 21, 23, 25, 27, 29, 31, 33, 35, 37, 39, 41 and 43. In another embodiment, an isolated nucleic acid molecule of the invention has a nucleotide sequence encoding a protein having an amino acid sequence shown in SEQ ID NOS:2, 4, 6, 8, 10, 12, 14, 16, 18, 20, 22, 24, 26, 28, 30, 32, 34, 26, 28, 40, 42 or 44.

In addition to the human NOVX nucleotide sequences shown in SEQ ID NOS:1, 3, 5, 7, 9, 11, 13, 15, 17, 19, 21, 23, 25, 27, 29, 31, 33, 35, 37, 39, 41 and 43, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding an NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from the human SEQ ID NOS:1, 3, 5, 7, 9, 11, 13, 15, 17, 19, 21, 23, 25, 27, 29, 31, 33, 35, 37, 39, 41 and 43 are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NOS:1, 3, 5, 7, 9, 11, 13, 15, 17, 19, 21, 23, 25, 27, 29, 31, 33, 35, 37, 39, 41 and 43. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to the sequences SEQ ID NOS:1, 3, 5, 7, 9, 11, 13, 15, 17, 19, 21, 23, 25, 27, 29, 31, 33, 35, 37, 39, 41 and 43, corresponds to a naturally-occurring nucleic acid molecule. As used herein, a “naturally-occuring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NOS:1, 3, 5, 7, 9, 11, 13, 15, 17, 19, 21, 23, 25, 27, 29, 31, 33, 35, 37, 39, 41 and 43, or fragments, analogs or derivatives thereof, under conditions of moderate stringency is provided. A non-limiting example of moderate stringency hybridization conditions are hybridization in 6×SSC, 5×Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, NY, and Kriegler, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences SEQ ID NOS:1, 3, 5, 7, 9, 11, 13, 15, 17, 19, 21, 23, 25, 27, 29, 31, 33, 35, 37, 39, 41 and 43, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

Conservative Mutations

In addition to naturally-occurring allelic variants of NOVX sequences that may exist in the population, the skilled artisan will further appreciate that changes can be introduced by mutation into the nucleotide sequences SEQ ID NOS:1, 3, 5, 7, 9, 11, 13, 15, 17, 19, 21, 23, 25, 27, 29, 31, 33, 35, 37, 39, 41 and 43, thereby leading to changes in the amino acid sequences of the encoded NOVX proteins, without altering the functional ability of said NOVX proteins. For example, nucleotide substitutions leading to amino acid substitutions at “non-essential” amino acid residues can be made in the sequence SEQ ID NOS:2, 4, 6, 8, 10, 12, 14, 16, 18, 20, 22, 24, 26, 28, 30, 32, 34, 26, 28, 40, 42 or 44. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are predicted to be particularly non-amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from SEQ ID NOS:1, 3, 5, 7, 9, 11, 13, 15, 17, 19, 21, 23, 25, 27, 29, 31, 33, 35, 37, 39, 41 and 43 yet retain biological activity. In one embodiment, the isolated nucleic acid molecule comprises a nucleotide sequence encoding a protein, wherein the protein comprises an amino acid sequence at least about 45% homologous to the amino acid sequences SEQ ID NOS:2, 4, 6, 8, 10, 12, 14, 16, 18, 20, 22, 24, 26, 28, 30, 32, 34, 26, 28, 40, 42 and 44. Preferably, the protein encoded by the nucleic acid molecule is at least about 60% homologous to SEQ ID NOS:2, 4, 6, 8, 10, 12, 14, 16, 18, 20, 22, 24, 26, 28, 30, 32, 34, 26, 28, 40, 42 and 44; more preferably at least about 70% homologous SEQ ID NOS:2, 4, 6, 8, 10, 12, 14, 16, 18, 20, 22, 24, 26, 28, 30, 32, 34, 26, 28, 40, 42 or 44; still more preferably at least about 80% homologous to SEQ ID NOS:2, 4, 6, 8, 10, 12, 14, 16, 18, 20, 22, 24, 26, 28, 30, 32, 34, 26, 28, 40, 42 or 44; even more preferably at least about 90% homologous to SEQ ID NOS:2, 4, 6, 8, 10, 12, 14, 16, 18, 20, 22, 24, 26, 28, 30, 32, 34, 26, 28, 40, 42 or 44; and most preferably at least about 95% homologous to SEQ ID NOS:2, 4, 6, 8, 10, 12, 14, 16, 18, 20, 22, 24, 26, 28, 30, 32, 34, 26, 28, 40, 42 or 44.

An isolated nucleic acid molecule encoding an NOVX protein homologous to the protein of SEQ ID NOS:2, 4, 6, 8, 10, 12, 14, 16, 18, 20, 22, 24, 26, 28, 30, 32, 34, 26, 28, 40, 42 or 44 can be created by introducing one or more nucleotide substitutions, additions or deletions into the nucleotide sequence of SEQ ID NOS:1, 3, 5, 7, 9, 11, 13, 15, 17, 19, 21, 23, 25, 27, 29, 31, 33, 35, 37, 39, 41 and 43, such that one or more amino acid substitutions, additions or deletions are introduced into the encoded protein.

Mutations can be introduced into SEQ ID NOS:1, 3, 5, 7, 9, 11, 13, 15, 17, 19, 21, 23, 25, 27, 29, 31, 33, 35, 37, 39, 41 and 43 by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, bistidine). Thus, a predicted non-essential amino acid residue in the NOVX protein is replaced with another amino acid residue from the same side chain family. Alternatively, in another embodiment, mutations can be introduced randomly along all or part of an NOVX coding sequence, such as by saturation mutagenesis, and the resultant mutants can be screened for NOVX biological activity to identify mutants that retain activity. Following mutagenesis SEQ ID NOS:1, 3, 5, 7, 9, 11, 13, 15, 17, 19, 21, 23, 25, 27, 29, 31, 33, 35, 37, 39, 41 and 43, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, VLIM, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and an NOVX ligand; or (iii) the ability of a mutant NOVX protein to bind to an intracellular target protein or biologically-active portion thereof; (e.g. avidin proteins).

In yet another embodiment, a mutant NOVX protein can be assayed for the ability to regulate a specific biological function (e.g., regulation of insulin release).

Antisense Nucleic Acids

Another aspect of the invention pertains to isolated antisense nucleic acid molecules that are hybridizable to or complementary to the nucleic acid molecule comprising the nucleotide sequence of SEQ ID NOS:1, 3, 5, 7, 9, 11, 13, 15, 17, 19, 21, 23, 25, 27, 29, 31, 33, 35, 37, 39, 41 and 43, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of an NOVX protein of SEQ ID NOS:2, 4, 6, 8, 10, 12, 14, 16, 18, 20, 22, 24, 26, 28, 30, 32, 34, 26, 28, 40, 42 or 44, or antisense nucleic acids complementary to an NOVX nucleic acid sequence of SEQ ID NOS:1, 3, 5, 7, 9, 11, 13, 15, 17, 19, 21, 23, 25, 27, 29, 31, 33, 35, 37, 39, 41 and 43, are additionally provided.

In one embodiment, an antisense nucleic acid molecule is antisense to a “coding region” of the coding strand of a nucleotide sequence encoding an NOVX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences which flank the coding region that are not translated into amino acids (i.e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an NOVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (See, e.g., Inoue, et al. 1987. Nucl. Acids Res. 15: 6131-6148) or a chimeric RNA-DNA analogue (See, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for an NOVX-encoding nucleic acid can be designed based upon the nucleotide sequence of an NOVX cDNA disclosed herein (i.e., SEQ ID NOS:1, 3, 5, 7, 9, 11, 13, 15, 17, 19, 21, 23, 25, 27, 29, 31, 33, 35, 37, 39, 41 and 43). For example, a derivative of a Tetrahymena L-19 IVS RNA can be constructed in which the nucleotide sequence of the active site is complementary to the nucleotide sequence to be cleaved in an NOVX-encoding mRNA. See, e.g., U.S. Pat. No. 4,987,071 to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Helene, 1991. Anticanicer Drug Des. 6: 569-84; Helene, et al. 1992. Ann. N.Y. Acad. Sci. 660: 27-36; Maher, 1992. Bioassays 14: 807-15.

In various embodiments, the NOVX nucleic acids can be modified at the base moiety, sugar moiety or phosphate backbone to improve, e.g. the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (See, Hyrup, et al., 1996.supra); or as probes or primers for DNA sequence and hybridization (See, Hyrup, et al., 1996, supra; Perry-O'Keefe, et al., 1996. supra).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide including the amino acid sequence of NOVX polypeptides whose sequences are provided in SEQ ID NOS:2, 4, 6, 8, 10, 12, 14, 16, 18, 20, 22, 24, 26, 28, 30, 32, 34, 26, 28, 40, 42 or 44. The invention also includes a mutant or variant protein any of whose residues may be changed from the corresponding residues shown in SEQ ID NOS:2, 4, 6, 8, 10, 12, 14, 16, 18, 20, 22, 24, 26, 28, 30, 32, 34, 26, 28, 40, 42 or 44 while still encoding a protein that maintains its NOVX activities and physiological functions, or a functional fragment thereof.

In general, an NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, an NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, i.e., culture medium represents less than about 20%, more preferably less than about 10%, and most preferably less than about 5% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence shown in SEQ ID NOS:2, 4, 6, 8, 10, 12, 14, 16, 18, 20, 22, 24, 26, 28, 30, 32, 34, 26, 28, 40, 42 or 44) that include fewer amino acids than the full-length NOVX proteins, and exhibit at least one activity of an NOVX protein. Typically, biologically-active portions comprise a domain or motif with at least one activity of the NOVX protein. A biologically-active portion of an NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence shown SEQ ID NOS:2, 4, 6, 8, 10, 12, 14, 16, 18, 20, 22, 24, 26, 28, 30, 32, 34, 26, 28, 40, 42 or 44. In other embodiments, the NOVX protein is substantially homologous to SEQ ID NOS:2, 4, 6, 8, 10, 12, 14, 16, 18, 20, 22, 24, 26, 28, 30, 32, 34, 26, 28, 40, 42 or 44, and retains the functional activity of the protein of SEQ ID NOS:2, 4, 6, 8, 10, 12, 14, 16, 18, 20, 22, 24, 26, 28, 30, 32, 34, 26, 28, 40, 42 or 44, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence SEQ ID NOS:2, 4, 6, 8, 10, 12, 14, 16, 18, 20, 22, 24, 26, 28, 30, 32, 34, 26, 28, 40, 42 or 44, and retains the functional activity of the NOVX proteins of SEQ ID NOS:2, 4, 6, 8, 10, 12, 14, 16, 18, 20, 22, 24, 26, 28, 30, 32, 34, 26, 28, 40, 42 or 44.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NOS:1, 3, 5, 7, 9, 11, 13, 15, 17, 19, 21, 23, 25, 27, 29, 31, 33, 35, 37, 39, 41 and 43.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOVX chimeric or fusion proteins. As used herein, an NOVX “chimeric protein” or “fusion protein” comprises an NOVX polypeptide operatively-linked to a non-NOVX polypeptide. An “NOVX polypeptide” refers to a polypeptide having an amino acid sequence corresponding to an NOVX protein SEQ ID NOS:2, 4, 6, 8, 10, 12, 14, 16, 18, 20, 22, 24, 26, 28, 30, 32, 34, 26, 28, 40, 42 or 44, whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within an NOVX fusion protein the NOVX polypeptide can correspond to all or a portion of an NOVX protein. In one embodiment, an NOVX fusion protein comprises at least one biologically-active portion of an NOVX protein. In another embodiment, an NOVX fusion protein comprises at least two biologically-active portions of an NOVX protein. In yet another embodiment, an NOVX fusion protein comprises at least three biologically-active portions of an NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is an NOVX protein containing a heterologous signal sequence at its N-terminus. In certain host cells (e.g., mammalian host cells), expression and/or secretion of NOVX can be increased through use of a heterologous signal sequence.

In yet another embodiment, the fusion protein is an NOVX-immunoglobulin fusion protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between an NOVX ligand and an NOVX protein on the surface of a cell, to thereby suppress NOVX-mediated signal transduction in vivo. The NOVX-immunoglobulin fusion proteins can be used to affect the bioavailability of an NOVX cognate ligand. Inhibition of the NOVX ligand/NOVX interaction may be useful therapeutically for both the treatment of proliferative and differentiative disorders, as well as modulating (e.g. promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with an NOVX ligand.

An NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). An NOVX-encoding nucleic acid can be cloned into such an expression vector such that the fusion moiety is linked in-frame to the NOVX protein.

NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the NOVX protein coding sequences can be used to generate a variegated population of NOVX fragments for screening and subsequent selection of variants of an NOVX protein. In one embodiment, a library of coding sequence fragments can be generated by treating a double stranded PCR fragment of an NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin and Yourvan, 1992. Proc. Natl. Acad. Sci. USA 89: 7811-7815; Delgrave, et al., 1993. Protein Engineering 6:327-331.

Anti-NOVX Antibodies

Also included in the invention are antibodies to NOVX proteins, or fragments of NOVX proteins. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′} and F_{(ab′)2} fragments, and an F_{ab} expression library. In general, an antibody molecule obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated NOVX-related protein of the invention may be intended to serve as an antigen, or a portion or fragment thereof, and additionally can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of NOVX-related protein that is located on the surface of the protein, e.g., a hydrophilic region. A hydrophobicity analysis of the human NOVX-related protein sequence will indicate which regions of a NOVX-related protein are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each of which is incorporated herein by reference in its entirety. Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow and Lane, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, MONOCLONAL ANTIBODIES: PRINCIPLES AND PRACTICE, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., MONOCLONAL ANTIBODY PRODUCTION TECHNIQUES AND APPLICATIONS, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Preferably, antibodies having a high degree of specificity and a high binding affinity for the target antigen are isolated.

After the desired hybridoma cells are identified, the clones can be subcloned by limiting dilution procedures and grown by standard methods. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies relate to antibody molecules in which essentially the entire sequences of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al, (Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)/2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., 1991 EMBO J., 10:3655-3659.

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol comRho-Interacting Proteing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol)propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of an NOVX protein is facilitated by generation of hybridomas that bind to the fragment of an NOVX protein possessing such a domain. Thus, antibodies that are specific for a desired domain within an NOVX protein, or derivatives, fragments, analogs or homologs thereof, are also provided herein.

Anti-NOVX antibodies may be used in methods known within the art relating to the localization and/or quantitation of an NOVX protein (e.g., for use in measuring levels of the NOVX protein within appropriate physiological samples, for use in diagnostic methods, for use in imaging the protein, and the like). In a given embodiment, antibodies for NOVX proteins, or derivatives, fragments, analogs or homologs thereof, that contain the antibody derived binding domain, are utilized as pharmacologically-active compounds (hereinafter “Therapeutics”).

An anti-NOVX antibody (e.g., monoclonal antibody) can be used to isolate an NOVX polypeptide by standard techniques, such as affinity chromatography or immunoprecipitation. An anti-NOVX antibody can facilitate the purification of natural NOVX polypeptide from cells and of recombinantly-produced NOVX polypeptide expressed in host cells. Moreover, an anti-NOVX antibody can be used to detect NOVX protein (e.g., in a cellular lysate or cell supernatant) in order to evaluate the abundance and pattern of expression of the NOVX protein. Anti-NOVX antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

NOVX Recombinant Expression Vectors and Host Cells

Another aspect of the invention pertains to vectors, preferably expression vectors, containing a nucleic acid encoding an NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporafion. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOVX-encoding nucleic acid into the male pronuclei of a fertilized oocyte (e.g., by microinjection, retroviral infection) and allowing the oocyte to develop in a pseudopregnant female foster animal. The human NOVX cDNA sequences SEQ ID NOS:1, 3, 5, 7, 9, 11, 13, 15, 17, 19, 21, 23, 25, 27, 29, 31, 33, 35, 37, 39, 41 and 43 can be introduced as a transgene into the genome of a non-human animal. Alternatively, a non-human homologue of the human NOVX gene, such as a mouse NOVX gene, can be isolated based on hybridization to the human NOVX cDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of an NOVX gene into which a deletion, addition or substitution has been introduced to thereby alter, e.g., functionally disrupt, the NOVX gene. The NOVX gene can be a human gene (e.g., the cDNA of SEQ ID NOS:1, 3, 5, 7, 9, 11, 13, 15, 17, 19, 21, 23, 25, 27, 29, 31, 33, 35, 37, 39, 41 and 43), but more preferably, is a non-human homologue of a human NOVX gene. For example, a mouse homologue of human NOVX gene of SEQ ID NOS:1, 3, 5, 7, 9, 11, 13, 15, 17, 19, 21, 23, 25, 27, 29, 31, 33, 35, 37, 39, 41 and 43 can be used to construct a homologous recombination vector suitable for altering an endogenous NOVX gene in the mouse genome. In one embodiment, the vector is designed such that, upon homologous recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in an NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease(possesses anti-microbial activity) and the various dyslipidemias. In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds which bind to or modulate the activity of the membrane-bound form of an NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37:1233.

Libraries of compounds may be presented in solution (e.g. Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to an NOVX protein determined. The cell, for example, can of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof can be accomplished, for example, by determining the ability of the NOVX protein to bind to or interact with an NOVX target molecule. As used herein, a “target molecule” is a molecule with which an NOVX protein binds or interacts in nature, for example, a molecule on the surface of a cell which expresses an NOVX interacting protein, a molecule on the surface of a second cell, a molecule in the extracellular milieu, a molecule associated with the internal surface of a cell membrane or a cytoplasmic molecule. An NOVX target molecule can be a non-NOVX molecule or an NOVX protein or polypeptide of the invention. In one embodiment, an NOVX target molecule is a component of a signal transduction pathway that facilitates transduction of an extracellular signal (e.g. a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with an NOVX target molecule can be accomplished by one of the methods described above for determining direct binding. In one embodiment, determining the ability of the NOVX protein to bind to or interact with an NOVX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising an NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting an NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g. stimulate or inhibit) the activity of the NOVX protein or biologically-active portion thereof. Determining the ability of the test compound to modulate the activity of NOVX can be accomplished, for example, by determining the ability of the NOVX protein to bind to an NOVX target molecule by one of the methods described above for determining direct binding. In an alternative embodiment, determining the ability of the test compound to modulate the activity of NOVX protein can be accomplished by determining the ability of the NOVX protein further modulate an NOVX target molecule. For example, the catalytic/enzymatic activity of the target molecule on an appropriate substrate can be determined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with an NOVX protein, wherein determining the ability of the test compound to interact with an NOVX protein comprises determining the ability of the NOVX protein to preferentially bind to or modulate the activity of an NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl--N,N-dimethyl-3-ammonio-1-propane sulfonate, 3-(3-cholamidopropyl)dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also likely to be involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g. GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the NOVX sequences, SEQ ID NOS:1, 3, 5, 7, 9, 11, 13, 15, 17, 19, 21, 23, 25, 27, 29, 31, 33, 35, 37, 39, 41 and 43, or fragments or derivatives thereof, can be used to map the location of the NOVX genes, respectively, on a chromosome. The mapping of the NOVX sequences to chromosomes is an important first step in correlating these sequences with genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The NOVX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If predicted coding sequences, such as those in SEQ ID NOS:1, 3, 5, 7, 9, 11, 13, 15, 17, 19, 21, 23, 25, 27, 29, 31, 33, 35, 37, 39, 41 and 43 are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with NOVX protein, nucleic acid expression or activity. For example, mutations in an NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g. the genotype of the individual examined to determine the ability of the individual to respond to a particular agent.)

Yet another aspect of the invention pertains to monitoring the influence of agents (e.g., drugs, compounds) on the expression or activity of NOVX in clinical trials.

These and other agents are described in further detail in the following sections.

Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NOS:1, 3, 5, 7, 9, 11, 13, 15, 17, 19, 21, 23, 25, 27, 29, 31, 33, 35, 37, 39, 41 and 43, or a portion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

An agent for detecting NOVX protein is an antibody capable of binding to NOVX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g. serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in an NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding an NOVX-protein, or the misexpression of the NOVX gene. For example, such genetic lesions can be detected by ascertaining the existence of at least one of: (i) a deletion of one or more nucleotides from an NOVX gene; (ii) an addition of one or more nucleotides to an NOVX gene; (iii) a substitution of one or more nucleotides of an NOVX gene, (iv) a chromosomal rearrangement of an NOVX gene; (v) an alteration in the level of a messenger RNA transcript of an NOVX gene, (vi) aberrant modification of an NOVX gene, such as of the methylation pattern of the genomic DNA, (vii) the presence of a non-wild-type splicing pattern of a messenger RNA transcript of an NOVX gene, (viii) a non-wild-type level of an NOVX protein, (ix) allelic loss of an NOVX gene, and (x) inappropriate post-translational modification of an NOVX protein. As described herein, there are a large number of assay techniques known in the art which can be used for detecting lesions in an NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to an NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in NOVX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOVX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to base pair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on an NOVX sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild type nucleic acids. See, e.g., Orita, et al., 1989. Proc Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The, sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders (The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers.) In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g. N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with an NOVX modulator, such as a modulator identified by one of the exemplary screening assays described herein.

Monitoring of Effects During Clinical Trials

Monitoring the influence of agents (e.g., drugs, compounds) on the expression or activity of NOVX (e.g., the ability to modulate aberrant cell proliferation and/or differentiation) can be applied not only in basic drug screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of an NOVX protein, mRNA, or genomic DNA in the preadministration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. The disorders include cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, prostate cancer, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, and other diseases, disorders and conditions of the like.

These methods of treatment will be discussed more fully, below.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof, (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, an NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of an NOVX protein, a peptide, an NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of an NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering an NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The NOVX nucleic acids and proteins of the invention are useful in potential prophylactic and therapeutic applications implicated in a variety of disorders including, but not limited to: metabolic disorders, diabetes, obesity, infectious disease, anorexia, cancer-associated cancer, neurodegenerative disorders, Alzheimer's Disease, Parkinson's Disorder, immune disorders, hematopoietic disorders, and the various dyslipidemias, metabolic disturbances associated with obesity, the metabolic syndrome X and wasting disorders associated with chronic diseases and various cancers.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from: metabolic disorders, diabetes, obesity, infectious disease, anorexia, cancer-associated cachexia, cancer, neurodegenerative disorders, Alzheimer's Disease, Parkinson's Disorder, immune disorders, hematopoietic disorders, and the various dyslipidemias.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies, which immunospecifically-bind to the novel substances of the invention for use in therapeutic or diagnostic methods.

The invention will be further described in the following examples, which do not limit the scope of the invention described in the claims.

### EXAMPLES

### Example 1

### Identification of NOVX Clones

The novel NOVX target sequences identified in the present invention were subjected to the exon linking process to confirm the sequence. PCR primers were designed by starting at the most upstream sequence available, for the forward primer, and at the most downstream sequence available for the reverse primer. Table 16A shows the sequences of the PCR primers used for obtaining different clones. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The PCR product derived from exon linking was cloned into the pCR2.1 vector from Invitrogen. The resulting bacterial clone has an insert covering the entire open reading frame cloned into the pCR2.1 vector. Table 16B shows a list of these bacterial clones. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported herein.

TABLE 12A

PCR Primers for Exon Linking

| | | | | |
|---|---|---|---|---|
| | | SEQ | | SEQ |
| NOVX | | ID | | ID |
| Clone | Primer 1 (5′-3′) | NO | Primer 2 (5′-3′) | NO |

| | | | | |
|---|---|---|---|---|
| NOV6 | CCATGTGGCAGCTGAGGCTTCAT | 105 | AAAGCCCCAGGTCCTCTTGCTAGCT | 106 |
| NOV7 | GGATGAACCAGACTTTGAATAGCAGTG | 107 | GGCTCTCAAGCCCCCATCTC | 108 |
| NOV8 | ATGCGAAGTCACTCTTACCTCTGATGAT | 109 | GGGAGCTGATCTTGAGTTATTTAACATAGC | 110 |
| NOV10 | CTGAATGGAACCATCACCAGC | 111 | ATCAGCACTATTTCTTCATGTGCAGG | 112 |

Physical clone: Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

TABLE 12B
Physical Clones for PCR products

| NOVX Clone | Bacterial Clone |
|---|---|
| NOV7 | Bacterial Clone: 120970::GMAP000808_A.698361.08 |

### Example 2

### Quantitative Expression Analysis of Clones in Various Cells and Tissues

The quantitative expression of various clones was assessed using microtiter plates containing RNA samples from a variety of normal and pathology-derived cells, cell lines and tissues using real time quantitative PCR (RTQ PCR). RTQ PCR was performed on an Applied Biosystems ABI PRISM® 7700 or an ABI PRISM® 7900 HT Sequence Detection System. Various collections of samples are assembled on the plates, and referred to as Panel 1 (containing normal tissues and cancer cell lines), Panel 2 (containing samples derived from tissues from normal and cancer sources), Panel 3 (containing cancer cell lines), Panel 4 (containing cells and cell lines from normal tissues and cells related to inflammatory conditions), Panel 5D/5I (containing human tissues and cell lines with an emphasis on metabolic diseases), AI_comprehensive_panel (containing normal tissue and samples from autoinflammatory diseases), Panel CNSD.01 (containing samples from normal and diseased brains) and CNS_neurodegeneration_panel (containing samples from normal and Alzheimer's diseased brains).

RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

First, the RNA samples were normalized to reference nucleic acids such as constitutively expressed genes (for example, β-actin and GAPDH). Normalized RNA (5 ul) was converted to cDNA and analyzed by RTQ-PCR using One Step RT-PCR Master Mix Reagents (Applied Biosystems; Catalog No. 4309169) and gene-specific primers according to the manufacturer's instructions.

In other cases, non-normalized RNA samples were converted to single strand cDNA (sscDNA) using Superscript II (Invitrogen Corporation; Catalog No. 18064-147) and random hexamers according to the manufacturer's instructions. Reactions containing up to 10 μg of total RNA were performed in a volume of 20 μl and incubated for 60 minutes at 42° C. This reaction can be scaled up to 50 μg of total RNA in a final volume of 100 μl. sscDNA samples are then normalized to reference nucleic acids as described previously, using 1×TaqMan® Universal Master mix (Applied Biosystems; catalog No. 4324020), following the manufacturer's instructions.

Probes and primers were designed for each assay according to Applied Biosystems Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tm) range=58°-60° C., primer optimal Tm=59° C., maximum primer difference=2° C., probe does not have 5′G, probe Tm must be 10° C. greater than primer Tm, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: When working with RNA samples, normalized RNA from each tissue and each cell line was spotted in each well of either a 96 well or a 384-well PCR plate (Applied Biosystems). PCR cocktails included either a single gene specific probe and primers set, or two multiplexed probe and primers sets (a set specific for the target clone and another gene-specific set multiplexed with the target probe). PCR reactions were set up using TaqMan® One-Step RT-PCR Master Mix (Applied Biosystems, Catalog No. 4313803) following manufacturer's instructions. Reverse transcription was performed at 48° C. for 30 minutes followed by amplification/PCR cycles as follows: 95° C. 10 min, then 40 cycles of 95° C. for 15 seconds, 60° C. for 1 minute. Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100.

When working with sscDNA samples, normalized sscDNA was used as described previously for RNA samples. PCR reactions containing one or two sets of probe and primers were set up as described previously, using 1×TaqMan® Universal Master mix (Applied Biosystems; catalog No. 4324020), following the manufacturer's instructions. PCR amplification was performed as follows: 95° C. 10 min, then 40 cycles of 95° C. for 15 seconds, 60° C. for 1 minute. Results were analyzed and processed as described previously.

Panels 1, 1.1, 1.2, and 1.3D

The plates for Panels 1, 1.1, 1.2 and 1.3D include 2 control wells (genomic DNA control and chemistry control) and 94 wells containing cDNA from various samples. The samples in these panels are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in these panels are widely available through the American Type Culture Collection (ATCC), a repository for cultured cell lines, and were cultured using the conditions recommended by the ATCC. The normal tissues found on these panels are comprised of samples derived from all major organ systems from single adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose.

In the results for Panels 1, 1.1, 1.2 and 1.3D, the following abbreviations are used:

ca.=carcinoma,

*=established from metastasis,

met=metastasis,

s cell var=small cell variant,

non-s=non-sm=non-small,

squam=squamous,

pl. eff=pl effusion=pleural effusion,

glio=glioma,

astro=astrocytoma, and

neuro=neuroblastoma.

General_screening_panel_v1.4

The plates for Panel 1.4 include 2 control wells (genomic DNA control and chemistry control) and 94 wells containing cDNA from various samples. The samples in Panel 1.4 are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in Panel 1.4 are widely available through the American Type Culture Collection (ATCC), a repository for cultured cell lines, and were cultured using the conditions recommended by the ATCC. The normal tissues found on Panel 1.4 are comprised of pools of samples derived from all major organ systems from 2 to 5 different adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose. Abbreviations are as described for Panels 1, 1.1, 1.2, and 1.3D.

Panels 2D and 2.2

The plates for Panels 2D and 2.2 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologist at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissues were ascertained to be free of disease and were purchased from various commercial sources such as Clontech (Palo Alto, Calif.), Research Genetics, and Invitrogen.

Panel 3D

The plates of Panel 3D are comprised of 94 cDNA samples and two control samples. Specifically, 92 of these samples are derived from cultured human cancer cell lines, 2 samples of human primary cerebellar tissue and 2 controls. The human cell lines are generally obtained from ATCC (American Type Culture Collection), NCI or the German tumor cell bank and fall into the following tissue groups: Squamous cell carcinoma of the tongue, breast cancer, prostate cancer, melanoma, epidermoid carcinoma, sarcomas, bladder carcinomas, pancreatic cancers, kidney cancers, leukemias/lymphomas, ovarian/uterine/cervical, gastric, colon, lung and CNS cancer cell lines. In addition, there are two independent samples of cerebellum. These cells are all cultured under standard recommended conditions and RNA extracted using the standard procedures. The cell lines in panel 3D and 1.3D are of the most common cell lines used in the scientific literature.

Panels 4D, 4R, and 4.1D

Panel 4 includes samples on a 96 well plate (2 control wells, 94 test samples) composed of RNA (Panel 4R) or cDNA (Panels 4D/4.1D) isolated from various human cell lines or tissues related to inflammatory conditions. Total RNA from control normal tissues such as colon and lung (Stratagene, La Jolla, Calif.) and thymus and kidney (Clontech) was employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1 -5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5×10⁻⁵M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶ cells/ml in DMEM 5% FCS (Hyclone), 100 μM non essential amino acids (Gibco), 1 mM sodium pyruvate (Gibco), mercaptoethanol (5.5×10⁻⁵M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 μM non essential amino acids (Gibco), 1 mM sodium pyruvate (Gibco), mercaptoethanol 5.5×10⁻⁵M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and positive selection. CD45RO beads were then used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and plated at 10⁶ cells/ml onto Falcon 6 well tissue culture plates that had been coated overnight with 0.5 μg/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶ cells/ml in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24, 48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶ cells/ml in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (4 ng/ml). IL-12 (5 ng/ml) and anti-IL4 (1 μg/ml) were used to direct to Th1, while IL-4 (5 ng/ml) and anti-IFN gamma (1 μg/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 μg/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵ cells/ml for 8 days, changing the media every 3 days and adjusting the cell concentration to 5×10⁵ cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 10⁷ cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20° C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7 μl RNAsin and 8 μl DNAse were added. The tube was incubated at 37° C. for 30 minutes to remove contaminating genomic DNA, extracted once with phenol chloroform and re-precipitated with 1/10 volume of 3M sodium acetate and 2 volumes of 100% ethanol. The RNA was spun down and placed in RNAse free water. RNA was stored at −80° C.

AI_Comprehensive Panel_v1.0

The plates for AI_comprehensive panel_v1.0 include two control wells and 89 test samples comprised of cDNA isolated from surgical and postmortem human tissues obtained from the Backus Hospital and Clinomics (Frederick, Md.). Total RNA was extracted from tissue samples from the Backus Hospital in the Facility at CuraGen. Total RNA from other tissues was obtained from Clinomics.

Joint tissues including synovial fluid, synovium, bone and cartilage were obtained from patients undergoing total knee or hip replacement surgery at the Backus Hospital. Tissue samples were immediately snap frozen in liquid nitrogen to ensure that isolated RNA was of optimal quality and not degraded. Additional samples of osteoarthritis and rheumatoid arthritis joint tissues were obtained from Clinomics. Normal control tissues were supplied by Clinomics and were obtained during autopsy of trauma victims.

Surgical specimens of psoriatic tissues and adjacent matched tissues were provided as total RNA by Clinomics. Two male and two female patients were selected between the ages of 25 and 47. None of the patients were taking prescription drugs at the time samples were isolated.

Surgical specimens of diseased colon from patients with ulcerative colitis and Crohns disease and adjacent matched tissues were obtained from Clinomics. Bowel tissue from three female and three male Crohn's patients between the ages of 41-69 were used. Two patients were not on prescription medication while the others were taking dexamethasone, phenobarbital, or tylenol. Ulcerative colitis tissue was from three male and four female patients. Four of the patients were taking lebvid and two were on phenobarbital.

Total RNA from post mortem lung tissue from trauma victims with no disease or with emphysema, asthma or COPD was purchased from Clinomics. Emphysema patients ranged in age from 40-70 and all were smokers, this age range was chosen to focus on patients with cigarette-linked emphysema and to avoid those patients with alpha-1anti-trypsin deficiencies. Asthma patients ranged in age from 36-75, and excluded smokers to prevent those patients that could also have COPD. COPD patients ranged in age from 35-80 and included both smokers and non-smokers. Most patients were taking corticosteroids, and bronchodilators.

In the labels employed to identify tissues in the AI_comprehensive panel_v1.0 panel, the following abbreviations are used:

AI=Autoimmunity

Syn=Synovial

Normal=No apparent disease

Rep22 /Rep20=individual patients

RA=Rheumatoid arthritis

Backus=From Backus Hospital

OA=Osteoarthritis

(SS) (BA) (MF)=Individual patients

Adj=Adjacent tissue

Match control=adjacent tissues

-M=Male

-F=Female

COPD=Chronic obstructive pulmonary disease

Panels 5D and 5I

The plates for Panel 5D and 5I include two control wells and a variety of cDNAs isolated from human tissues and cell lines with an emphasis on metabolic diseases. Metabolic tissues were obtained from patients enrolled in the Gestational Diabetes study. Cells were obtained during different stages in the differentiation of adipocytes from human mesenchymal stem cells. Human pancreatic islets were also obtained.

In the Gestational Diabetes study subjects are young (18-40 years), otherwise healthy women with and without gestational diabetes undergoing routine (elective) Caesarean section. After delivery of the infant, when the surgical incisions were being repaired/closed, the obstetrician removed a small sample.

Patient 2: Diabetic Hispanic, overweight, not on insulin

Patient 7-9: Nondiabetic Caucasian and obese (BMI>30)

Patient 10: Diabetic Hispanic, overweight, on insulin

Patient 11: Nondiabetic African American and overweight

Patient 12: Diabetic Hispanic on insulin

Adipocyte differentiation was induced in donor progenitor cells obtained from Osirus (a division of Clonetics/BioWhittaker) in triplicate, except for Donor 3U which had only two replicates. Scientists at Clonetics isolated, grew and differentiated human mesenchymal stem cells (HuMSCs) for CuraGen based on the published protocol found in Mark F. Pittenger, et al., Multilineage Potential of Adult Human Mesenchymal Stem Cells Science Apr. 2, 1999: 143-147. Clonetics provided Trizol lysates or frozen pellets suitable for mRNA isolation and ds cDNA production. A general description of each donor is as follows:

Donor 2 and 3 U: Mesenchymal Stem cells, Undifferentiated Adipose

Donor 2 and 3 AM: Adipose, AdiposeMidway Differentiated

Donor 2 and 3 AD: Adipose, Adipose Differentiated

Human cell lines were generally obtained from ATCC (American Type Culture Collection), NCI or the German tumor cell bank and fall into the following tissue groups: kidney proximal convoluted tubule, uterine smooth muscle cells, small intestine, liver HepG2 cancer cells, heart primary stromal cells, and adrenal cortical adenoma cells. These cells are all cultured under standard recommended conditions and RNA extracted using the standard procedures. All samples were processed at CuraGen to produce single stranded cDNA.

Panel 5I contains all samples previously described with the addition of pancreatic islets from a 58 year old female patient obtained from the Diabetes Research Institute at the University of Miami School of Medicine. Islet tissue was processed to total RNA at an outside source and delivered to CuraGen for addition to panel 5I.

In the labels employed to identify tissues in the 5D and 5I panels, the following abbreviations are used:

GO Adipose=Greater Omentum Adipose

SK=Skeletal Muscle

UT=Uterus

PL=Placenta

AD=Adipose Differentiated

AM=Adipose Midway Differentiated

U=Undifferentiated Stem Cells

Panel CNSD.01

The plates for Panel CNSD.01 include two control wells and 94 test samples comprised of cDNA isolated from postmortem human brain tissue obtained from the Harvard Brain Tissue Resource Center. Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains two brains from each of the following diagnoses: Alzheimer's disease, Parkinson's disease, Huntington's disease, Progressive Supernuclear Palsy, Depression, and “Normal controls”. Within each of these brains, the following regions are represented: cingulate gyrus, temporal pole, globus palladus, substantia nigra, Brodman Area 4 (primary motor strip), Brodman Area 7 (parietal cortex), Brodman Area 9 (prefrontal cortex), and Brodman area 17 (occipital cortex). Not all brain regions are represented in all cases; e.g., Huntington's disease is characterized in part by neurodegeneration in the globus palladus, thus this region is impossible to obtain from confirmed Huntington's cases. Likewise Parkinson's disease is characterized by degeneration of the substantia nigra making this region more difficult to obtain. Normal control brains were examined for neuropathology and found to be free of any pathology consistent with neurodegeneration.

In the labels employed to identify tissues in the CNS panel, the following abbreviations are used:

PSP Progressive supranuclear palsy

Sub Nigra=Substantia nigra

Glob Palladus=Globus palladus

Temp Pole=Temporal pole

Cing Gyr=Cingulate gyrus

BA 4=Brodman Area 4

Panel CNS_Neurodegeneration_V1.0

The plates for Panel CNS_Neurodegeneration_V1.0 include two control wells and 47 test samples comprised of cDNA isolated from postmortem human brain tissue obtained from the Harvard Brain Tissue Resource Center (McLean Hospital) and the Human Brain and Spinal Fluid Resource Center (VA Greater Los Angeles Healthcare System). Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains six brains from Alzheimer's disease (AD) patients, and eight brains from “Normal controls” who showed no evidence of dementia prior to death. The eight normal control brains are divided into two categories: Controls with no dementia and no Alzheimer's like pathology (Controls) and controls with no dementia but evidence of severe Alzheimer's like pathology, (specifically senile plaque load rated as level 3 on a scale of 0-3; 0=no evidence of plaques, 3=severe AD senile plaque load). Within each of these brains, the following regions are represented: hippocampus, temporal cortex (Brodman Area 21), parietal cortex (Brodman area 7), and occipital cortex (Brodman area 17). These regions were chosen to encompass all levels of neurodegeneration in AD. The hippocampus is a region of early and severe neuronal loss in AD; the temporal cortex is known to show neurodegeneration in AD after the hippocampus; the parietal cortex shows moderate neuronal death in the late stages of the disease; the occipital cortex is spared in AD and therefore acts as a “control” region within AD patients. Not all brain regions are represented in all cases.

In the labels employed to identify tissues in the CNS_Neurodegeneration_V1.0 panel, the following abbreviations are used:

AD=Alzheimer's disease brain; patient was demented and showed AD-like pathology upon autopsy

Control=Control brains; patient not demented, showing no neuropathology

Control (Path)=Control brains; pateint not demented but showing sever AD-like pathology

Sup Temporal Ctx=Superior Temporal Cortex

Inf Temporal Ctx=Inferior Temporal Cortex

A. NOV1 CG50377-01/146642892 and CG50377-02: Cub and Sushi Domain-Containing Protein

Expression of gene CG50377-01 and variant CG50377-02 was assessed using the primer-probe sets Ag2420, Ag169, Ag65 and Ag575, described in Tables 13AA, 13AB, 13AC and 13AD. Results of the RTQ-PCR runs are shown in Tables 13AE, 13AF, 13AG, 13AH, 13AI, 13AJ, 13AK and 13AL.

TABLE 13AA

Probe Name Ag2420

| | | | |
|---|---|---|---|
| Primers | Sequences | Length | Start Position |

| | | | |
|---|---|---|---|
| Forward | 5′-ctgcacttggctggaactta-3′ (SEQ ID NO: 113) | 20 | 9465 |
| Probe | TET-5′-tttcatctcctactccaggtgtacca-3′-TAMRA (SEQ ID NO:114) | 26 | 9498 |
| Reverse | 5′-atctccacaggccctgtaat-3′ (SEQ ID NO: 115) | 20 | 9525 |

TABLE 13AB

Probe Name Ag169

| | | | |
|---|---|---|---|
| Primers | Sequences | Length | Start Position |

| | | | |
|---|---|---|---|
| Foward | 5′-ccagccatgctcagagtgact-3′ (SEQ ID NO: 116) | 21 | 9384 |
| Probe | TET-5′-ttgccaacagcaaggtcaatgccac-3′-TAMRA (SEQ ID NO: 117) | 25 | 9415 |
| Reverse | 5′-cgccactgtggtcgatcat-3′ (SEQ ID NO: 118) | 19 | 9441 |

TABLE 13AC

Probe Name Ag65

| | | | |
|---|---|---|---|
| Primers | Sequences | Length | Start Position |

| | | | |
|---|---|---|---|
| Forward | 5′-ccacagtttgggatacagaacaatt-3′ (SEQ ID NO: 119) | 25 | 8923 |
| Probe | TET-5′-actgtgcttccaacctggtagccctga-3′-TAMRA (SEQ ID NO: 120) | 27 | 8949 |
| Reverse | 5′-agcctttttgacaacggaagag-3′ (SEQ ID NO: 121) 22 | 8977 | |

TABLE 13AD

Probe Name Ag575

| | | | |
|---|---|---|---|
| Primers | Sequences | Length | Start Position |

| | | | |
|---|---|---|---|
| Forward | 5′-aagctggagtatcaggcctatga-3′ (SEQ ID NO: 122) | 23 | 5485 |
| Probe | TET-5′-agagtgcccagacccagagcccttt-3′-TAMRA (SEQ ID NO: 123) | 25 | 5514 |
| Reverse | 5′-ctcccctcacaatgccattg-3′ (SEQ ID NO: 124) | 20 | 5541 |

TABLE 13AE
CNS_neurodegeneration_v1.0

| | | | Rel. Exp. |
|---|---|---|---|
| | Rel. Exp. (%) | | (%) Ag2420, |
| | Ag2420, Run | | Run |
| Tissue Name | 208714879 | Tissue Name | 208714879 |
| AD 1 Hippo | 8.4 | Control (Path) 3 | 1.8 |
| | | Temporal Ctx | |
| AD 2 Hippo | 14.9 | Control (Path) 4 | 15.7 |
| | | Temporal Ctx | |
| AD 3 Hippo | 1.8 | AD 1 Occipital Ctx | 11.0 |
| AD 4 Hippo | 3.0 | AD 2 Occipital Ctx | 0.0 |
| | | (Missing) | |
| AD 5 Hippo | 100.0 | AD 3 Occipital Ctx | 1.9 |
| AD 6 Hippo | 32.3 | AD 4 Occipital Ctx | 9.1 |
| Control 2 Hippo | 11.1 | AD 5 Occipital Ctx | 17.1 |
| Control 4 Hippo | 3.3 | AD 5 Occipital Ctx | 24.7 |
| Control (Path) 3 | 2.6 | Control 1 Occipital | 1.7 |
| Hippo | | Ctx | |
| AD 1 Temporal Ctx | 5.7 | Control 2 Occipital | 49.0 |
| | | Ctx | |
| AD 2 Temporal Ctx | 10.5 | Control 3 Occipital | 11.2 |
| | | Ctx | |
| AD 3 Temporal Ctx | 2.8 | Control 4 Occipital | 1.2 |
| | | Ctx | |
| AD 4 Temporal Ctx | 10.1 | Control (Path) 1 | 48.6 |
| | | Occipital Ctx | |
| AD 5 Inf Temporal | 36.3 | Control (Path) 2 | 8.6 |
| Ctx | | Occipital Ctx | |
| AD 5 Sup Temporal | 33.2 | Control (Path) 3 | 1.2 |
| Ctx | | Occipital Ctx | |
| AD 6 Inf Temporal | 33.9 | Control (Path) 4 | 9.7 |
| Ctx | | Occipital Ctx | |
| AD 6 Sup Temporal | 35.4 | Control 1 Parietal | 0.8 |
| Ctx | | Ctx | |
| Control 1 Temporal | 3.1 | Control 2 Parietal | 14.9 |
| Ctx | | Ctx | |
| Control 2 Temporal | 16.4 | Control 3 Parietal | 9.2 |
| Ctx | | Ctx | |
| Control 3 Temporal | 8.0 | Control (Path) 1 | 39.5 |
| Ctx | | Parietal Ctx | |
| Control 3 Temporal | 2.5 | Control (Path) 2 | 16.5 |
| Ctx | | Parietal Ctx | |
| Control (Path) 1 | 31.9 | Control (Path) 3 | 0.7 |
| Temporal Ctx | | Parietal Ctx | |
| Control (Path) 2 | 23.3 | Control (Path) 4 | 38.7 |
| Temporal Ctx | | Parietal Ctx | |

TABLE 13AF
Panel 1

| | Rel. | Rel. | Rel. | | Rel. | Rel. | Rel. |
|---|---|---|---|---|---|---|---|
| | Exp. (%) | Exp. (%) | Exp. (%) | | Exp. (%) | Exp. (%) | Exp. (%) |
| | Ag169, | Ag169, | Ag65, | | Ag169, | Ag169, | Ag65, |
| | Run | Run | Run | Tissue | Run | Run | Run |
| Tissue Name | 87590884 | 87591554 | 87352491 | Name | 87590884 | 87591554 | 87352491 |
| Endothelial | 0.0 | 0.0 | 0.0 | Renal ca. | 0.0 | 0.0 | 0.0 |
| cells | | | | 786-0 | | | |
| Endothelial | 0.0 | 0.0 | 0.0 | Renal ca. | 0.0 | 0.0 | 0.0 |
| cells (treated) | | | | A498 | | | |
| Pancreas | 0.0 | 0.0 | 0.0 | Renal ca. | 0.0 | 0.0 | 0.0 |
| | | | | RXF 393 | | | |
| Pancreatic ca. | 0.0 | 0.0 | 0.0 | Renal ca. | 0.0 | 0.0 | 0.0 |
| CAPAN 2 | | | | ACHN | | | |
| Adrenal gland | 0.0 | 0.0 | 0.0 | Renal ca. | 0.0 | 0.0 | 0.0 |
| | | | | UO-31 | | | |
| Thyroid | 0.0 | 0.0 | 0.0 | Renal ca. | 0.0 | 0.0 | 0.0 |
| | | | | TK-10 | | | |
| Salivary gland | 0.0 | 0.0 | 0.0 | Liver | 0.0 | 0.0 | 0.0 |
| Pituitary gland | 0.0 | 0.0 | 0.0 | Liver (fetal) | 0.0 | 0.0 | 0.0 |
| Brain (fetal) | 46.7 | 100.0 | 100.0 | Liver ca. | 0.0 | 0.0 | 0.0 |
| | | | | (hepatoblast) | | | |
| | | | | HepG2 | | | |
| Brain (whole) | 3.3 | 0.0 | 24.5 | Lung | 0.0 | 0.0 | 0.0 |
| Brain | 0.0 | 0.0 | 11.1 | Lung (fetal) | 0.0 | 0.0 | 0.0 |
| (amygdala) | | | | | | | |
| Brain | 100.0 | 41.5 | 22.4 | Lung ca. | 0.0 | 0.0 | 0.0 |
| (cerebellum) | | | | (small cell) | | | |
| | | | | LX-1 | | | |
| Brain | 0.0 | 0.0 | 22.8 | Lung ca. | 0.0 | 0.0 | 0.0 |
| (hippocampus) | | | | (small cell) | | | |
| | | | | NCI-H69 | | | |
| Brain | 0.0 | 0.0 | 8.5 | Lung ca. | 0.0 | 0.0 | 0.0 |
| (substantia | | | | (s.cell var.) | | | |
| nigra) | | | | SHP-77 | | | |
| Brain | 0.0 | 0.0 | 2.5 | Lung ca. | 0.0 | 0.0 | 0.0 |
| (thalamus) | | | | (large | | | |
| | | | | cell)NCI- | | | |
| | | | | H460 | | | |
| Brain | 0.0 | 0.0 | 0.0 | Lung ca. | 0.0 | 0.0 | 0.0 |
| (hypothalamus) | | | | (non-sm. | | | |
| | | | | cell) A549 | | | |
| Spinal cord | 0.0 | 0.0 | 2.2 | Lung ca. | 0.0 | 0.0 | 0.0 |
| | | | | (non-s.cell) | | | |
| | | | | NCI-H23 | | | |
| gilo/astro U87- | 34.4 | 0.5 | 29.9 | Lung ca. | 0.0 | 0.0 | 0.0 |
| MG | | | | (non-s.cell) | | | |
| | | | | HOP-62 | | | |
| glio/astro U- | 0.0 | 0.0 | 0.0 | Lung ca. | 0.0 | 0.0 | 0.0 |
| 118-MG | | | | (non-s.cl) | | | |
| | | | | NCI-H522 | | | |
| astrocytoma | 0.0 | 0.0 | 0.9 | Lung ca. | 0.0 | 0.0 | 0.0 |
| SW1783 | | | | (squam.) SW | | | |
| | | | | 900 | | | |
| neuro*; met | 0.0 | 0.0 | 0.0 | Lung ca. | 0.0 | 0.0 | 0.0 |
| SK-N-AS | | | | (squam.) | | | |
| | | | | NCI-H596 | | | |
| astrocytoma | 0.0 | 0.0 | 0.0 | Mammary | 0.0 | 0.0 | 0.0 |
| SF-539 | | | | gland | | | |
| astrocytoma | 0.0 | 0.0 | 0.0 | Breast ca.* | 0.0 | 0.0 | 0.0 |
| SNB-75 | | | | (pl.ef) MCF-7 | | | |
| glioma SNB-19 | 0.0 | 0.0 | 0.0 | Breast ca.* | 0.0 | 0.0 | 0.0 |
| | | | | (pl.ef) | | | |
| | | | | MDA-MB- | | | |
| | | | | 231 | | | |
| glioma U251 | 0.0 | 0.0 | 0.1 | Breast ca.* | 0.0 | 0.0 | 0.0 |
| | | | | (pl. ef) | | | |
| | | | | T47D | | | |
| glioma SF-295 | 0.0 | 0.0 | 0.9 | Breast ca. | 0.0 | 0.0 | 0.0 |
| | | | | BT-549 | | | |
| Heart | 0.0 | 0.0 | 0.0 | Breast ca. | 0.0 | 0.0 | 0.0 |
| | | | | MDA-N | | | |
| Skeletal muscle | 0.0 | 0.0 | 0.0 | Ovary | 0.0 | 0.0 | 0.0 |
| Bone marrow | 0.0 | 0.0 | 0.0 | Ovarian ca. | 0.0 | 0.0 | 0.0 |
| | | | | OVCAR-3 | | | |
| Thymus | 0.0 | 0.0 | 0.0 | Ovarian ca. | 0.0 | 0.0 | 0.0 |
| | | | | OVCAR-4 | | | |
| Spleen | 0.0 | 0.0 | 0.0 | Ovarian ca. | 0.0 | 0.0 | 0.0 |
| | | | | OVCAR-5 | | | |
| Lymph node | 0.0 | 0.0 | 0.0 | Ovarian ca. | 0.0 | 0.0 | 0.0 |
| | | | | OVCAR-8 | | | |
| Colon | 0.0 | 0.0 | 0.0 | Ovarian ca. | 0.0 | 0.0 | 0.0 |
| (ascending) | | | | IGROV-1 | | | |
| Stomach | 0.0 | 0.0 | 0.0 | Ovarian ca. | 0.0 | 0.0 | 0.0 |
| | | | | (ascites) SK- | | | |
| | | | | OV-3 | | | |
| Small intestine | 0.0 | 0.0 | 0.0 | Uterus | 0.0 | 0.0 | 0.0 |
| Colon ca. | 0.0 | 0.0 | 0.0 | Placenta | 0.0 | 0.0 | 0.0 |
| SW480 | | | | | | | |
| Colon ca.* | 0.0 | 0.0 | 0.0 | Prostate | 0.0 | 0.0 | 0.0 |
| SW620 | | | | | | | |
| (SW480 met) | | | | | | | |
| Colon ca. | 0.0 | 0.0 | 0.0 | Prostate ca.* | 0.0 | 0.0 | 0.0 |
| HT29 | | | | (bone met) | | | |
| | | | | PC-3 | | | |
| Colon ca. | 0.0 | 0.0 | 0.0 | Testis | 0.0 | 0.0 | 0.5 |
| HCT-116 | | | | | | | |
| Colon ca. | 0.0 | 0.0 | 0.0 | Melanoma | 0.0 | 0.0 | 0.0 |
| CaCo-2 | | | | Hs688(A).T | | | |
| Colon ca. | 0.0 | 0.0 | 0.0 | Melanoma* | 0.0 | 0.0 | 0.0 |
| HCT-15 | | | | (met) | | | |
| | | | | Hs688(B).T | | | |
| Colon ca. | 0.0 | 0.0 | 0.0 | Melanoma | 0.0 | 0.0 | 0.0 |
| HCC-2998 | | | | UACC-62 | | | |
| Gastric ca. | 0.0 | 0.0 | 0.0 | Melanoma | 0.0 | 0.0 | 0.0 |
| (liver met) | | | | M14 | | | |
| NCI-N87 | | | | | | | |
| Bladder | 0.0 | 0.0 | 0.0 | Melanoma | 0.0 | 0.0 | 0.0 |
| | | | | LOX IMVI | | | |
| Trachea | 0.0 | 0.0 | 0.0 | Melanoma* | 0.0 | 0.0 | 0.0 |
| | | | | (met) SK- | | | |
| | | | | MEL-5 | | | |
| Kidney | 0.0 | 0.0 | 0.0 | Melanoma | 0.0 | 0.0 | 0.0 |
| | | | | SK-MEL-28 | | | |
| Kidney (fetal) | 0.0 | 0.0 | 0.0 | | | | |

TABLE 13AG
Panel 1.1

| | Rel. Exp. (%) Ag575, | | Rel. Exp. (%) Ag575, |
|---|---|---|---|
| Tissue Name | Run 109646812 | Tissue Name | Run 109646812 |
| Adrenal gland | 0.4 | Renal ca. UO-31 | 0.0 |
| Bladder | 9.8 | Renal ca. RXF 393 | 0.0 |
| Brain (amygdala) | 3.7 | Liver | 0.0 |
| Brain (cerebellum) | 18.6 | Liver (fetal) | 0.0 |
| Brain (hippocampus) | 15.9 | Liver ca. | 0.0 |
| | | (hepatoblast) HepG2 | |
| Brain (substantia nigra) | 31.9 | Lung | 0.0 |
| Brain (thalamus) | 3.6 | Lung (fetal) | 0.0 |
| Cerebral Cortex | 34.6 | Lung ca. (non-s.cell) | 0.0 |
| | | HOP-62 | |
| Brain (fetal) | 69.7 | Lung ca. (large | 0.0 |
| | | cell)NCI-H460 | |
| Brain (whole) | 15.6 | Lung ca. (non-s.cell) | 0.0 |
| | | NCI-H23 | |
| glio/astro U-118-MG | 1.4 | Lung ca. (non-s.cl) | 0.8 |
| | | NCI-H522 | |
| astrocytoma SF-539 | 0.0 | Lung ca. (non-sm. | 0.0 |
| | | cell) A549 | |
| astrocytoma SNB-75 | 0.0 | Lung ca. (s.cell var.) | 0.0 |
| | | SHP-77 | |
| astrocytoma SW1783 | 11.4 | Lung ca. (small cell) | 0.0 |
| | | LX-1 | |
| glioma U251 | 2.2 | Lung ca. (small cell) | 0.0 |
| | | NCI-H69 | |
| glioma SF-295 | 20.6 | Lung ca. (squam.) | 0.0 |
| | | SW 900 | |
| glioma SNB-19 | 1.9 | Lung ca. (squam.) | 0.0 |
| | | NCI-H596 | |
| glio/astro U87-MG | 100.0 | Lymph node | 0.0 |
| neuro*; met SK-N-AS | 0.0 | Spleen | 0.0 |
| Mammary gland | 0.0 | Thymus | 0.0 |
| Breast ca. BT-549 | 0.0 | Ovary | 0.0 |
| Breast ca. MDA-N | 0.0 | Ovarian ca. IGROV-1 | 0.0 |
| Breast ca.* (pl. ef) | 0.0 | Ovarian ca. | 0.0 |
| T47D | | OVCAR-3 | |
| Breast ca.* (pl.ef) | 0.0 | Ovarian ca. | 0.0 |
| MCF-7 | | OVCAR-4 | |
| Breast ca.* (pl.ef) | 0.0 | Ovarian ca. | 0.0 |
| MDA-MB-231 | | OVCAR-5 | |
| Small intestine | 0.0 | Ovarian ca. | 0.0 |
| | | OVCAR-8 | |
| Colorectal | 0.0 | Ovarian ca. (ascites) | 0.0 |
| | | SK-OV-3 | |
| Colon ca. HT29 | 0.0 | Pancreas | 0.0 |
| Colon ca. CaCo-2 | 0.0 | Pancreatic ca. | 0.0 |
| | | CAPAN 2 | |
| Colon ca. HCT-15 | 0.0 | Pituitary gland | 0.1 |
| Colon ca. HCT-116 | 0.0 | Placenta | 0.0 |
| Colon ca. HCC-2998 | 0.0 | Prostate | 0.0 |
| Colon ca. SW480 | 0.0 | Prostate ca.* (bone | 0.0 |
| | | met) PC-3 | |
| Colon ca.* SW620 | 0.0 | Salivary gland | 0.0 |
| (SW480 met) | | | |
| Stomach | 0.1 | Trachea | 0.0 |
| Gastric ca. (liver met) | 0.0 | Spinal cord | 3.3 |
| NCI-N87 | | | |
| Heart | 0.1 | Testis | 0.1 |
| Skeletal muscle (Fetal) | 0.1 | Thyroid | 0.0 |
| Skeletal muscle | 0.0 | Uterus | 0.0 |
| Endothelial cells | 0.0 | Melanoma M14 | 0.0 |
| Heart (Fetal) | 0.0 | Melanoma LOX | 0.0 |
| | | IMVI | |
| Kidney | 0.0 | Melanoma UACC- | 0.0 |
| | | 62 | |
| Kidney (fetal) | 0.0 | Melanoma SK- | 0.0 |
| | | MEL-28 | |
| Renal ca. 786-0 | 0.0 | Melanoma* (met) | 0.0 |
| | | SK-MEL-5 | |
| Renal ca. A498 | 0.0 | Melanoma | 1.9 |
| | | Hs688(A).T | |
| Renal ca. ACHN | 0.0 | Melanoma* (met) | 1.8 |
| | | Hs688(B).T | |
| Renal ca. TK-10 | 0.0 | | |

TABLE 13AH
Panel 1.2

| | Rel. Exp. (%) Ag575, | | Rel. Exp. (%) Ag575, |
|---|---|---|---|
| Tissue Name | Run 116351999 | Tissue Name | Run 116351999 |
| Endothelial cells | 0.1 | Renal ca. 786-0 | 0.0 |
| Heart (Fetal) | 0.1 | Renal ca. A498 | 0.0 |
| Pancreas | 0.5 | Renal ca. RXF 393 | 0.0 |
| Pancreatic ca. CAPAN2 | 0.0 | Renal ca. ACHN | 0.0 |
| Adrenal gland | 1.9 | Renal ca. UO-31 | 0.0 |
| Thyroid | 0.3 | Renal ca. TK-10 | 0.0 |
| Salivary gland | 0.2 | Liver | 0.1 |
| Pituitary gland | 1.1 | Liver (fetal) | 0.1 |
| Brain (fetal) | 100.0 | Liver ca. | 0.0 |
| | | (hepatoblast) HepG2 | |
| Brain (whole) | 9.1 | Lung | 0.0 |
| Brain (amygdala) | 7.1 | Lung (fetal) | 0.0 |
| Brain (cerebellum) | 5.3 | Lung ca. (small cell) | 0.0 |
| | | LX-1 | |
| Brain (hippocampus) | 10.2 | Lung ca. (small cell) | 0.0 |
| | | NCI-H69 | |
| Brain (thalamus) | 1.8 | Lung ca. (s.cell var.) | 0.0 |
| | | SHP-77 | |
| Cerebral Cortex | 8.8 | Lung ca. (large | 0.0 |
| | | cell)NCI-H460 | |
| Spinal cord | 1.9 | Lung ca. (non-sm. | 0.0 |
| | | cell) A549 | |
| glio/astro U87-MG | 44.8 | Lung ca. (non-s.cell) | 0.0 |
| | | NCI-H23 | |
| glio/astro U-118-MG | 0.8 | Lung ca. (non-s.cell) | 0.1 |
| | | HOP-62 | |
| astrocytoma SW1783 | 4.3 | Lung ca. (non-s.cl) | 1.0 |
| | | NCI-H522 | |
| neuro*; met SK-N-AS | 0.0 | Lung ca. (squam.) | 0.0 |
| | | SW 900 | |
| astrocytoma SF-539 | 0.0 | Lung ca. (squam.) | 0.0 |
| | | NCI-H596 | |
| astrocytoma SNB-75 | 0.0 | Mammary gland | 0.4 |
| glioma SNB-19 | 2.0 | Breast ca.* (pl.ef) | 0.0 |
| | | MCF-7 | |
| glioma U251 | 3.8 | Breast ca.* (pl.ef) | 0.0 |
| | | MDA-MB-231 | |
| glioma SF-295 | 5.4 | Breast ca.* (pl.ef) | 0.0 |
| | | T47D | |
| Heart | 1.1 | Breast ca. BT-549 | 0.2 |
| Skeletal muscle | 0.7 | Breast ca. MDA-N | 0.0 |
| Bone marrow | 0.0 | Ovary | 0.2 |
| Thymus | 0.0 | Ovarian ca. OVCAR-3 | 0.0 |
| Spleen | 0.3 | Ovarian ca. OVCAR-4 | 0.0 |
| Lymph node | 0.4 | Ovarian ca. OVCAR-5 | 0.0 |
| Colorectal | 0.0 | Ovarian ca. OVCAR-8 | 0.0 |
| Stomach | 0.4 | Ovarian ca. IGROV-1 | 0.0 |
| Small intestine | 0.4 | Ovarian ca. (ascites) | 0.0 |
| | | SK-OV-3 | |
| Colon ca. SW480 | 0.0 | Uterus | 0.2 |
| Colon ca.* SW620 | 0.0 | Placenta | 0.0 |
| (SW480 met) | | | |
| Colon ca. HT29 | 0.0 | Prostate | 1.0 |
| Colon ca. HCT-116 | 0.1 | Prostate ca.* (bone | 0.0 |
| | | met) PC-3 | |
| Colon ca. CaCo-2 | 0.0 | Testis | 1.9 |
| CC Well to Mod Diff | 0.4 | Melanoma | 1.5 |
| (ODO3866) | | Hs688(A).T | |
| Colon ca. HCC-2998 | 0.0 | Melanoma* (met) | 0.9 |
| | | Hs688(B).T | |
| Gastric ca. (liver met) | 0.0 | Melanoma UACC-62 | 0.0 |
| NCI-N87 | | | |
| Bladder | 4.0 | Melanoma M14 | 0.0 |
| Trachea | 0.1 | Melanoma LOX | 0.0 |
| | | IMVI | |
| Kidney | 0.4 | Melanoma* (met) | 0.0 |
| | | SK-MEL-5 | |
| Kidney (fetal) | 1.1 | | |

TABLE 13AI
Panel 1.3D

| | Rel. Exp. (%) | | Rel. Exp. (%) |
|---|---|---|---|
| | Ag2420, | | Ag2420, |
| Tissue Name | Run 159253002 | Tissue Name | Run 159253002 |
| Liver adenocarcinoma | 0.0 | Kidney (fetal) | 0.6 |
| Pancreas | 0.9 | Renal ca. 786-0 | 0.0 |
| Pancreatic ca. CAPAN 2 | 0.0 | Renal ca. A498 | 0.2 |
| Adrenal gland | 1.1 | Renal ca. RXF 393 | 0.0 |
| Thyroid | 0.2 | Renal ca. ACHN | 0.0 |
| Salivary gland | 0.0 | Renal ca. UO-31 | 0.0 |
| Pituitary gland | 0.4 | Renal ca. TK-10 | 0.0 |
| Brain (fetal) | 100.0 | Liver | 0.0 |
| Brain (whole) | 13.6 | Liver (fetal) | 0.2 |
| Brain (amygdala) | 27.4 | Liver ca. | 0.0 |
| | | (hepatoblast) HepG2 | |
| Brain (cerebellum) | 3.9 | Lung | 0.6 |
| Brain (hippocampus) | 95.3 | Lung (fetal) | 0.2 |
| Brain (substantia nigra) | 5.0 | Lung ca. (small cell) | 0.0 |
| | | LX-1 | |
| Brain (thalamus) | 7.9 | Lung ca. (small cell) | 0.0 |
| | | NCI-H69 | |
| Cerebral Cortex | 37.6 | Lung ca. (s. cell var.) | 0.0 |
| | | SHP-77 | |
| Spinal cord | 2.5 | Lung ca. (large | 0.0 |
| | | cell)NCI-H460 | |
| glio/astro U87-MG | 50.3 | Lung ca. (non-sm. | 0.0 |
| | | cell) A549 | |
| glio/astro U-118-MG | 2.7 | Lung ca. (non-s. cell) | 0.0 |
| | | NCI-H23 | |
| astrocytoma SW1783 | 10.9 | Lung ca. (non-s. cell) | 0.0 |
| | | HOP-62 | |
| neuro*; met SK-N-AS | 0.0 | Lung ca. (non-s. cl) | 0.2 |
| | | NCI-H522 | |
| astrocytoma SF-539 | 0.0 | Lung ca. (squam.) | 0.0 |
| | | SW 900 | |
| astrocytoma SNB-75 | 0.0 | Lung ca. (squam.) | 0.0 |
| | | NCI-H596 | |
| glioma SNB-19 | 0.8 | Mammary gland | 0.4 |
| glioma U251 | 1.4 | Breast ca.* (pl. ef) | 0.0 |
| | | MCF-7 | |
| glioma SF-295 | 7.8 | Breast ca.* (pl. ef) | 0.0 |
| | | MDA-MB-231 | |
| Heart (Fetal) | 0.0 | Breast ca.* (pl. ef) | 0.0 |
| | | T47D | |
| Heart | 0.2 | Breast ca. BT-549 | 1.3 |
| Skeletal muscle (Fetal) | 1.3 | Breast ca. MDA-N | 0.0 |
| Skeletal muscle | 0.0 | Ovary | 0.5 |
| Bone marrow | 0.0 | Ovarian ca. OVCAR-3 | 0.0 |
| Thymus | 0.0 | Ovarian ca. OVCAR-4 | 0.0 |
| Spleen | 1.2 | Ovarian ca. OVCAR-5 | 0.0 |
| Lymph node | 0.2 | Ovarian ca. OVCAR-8 | 0.0 |
| Colorectal | 0.0 | Ovarian ca. IGROV-1 | 0.0 |
| Stomach | 0.2 | Ovarian ca. (ascites) | 0.0 |
| | | SK-OV-3 | |
| Small intestine | 1.7 | Uterus | 0.0 |
| Colon ca. SW480 | 0.3 | Placenta | 0.2 |
| Colon ca.* SW620 | 0.0 | Prostate | 0.4 |
| (SW480 met) | | | |
| Colon ca. HT29 | 0.0 | Prostate ca.* (bone | 0.0 |
| | | met) PC-3 | |
| Colon ca. HCT-116 | 0.0 | Testis | 1.3 |
| Colon ca. CaCo-2 | 0.0 | Melanoma | 2.4 |
| | | Hs688(A).T | |
| CC Well to Mod Diff | 1.1 | Melanoma* (met) | 0.3 |
| (ODO3866) | | Hs688(B).T | |
| Colon ca. HCC-2998 | 0.0 | Melanoma UACC-62 | 0.0 |
| Gastric ca. (liver met) | 0.0 | Melanoma M14 | 0.0 |
| NCI-N87 | | | |
| Bladder | 0.6 | Melanoma LOX | 0.0 |
| | | IMVI | |
| Trachea | 0.2 | Melanoma* (met) | 0.0 |
| | | SK-MEL-5 | |
| Kidney | 0.0 | Adipose | 0.5 |

TABLE 13AJ
Panel 2D

| | Rel. Exp. (%) | | Rel. Exp. (%) |
|---|---|---|---|
| | Ag2420, | | Ag2420, |
| Tissue Name | Run 159254587 | Tissue Name | Run 159254587 |
| Normal Colon | 10.0 | Kidney Margin | 3.3 |
| | | 8120608 | |
| CC Well to Mod Diff | 11.1 | Kidney Cancer | 2.9 |
| (ODO3866) | | 8120613 | |
| CC Margin (ODO3866) | 7.3 | Kidney Margin | 1.1 |
| | | 8120614 | |
| CC Gr.2 rectosigmoid | 3.2 | Kidney Cancer | 3.1 |
| (ODO3868) | | 9010320 | |
| CC Margin (ODO3868) | 1.8 | Kidney Margin | 3.8 |
| | | 9010321 | |
| CC Mod Diff (ODO3920) | 4.1 | Normal Uterus | 0.0 |
| CC Margin (ODO3920) | 2.3 | Uterine Cancer | 1.9 |
| | | 064011 | |
| CC Gr.2 ascend colon | 5.2 | Normal Thyroid | 1.7 |
| (ODO3921) | | | |
| CC Margin (ODO3921) | 5.0 | Thyroid Cancer | 1.8 |
| CC from Partial | 67.4 | Thyroid Cancer | 5.2 |
| Hepatectomy (ODO4309) | | A302152 | |
| Mets | | | |
| Liver Margin (ODO4309) | 0.0 | Thyroid Margin | 0.0 |
| | | A302153 | |
| Colon mets to lung | 4.5 | Normal Breast | 1.4 |
| (OD04451-01) | | | |
| Lung Margin (OD04451- | 0.0 | Breast Cancer | 1.6 |
| 02) | | | |
| Normal Prostate 6546-1 | 5.4 | Breast Cancer | 3.8 |
| | | (OD04590-01) | |
| Prostate Cancer | 11.7 | Breast Cancer Mets | 11.3 |
| (OD04410) | | (OD04590-03) | |
| Prostate Margin | 3.5 | Breast Cancer | 0.0 |
| (OD04410) | | Metastasis | |
| Prostate Cancer | 4.7 | Breast Cancer | 8.0 |
| (OD04720-01) | | | |
| Prostate Margin | 4.5 | Breast Cancer | 3.3 |
| (OD04720-02) | | | |
| Normal Lung | 11.1 | Breast Cancer | 3.1 |
| | | 9100266 | |
| Lung Met to Muscle | 0.0 | Breast Margin | 9.5 |
| (ODO4286) | | 9100265 | |
| Muscle Margin | 2.3 | Breast Cancer | 13.7 |
| (ODO4286) | | A209073 | |
| Lung Malignant Cancer | 33.2 | Breast Margin | 0.0 |
| (OD03126) | | A2090734 | |
| Lung Margin (OD03126) | 11.4 | Normal Liver | 0.0 |
| Lung Cancer (OD04404) | 19.3 | Liver Cancer | 8.5 |
| Lung Margin (OD04404) | 2.1 | Liver Cancer 1025 | 0.0 |
| Lung Cancer (OD04565) | 15.8 | Liver Cancer 1026 | 12.1 |
| Lung Margin (OD04565) | 0.0 | Liver Cancer 6004-T | 1.7 |
| Lung Cancer (OD04237- | 13.5 | Liver Tissue 6004-N | 12.9 |
| 01) | | | |
| Lung Margin (OD04237- | 1.8 | Liver Cancer 6005-T | 10.1 |
| 02) | | | |
| Ocular Mel Met to Liver | 0.0 | Liver Tissue 6005-N | 0.0 |
| (ODO4310) | | | |
| Liver Margin (ODO4310) | 0.0 | Normal Bladder | 31.6 |
| Melanoma Metastasis | 0.0 | Bladder Cancer | 10.7 |
| Lung Margin (OD04321) | 0.0 | Bladder Cancer | 100.0 |
| Normal Kidney | 5.2 | Bladder Cancer | 15.2 |
| | | (OD04718-01) | |
| Kidney Ca, Nuclear grade | 0.0 | Bladder Normal | 7.4 |
| 2 (OD04338) | | Adjacent (OD04718- | |
| | | 03) | |
| Kidney Margin | 3.9 | Normal Ovary | 1.7 |
| (OD04338) | | | |
| Kidney Ca Nuclear grade | 1.7 | Ovarian Cancer | 52.1 |
| 1/2 (OD04339) | | | |
| Kidney Margin | 1.0 | Ovarian Cancer | 0.0 |
| (OD04339) | | (OD04768-07) | |
| Kidney Ca, Clear cell | 16.2 | Ovary Margin | 0.0 |
| type (OD04340) | | (OD04768-08) | |
| Kidney Margin | 2.2 | Normal Stomach | 7.9 |
| (OD04340) | | | |
| Kidney Ca, Nuclear grade | 9.4 | Gastric Cancer | 0.0 |
| 3 (OD04348) | | 9060358 | |
| Kidney Margin | 0.0 | Stomach Margin | 2.5 |
| (OD04348) | | 9060359 | |
| Kidney Cancer | 7.0 | Gastric Cancer | 15.4 |
| (OD04622-01) | | 9060395 | |
| Kidney Margin | 0.0 | Stomach Margin | 2.6 |
| (OD04622-03) | | 9060394 | |
| Kidney Cancer | 0.0 | Gastric Cancer | 28.9 |
| (OD04450-01) | | 9060397 | |
| Kidney Margin | 1.8 | Stomach Margin | 1.8 |
| (OD04450-03) | | 9060396 | |
| Kidney Cancer 8120607 | 1.7 | Gastric Cancer | 9.8 |
| | | 064005 | |

TABLE 13AK
Panel 4D

| | Rel. Exp. (%) | | Rel. Exp. (%) |
|---|---|---|---|
| | Ag2420, Run | | Ag2420, Run |
| Tissue Name | 159255381 | | 159255381 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 0.0 |
| | | TNF alpha + IL-1beta | |
| Primary Th2 act | 0.0 | Microvascular Dermal EC | 0.0 |
| | | none | |
| Primary Tr1 act | 0.0 | Microsvasular Dermal EC | 0.0 |
| | | TNF alpha + IL-1beta | |
| Primary Th1 rest | 0.0 | Bronchial epithelium | 0.0 |
| | | TNF alpha + IL1beta | |
| Primary Th2 rest | 0.0 | Small airway epithelium | 1.0 |
| | | none | |
| Primary Tr1 rest | 0.0 | Small airway epithelium | 0.0 |
| | | TNF alpha + IL-1beta | |
| CD45RA CD4 | 11.3 | Coronery artery SMC rest | 3.9 |
| lymphocyte act | | | |
| CD45RO CD4 | 0.0 | Coronery artery SMC | 3.2 |
| lymphocyte act | | TNF alpha + IL-1beta | |
| CD8 lymphocyte act | 0.0 | Astrocytes rest | 100.0 |
| Secondary CD8 | 0.0 | Astrocytes TNF alpha + | 32.8 |
| lymphocyte rest | | IL-1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 0.0 | CCD1106 (Keratinocytes) | 0.0 |
| | | TNF alpha + IL-1beta | |
| LAK cells IL-2 | 0.0 | Liver cirrhosis | 0.0 |
| LAK cells IL-2 + IL-12 | 0.0 | Lupus kidney | 0.0 |
| LAK cells IL-2 + IFN | 0.0 | NCI-H292 none | 0.0 |
| gamma | | | |
| LAK cells IL-2 + IL-18 | 0.0 | NCI-H292 IL-4 | 0.0 |
| LAK cells | 0.0 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL- | 0.0 |
| | | 1beta | |
| PBMC rest | 0.0 | Lung fibroblast none | 36.3 |
| PBMC PWM | 0.0 | Lung fibroblast TNF alpha + | 4.6 |
| | | IL-1beta | |
| PBMC PHA-L | 0.0 | Lung fibroblast IL-4 | 52.9 |
| Ramos (B cell) none | 0.0 | Lung fibroblast IL-9 | 78.5 |
| Ramos (B cell) | 0.0 | Lung fibroblast IL-13 | 37.4 |
| ionomycin | | | |
| B lymphocytes PWM | 0.0 | Lung fibroblast IFN | 37.6 |
| | | gamma | |
| B lymphocytes CD40L | 0.0 | Dermal fibroblast | 63.3 |
| and IL-4 | | CCD1070 rest | |
| EOL-1 dbcAMP | 0.0 | Dermal fibroblast | 27.0 |
| | | CCD1070 TNF alpha | |
| EOL-1 dbcAMP | 0.0 | Dermal fibroblast | 20.3 |
| PMA/ionomycin | | CCD1070 IL-1beta | |
| Dendritic cells none | 0.0 | Dermal fibroblast IFN | 8.6 |
| | | gamma | |
| Dendritic cells LPS | 0.0 | Dermal fibroblast IL-4 | 4.2 |
| Dendritic cells anti- | 0.0 | IBD Colitis 2 | 0.0 |
| CD40 | | | |
| Monocytes rest | 0.0 | IBD Crohn's | 0.0 |
| Monocytes LPS | 0.0 | Colon | 0.7 |
| Macrophages rest | 0.0 | Lung | 0.8 |
| Macrophages LPS | 0.0 | Thymus | 1.9 |
| HUVEC none | 0.0 | Kidney | 0.0 |
| HUVEC starved | 0.0 | | |

TABLE 13AL
Panel CNS_1

| | Rel. Exp. (%) | Rel. Exp. (%) | | Rel. Exp. (%) | Rel. Exp. (%) |
|---|---|---|---|---|---|
| | Ag169, Run | Ag2420, Run | | Ag169, Run | Ag2420, Run |
| Tissue Name | 171628635 | 171648980 | Tissue Name | 171628635 | 171648980 |
| BA4 Control | 15.8 | 11.7 | BA17 PSP | 49.7 | 76.8 |
| BA4 Control2 | 24.7 | 39.5 | BA17 PSP2 | 36.3 | 9.5 |
| BA4 | 6.1 | 4.3 | Sub Nigra | 23.8 | 17.6 |
| Alzheimer's2 | | | Control | | |
| BA4 | 27.0 | 39.2 | Sub Nigra | 37.9 | 7.8 |
| Parkinson's | | | Control2 | | |
| BA4 | 54.0 | 66.4 | Sub Nigra | 13.2 | 11.7 |
| Parkinson's2 | | | Alzheimer's2 | | |
| BA4 | 24.7 | 20.4 | Sub Nigra | 37.6 | 17.3 |
| Huntington's | | | Parkinson's2 | | |
| BA4 | 12.8 | 4.0 | Sub Nigra | 27.4 | 21.2 |
| Huntington's2 | | | Huntington's | | |
| BA4 PSP | 0.0 | 0.0 | Sub Nigra | 17.2 | 42.3 |
| | | | Huntington's2 | | |
| BA4 PSP2 | 12.0 | 4.7 | Sub Nigra | 9.3 | 5.2 |
| | | | PSP2 | | |
| BA4 | 11.9 | 4.8 | Sub Nigra | 9.7 | 21.9 |
| Depression | | | Depression | | |
| BA4 | 13.2 | 0.0 | Sub Nigra | 15.0 | 4.3 |
| Depression2 | | | Depression2 | | |
| BA7 Control | 10.8 | 24.8 | Glob Palladus | 12.1 | 19.6 |
| | | | Control | | |
| BA7 Control2 | 8.7 | 15.6 | Glob Palladus | 42.6 | 5.6 |
| | | | Control2 | | |
| BA7 | 8.6 | 13.9 | Glob Palladus | 0.0 | 7.1 |
| Alzheimer's2 | | | Alzheimer's | | |
| BA7 | 33.4 | 4.7 | Glob Palladus | 4.2 | 0.0 |
| Parkinson's | | | Alzheimer's2 | | |
| BA7 | 55.5 | 37.4 | Glob Palladus | 59.0 | 86.5 |
| Parkinson's2 | | | Parkinson's | | |
| BA7 | 35.4 | 35.8 | Glob Palladus | 4.8 | 5.0 |
| Huntington's | | | Parkinson's2 | | |
| BA7 | 100.0 | 37.9 | Glob Palladus | 8.1 | 0.0 |
| Huntington's2 | | | PSP | | |
| BA7 PSP | 25.3 | 32.8 | Glob Palladus | 0.0 | 0.0 |
| | | | PSP2 | | |
| BA7 PSP2 | 26.4 | 22.2 | Glob Palladus | 1.6 | 4.5 |
| | | | Depression | | |
| BA7 | 18.4 | 2.5 | Temp Pole | 0.0 | 12.9 |
| Depression | | | Control | | |
| BA9 Control | 11.7 | 4.4 | Temp Pole | 32.8 | 44.4 |
| | | | Control2 | | |
| BA9 Control2 | 78.5 | 31.6 | Temp Pole | 10.3 | 0.0 |
| | | | Alzheimer's | | |
| BA9 | 8.2 | 0.0 | Temp Pole | 4.9 | 0.0 |
| Alzheimer's | | | Alzheimer's2 | | |
| BA9 | 7.7 | 4.7 | Temp Pole | 22.7 | 34.4 |
| Alzheimer's2 | | | Parkinson's | | |
| BA9 | 13.6 | 27.0 | Temp Pole | 27.9 | 18.6 |
| Parkinson's | | | Parkinson's2 | | |
| BA9 | 76.8 | 51.1 | Temp Pole | 39.5 | 12.3 |
| Parkinson's2 | | | Huntington's | | |
| BA9 | 60.7 | 28.1 | Temp Pole PSP | 0.0 | 7.7 |
| Huntington's | | | | | |
| BA9 | 36.9 | 0.0 | Temp Pole | 0.0 | 0.0 |
| Huntington's2 | | | PSP2 | | |
| BA9 PSP | 24.0 | 10.2 | Temp Pole | 12.7 | 0.0 |
| | | | Depression2 | | |
| BA9 PSP2 | 5.5 | 0.0 | Cing Gyr | 48.6 | 20.4 |
| | | | Control | | |
| BA9 | 13.5 | 0.0 | Cing Gyr | 32.3 | 39.2 |
| Depression | | | Control2 | | |
| BA9 | 18.7 | 0.0 | Cing Gyr | 0.0 | 26.1 |
| Depression2 | | | Alzheimer's | | |
| BA17 Control | 92.7 | 56.6 | Cing Gyr | 22.8 | 4.8 |
| | | | Alzheimer's2 | | |
| BA17 | 67.4 | 39.0 | Cing Gyr | 31.2 | 31.6 |
| Control2 | | | Parkinson's | | |
| BA17 | 17.8 | 5.2 | Cing Gyr | 46.7 | 17.8 |
| Alzheimer's2 | | | Parkinson's2 | | |
| BA17 | 65.5 | 23.7 | Cing Gyr | 36.9 | 33.2 |
| Parkinson's | | | Huntington's | | |
| BA17 | 92.7 | 100.0 | Cing Gyr | 41.5 | 29.7 |
| Parkinson's2 | | | Huntington's2 | | |
| BA17 | 47.0 | 29.9 | Cing Gyr PSP | 5.4 | 18.7 |
| Huntington's | | | | | |
| BA17 | 33.4 | 21.6 | Cing Gyr PSP2 | 5.5 | 0.0 |
| Huntington's2 | | | | | |
| BA17 | 34.4 | 16.7 | Cing Gyr | 9.2 | 4.5 |
| Depression | | | Depression | | |
| BA17 | 27.0 | 36.3 | Cing Gyr | 20.3 | 24.8 |
| Depression2 | | | Depression2 | | |

CNS_neurodegeneration_v1.0 Summary: Ag2420 Panel CNS_Neurodegeneration does not show any difference in the expression of this gene between the postmortem brains of controls or Alzheimer's disease patients. This panel does, however, confirm the expression of this gene at moderate to high levels in the brains of an independent group of patients. See Panel 1.3d for discussion of utility in the central nervous system.

Panel 1 Summary: Ag65/Ag169 Three experiments with two different probe and primer sets show expression of this gene to be specific to normal brain derived tissue. In addition, there appears to be expression associated with a sample derived from a brain cancer cell line. Thus, the expression of this gene could be used to distinguish these brain derived tissues from other samples in the panel. Moreover, therapeutic modulation of this gene, through the use of antibodies, small molecule drugs or protein therapeutics might be of benefit in the treatment of brain cancer.

Panels 1.1 and 1.2 Summary: Ag575 Expression of this gene appears to be restricted to normal brain derived tissue. In addition, there appears to be expression associated with a number of samples derived from brain cancer cell lines, with highest expression seen in the brain cancer cell line U87-MG (CT=23.5). Thus, the expression of this gene could be used to distinguish these brain derived tissues from other samples in the panel. Moreover, therapeutic modulation of this gene, through the use of antibodies, small molecule drugs or protein therapeutics might be of benefit in the treatment of brain cancer.

This gene also has moderate levels of expression in a number of metabolic tissues including adrenal, pituitary, heart, and fetal skeletal muscle. Thus, this gene product may be important for the pathogenesis, diagnosis and/or treatment of metabolic disease, including obesity. Furthermore, this gene is expressed at higher levels in fetal skeletal muscle (CT=34.2) than in adult skeletal muscle (CT=37.7). Thus, expression of this gene could be used to differentiate between adult and fetal sources of this tissue. In addition, the higher levels of expression of the gene in fetal skeletal muscle suggests that the gene product could be used to restore muscle mass or function in the adult.

Panel 1.3D Summary: Ag2420 Expression of thie gene appears to be restricted to normal brain derived tissue, with highest expression seen in the fetal brain (CT=29.8). In addition, there apears to be expression associated with a number of samples derived from brain cancer cell lines. Thus, the expression of this gene could be used to distinguish these brain derived tissues from other samples in the panel. Moreover, therepeutic modulation of this gene, through the use of antibodies, small molecule drugs or protein therapeutics might be of benefit in the treatment of brain cancer.

This gene represents a novel protein containing CUB and sushi domains. Its expression profile is highly brain-preferential; levels in the CNS appear 10-fold greater than in other tissues. At least one brain-specific protein containing CUB and sushi domains has been linked to seizures, and shows differential expression in response to pentylentetrazole. This protein is therefore a drug target for the treatment of epilepsy or any seizure disorder.

References:

Shimizu-Nishikawa K, Kajiwara K, Kimura M, Katsuki M, Sugaya E. Cloning and expression of SEZ-6, a brain-specific and seizure-related cDNA. Brain Res Mol Brain Res February 1995; 28(2):201-10

To clarify the molecular mechanism of neuronal bursting activity of seizures, we have constructed a cDNA library from mouse cerebrum cortex-derived cells treated with pentylentetrazole (PTZ), one of the convulsant drugs. Using a differential screening technique, several cDNA clones whose expressions change with PTZ-treatment were obtained. Among these clones, SEZ-6 was characterized by increased expression with PTZ. Detailed northern analysis showed that expression of SEZ-6 was limited to the brain and increased by the administration of PTZ not only in in vitro cultured cells but also in vivo. Analysis of SEZ-6 cDNA revealed multiple motifs, including typical signal sequence, threonine-rich domain, five copies of short consensus repeats (SCRs) or sushi domain (complement C3b/C4b binding site), two repeated sequences which were partially similar to the CUB domain or complement C1r/s-like repeat, one transmembrane domain and a short cytoplasmic segment in the C-terminal region. Although many proteins with multiple SCRs or CUB domains other than complement-related proteins have been found, this is the first report about a brain-specific cDNA which encodes membrane protein with both SCRs and CUB domain-like segments. Based on these findings, it is evident that SEZ-6 encodes a novel type of protein which may be related to seizure.

Panel 2D Summary: The expression of this gene in panel 2D appears to be highest in a sample derived from a bladder cancer. Further the expression of thie gene appears fairly selective for certian tissues, more specifically, gastric cancer, ovarian cancer, bladder cancer and lung cancer. Thus, the expression of this gene could be used to distinguish these samples for other samples in the panel. Moreover, therapeutic modulation of this gene, through the use of antibodies, small molecule drugs or protein therapeutics might be of benefit in the treatment of these cancer types.

Panel 4D Summary: Ag2420 This gene encodes a cub-domain and sushi-domain containing single-pass membrane protein and is expressed at a moderate level (CT=32.21) in TNF-alpha+IL-1-beta-stimulated astrocytes and at a higher level (CT=30.6) in resting astrocytes. This gene is also expressed at moderate to low levels (CT=30-34) in resting and cytokine-stimulated lung fibroblasts and dermal fibroblasts. The isolated extracellular domain of the protein encoded by this gene may be useful as a therapeutic protein to reduce or eliminate the symptoms of multiple sclerosis, chronic obstructive pulmonary disease, asthma, or emphysema, and psoriasis. Furthermore, agonist or antagonist antibodies that stimulate or inhibit the function of this gene may also be useful as therapeutics to reduce or eliminate the symptoms of multiple sclerosis, chronic obstructive pulmonary disease, asthma, or emphysema, and psoriasis.

Panel CNS_{—}1 Summary: Ag169/Ag2420 This panel confirms the expression of this CUB and Sushi domain protein in the adult CNS. See Panel 1.3d for a discussion of utility in the central nervous system.

B. NOV4 (SC70504370_A/CG59253-01 and CG59253-02 and CG59253-05 and CG59253-06 and CG59253-07 and CG59253-08)

Expression of gene SC70504370_A and variants CG59253-02 and CG59253-05 and CG59253-06 and CG59253-07 and CG59253-08 was assessed using the primer-probe sets Ag1492 and Ag2441, described in Tables 14BA and 14BB. Results of the RTQ-PCR runs are shown in Tables 14BC, 14BD, 14BE, 14BF, 14BG and 14BH.

TABLE 14BA

Probe Name Ag1492

| | | | |
|---|---|---|---|
| Primers | Sequences | Length | Start Position |

| | | | |
|---|---|---|---|
| Forward | 5′-ctgaagctggcatggtacttaa-3′ (SEQ ID NO: 125) | 22 | 1373 |
| Probe | TET-5′-cagtcctttctctttgaacgacagcg-3′-TAMRA (SEQ ID NO: 126) | 26 | 1410 |
| Reverse | 5′-ttgtaggcttcaatctcttcca-3′ (SEQ ID NO: 127) | 22 | 1442 |

TABLE 14BB

Probe Name Ag2441

| | | | |
|---|---|---|---|
| Primers | Sequences | Length | Start Position |

| | | | |
|---|---|---|---|
| Forward | 5′-tgctatgaaaggcaagcataa-3′ (SEQ ID NO: 128) | 21 | 369 |
| Probe | TET-5′-tgaatgccacaactttatcaaagtatttg-3′-TAMRA (SEQ ID NO: 129) | 29 | 393 |
| Reverse | 5′-aaaaccatctcatcgttcttg-3′ (SEQ ID NO: 130) | 22 | 425 |

TABLE 14BC
CNS_neurodegeneration_v1.0

| | Rel. | Rel. | Rel. | | Rel. | Rel. | Rel. |
|---|---|---|---|---|---|---|---|
| | Exp. (%) | Exp. (%) | Exp. (%) | | Exp. (%) | Exp. (%) | Exp. (%) |
| | Ag1492, | Ag2441, | Ag2441, | | Ag1492, | Ag2441, | Ag2441, |
| Tissue | Run | Run | Run | Tissue | Run | Run | Run |
| Name | 207567462 | 208272964 | 228397027 | Name | 207567462 | 208272964 | 228397027 |
| AD 1 | 8.4 | 0.0 | 7.3 | Control | 4.0 | 0.0 | 2.6 |
| Hippo | | | | (Path) 3 | | | |
| | | | | Temporal | | | |
| | | | | Ctx | | | |
| AD 2 | 18.3 | 0.1 | 17.4 | Control | 23.7 | 61.6 | 16.4 |
| Hippo | | | | (Path) 4 | | | |
| | | | | Temporal | | | |
| | | | | Ctx | | | |
| AD 3 | 4.3 | 0.0 | 2.7 | AD 1 | 19.9 | 0.0 | 12.7 |
| Hippo | | | | Occipital | | | |
| | | | | Ctx | | | |
| AD 4 | 6.3 | 0.0 | 2.4 | AD 2 | 0.0 | 45.4 | 0.0 |
| Hippo | | | | Occipital | | | |
| | | | | Ctx | | | |
| | | | | (Missing) | | | |
| AD 5 | 100.0 | 29.9 | 100.0 | AD 3 | 5.7 | 0.0 | 2.8 |
| Hippo | | | | Occipital | | | |
| | | | | Ctx | | | |
| AD 6 | 28.5 | 36.9 | 27.9 | AD 4 | 20.0 | 0.0 | 16.0 |
| Hippo | | | | Occipital | | | |
| | | | | Ctx | | | |
| Control 2 | 23.0 | 0.1 | 21.8 | AD 5 | 34.4 | 100.0 | 49.0 |
| Hippo | | | | Occipital | | | |
| | | | | Ctx | | | |
| Control 4 | 8.3 | 0.0 | 6.8 | AD 5 | 29.1 | 51.1 | 26.6 |
| Hippo | | | | Occipital | | | |
| | | | | Ctx | | | |
| Control | 4.7 | 0.0 | 2.7 | Control 1 | 3.7 | 0.0 | 2.4 |
| (Path) 3 | | | | Occipital | | | |
| Hippo | | | | Ctx | | | |
| AD 1 | 11.6 | 0.0 | 8.0 | Control 2 | 65.1 | 0.3 | 99.3 |
| Temporal | | | | Occipital | | | |
| Ctx | | | | Ctx | | | |
| AD 2 | 21.0 | 0.1 | 20.6 | Control 3 | 21.6 | 0.1 | 11.6 |
| Temporal | | | | Occipital | | | |
| Ctx | | | | Ctx | | | |
| AD 3 | 4.5 | 0.0 | 2.2 | Control 4 | 5.9 | 0.0 | 4.8 |
| Temporal | | | | Occipital | | | |
| Ctx | | | | Ctx | | | |
| AD 4 | 18.3 | 0.1 | 17.6 | Control | 74.7 | 0.3 | 79.0 |
| Temporal | | | | (Path) 1 | | | |
| Ctx | | | | Occipital | | | |
| | | | | Ctx | | | |
| AD 5 Inf | 72.2 | 27.5 | 79.6 | Control | 16.0 | 0.0 | 9.3 |
| Temporal | | | | (Path) 2 | | | |
| Ctx | | | | Occipital | | | |
| | | | | Ctx | | | |
| AD 5 Sup | 26.1 | 61.6 | 24.0 | Control | 3.2 | 0.0 | 1.8 |
| Temporal | | | | (Path) 3 | | | |
| Ctx | | | | Occipital | | | |
| | | | | Ctx | | | |
| AD 6 Inf | 27.9 | 0.0 | 34.4 | Control | 18.7 | 39.8 | 10.4 |
| Temporal | | | | (Path) 4 | | | |
| Ctx | | | | Occipital | | | |
| | | | | Ctx | | | |
| AD 6 Sup | 32.8 | 21.3 | 39.2 | Control 1 | 7.3 | 0.0 | 5.8 |
| Temporal | | | | Parietal | | | |
| Ctx | | | | Ctx | | | |
| Control 1 | 8.2 | 0.0 | 5.6 | Control 2 | 28.3 | 90.1 | 26.1 |
| Temporal | | | | Parietal | | | |
| Ctx | | | | Ctx | | | |
| Control 2 | 27.9 | 0.1 | 42.3 | Control 3 | 16.3 | 0.1 | 12.9 |
| Temporal | | | | Parietal | | | |
| Ctx | | | | Ctx | | | |
| Control 3 | 11.1 | 0.0 | 9.3 | Control | 53.6 | 0.3 | 74.2 |
| Temporal | | | | (Path) 1 | | | |
| Ctx | | | | Parietal | | | |
| | | | | Ctx | | | |
| Control 3 | 7.8 | 0.0 | 3.9 | Control | 21.0 | 0.1 | 17.6 |
| Temporal | | | | (Path) 2 | | | |
| Ctx | | | | Parietal | | | |
| | | | | Ctx | | | |
| Control | 33.4 | 0.2 | 40.3 | Control | 4.2 | 0.0 | 2.8 |
| (Path) 1 | | | | (Path) 3 | | | |
| Temporal | | | | Parietal | | | |
| Ctx | | | | Ctx | | | |
| Control | 25.0 | 0.1 | 21.6 | Control | 33.9 | 75.3 | 31.0 |
| (Path) 2 | | | | (Path) 4 | | | |
| Temporal | | | | Parietal | | | |
| Ctx | | | | Ctx | | | |

TABLE 14BD
Panel 1.3D

| | Rel. | Rel. | Rel. | | Rel. | Rel. | Rel. |
|---|---|---|---|---|---|---|---|
| | Exp. (%) | Exp. (%) | Exp. (%) | | Exp. (%) | Exp. (%) | Exp. (%) |
| | Ag1492, | Ag2441, | Ag2441, | | Ag1492, | Ag2441, | Ag2441, |
| | Run | Run | Run | Tissue | Run | Run | Run |
| Tissue Name | 165529502 | 159616039 | 165534561 | Name | 165529502 | 159616039 | 165534561 |
| Liver | 0.0 | 0.0 | 0.0 | Kidney | 14.9 | 7.8 | 12.2 |
| adenocarcinoma | | | | (fetal) | | | |
| Pancreas | 4.5 | 1.4 | 4.5 | Renal ca. | 3.9 | 1.8 | 1.2 |
| | | | | 786-0 | | | |
| Pancreatic ca. | 0.0 | 0.0 | 0.0 | Renal ca. | 0.6 | 0.3 | 0.0 |
| CAPAN 2 | | | | A498 | | | |
| Adrenal gland | 2.8 | 0.8 | 2.5 | Renal ca. | 9.8 | 2.2 | 6.8 |
| | | | | RXF 393 | | | |
| Thyroid | 4.9 | 3.3 | 2.1 | Renal ca. | 0.0 | 0.0 | 0.0 |
| | | | | ACHN | | | |
| Salivary gland | 2.0 | 1.1 | 2.3 | Renal ca. | 0.2 | 0.2 | 0.7 |
| | | | | UO-31 | | | |
| Pituitary gland | 9.2 | 6.6 | 2.9 | Renal ca. | 0.0 | 0.0 | 0.0 |
| | | | | TK-10 | | | |
| Brain (fetal) | 44.4 | 12.1 | 26.4 | Liver | 4.4 | 0.9 | 2.9 |
| Brain (whole) | 100.0 | 20.0 | 81.2 | Liver (fetal) | 4.4 | 1.3 | 3.1 |
| Brain | 27.7 | 16.8 | 25.5 | Liver ca. | 0.0 | 0.0 | 0.0 |
| (amygdala) | | | | (hepatoblast) | | | |
| | | | | HepG2 | | | |
| Brain | 42.3 | 8.8 | 27.2 | Lung | 5.0 | 11.4 | 4.4 |
| (cerebellum) | | | | | | | |
| Brain | 50.0 | 77.9 | 26.2 | Lung (fetal) | 7.3 | 7.4 | 13.5 |
| (hippocampus) | | | | | | | |
| Brain | 42.9 | 7.6 | 24.8 | Lung ca. | 0.0 | 0.0 | 0.0 |
| (substantia | | | | (small cell) | | | |
| nigra) | | | | LX-1 | | | |
| Brain | 52.1 | 15.3 | 30.1 | Lung ca. | 4.0 | 23.0 | 18.4 |
| (thalamus) | | | | (small cell) | | | |
| | | | | NCI-H69 | | | |
| Cerebral Cortex. | 43.2 | 70.2 | 23.0 | Lung ca. | 14.7 | 21.5 | 15.6 |
| | | | | (s.cell var.) | | | |
| | | | | SHP-77 | | | |
| Spinal cord | 18.7 | 8.0 | 14.2 | Lung ca. | 2.2 | 0.3 | 0.7 |
| | | | | (large | | | |
| | | | | cell)NCI- | | | |
| | | | | H460 | | | |
| glio/astro U87- | 2.4 | 3.2 | 1.5 | Lung ca. | 0.0 | 0.0 | 0.0 |
| MG | | | | (non-sm. | | | |
| | | | | cell) A549 | | | |
| glio/astro U- | 77.4 | 100.0 | 100.0 | Lung ca. | 0.0 | 0.0 | 0.0 |
| 118-MG | | | | (non-s.cell) | | | |
| | | | | NCI-H23 | | | |
| astrocytoma | 0.0 | 0.6 | 0.3 | Lung ca. | 1.3 | 0.9 | 1.4 |
| SW1783 | | | | (non-s.cell) | | | |
| | | | | HOP-62 | | | |
| neuro*; met | 1.3 | 9.8 | 2.2 | Lung ca. | 0.0 | 0.0 | 0.0 |
| SK-N-AS | | | | (non-s.cl) | | | |
| | | | | NCI-H522 | | | |
| astrocytoma | 0.0 | 0.0 | 0.0 | Lung ca. | 5.0 | 4.1 | 5.1 |
| SF-539 | | | | (squam.) | | | |
| | | | | SW 900 | | | |
| astrocytoma | 5.4 | 3.8 | 6.2 | Lung ca. | 9.9 | 7.1 | 13.7 |
| SNB-75 | | | | (squam.) | | | |
| | | | | NCI-H596 | | | |
| glioma SNB-19 | 3.3 | 4.6 | 3.9 | Mammary | 20.2 | 10.1 | 6.9 |
| | | | | gland | | | |
| glioma U251 | 15.8 | 3.3 | 10.6 | Breast ca.* | 0.0 | 0.0 | 0.1 |
| | | | | (pl.ef) MCF-7 | | | |
| glioma SF-295 | 10.2 | 10.7 | 14.3 | Breast ca.* | 0.0 | 0.3 | 0.3 |
| | | | | (pl.ef) | | | |
| | | | | MDA-MB- | | | |
| | | | | 231 | | | |
| Heart (Fetal) | 4.4 | 10.8 | 2.5 | Breast ca.* | 0.0 | 0.0 | 0.0 |
| | | | | (pl.ef) | | | |
| | | | | T47D | | | |
| Heart | 4.1 | 1.3 | 5.1 | Breast ca. | 0.0 | 0.0 | 0.0 |
| | | | | BT-549 | | | |
| Skeletal muscle | 4.1 | 35.8 | 1.3 | Breast ca. | 0.6 | 2.3 | 0.3 |
| (Fetal) | | | | MDA-N | | | |
| Skeletal muscle | 34.2 | 4.5 | 28.3 | Ovary | 9.8 | 20.6 | 3.8 |
| Bone marrow | 1.3 | 1.7 | 0.3 | Ovarian ca. | 5.6 | 3.3 | 6.3 |
| | | | | OVCAR-3 | | | |
| Thymus | 1.3 | 0.7 | 1.7 | Ovarian ca. | 0.0 | 0.0 | 0.0 |
| | | | | OVCAR-4 | | | |
| Spleen | 1.2 | 1.1 | 2.5 | Ovarian ca. | 0.3 | 0.0 | 0.0 |
| | | | | OVCAR-5 | | | |
| Lymph node | 3.9 | 1.8 | 3.0 | Ovarian ca. | 0.8 | 1.8 | 0.7 |
| | | | | OVCAR-8 | | | |
| Colorectal | 15.2 | 10.4 | 6.3 | Ovarian ca. | 0.0 | 0.0 | 0.0 |
| | | | | IGROV-1 | | | |
| Stomach | 6.0 | 2.3 | 4.3 | Ovarian ca. | 0.0 | 0.0 | 0.0 |
| | | | | (ascites) SK- | | | |
| | | | | OV-3 | | | |
| Small intestine | 19.3 | 10.6 | 10.7 | Uterus | 3.3 | 0.9 | 3.7 |
| Colon ca. | 0.0 | 0.0 | 0.0 | Placenta | 17.8 | 14.8 | 8.1 |
| SW480 | | | | | | | |
| Colon ca.* | 0.2 | 0.0 | 0.0 | Prostate | 3.7 | 0.6 | 1.4 |
| SW620 | | | | | | | |
| (SW480 met) | | | | | | | |
| Colon ca. HT29 | 0.0 | 0.0 | 0.0 | Prostate ca.* | 1.4 | 2.2 | 4.8 |
| | | | | (bone met) | | | |
| | | | | PC-3 | | | |
| Colon ca. HCT- | 0.5 | 0.0 | 0.0 | Testis | 1.8 | 0.8 | 1.3 |
| 116 | | | | | | | |
| Colon ca. | 0.5 | 0.6 | 0.0 | Melanoma | 0.0 | 0.0 | 0.1 |
| CaCo-2 | | | | Hs688(A).T | | | |
| CC Well to | 0.2 | 1.9 | 2.1 | Melanoma* | 0.0 | 0.5 | 0.6 |
| Mod Diff | | | | (met) | | | |
| (ODO3866) | | | | Hs688(B).T | | | |
| Colon ca. HCC- | 0.0 | 0.0 | 0.0 | Melanoma | 5.7 | 0.9 | 2.3 |
| 2998 | | | | UACC-62 | | | |
| Gastric ca. | 0.0 | 0.0 | 0.0 | Melanoma | 6.1 | 1.3 | 9.2 |
| (liver met) NCI- | | | | M14 | | | |
| N87 | | | | | | | |
| Bladder | 3.4 | 1.7 | 3.4 | Melanoma | 0.0 | 0.0 | 0.0 |
| | | | | LOX IMVI | | | |
| Trachea | 1.5 | 2.4 | 1.4 | Melanoma* | 2.0 | 2.3 | 1.3 |
| | | | | (met) SK- | | | |
| | | | | MEL-5 | | | |
| Kidney | 13.0 | 4.2 | 20.6 | Adipose | 8.3 | 5.7 | 9.8 |

TABLE 14
Panel 2.2

| | Rel. Exp. (%) | Rel. Exp. (%) | | Rel. Exp. (%) | Rel. Exp. (%) |
|---|---|---|---|---|---|
| | Ag1492, Run | Ag2441, Run | | Ag1492, Run | Ag2441, Run |
| Tissue Name | 173816674 | 174477149 | Tissue Name | 173816674 | 174477149 |
| Normal Colon | 18.7 | 22.1 | Kidney Margin | 100.0 | 100.0 |
| | | | (OD04348) | | |
| Colon cancer | 4.5 | 4.5 | Kidney | 0.0 | 0.0 |
| (OD06064) | | | malignant | | |
| | | | cancer | | |
| | | | (OD06204B) | | |
| Colon Margin | 46.7 | 34.9 | Kidney normal | 10.2 | 9.9 |
| (OD06064) | | | adjacent tissue | | |
| | | | (OD06204E) | | |
| Colon cancer | 2.2 | 4.1 | Kidney Cancer | 37.9 | 18.9 |
| (OD06159) | | | (OD04450-01) | | |
| Colon Margin | 30.8 | 36.9 | Kidney Margin | 28.9 | 39.8 |
| (OD06159) | | | (OD04450-03) | | |
| Colon cancer | 1.8 | 3.9 | Kidney Cancer | 0.6 | 1.2 |
| (OD06297-04) | | | 8120613 | | |
| Colon Margin | 39.0 | 52.5 | Kidney Margin | 7.6 | 8.7 |
| (OD06297-015) | | | 8120614 | | |
| CC Gr.2 ascend | 4.0 | 2.0 | Kidney Cancer | 1.7 | 1.4 |
| colon | | | 9010320 | | |
| (ODO3921) | | | | | |
| CC Margin | 4.6 | 11.2 | Kidney Margin | 4.4 | 6.3 |
| (ODO3921) | | | 9010321 | | |
| Colon cancer | 1.7 | 0.5 | Kidney Cancer | 0.9 | 0.0 |
| metastasis | | | 8120607 | | |
| (OD06104) | | | | | |
| Lung Margin | 33.4 | 34.9 | Kidney Margin | 2.1 | 3.8 |
| (OD06104) | | | 8120608 | | |
| Colon mets to | 1.9 | 3.0 | Normal Uterus | 9.9 | 6.6 |
| lung (OD04451- | | | | | |
| 01) | | | | | |
| Lung Margin | 21.0 | 7.4 | Uterine Cancer | 2.0 | 2.4 |
| (OD04451-02) | | | 064011 | | |
| Normal Prostate | 3.6 | 1.9 | Normal Thyroid | 1.7 | 1.1 |
| Prostate Cancer | 1.5 | 0.0 | Thyroid Cancer | 1.5 | 1.3 |
| (OD04410) | | | | | |
| Prostate Margin | 4.5 | 5.1 | Thyroid Cancer | 2.1 | 0.3 |
| (OD04410) | | | A302152 | | |
| Normal Ovary | 4.2 | 8.5 | Thyroid Margin | 4.0 | 1.8 |
| | | | A302153 | | |
| Ovarian cancer | 4.6 | 0.3 | Normal Breast | 18.9 | 15.8 |
| (OD06283-03) | | | | | |
| Ovarian Margin | 10.2 | 5.6 | Breast Cancer | 0.0 | 0.5 |
| (OD06283-07) | | | | | |
| Ovarian Cancer | 4.4 | 3.1 | Breast Cancer | 4.9 | 4.4 |
| Ovarian cancer | 4.5 | 5.0 | Breast Cancer | 2.0 | 2.4 |
| (OD06145) | | | (OD04590-01) | | |
| Ovarian Margin | 9.3 | 12.6 | Breast Cancer | 4.0 | 4.1 |
| (OD06145) | | | Mets | | |
| | | | (OD04590-03) | | |
| Ovarian cancer | 1.5 | 1.6 | Breast Cancer | 4.3 | 9.2 |
| (OD06455-03) | | | Metastasis | | |
| Ovarian Margin | 9.7 | 4.4 | Breast Cancer | 3.6 | 1.4 |
| (OD06455-07) | | | | | |
| Normal Lung | 7.0 | 5.6 | Breast Cancer | 2.1 | 1.5 |
| | | | 9100266 | | |
| Invasive poor | 0.6 | 0.0 | Breast Margin | 17.2 | 4.7 |
| diff. lung adeno | | | 9100265 | | |
| (ODO4945-01) | | | | | |
| Lung Margin | 11.2 | 9.2 | Breast Cancer | 3.5 | 2.4 |
| (ODO4945-03) | | | A209073 | | |
| Lung Malignant | 2.8 | 0.5 | Breast Margin | 17.1 | 19.8 |
| Cancer | | | A2090734 | | |
| (OD03126) | | | | | |
| Lung Margin | 2.0 | 3.8 | Breast cancer | 8.4 | 8.6 |
| (OD03126) | | | (OD06083) | | |
| Lung Cancer | 2.0 | 3.6 | Breast cancer | 2.5 | 3.1 |
| (OD05014A) | | | node metastasis | | |
| | | | (OD06083) | | |
| Lung Margin | 11.3 | 8.4 | Normal Liver | 9.7 | 5.8 |
| (OD05014B) | | | | | |
| Lung cancer | 0.3 | 0.8 | Liver Cancer | 0.6 | 0.0 |
| (OD06081) | | | 1026 | | |
| Lung Margin | 13.2 | 10.5 | Liver Cancer | 4.3 | 6.8 |
| (OD06081) | | | 1025 | | |
| Lung Cancer | 0.0 | 0.5 | Liver Cancer | 3.3 | 4.9 |
| (OD04237-01) | | | 6004-T | | |
| Lung Margin | 15.9 | 9.2 | Liver Tissue | 0.8 | 0.6 |
| (OD04237-02) | | | 6004-N | | |
| Ocular Mel Met | 5.3 | 2.3 | Liver Cancer | 1.1 | 0.6 |
| to Liver | | | 6005-T | | |
| (ODO4310) | | | | | |
| Liver Margin | 2.2 | 4.0 | Liver Tissue | 12.5 | 4.2 |
| (ODO4310) | | | 6005-N | | |
| Melanoma | 0.0 | 0.2 | Liver Cancer | 1.0 | 0 6 |
| Metastasis | | | | | |
| Lung Margin | 8.0 | 8.4 | Normal Bladder | 2.1 | 3.3 |
| (OD04321) | | | | | |
| Normal Kidney | 23.2 | 25.2 | Bladder Cancer | 0.8 | 0.0 |
| Kidney Ca, | 55.1 | 65.5 | Bladder Cancer | 2.2 | 2.6 |
| Nuclear grade 2 | | | | | |
| (OD04338) | | | | | |
| Kidney Margin | 2.5 | 2.6 | Normal | 15.4 | 9.6 |
| (OD04338) | | | Stomach | | |
| Kidney Ca | 2.2 | 0.8 | Gastric Cancer | 0.3 | 0.3 |
| Nuclear grade | | | 9060397 | | |
| 1/2 (OD04339) | | | | | |
| Kidney Margin | 18.0 | 14.0 | Stomach | 1.9 | 2.4 |
| (OD04339) | | | Margin | | |
| | | | 9060396 | | |
| Kidney Ca, | 0.8 | 1.2 | Gastric Cancer | 2.9 | 2.1 |
| Clear cell type | | | 9060395 | | |
| (OD04340) | | | | | |
| Kidney Margin | 42.0 | 36.6 | Stomach | 7.7 | 9.7 |
| (OD04340) | | | Margin | | |
| | | | 9060394 | | |
| Kidney Ca, | 0.3 | 0.7 | Gastric Cancer | 2.7 | 3.6 |
| Nuclear grade 3 | | | 064005 | | |
| (OD04348) | | | | | |

TABLE 14BF
Panel 2D

| | Rel. Exp. (%) | | Rel. Exp. (%) |
|---|---|---|---|
| | Ag2441, | | Ag2441, |
| Tissue Name | Run 159616246 | Tissue Name | Run 159616246 |
| Normal Colon | 48.6 | Kidney Margin | 2.6 |
| | | 8120608 | |
| CC Well to Mod Diff | 0.6 | Kidney Cancer | 0.4 |
| (ODO3866) | | 8120613 | |
| CC Margin (ODO3866) | 6.6 | Kidney Margin | 11.5 |
| | | 8120614 | |
| CC Gr.2 rectosigmoid | 0.9 | Kidney Cancer | 1.7 |
| (ODO3868) | | 9010320 | |
| CC Margin (ODO3868) | 1.2 | Kidney Margin | 11.3 |
| | | 9010321 | |
| CC Mod Diff (ODO3920) | 0.5 | Normal Uterus | 0.9 |
| CC Margin (ODO3920) | 9.1 | Uterine Cancer | 3.4 |
| | | 064011 | |
| CC Gr.2 ascend colon | 10.9 | Normal Thyroid | 3.9 |
| (ODO3921) | | | |
| CC Margin (ODO3921) | 6.7 | Thyroid Cancer | 2.0 |
| CC from Partial | 2.0 | Thyroid Cancer | 0.6 |
| Hepatectomy (ODO4309) | | A302152 | |
| Mets | | | |
| Liver Margin (ODO4309) | 3.5 | Thyroid Margin | 10.8 |
| | | A302153 | |
| Colon mets to lung | 0.6 | Normal Breast | 12.2 |
| (OD04451-01) | | | |
| Lung Margin (OD04451- | 3.5 | Breast Cancer | 0.4 |
| 02) | | | |
| Normal Prostate 6546-1 | 1.4 | Breast Cancer | 7.3 |
| | | (OD04590-01) | |
| Prostate Cancer | 2.9 | Breast Cancer Mets | 4.8 |
| (OD04410) | | (OD04590-03) | |
| Prostate Margin | 8.0 | Breast Cancer | 3.6 |
| (OD04410) | | Metastasis | |
| Prostate Cancer | 6.6 | Breast Cancer | 2.0 |
| (OD04720-01) | | | |
| Prostate Margin | 13.3 | Breast Cancer | 5.4 |
| (OD04720-02) | | | |
| Normal Lung | 14.4 | Breast Cancer | 2.1 |
| | | 9100266 | |
| Lung Met to Muscle | 0.1 | Breast Margin | 7.4 |
| (ODO4286) | | 9100265 | |
| Muscle Margin | 4.5 | Breast Cancer | 8.5 |
| (ODO4286) | | A209073 | |
| Lung Malignant Cancer | 4.3 | Breast Margin | 13.8 |
| (OD03126) | | A2090734 | |
| Lung Margin (OD03126) | 15.0 | Normal Liver | 2.7 |
| Lung Cancer (OD04404) | 8.4 | Liver Cancer | 0.1 |
| Lung Margin (OD04404) | 3.7 | Liver Cancer 1025 | 2.3 |
| Lung Cancer (OD04565) | 1.1 | Liver Cancer 1026 | 0.7 |
| Lung Margin (OD04565) | 4.7 | Liver Cancer 6004-T | 4.0 |
| Lung Cancer (OD04237- | 1.2 | Liver Tissue 6004-N | 0.3 |
| 01) | | | |
| Lung Margin (OD04237- | 5.6 | Liver Cancer 6005-T | 0.5 |
| 02) | | | |
| Ocular Mel Met to Liver | 2.7 | Liver Tissue 6005-N | 0.6 |
| (ODO4310) | | | |
| Liver Margin (ODO4310) | 3.0 | Normal Bladder | 4.7 |
| Melanoma Metastasis | 0.7 | Bladder Cancer | 0.1 |
| Lung Margin (OD04321) | 8.0 | Bladder Cancer | 4.9 |
| Normal Kidney | 100.0 | Bladder Cancer | 0.0 |
| | | (OD04718-01) | |
| Kidney Ca, Nuclear grade 2 | 3.6 | Bladder Normal | 2.8 |
| (OD04338) | | Adjacent (OD04718- | |
| | | 03) | |
| Kidney Margin | 32.5 | Normal Ovary | 7.2 |
| (OD04338) | | | |
| Kidney Ca, Nuclear grade | 0.5 | Ovarian Cancer | 6.8 |
| 1/2 (OD04339) | | | |
| Kidney Margin | 26.8 | Ovarian Cancer | 0.2 |
| (OD04339) | | (OD04768-07) | |
| Kidney Ca, Clear cell | 3.8 | Ovary Margin | 1.0 |
| type (OD04340) | | (OD04768-08) | |
| Kidney Margin | 35.4 | Normal Stomach | 6.3 |
| (OD04340) | | | |
| Kidney Ca, Nuclear grade | 0.2 | Gastric Cancer | 1.6 |
| 3 (OD04348) | | 9060358 | |
| Kidney Margin | 15.7 | Stomach Margin | 2.1 |
| (OD04348) | | 9060359 | |
| Kidney Cancer | 1.1 | Gastric Cancer | 4.2 |
| (OD04622-01) | | 9060395 | |
| Kidney Margin | 4.2 | Stomach Margin | 4.2 |
| (OD04622-03) | | 9060394 | |
| Kidney Cancer | 8.0 | Gastric Cancer | 1.6 |
| (OD04450-01) | | 9060397 | |
| Kidney Margin | 25.0 | Stomach Margin | 0.5 |
| (OD04450-03) | | 9060396 | |
| Kidney Cancer 8120607 | 0.6 | Gastric Cancer | 8.9 |
| | | 064005 | |

TABLE 14BG
Panel 4D

| | Rel. | Rel. | | Rel. | Rel. |
|---|---|---|---|---|---|
| | Exp. (%) | Exp. (%) | | Exp. (%) | Exp. (%) |
| | Ag1492, | Ag2441, | | Ag1492, | Ag2441, |
| | Run | Run | | Run | Run |
| Tissue Name | 162778150 | 159616279 | Tissue Name | 162778150 | 159616279 |
| Secondary Th1 act | 0.0 | 0.0 | HUVEC IL-1beta | 12.5 | 9.6 |
| Secondary Th2 act | 0.0 | 0.0 | HUVEC IFN | 5.5 | 6.2 |
| | | | gamma | | |
| Secondary Tr1 act | 0.0 | 0.0 | HUVEC TNF | 3.6 | 1.9 |
| | | | alpha + IFN | | |
| | | | gamma | | |
| Secondary Th1 rest | 0.0 | 0.0 | HUVEC TNF | 5.6 | 5.0 |
| | | | alpha + IL4 | | |
| Secondary Th2 rest | 0.0 | 0.0 | HUVEC IL-11 | 8.5 | 6.5 |
| Secondary Tr1 rest | 0.0 | 0.0 | Lung | 0.4 | 0.1 |
| | | | Microvascular EC | | |
| | | | none | | |
| Primary Th1 act | 0.0 | 0.0 | Lung | 0.0 | 0.0 |
| | | | Microvascular EC | | |
| | | | TNFalpha + IL- | | |
| | | | 1beta | | |
| Primary Th2 act | 0.0 | 0.0 | Microvascular | 0.1 | 0.1 |
| | | | Dermal EC none | | |
| Primary Tr1 act | 0.0 | 0.0 | Microsvasular | 0.0 | 0.0 |
| | | | Dermal EC | | |
| | | | TNFalpha + IL- | | |
| | | | 1beta | | |
| Primary Th1 rest | 0.0 | 0.0 | Bronchial | 2.1 | 2.7 |
| | | | epithelium | | |
| | | | TNFalpha + | | |
| | | | IL1beta | | |
| Primary Th2 rest | 0.0 | 0.0 | Small airway | 0.5 | 0.5 |
| | | | epithelium none | | |
| Primary Tr1 rest | 0.0 | 0.0 | Small airway | 1.1 | 0.8 |
| | | | epithelium | | |
| | | | TNFalpha + IL- | | |
| | | | 1beta | | |
| CD45RA CD4 | 0.4 | 0.6 | Coronery artery | 1.2 | 1.5 |
| lymphocyte act | | | SMC rest | | |
| CD45RO CD4 | 0.0 | 0.0 | Coronery artery | 0.4 | 0.3 |
| lymphocyte act | | | SMC TNFalpha + | | |
| | | | IL-1beta | | |
| CD8 lymphocyte | 0.0 | 0.0 | Astrocytes rest | 1.5 | 1.3 |
| act | | | | | |
| Secondary CD8 | 0.0 | 0.0 | Astrocytes | 0.0 | 0.1 |
| lymphocyte rest | | | TNFalpha + IL- | | |
| | | | 1beta | | |
| Secondary CD8 | 0.0 | 0.0 | KU-812 | 0.0 | 0.0 |
| lymphocyte act | | | (Basophil) rest | | |
| CD4 lymphocyte | 0.0 | 0.0 | KU-812 | 0.0 | 0.0 |
| none | | | (Basophil) | | |
| | | | PMA/ionomycin | | |
| 2ry | 0.0 | 0.0 | CCD1106 | 0.9 | 0.5 |
| Th1/Th2/Tr1_anti- | | | (Keratinocytes) | | |
| CD95 CH11 | | | none | | |
| LAK cells rest | 0.0 | 0.0 | CCD1106 | 0.0 | 0.1 |
| | | | (Keratinocytes) | | |
| | | | TNFalpha + IL- | | |
| | | | 1beta | | |
| LAK cells IL-2 | 0.0 | 0.0 | Liver cirrhosis | 5.7 | 2.6 |
| LAK cells IL-2+IL- | 0.0 | 0.0 | Lupus kidney | 4.5 | 2.4 |
| 12 | | | | | |
| LAK cells IL- | 0.0 | 0.0 | NCI-H292 none | 5.3 | 4.9 |
| 2+IFN gamma | | | | | |
| LAK cells IL-2+ | 0.0 | 0.0 | NCI-H292 IL-4 | 3.0 | 4.9 |
| IL-18 | | | | | |
| LAK cells | 0.0 | 0.0 | NCI-H292 IL-9 | 5.2 | 5.3 |
| PMA/ionomycin | | | | | |
| NK Cells IL-2 rest | 0.0 | 0.0 | NCI-H292 IL-13 | 2.5 | 1.6 |
| Two Way MLR 3 | 0.0 | 0.0 | NCI-H292 IFN | 1.8 | 0.0 |
| day | | | gamma | | |
| Two Way MLR 5 | 0.0 | 0.0 | HPAEC none | 3.7 | 5.2 |
| day | | | | | |
| Two Way MLR 7 | 0.0 | 0.0 | HPAEC TNF | 6.5 | 4.3 |
| day | | | alpha + IL-1 beta | | |
| PBMC rest | 0.0 | 0.0 | Lung fibroblast | 16.2 | 10.4 |
| | | | none | | |
| PBMC PWM | 0.2 | 0.0 | Lung fibroblast | 81.2 | 65.5 |
| | | | TNF alpha + IL-1 | | |
| | | | beta | | |
| PBMC PHA-L | 0.0 | 0.0 | Lung fibroblast | 12.0 | 12.9 |
| | | | IL-4 | | |
| Ramos (B cell) | 0.0 | 0.0 | Lung fibroblast | 22.2 | 13.3 |
| none | | | IL-9 | | |
| Ramos (B cell) | 0.0 | 0.0 | Lung fibroblast | 7.9 | 5.9 |
| ionomycin | | | IL-13 | | |
| B lymphocytes | 0.3 | 0.0 | Lung fibroblast | 10.2 | 7.8 |
| PWM | | | IFN gamma | | |
| B lymphocytes | 0.4 | 0.3 | Dermal fibroblast | 3.3 | 2.4 |
| CD40L and IL-4 | | | CCD1070 rest | | |
| EOL-1 dbcAMP | 0.0 | 0.0 | Dermal fibroblast | 2.5 | 4.5 |
| | | | CCD1070 TNF | | |
| | | | alpha | | |
| EOL-1 dbcAMP | 0.0 | 0.0 | Dermal fibroblast | 6.0 | 5.1 |
| PMA/ionomycin | | | CCD1070 IL-1 | | |
| | | | beta | | |
| Dendritic cells | 0.0 | 0.0 | Dermal fibroblast | 2.1 | 0.7 |
| none | | | IFN gamma | | |
| Dendritic cells LPS | 0.1 | 0.0 | Dermal fibroblast | 9.7 | 7.7 |
| | | | IL-4 | | |
| Dendritic cells anti- | 0.0 | 0.1 | IBD Colitis 2 | 1.7 | 0.2 |
| CD40 | | | | | |
| Monocytes rest | 0.0 | 0.0 | IBD Crohn's | 13.7 | 7.7 |
| Monocytes LPS | 0.0 | 0.0 | Colon | 98.6 | 95.3 |
| Macrophages rest | 0.0 | 0.3 | Lung | 17.2 | 16.0 |
| Macrophages LPS | 0.0 | 0.0 | Thymus | 100.0 | 100.0 |
| HUVEC none | 11.6 | 5.8 | Kidney | 6.3 | 4.2 |
| HUVEC starved | 19.3 | 18.0 | | | |

TABLE 14BH
Panel CNS_1

| | Rel. Exp. | | Rel. Exp. |
|---|---|---|---|
| | (%) | | (%) |
| | Ag1492, | | Ag1492, |
| | Run | | Run |
| Tissue Name | 171634550 | Tissue Name | 171634550 |
| BA4 Control | 31.2 | BA17 PSP | 30.8 |
| BA4 Control2 | 26.8 | BA17 PSP2 | 31.9 |
| BA4 | 6.8 | Sub Nigra Control | 61.6 |
| Alzheimer's2 | | | |
| BA4 Parkinson's | 47.3 | Sub Nigra Control2 | 27.0 |
| BA4 | 54.7 | Sub Nigra | 15.9 |
| Parkinson's2 | | Alzheimer's2 | |
| BA4 | 23.0 | Sub Nigra | 48.3 |
| Huntington's | | Parkinson's2 | |
| BA4 | 15.2 | Sub Nigra | 72.2 |
| Huntington's2 | | Huntington's | |
| BA4 PSP | 12.3 | Sub Nigra | 35.1 |
| | | Huntington's2 | |
| BA4 PSP2 | 37.4 | Sub Nigra PSP2 | 17.4 |
| BA4 Depression | 20.9 | Sub Nigra | 8.9 |
| | | Depression | |
| BA4 | 17.3 | Sub Nigra | 12.7 |
| Depression2 | | Depression2 | |
| BA7 Control | 56.3 | Glob Palladus | 18.3 |
| | | Control | |
| BA7 Control2 | 25.2 | Glob Palladus | 11.6 |
| | | Control2 | |
| BA7 | 10.3 | Glob Palladus | 22.7 |
| Alzheimer's2 | | Alzheimer's | |
| BA7 Parkinson's | 17.7 | Glob Palladus | 9.2 |
| | | Alzheimer's2 | |
| BA7 | 25.5 | Glob Palladus | 81.2 |
| Parkinson's2 | | Parkinson's | |
| BA7 | 39.5 | Glob Palladus | 14.7 |
| Huntington's | | Parkinson's2 | |
| BA7 | 46.7 | Glob Palladus PSP | 3.6 |
| Huntington's2 | | | |
| BA7 PSP | 42.9 | Glob Palladus PSP2 | 12.2 |
| BA7 PSP2 | 42.6 | Glob Palladus | 14.5 |
| | | Depression | |
| BA7 Depression | 16.0 | Temp Pole Control | 9.5 |
| BA9 Control | 45.4 | Temp Pole Control2 | 21.9 |
| BA9 Control2 | 49.3 | Temp Pole | 14.2 |
| | | Alzheimer's | |
| BA9 Alzheimer's | 2.0 | Temp Pole | 4.8 |
| | | Alzheimer's2 | |
| BA9 | 19.9 | Temp Pole | 25.7 |
| Alzheimer's2 | | Parkinson's | |
| BA9 Parkinson's | 37.6 | Temp Pole | 24.5 |
| | | Parkinson's2 | |
| BA9 | 40.1 | Temp Pole | 17.3 |
| Parkinson's2 | | Huntington's | |
| BA9 | 46.0 | Temp Pole PSP | 7.2 |
| Huntington's | | | |
| BA9 | 11.9 | Temp Pole PSP2 | 2.8 |
| Huntington's2 | | | |
| BA9 PSP | 21.8 | Temp Pole | 8.6 |
| | | Depression2 | |
| BA9 PSP2 | 4.3 | Cing Gyr Control | 41.2 |
| BA9 Depression | 8.5 | Cing Gyr Control2 | 24.1 |
| BA9 | 14.7 | Cing Gyr | 12.9 |
| Depression2 | | Alzheimer's | |
| BA17 Control | 100.0 | Cing Gyr | 8.4 |
| | | Alzheimer's2 | |
| BA17 Control2 | 37.1 | Cing Gyr Parkinson's | 25.0 |
| BA17 | 15.3 | Cing Gyr | 23.5 |
| Alzheimer's2 | | Parkinson's2 | |
| BA17 | 40.3 | Cing Gyr | 54.7 |
| Parkinson's | | Huntington's | |
| BA17 | 62.0 | Cing Gyr | 31.4 |
| Parkinson's2 | | Huntington's2 | |
| BA17 | 33.2 | Cing Gyr PSP | 25.7 |
| Huntington's | | | |
| BA17 | 25.7 | Cing Gyr PSP2 | 4.6 |
| Huntington's2 | | | |
| BA17 | 18.6 | Cing Gyr Depression | 12.4 |
| Depression | | | |
| BA17 | 56.3 | Cing Gyr | 27.7 |
| Depression2 | | Depression2 | |

CNS_neurodegeneration_v1.0 Summary: Ag1492/Ag2441 Panel CNS_Neurodegeneration does not detect any difference in the expression of this gene between the postmortem brains of controls or Alzheimer's disease patients. This panel does, however, confirm the expression of this gene at moderate to high levels in the brains of an independent group of patients. See panel 1.3d for discussion of utility in the central nervous system.

Panel 1.3D Summary: Ag1492/Ag2441 The expression of this gene was assessed across 3 independent runs of panel 1.3D utilizing 2 different probe/primer sets. The runs had excellect concordance. This gene encodes a semaphorin homolog that shows an expression profile that is brain-preferential. Highest expression is seen in the brain and a cell line derived from brain cancer (CTs=28-29). Semaphorins can act as axon guidance proteins, specifically as chemorepellents that inhibit CNS regenerative capacity. Manipulation of levels of this protein may be of use in inducing a compensatory synaptogenic response to neuronal death in Alzheimer's disease, Parkinson's disease, Huntington's disease, spinocerebellar ataxia, progressive supranuclear palsy, multiple sclerosis, ALS, head trauma, stroke, or any other disease/condition associated with neuronal loss. Moreover, therapeutic modulation of this gene, through the use of small molecule drugs, antibodies or protein therapeutics might be of use in the treatment of brain cancer.

This gene is also moderately expressed in a wide variety of metabolic tissues, including pancreas, adrenal, thyroid, pituitary, adult and fetal heart, adult and fetal skeletal muscle, adult and fetal liver, and adipose. This suggests that this gene product may be important for the pathogenesis, diagnosis, and/or treatment of metabolic diseases including obesity and Types 1 and 2 diabetes.

Panel 2.2 Summary: Ag1492/2441

The expression of this gene was assessed in two independent runs in panel 2.2 using different probe/primer pairs with good concordance. This gene was found to show highest expression in a sample derived from normal kidney adjacent to a kidney cancer. This pattern of expression was consistent for other normal kidney/kidney cancer pairs as well as for normal colon/colon cancer pairs. Thus, the expression of this gene could be used to distinguish normal colon and kidney tissue from the other samples in the panel, and in particular, their genetically related malignant counterparts. Morover, therapeutic modulation of this gene, through the use of small molecule drugs, antibodies or protein therapeutics might be of use in the treatment of kidney or colon cancer.

Panel 2D Summary: Ag2441 This gene is most highly expressed in a sample derived from normal kidney tissue. This pattern of expression is consistent for other normal kidney/kidney cancer pairs as well as being consistent with Panel 2.2. Thus, the expression of this gene could be used to distinguish normal kidney tissue from the other samples in the panel, and in particular, their genetically related malignant counterparts. Moreover, therapeutic modulation of this gene, through the use of small molecule drugs, antibodies or protein therapeutics might be of use in the treatment of kidney cancer.

Panel 4D Summary: Ag1492/2441 This gene encodes a semaphoring homolog and is expressed at a high level (CTs=28) in TNF-alpha+IL-1-beta-stimulated lung epithelial cells, colon, and thymus. Thus, this gene product be a useful protein therapeutic to reduce or eliminate the symptoms of chronic obstructive pulmonary disease, asthma, emphysema, and ulcerative colitis.

Panel CNS_{—}1 Summary: Ag1492 This panel confirms the expression of this semaphorin precursor in the adult central nervous system. See panel 1.3d for a discussion of utility in the central nervous system.

C. NOV5 (CG50211-01 and CG50211-02: Serine/threonine Kinase)

Expression of gene CG50211-01 and variant CG50211-02 was assessed using the primer-probe set Ag2492, described in Table 15CA. Results of the RTO-PCR runs are shown in Tables 15CB, 15CC, 15CD and 15CE.

TABLE 15CA

Probe Name Ag2492

| | | | |
|---|---|---|---|
| Primers | Sequences | Length | Start Position |

| | | | |
|---|---|---|---|
| Forward | 5′-cagaagctgttccgagaagtc-3′ (SEQ ID NO: 131) | 21 | 501 |
| Probe | TET-5′-atgaagggcctaaaccaccccaacat-3′-TAMRA (SEQ ID NO: 132) | 26 | 528 |
| Reverse | 5′-caatcacctcaaagagcttcac-3′ (SEQ ID NO: 133) | 22 | 555 |

TABLE 15CB
CNS_neurodegeneration_v1.0

| | Rel. Exp. | | Rel. Exp. |
|---|---|---|---|
| | (%) | | (%) |
| | Ag2492, | | Ag2492, |
| | Run | | Run |
| Tissue Name | 208778160 | Tissue Name | 208778160 |
| AD 1 Hippo | 18.4 | Control (Path) 3 | 6.0 |
| | | Temporal Ctx | |
| AD 2 Hippo | 33.4 | Control (Path) 4 | 27.0 |
| | | Temporal Ctx | |
| AD 3 Hippo | 10.4 | AD 1 Occipital Ctx | 14.6 |
| AD 4 Hippo | 9.4 | AD 2 Occipital Ctx | 0.0 |
| | | (Missing) | |
| AD 5 Hippo | 96.6 | AD 3 Occipital Ctx | 6.6 |
| AD 6 Hippo | 85.3 | AD 4 Occipital Ctx | 19.5 |
| Control 2 Hippo | 52.5 | AD 5 Occipital Ctx | 37.1 |
| Control 4 Hippo | 9.9 | AD 5 Occipital Ctx | 87.1 |
| Control (Path) 3 | 5.4 | Control 1 Occipital | 3.5 |
| Hippo | | Ctx | |
| AD 1 Temporal Ctx | 18.8 | Control 2 Occipital | 85.3 |
| | | Ctx | |
| AD 2 Temporal Ctx | 34.9 | Control 3 Occipital | 13.7 |
| | | Ctx | |
| AD 3 Temporal Ctx | 8.2 | Control 4 Occipital | 7.2 |
| | | Ctx | |
| AD 4 Temporal Ctx | 18.9 | Control (Path) 1 | 82.9 |
| | | Occipital Ctx | |
| AD 5 Inf Temporal | 94.0 | Control (Path) 2 | 6.7 |
| Ctx | | Occipital Ctx | |
| AD 5 Sup Temporal | 52.9 | Control (Path) 3 | 4.0 |
| Ctx | | Occipital Ctx | |
| AD 6 Inf Temporal | 69.3 | Control (Path) 4 | 11.6 |
| Ctx | | Occipital Ctx | |
| AD 6 Sup Temporal | 64.6 | Control 1 Parietal | 4.7 |
| Ctx | | Ctx | |
| Control 1 Temporal | 6.3 | Control 2 Parietal | 47.0 |
| Ctx | | Ctx | |
| Control 2 Temporal | 69.3 | Control 3 Parietal | 18.4 |
| Ctx | | Ctx | |
| Control 3 Temporal | 14.9 | Control (Path) 1 | 100.0 |
| Ctx | | Parietal Ctx | |
| Control 3 Temporal | 13.2 | Control (Path) 2 | 21.2 |
| Ctx | | Parietal Ctx | |
| Control (Path) 1 | 82.4 | Control (Path) 3 | 5.5 |
| Temporal Ctx | | Parietal Ctx | |
| Control (Path) 2 | 34.6 | Control (Path) 4 | 36.9 |
| Temporal Ctx | | Parietal Ctx | |

TABLE 15CC
Panel 1.3D

| | Rel. Exp. (%) | | Rel. Exp. (%) |
|---|---|---|---|
| | Ag2492, | | Ag2492, |
| Tissue Name | Run 159841006 | Tissue Name | Run 159841006 |
| Liver adenocarcinoma | 22.7 | Kidney (fetal) | 11.0 |
| Pancreas | 2.3 | Renal ca. 786-0 | 8.1 |
| Pancreatic ca. CAPAN 2 | 2.2 | Renal ca. A498 | 23.8 |
| Adrenal gland | 5.0 | Renal ca. RXF 393 | 3.7 |
| Thyroid | 5.7 | Renal ca. ACHN | 4.1 |
| Salivary gland | 3.9 | Renal ca. UO-31 | 9.7 |
| Pituitary gland | 8.7 | Renal ca.TK-10 | 3.9 |
| Brain (fetal) | 22.8 | Liver | 3.4 |
| Brain (whole) | 18.7 | Liver (fetal) | 7.7 |
| Brain (amygdala) | 24.1 | Liver ca. | 13.4 |
| | | (hepatoblast) HepG2 | |
| Brain (cerebellum) | 7.9 | Lung | 12.7 |
| Brain (hippocampus) | 88.9 | Lung (fetal) | 8.4 |
| Brain (substantia nigra) | 4.7 | Lung ca. (small cell) | 4.2 |
| | | LX-1 | |
| Brain (thalamus) | 26.1 | Lung ca. (small cell) | 14.1 |
| | | NCI-H69 | |
| Cerebral Cortex | 100.0 | Lung ca. (s. cell var.) | 12.8 |
| | | SHP-77 | |
| Spinal cord | 6.6 | Lung ca. (large | 16.5 |
| | | cell)NCI-H460 | |
| glio/astro U87-MG | 9.5 | Lung ca. (non-sm. | 4.4 |
| | | cell) A549 | |
| glio/astro U-118-MG | 17.4 | Lung ca. (non-s. cell) | 21.2 |
| | | NCI-H23 | |
| astrocytoma SW1783 | 14.1 | Lung ca. (non-s. cell) | 26.2 |
| | | HOP-62 | |
| neuro*; met SK-N-AS | 54.0 | Lung ca. (non-s. cl) | 5.5 |
| | | NCI-H522 | |
| astrocytoma SF-539 | 15.5 | Lung ca. (squam.) | 6.7 |
| | | SW 900 | |
| astrocytoma SNB-75 | 16.8 | Lung ca. (squam.) | 2.9 |
| | | NCI-H596 | |
| glioma SNB-19 | 14.9 | Mammary gland | 15.0 |
| glioma U251 | 6.7 | Breast ca.* (pl. ef) | 8.2 |
| | | MCF-7 | |
| glioma SF-295 | 11.9 | Breast ca.* (pl. ef) | 25.9 |
| | | MDA-MB-231 | |
| Heart (Fetal) | 42.0 | Breast ca.* (pl. ef) | 3.7 |
| | | T47D | |
| Heart | 3.3 | Breast ca. BT-549 | 26.6 |
| Skeletal muscle (Fetal) | 54.7 | Breast ca. MDA-N | 7.6 |
| Skeletal muscle | 3.4 | Ovary | 40.6 |
| Bone marrow | 2.2 | Ovarian ca. OVCAR-3 | 6.6 |
| Thymus | 5.9 | Ovarian ca. OVCAR-4 | 4.2 |
| Spleen | 8.0 | Ovarian ca. OVCAR-5 | 6.7 |
| Lymph node | 3.3 | Ovarian ca. OVCAR-8 | 8.5 |
| Colorectal | 8.0 | Ovarian ca. IGROV-1 | 3.4 |
| Stomach | 6.2 | Ovarian ca. (ascites) | 12.2 |
| | | SK-OV-3 | |
| Small intestine | 5.3 | Uterus | 5.5 |
| Colon ca. SW480 | 5.5 | Placenta | 4.9 |
| Colon ca.* SW620 | 2.5 | Prostate | 3.9 |
| (SW480 met) | | | |
| Colon ca. HT29 | 6.0 | Prostate ca.* (bone | 4.5 |
| | | met) PC-3 | |
| Colon ca. HCT-116 | 6.6 | Testis | 31.4 |
| Colon ca. CaCo-2 | 9.9 | Melanoma | 2.6 |
| | | Hs688(A).T | |
| CC Well to Mod Diff | 7.9 | Melanoma* (met) | 3.9 |
| (ODO3866) | | Hs688(B).T | |
| Colon ca. HCC-2998 | 25.9 | Melanoma UACC-62 | 2.7 |
| Gastric ca. (liver met) | 22.4 | Melanoma M14 | 4.3 |
| NCI-N87 | | | |
| Bladder | 4.6 | Melanoma LOX | 9.9 |
| | | IMVI | |
| Trachea | 10.0 | Melanoma* (met) | 5.9 |
| | | SK-MEL-5 | |
| Kidney | 2.5 | Adipose | 4.5 |

TABLE 15CD
Panel 2D

| | Rel. Exp. (%) | | Rel. Exp. (%) |
|---|---|---|---|
| | Ag2492, Run | | Ag2492, |
| Tissue Name | 159841210 | Tissue Name | Run 159841210 |
| Normal Colon | 46.7 | Kidney Margin | 17.4 |
| | | 8120608 | |
| CC Well to Mod Diff | 9.7 | Kidney Cancer | 36.1 |
| (ODO3866) | | 8120613 | |
| CC Margin (ODO3866) | 11.6 | Kidney Margin | 29.5 |
| | | 8120614 | |
| CC Gr.2 rectosigmoid | 11.6 | Kidney Cancer | 31.2 |
| (ODO3868) | | 9010320 | |
| CC Margin (ODO3868) | 5.1 | Kidney Margin | 31.0 |
| | | 9010321 | |
| CC Mod Diff (ODO3920) | 21.2 | Normal Uterus | 9.0 |
| CC Margin (ODO3920) | 20.4 | Uterine Cancer | 29.1 |
| | | 064011 | |
| CC Gr.2 ascend colon | 27.7 | Normal Thyroid | 17.4 |
| (ODO3921) | | | |
| CC Margin (ODO3921 ) | 8.9 | Thyroid Cancer | 23.8 |
| CC from Partial | 17.9 | Thyroid Cancer | 15.7 |
| Hepatectomy (ODO4309) | | A302152 | |
| Mets | | | |
| Liver Margin (ODO4309) | 19.1 | Thyroid Margin | 18.3 |
| | | A302153 | |
| Colon mets to lung | 16.6 | Normal Breast | 27.0 |
| (OD04451-01) | | | |
| Lung Margin (0D04451- | 13.1 | Breast Cancer | 53.2 |
| 02) | | | |
| Normal Prostate 6546-1 | 12.2 | Breast Cancer | 100.0 |
| | | (OD04590-01) | |
| Prostate Cancer | 35.1 | Breast Cancer Mets | 66.4 |
| (OD04410) | | (OD04590-03) | |
| Prostate Margin | 37.4 | Breast Cancer | 92.0 |
| (OD04410) | | Metastasis | |
| Prostate Cancer | 33.2 | Breast Cancer | 20.7 |
| (OD04720-01) | | | |
| Prostate Margin | 41.8 | Breast Cancer | 41.5 |
| (OD04720-02) | | | |
| Normal Lung | 35.4 | Breast Cancer | 38.2 |
| | | 9100266 | |
| Lung Met to Muscle | 17.2 | Breast Margin | 24.0 |
| (ODO4286) | | 9100265 | |
| Muscle Margin | 22.8 | Breast Cancer | 44.8 |
| (ODO4286) | | A209073 | |
| Lung Malignant Cancer | 17.2 | Breast Margin | 25.9 |
| (OD03126) | | A2090734 | |
| Lung Margin (OD03126) | 33.4 | Normal Liver | 11.1 |
| Lung Cancer (OD04404) | 41.5 | Liver Cancer | 15.3 |
| Lung Margin (OD04404) | 17.2 | Liver Cancer 1025 | 13.6 |
| Lung Cancer (OD04565) | 25.5 | Liver Cancer 1026 | 16.6 |
| Lung Margin (OD04565) | 13.9 | Liver Cancer 6004-T | 15.4 |
| Lung Cancer (OD04237- | 31.2 | Liver Tissue 6004-N | 11.4 |
| 01) | | | |
| Lung Margin (OD04237- | 23.0 | Liver Cancer 6005-T | 17.9 |
| 02) | | | |
| Ocular Mel Met to Liver | 35.1 | Liver Tissue 6005-N | 7.6 |
| (ODO4310) | | | |
| Liver Margin (ODO4310) | 14.7 | Normal Bladder | 36.6 |
| Melanoma Metastasis | 21.3 | Bladder Cancer | 12.9 |
| Lung Margin (OD04321) | 28.3 | Bladder Cancer | 19.1 |
| Normal Kidney | 46.3 | Bladder Cancer | 34.6 |
| | | (OD04718-01) | |
| Kidney Ca, Nuclear grade | 37.4 | Bladder Normal | 34.2 |
| 2 (OD04338) | | Adjacent (OD04718- | |
| | | 03) | |
| Kidney Margin | 32.3 | Normal Ovary | 31.4 |
| (OD04338) | | | |
| Kidney Ca Nuclear grade | 20.6 | Ovarian Cancer | 36.1 |
| 1/2 (OD04339) | | | |
| Kidney Margin | 32.1 | Ovarian Cancer | 30.1 |
| (OD04339) | | (OD04768-07) | |
| Kidney Ca, Clear cell | 52.5 | Ovary Margin | 12.9 |
| type (OD04340) | | (OD04768-08) | |
| Kidney Margin | 40.9 | Normal Stomach | 22.2 |
| (OD04340) | | | |
| Kidney Ca, Nuclear grade | 15.9 | Gastric Cancer | 8.5 |
| 3 (OD04348) | | 9060358 | |
| Kidney Margin | 20.9 | Stomach Margin | 20.2 |
| (OD04348) | | 9060359 | |
| Kidney Cancer | 23.5 | Gastric Cancer | 27.0 |
| (OD04622-01) | | 9060395 | |
| Kidney Margin | 9.3 | Stomach Margin | 23.2 |
| (OD04622-03) | | 9060394 | |
| Kidney Cancer | 14.8 | Gastric Cancer | 17.7 |
| (OD04450-01) | | 9060397 | |
| Kidney Margin | 27.5 | Stomach Margin | 12.8 |
| (OD04450-03) | | 9060396 | |
| Kidney Cancer 8120607 | 19.5 | Gastric Cancer | 31.2 |
| | | 064005 | |

TABLE 15CE
Panel 4D

| | Rel. Exp. (%) | | Rel. Exp. (%) |
|---|---|---|---|
| | Ag2492, Run | | Ag2492, Run |
| Tissue Name | 164033742 | Tissue Name | 164033742 |
| Secondary Th1 act | 17.9 | HUVEC IL-1beta | 10.7 |
| Secondary Th2 act | 19.2 | HUVEC IFN gamma | 23.0 |
| Secondary Tr1 act | 23.8 | HUVEC TNF alpha + IFN | 14.3 |
| | | gamma | |
| Secondary Th1 rest | 4.9 | HUVEC TNF alpha + IL4 | 14.1 |
| Secondary Th2 rest | 9.3 | HUVEC IL-11 | 18.3 |
| Secondary Tr1 rest | 7.2 | Lung Microvascular EC | 18.8 |
| | | none | |
| Primary Th1 act | 18.0 | Lung Microvascular EC | 19.9 |
| | | TNF alpha + IL-1beta | |
| Primary Th2 act | 12.2 | Microvascular Dermal EC | 30.4 |
| | | none | |
| Primary Tr1 act | 22.5 | Microsvasular Dermal EC | 16.8 |
| | | TNF alpha + IL-1beta | |
| Primary Th1 rest | 27.2 | Broncial epithelium | 8.7 |
| | | TNF alpha + IL-1beta | |
| Primary Th2 rest | 23.3 | small airway epithelium | 18.3 |
| | | none | |
| Primary Tr1 rest | 11.7 | Small airway epithelium | 100.0 |
| | | TNF alpha + IL-1beta | |
| CD45RA CD4 | 8.8 | Coronery artery SMC rest | 31.9 |
| lymphocyte act | | | |
| CD45RO CD4 | 19.5 | Coronery artery SMC | 22.7 |
| lymphocyte act | | TNF alpha + IL-1beta | |
| CD8 lymphocyte act | 9.3 | Astrocytes rest | 27.9 |
| Secondary CD8 | 12.7 | Astrocytes TNF alpha + | 21.8 |
| lymphocyte rest | | IL-1beta | |
| Secondary CD8 | 9.7 | KU-812 (Basophil) rest | 10.4 |
| lymphocyte act | | | |
| CD4 lymphocyte none | 4.7 | KU-812 (Basophil) | 28.3 |
| | | PMA/ionomycin | |
| 2ry Th1/Th2/Tr1_anti- | 9.0 | CCD1106 (Keratinocytes) | 15.0 |
| CD95 CH11 | | none | |
| LAK cells rest | 7.7 | CCD1106 (Keratinocytes) | 3.2 |
| | | TNF alpha + IL-1beta | |
| LAK cells IL-2 | 15.5 | Liver cirrhosis | 3.7 |
| LAK cells IL-2 + IL-12 | 11.0 | Lupus kidney | 3.4 |
| LAK cells IL-2 + IFN | 14.9 | NCI-H292 none | 30.6 |
| gamma | | | |
| LAK cells IL-2 + IL-18 | 12.0 | NCI-H292 IL-4 | 39.8 |
| LAK cells | 5.6 | NCI-H292 IL-9 | 70.2 |
| PMA/ionomycin | | | |
| NK Cells IL-2 rest | 10.2 | NCI-H292 IL-13 | 18.4 |
| Two Way MLR 3 day | 11.7 | NCI-H292 IFN gamma | 27.7 |
| Two Way MLR 5 day | 6.2 | HPAEC none | 24.1 |
| Two Way MLR 7 day | 6.9 | HPAEC TNF alpha + IL- | 25.3 |
| | | 1beta | |
| PBMC rest | 7.2 | Lung fibroblast none | 15.3 |
| PBMC PWM | 29.5 | Lung fibroblast | 14.1 |
| | | TNF alpha + IL-1beta | |
| PBMC PHA-L | 13.8 | Lung fibroblast IL-4 | 36.9 |
| Ramos (B cell) none | 6.4 | Lung fibroblast IL-9 | 28.9 |
| Ramos (B cell) | 36.6 | Lung fibroblast IL-13 | 22.4 |
| ionomycin | | | |
| B lymphocytes PWM | 48.0 | Lung fibroblast IFN | 53.6 |
| | | gamma | |
| B lymphocytes CD40L | 20.2 | Dermal fibroblast | 40.1 |
| and IL-4 | | CCD1070 rest | |
| EOL-1 dbcAMP | 15.8 | Dermal fibroblast | 74.7 |
| | | CCD1070 TNF alpha | |
| EOL-1 dbcAMP | 31.9 | Dermal fibroblast | 19.5 |
| PMA/ionomycin | | CCD1070 IL-1beta | |
| Dendritic cells none | 10.9 | Dermal fibroblast IFN | 11.5 |
| | | gamma | |
| Dendritic cells LPS | 10.0 | Dermal fibroblast IL-4 | 31.0 |
| Dendritic cells anti- | 6.8 | IBD Colitis 2 | 0.9 |
| CD40 | | | |
| Monocytes rest | 9.9 | IBD Crohn's | 1.9 |
| Monocytes LPS | 13.5 | Colon | 14.6 |
| Macrophages rest | 11.1 | Lung | 21.2 |
| Macrophages LPS | 7.6 | Thymus | 25.2 |
| HUVEC none | 26.2 | Kidney | 49.3 |
| HUVEC starved | 40.6 | | |

CNS_neurodegeneration_v1.0 Summary: Ag2492 Panel CNS_Neurodegeneration does not detect any difference in the expression of this gene between the postmortem brains of controls or Alzheimer's disease patients. This panel does, however, confirm the expression of this gene at moderate levels in the brains of an independent group of patients. See panel 1.3d for discussion of utility in the central nervous system.

Panel 1.3D Summary: Ag2492 This gene encodes a serine/threonine kinase homolog that is expressed in moderate to high levels in the CNS, with highest expression in the cerebral cortex (CT=26.5). Serine/threonine kinases are activated by antidepressants; this gene may therefore be a small molecule target for the treatment of depression or bipolar disorder.

This gene is moderately expressed in a number of metabolic tissues including pancreas, adrenal, pituitary, thyroid, adult and fetal heart, adult and fetal skeletal muscle, adult and fetal liver, and adipose. This suggests that this kinase may be a small molecule target for the treatment of metabolic disease, including obesity and Types 1 and 2 diabetes. This gene is also expressed at higher levels in fetal heart and skeletal muscle (CTs=27.5) than in adult heart and skeletal muscle (CTs=31.5). This suggests that the expression of this gene could be used to differentiate between the adult and fetal sources of this tissue. Furthermore, the higher levels of expression in the fetal tissue suggest that the protein encoded by this gene may be involved in the development of these organs. Thus, therapeutic modulation of the expression or function of the gene product may be useful in treating disease that effect the heart and skeletal muscle.

There is also consistent expression in tissues derived from brain cancer cell lines, in addition to the expression in normal brain. Thus, the expression of this gene could be used to distinguish tissues or cell lines derived from brain from other samples in the panel. Moreover, therapeutic modulation of this gene, through the use of small molecule drugs, antibodies or protein therapeutics might be of benefit in the treatment of brain cancer.

References:

Popoli M, Mori S, Brunello N, Perez J, Gennarelli M, Racagni G.Serine/threonine kinases as molecular targets of antidepressants: implications for pharmacological treatment and pathophysiology of affective disorders. Pharmacol Ther February 2001;89(2):149-70

It is currently a widely accepted opinion that adaptive, plastic changes in the molecular and cellular components of neuronal signaling systems correlate with the effects on mood and cognition observed after long-term treatment with antidepressant drugs. Protein phosphorylation represents a key step for most signaling systems, and it is involved in the regulation of virtually all cellular functions. Two serine/threonine kinases, Ca2+/calmodulin-dependent protein kinase II and cyclic AMP-dependent protein kinase, have been shown to be activated in the brain following antidepressant treatment. The changes in kinase activity are mirrored by changes in the phosphorylation of selected protein substrates in subcellular compartments (presynaptic terminals and microtubules), which, in turn, may contribute to the modulation of synaptic transmission observed with antidepressants. The molecular consequences of protein kinase activation may account for some of the alterations in neural function induced by antidepressants, and may suggest novel possible strategies of pharmacological intervention.

Panel 2D Summary: Ag2492 The expression of this gene in panel 2D appears to be highest in a sample derived from a breast cancer (CT=28.6). There is also substantial expression in other breast cancers as well. Thus, the expression of this gene could be used to distinguish breast cancer samples from other samples in the panel. Moreover, therapeutic modulation of this gene, through the use of small molecule drugs, antibodies or protein therapeutics might be of benefit in the treatment of breast cancer.

Panel 4D Summary: Ag2492 This gene encodes a serine/threonine protein kinasehomolog and is expressed at a moderate level in all cells and tissues in this panel, with highest expression in TNF-alpha+IL-1-beta stimulated small airway epithelium, IL-9-stimulated NCI-H292 pulmonary mucoepidermoid cells, and TNF-alpha-stimulated CCD1070 dermal fibroblasts (CTs=27.5). This expression profile suggests that small molecule drugs that inhibit this novel serine/threonine protein kinase-like protein may be useful therapeutics that reduce or eliminate the symptoms of chronic obstructive pulmonary disease, asthma, emphysema, and psoriasis.

D. NOV6 (CG50215-01 and CG50215-04)

Expression of gene CG50215-01 and variant CG50215-04 was assessed using the primer-probe set Ag2493, described in Table 16DA. Results of the RTQ-PCR runs are shown in Tables 16DB, 16DC and 16DD.

TABLE 16DA

Probe Name Ag2493

| | | | |
|---|---|---|---|
| Primers | Sequences | Length | Start Position |

| | | | |
|---|---|---|---|
| Forward | 5′-agaacacccctggctccta-3′ (SEQ ID NO: 134) | 19 | 2865 |
| Probe | TET-5′-acaccagcctgtgaccctggctat-3′-TAMRA (SEQ ID NO: 135) | 24 | 2891 |
| Reverse | 5′-gtttcacactcgttcacatcct-3′ (SEQ ID NO: 136) | 22 | 2940 |

TABLE 16DB
Panel 1.3D

| | Rel. Exp. (%) | | Rel. Exp. (%) |
|---|---|---|---|
| | Ag2493, | | Ag2493, |
| Tissue Name | Run 165630586 | Tissue Name | Run 165630586 |
| Liver adenocarcinoma | 0.0 | Kidney (fetal) | 3.9 |
| Pancreas | 0.0 | Renal ca. 786-0 | 0.0 |
| Pancreatic ca. CAPAN 2 | 36.6 | Renal ca. A498 | 0.0 |
| Adrenal gland | 0.0 | Renal ca. RXF 393 | 0.0 |
| Thyroid | 0.0 | Renal ca. ACHN | 0.0 |
| Salivary gland | 0.0 | Renal ca. UO-31 | 0.0 |
| Pituitary gland | 0.0 | Renal ca. TK-10 | 21.9 |
| Brain (fetal) | 5.6 | Liver | 0.0 |
| Brain (whole) | 0.0 | Liver (fetal) | 7.6 |
| Brain (amygdala) | 6.6 | Liver ca. | 0.0 |
| | | (hepatoblast) HepG2 | |
| Brain (cerebellum) | 0.0 | Lung | 0.0 |
| Brain (hippocampus) | 0.0 | Lung (fetal) | 0.0 |
| Brain (substantia nigra) | 0.0 | Lung ca. (small cell) | 0.0 |
| | | LX-1 | |
| Brain (thalamus) | 0.0 | Lung ca. (small cell) | 0.0 |
| | | NCI-H69 | |
| Cerebral Cortex | 13.5 | Lung ca. (s. cell var.) | 0.0 |
| | | SHP-77 | |
| Spinal cord | 0.0 | Lung ca. (large | 2.3 |
| | | cell)NCI-H460 | |
| glio/astro U87-MG | 5.3 | Lung ca. (non-sm. | 13.6 |
| | | cell) A549 | |
| glio/astro U-118-MG | 7.7 | Lung ca. (non-s. cell) | 7.5 |
| | | NCI-H23 | |
| astrocytoma SW1783 | 5.6 | Lung ca. (non-s. cell) | 0.0 |
| | | HOP-62 | |
| neuro*; met SK-N-AS | 0.0 | Lung ca. (non-s. cl) | 0.0 |
| | | NCI-H522 | |
| astrocytoma SF-539 | 0.0 | Lung ca. (squam.) | 0.0 |
| | | SW 900 | |
| astrocytoma SNB-75 | 0.0 | Lung ca. (squam.) | 0.0 |
| | | NCI-H596 | |
| glioma SNB-19 | 0.0 | Mammary gland | 0.0 |
| glioma U251 | 13.3 | Breast ca.* (pl. ef) | 26.4 |
| | | MCF-7 | |
| glioma SF-295 | 0.0 | Breast ca.* (pl. ef) | 0.0 |
| | | MDA-MB-231 | |
| Heart (Fetal) | 0.0 | Breast ca.* (pl. ef) | 8.8 |
| | | T47D | |
| Heart | 0.0 | Breast ca. BT-549 | 0.0 |
| Skeletal muscle (Fetal) | 0.0 | Breast ca. MDA-N | 13.7 |
| Skeletal muscle | 0.0 | Ovary | 0.0 |
| Bone marrow | 0.0 | Ovarian ca. OVCAR-3 | 27.9 |
| Thymus | 0.0 | Ovarian ca. OVCAR-4 | 0.0 |
| Spleen | 0.0 | Ovarian ca. OVCAR-5 | 17.7 |
| Lymph node | 11.0 | Ovarian ca. OVCAR-8 | 0.0 |
| Colorectal | 10.2 | Ovarian ca. IGROV-1 | 0.0 |
| Stomach | 0.0 | Ovarian ca. (ascites) | 0.0 |
| | | SK-OV-3 | |
| Small intestine | 0.0 | Uterus | 0.0 |
| Colon ca. SW480 | 0.0 | Placenta | 0.0 |
| Colon ca.* SW620 | 0.0 | Prostate | 100.0 |
| (SW480 met) | | | |
| Colon ca. HT29 | 0.0 | Prostate ca.* (bone | 8.8 |
| | | met) PC-3 | |
| Colon ca. HCT-116 | 0.0 | Testis | 0.0 |
| Colon ca. CaCo-2 | 22.1 | Melanoma | 0.0 |
| | | Hs688(A).T | |
| CC Well to Mod Diff | 7.0 | Melanoma* (met) | 0.0 |
| (ODO3866) | | Hs688(B).T | |
| Colon ca. HCC-2998 | 0.0 | Melanoma UACC-62 | 3.9 |
| Gastric ca. (liver met) | 0.0 | Melanoma M14 | 0.0 |
| NCI-N87 | | | |
| Bladder | 0.0 | Melanoma LOX | 0.0 |
| | | IMVI | |
| Trachea | 0.0 | Melanoma* (met) | 0.0 |
| | | SK-MEL-5 | |
| Kidney | 0.0 | Adipose | 28.1 |

TABLE 16DC
Panel 2.2

| | Rel. Exp. (%) | | Rel. Exp. (%) |
|---|---|---|---|
| | Ag2493, Run | | Ag2493, |
| Tissue Name | 174926557 | Tissue Name | Run 174926557 |
| Normal Colon | 10.0 | Kidney Margin | 7.2 |
| | | (OD04348) | |
| Colon cancer | 2.4 | Kidney malignant | 0.6 |
| (OD06064) | | cancer (OD06204B) | |
| Colon Margin | 7.2 | Kidney normal adjacent | 6.7 |
| (OD06064) | | tissue (OD06204E) | |
| Colon cancer | 2.7 | Kidney Cancer | 0.5 |
| (OD06159) | | (OD04450-01) | |
| Colon Margin | 7.2 | Kidney Margin | 3.0 |
| (OD06159) | | (OD04450-03) | |
| Colon cancer | 1.0 | Kidney Cancer 8120613 | 0.4 |
| (OD06297-04) | | | |
| Colon Margin | 10.2 | Kidney Margin | 4.6 |
| (OD06297-015) | | 8120614 | |
| CC Gr.2 ascend colon | 3.7 | Kidney Cancer 9010320 | 2.0 |
| (ODO3921) | | | |
| CC Margin (ODO3921) | 0.6 | Kidney Margin | 1.8 |
| | | 9010321 | |
| Colon cancer metastasis | 0.8 | Kidney Cancer 8120607 | 6.9 |
| (OD06104) | | | |
| Lung Margin | 4.3 | Kidney Margin | 2.6 |
| (OD06104) | | 8120608 | |
| Colon mets to lung | 4.0 | Normal Uterus | 16.4 |
| (OD04451-01) | | | |
| Lung Margin | 5.8 | Uterine Cancer 064011 | 13.7 |
| (OD04451-02) | | | |
| Normal Prostate | 11.7 | Normal Thyroid | 6.5 |
| Prostate Cancer | 3.2 | Thyroid Cancer | 4.5 |
| (OD04410) | | | |
| Prostate Margin | 7.5 | Thyroid Cancer | 8.8 |
| (OD04410) | | A302152 | |
| Normal Ovary | 100.0 | Thyroid Margin | 4.2 |
| | | A302153 | |
| Ovarian cancer | 1.6 | Normal Breast | 7.1 |
| (OD06283-03) | | | |
| Ovarian Margin | 3.5 | Breast Cancer | 2.2 |
| (OD06283-07) | | | |
| Ovarian Cancer | 2.2 | Breast Cancer | 6.8 |
| Ovarian cancer | 2.9 | Breast Cancer | 16.7 |
| (OD06145) | | (OD04590-01) | |
| Ovarian Margin | 20.9 | Breast Cancer Mets | 5.4 |
| (OD06145) | | (OD04590-03) | |
| Ovarian cancer | 1.9 | Breast Cancer | 8.0 |
| (OD06455-03) | | Metastasis | |
| Ovarian Margin | 9.7 | Breast Cancer | 0.7 |
| (OD06455-07) | | | |
| Normal Lung | 12.1 | Breast Cancer 9100266 | 2.2 |
| Invasive poor diff. lung | 0.8 | Breast Margin 9100265 | 1.3 |
| adeno (ODO4945-01) | | | |
| Lung Margin | 4.8 | Breast Cancer A209073 | 1.7 |
| (ODO4945-03) | | | |
| Lung Malignant Cancer | 2.3 | Breast Margin | 6.3 |
| (OD03126) | | A2090734 | |
| Lung Margin | 4.1 | Breast cancer | 3.1 |
| (OD03126) | | (OD06083) | |
| Lung Cancer | 4.1 | Breast cancer node | 1.2 |
| (OD05014A) | | metastasis (OD06083) | |
| Lung Margin | 2.8 | Normal Liver | 0.7 |
| (OD05014B) | | | |
| Lung cancer (OD06081) | 10.2 | Liver Cancer 1026 | 3.0 |
| Lung Margin | 4.7 | Liver Cancer 1025 | 3.4 |
| (OD06081) | | | |
| Lung Cancer | 0.6 | Liver Cancer 6004-T | 1.6 |
| (OD04237-01) | | | |
| Lung Margin | 12.1 | Liver Tissue 6004-N | 0.6 |
| (OD04237-02) | | | |
| Ocular Mel Met to Liver | 1.5 | Liver Cancer 6005-T | 8.0 |
| (ODO4310) | | | |
| Liver Margin | 2.9 | Liver Tissue 6005-N | 9.2 |
| (ODO4310) | | | |
| Melanoma Metastasis | 0.5 | Liver Cancer | 0.3 |
| Lung Margin | 6.6 | Normal Bladder | 0.4 |
| (OD04321) | | | |
| Normal Kidney | 1.9 | Bladder Cancer | 0.6 |
| Kidney Ca, Nuclear | 6.8 | Bladder Cancer | 1.5 |
| grade 2 (OD04338) | | | |
| Kidney Margin | 0.0 | Normal Stomach | 30.4 |
| (OD04338) | | | |
| Kidney Ca Nuclear | 1.5 | Gastric Cancer 9060397 | 1.9 |
| grade 1/2 (OD04339) | | | |
| Kidney Margin | 1.8 | Stomach Margin | 7.4 |
| (OD04339) | | 9060396 | |
| Kidney Ca, Clear cell | 2.0 | Gastric Cancer 9060395 | 9.1 |
| type (OD04340) | | | |
| Kidney Margin | 1.1 | Stomach Margin | 12.6 |
| (OD04340) | | 9060394 | |
| Kidney Ca, Nuclear | 0.9 | Gastric Cancer 064005 | 3.5 |
| grade 3 (OD04348) | | | |

TABLE 16DD
Panel 4D

| | Rel. Exp. (%) | | Rel. Exp. (%) |
|---|---|---|---|
| | Ag2493, Run | | Ag2493, Run |
| Tissue Name | 164317906 | Tissue Name | 164317906 |
| Secondary Th1 act | 7.1 | HUVEC IL-1 beta | 3.7 |
| Secondary Th2 act | 13.6 | HUVEC IFN gamma | 3.2 |
| Secondary Tr1 act | 12.4 | HUVEC TNF alpha + IFN | 5.7 |
| | | gamma | |
| Secondary Th1 rest | 4.1 | HUVEC TNF alpha + IL4 | 6.5 |
| Secondary Th2 rest | 7.7 | HUVEC IL-11 | 5.5 |
| Secondary Tr1 rest | 4.6 | Lung Microvascular EC | 10.7 |
| | | none | |
| Primary Th1 act | 14.7 | Lung Microvascular EC | 3.9 |
| | | TNF alpha + IL-1 beta | |
| Primary Th2 act | 19.6 | Microvascular Dermal EC | 13.6 |
| | | none | |
| Primary Tr1 act | 25.7 | Microsvasular Dermal EC | 16.3 |
| | | TNF alpha + IL-1 beta | |
| Primary Th1 rest | 22.7 | Bronchial epithelium | 45.7 |
| | | TNF alpha + IL1 beta | |
| Primary Th2 rest | 17.3 | Small airway epithelium | 10.8 |
| | | none | |
| Primary Tr1 rest | 12.5 | Small airway epithelium | 54.7 |
| | | TNF alpha + IL-1 beta | |
| CD45RA CD4 | 7.0 | Coronery artery SMC rest | 63.7 |
| lymphocyte act | | | |
| CD45RO CD4 | 14.4 | Coronery artery SMC | 22.8 |
| lymphocyte act | | TNF alpha + IL-1 beta | |
| CD8 lymphocyte act | 10.7 | Astrocytes rest | 3.1 |
| Secondary CD8 | 16.5 | Astrocytes TNF alpha + | 0.8 |
| lymphocyte rest | | IL-1 beta | |
| Secondary CD8 | 7.4 | KU-812 (Basophil) rest | 16.8 |
| lymphocyte act | | | |
| CD4 lymphocyte none | 10.6 | KU-812 (Basophil) | 46.0 |
| | | PMA/ionomycin | |
| 2ry Th1/Th2/Tr1_anti- | 7.3 | CCD1106 (Keratinocytes) | 28.1 |
| CD95 CH11 | | none | |
| LAK cells rest | 7.3 | CCD1106 (Keratinocytes) | 21.3 |
| | | TNF alpha + IL-1 beta | |
| LAK cells IL-2 | 7.0 | Liver cirrhosis | 9.5 |
| LAK cells IL-2 + IL-12 | 6.9 | Lupus kidney | 5.0 |
| LAK cells IL-2 + IFN | 18.4 | NCI-H292 none | 3.3 |
| gamma | | | |
| LAK cells IL-2 + IL-18 | 8.4 | NCI-H292 IL-4 | 1.9 |
| LAK cells | 20.0 | NCI-H292 IL-9 | 3.2 |
| PMA/ionomycin | | | |
| NK Cells IL-2 rest | 4.8 | NCI-H292 IL-13 | 1.7 |
| Two Way MLR 3 day | 6.6 | NCI-H292 IFN gamma | 0.6 |
| Two Way MLR 5 day | 4.5 | HPAEC none | 3.7 |
| Two Way MLR 7 day | 6.6 | HPAEC TNF alpha + IL-1 | 4.8 |
| | | beta | |
| PBMC rest | 8.7 | Lung fibroblast none | 81.8 |
| PBMC PWM | 27.7 | Lung fibroblast TNF | 20.3 |
| | | alpha + IL-1 beta | |
| PBMC PHA-L | 17.7 | Lung fibroblast IL-4 | 100.0 |
| Ramos (B cell) none | 5.3 | Lung fibroblast IL-9 | 59.5 |
| Ramos (B cell) | 27.0 | Lung fibroblast IL-13 | 49.0 |
| ionomycin | | | |
| B lymphocytes PWM | 46.3 | Lung fibroblast IFN | 95.3 |
| | | gamma | |
| B lymphocytes CD40L | 15.4 | Dermal fibroblast | 15.7 |
| and IL-4 | | CCD1070 rest | |
| EOL-1 dbcAMP | 7.2 | Dermal fibroblast | 23.3 |
| | | CCD1070 TNF alpha | |
| EOL-1 dbcAMP | 2.3 | Dermal fibroblast | 8.8 |
| PMA/ionomycin | | CCD1070 IL-1 beta | |
| Dendritic cells none | 4.1 | Dermal fibroblast IFN | 24.5 |
| | | gamma | |
| Dendritic cells LPS | 0.0 | Dermal fibroblast IL-4 | 0.0 |
| Dendritic cells anti- | 3.3 | IBD Colitis 2 | 0.7 |
| CD40 | | | |
| Monocytes rest | 3.6 | IBD Crohn's | 2.6 |
| Monocytes LPS | 0.8 | Colon | 61.6 |
| Macrophages rest | 4.9 | Lung | 52.5 |
| Macrophages LPS | 2.2 | Thymus | 25.9 |
| HUVEC none | 7.3 | Kidney | 28.9 |
| HUVEC starved | 15.7 | | |

Panel 1.3D Summary: Ag2493 Expression of this gene is restricted to the prostate (CT=34.4). Thus, expression of this gene could be used to differentiate prostate tissue from other tissues.

Panel 2.2 Summary: Ag2493 The expression of this gene appears to be restricted to a sample derived from normal ovary and a sample of normal ovary adjacent to an ovarian cancer. Of note is the observed lack of expression in ovarian cancer tissues. Thus, the expression of this gene could be used to distinguish normal ovarian tissues from other tissues in the panel and specifically ovarian cancer tissue. Therefore, expression of this gene could be used in the diagnosis or prognosis of ovarian cancer. Moreover, therapeutic modulation of this gene, through the use of small molecule drugs, antibodies or protein therapeutics could benefit in the treatment of ovarian cancer.

Panel 4D Summary: Ag2493 This gene encodes a TGF-beta binding protein 4 homolog. TGF-beta binding protein 4 is a secreted protein that regulates the activity of members of the TGF-beta family of growth factors. This gene is expressed at a moderate level in TNF-alpha+IL-1-beta-activated bronchial epithelium, TNF-alpha+IL-1-beta-activated small airway epithelium, resting lung fibroblasts, and IL-4 or IL-9 or IL-13 or IFN-gamma-activated lung fibroblasts. Thus, this gene product may be a useful therapeutic protein to reduce or eliminate the symptoms of chronic obstructive pulmonary disease. Furthermore, the protein encoded by this gene may also be useful as a therapeutic to reduce or eliminate the symptoms of other diseases whose pathophysiology is controlled in part by TGF-beta family members, such as osteoarthritis and rheumatoid arthritis.

References:

Iemura S, Yamamoto T S, Takagi C, Kobayashi H, Ueno N. J Biol Chem Sep. 17, 1999; 274(38):26843-9 Isolation and characterization of bone morphogenetic protein-binding proteins from the early Xenopus embryo.

Using a surface plasmon resonance biosensor as a sensitive and specific monitor, we have isolated two distinct bone morphogenetic protein (BMP)-binding proteins, and identified them as lipovitellin 1 and Ep45, respectively. Lipovitellin 1 is an egg yolk protein that is processed from vitellogenin. Both vitellogenin and Ep45 are synthesized under estrogen control in the liver, secreted, and taken up by developing oocytes. In this paper, we have shown that of the TGF-beta family members tested, Ep45 can bind only to BMP-4, whereas lipovitellin 1 can bind to both BMP-4 and activin A. Because of this difference in specificity, we have focused on and further studied Ep45. Kinetic parameters were determined by surface plasmon resonance studies and showed that Ep45 associated rapidly with BMP-4 (k(a)=1.06×10(4) M(−1)s(−1)) and dissociated slowly (k(d)=1.6×10(−4) s(−1)). In Xenopus embryos microinjected with Ep45 mRNA, Ep45 blocked the ability of follistatin to inhibit BMP activity and to induce a secondary body axis in a dose-dependent manner, whereas it had no effect on other BMP antagonists, chordin and noggin. These results support the possibility that Ep45 interacts with BMP to modulate its activities in vivo.

Dale L, Wardle F C. Semin Cell Dev Biol June 1999; 10(3):319-26 A gradient of BMP activity specifies dorsal-ventral fates in early Xenopus embryos.

BMP-4 is an extracellular signalling molecule belonging to the TGF-beta superfamily that plays a central role in dorsoventral patterning in vertebrate gastrulae. We review the evidence indicating that BMP-4 acts as a morphogen, specifying dorsoventral positional values in a concentration-dependent manner. An activity gradient of BMP-4 is established not by simple diffusion from a localised source, but by diffusion of inhibitory binding proteins that act on a uniform level of BMP-4 protein. These in turn are regulated by the activity of tolloid-related metalloproteases, such as Xenopus xolloid and zebrafish tolloid. Khalil N, Parekh T V, O'Connor R, Antman N, Kepron W, Yehaulaeshet T, Xu Y D, Gold L I. Thorax December 2001;56(12):907-15 Regulation of the effects of TGF-beta1 by activation of latent TGF-beta1 and differential expression of TGF-beta receptors (TbetaR-I and TbetaR-II) in idiopathic pulmonary fibrosis.

BACKGROUND: Idiopathic pulmonary fibrosis (IPF) is characterised by subpleural fibrosis that progresses to involve all areas of the lung. The expression of transforming growth factor-beta1 (TGF-beta1), a potent regulator of connective tissue synthesis, is increased in lung sections of patients with IPF. TGF-beta1 is generally released in a biologically latent form (L-TGF-beta1). Before being biologically active, TGF-beta must be converted to its active form and interact with both TGF-beta receptors type I and II (TbetaR-I and TbetaR-II). TGF-beta latency binding protein 1 (LTBP-1), which facilitates the release and activation of L-TGF-beta1, is also important in the biology of TGF-beta1. METHODS: Open lung biopsy samples from patients with IPF and normal controls were examined to localise TbetaR-I, TbetaR-II, and LTBP-1. Alveolar macrophages (AM) and bronchoalveolar lavage (BAL) fluid were examined using the CCL-64 bioassay to determine if TGF-beta is present in its active form in the lungs of patients with IPF. RESULTS: Immunoreactive L-TGF-beta1 was present in all lung cells of patients with IPF except for fibroblasts in the subepithelial regions of honeycomb cysts. LTBP-1 was detected primarily in AM and epithelial cells lining honeycomb cysts in areas of advanced IPF. In normal lungs LTBP-1 immunoreactivity was observed in a few AM. AM from the upper and lower lobes of patients with IPF secreted 1.6 (0.6) fmol and 4.1 (1.9) finol active TGF-beta, respectively, while AM from the lower lobes of control patients secreted no active TGF-beta (p

Roth-Eichhorn S, Heitmann B, Flemming P, Kubicka S, Trautwein C. Scand J Gastroenterol November 2001;36(11): 1204-10 Evidence for the decreased expression of the latent TGF-beta binding protein and its splice form in human liver tumours.

BACKGROUND: Recently, a splice form of the latent TGF-beta binding protein (LTBP-1) was identified in the liver lacking potential important sequences for matrix association and proteinase cleavage (LTBP-1D, -1delta53). For a better understanding of the unknown (patho)physiological role, the expression levels of LTBP-1D and LTBP-1 (full length) were investigated in normal and malignant human liver on the mRNA and protein level. METHODS: Normal liver (5 specimens), hepatocellular carcinoma (4 specimens) and fibrolamellar carcinoma (2 specimens) were examined by quantitative reverse transcription-polymerase chain reaction and immunohistochemistry, for which specific antibodies were generated. RESULTS: The mRNA levels of LTBP-1/-1D in malignant liver tissues are decreased in comparison to normal liver—more so in HCC than in FLC. This finding was confirmed by a strong decrease of immunostaining of LTBP-1/-1D in neoplastic parenchymal cells of HCC and FLC. However, the intensity of LTBP-1 (full length) protein staining was increased in the extracellular matrix of the carcinomas, while LTBP-1 D was not detectable in the matrix. CONCLUSION: Since TGF-beta is known to be over-expressed in liver tumours, the results suggest its enhanced synthesis without binding to LTBP-1. This probably influences the availability of bioactive TGF-beta in the tumour tissue. The missing matrix localization of LTBP-1D indicates that the hinge region containing a heparin-binding site is essential for the binding of LTBP-1 in the extracellular matrix. LTBP-1D may fulfil specific functions for the latency of matrix-unbound TGF-beta.

Barcellos-Hoff M H. J Mammary Gland Biol Neoplasia 1996;1(4):353-63 Latency and activation in the control of TGF-beta.

The biological activity of the transforming growth factor-beta's (TGF-beta)3 is tightly controlled by their persistence in the extracellular compartment as latent complexes. Each of the three mammalian isoform genes encodes a product that is cleaved intracellularly to form two polypeptides, each of which dimerizes. Mature TGF-beta, a 24 kD homodimer, is noncovalently associated with the 80 kD latency-associated peptide (LAP). LAP is a fundamental component of TGF-beta that is required for its efficient secretion, prevents it from binding to ubiquitous cell surface receptors, and maintains its availability in a large extracellular reservoir that is readily accessed by activation. This latent TGF-beta complex (LTGF-beta) is secreted by all cells and is abundant both in circulating forms and bound to the extracellular matrix. Activation describes the collective events leading to the release of TGF-beta. Despite the importance of TGF-beta regulation of growth and differentiation in physiological and malignant tissue processes, remarkably little is known about the mechanisms of activation in situ. Recent studies of irradiated mammary gland reveal certain features of TGF-beta 1 activation that may shed light on its regulation and potential roles in the normal and neoplastic mammary gland.

Barry F, Boynton R E, Liu B, Murphy J M. Exp Cell Res Aug. 15, 2000;268(2):189-200 Chondrogenic differentiation of mesenchymal stem cells from bone marrow: differentiation-dependent gene expression of matrix components.

Transforming growth factor (TGF)-beta-induced chondrogenesis of mesenchymal stem cells derived from bone marrow involves the rapid deposition of a cartilage-specific extracellular matrix. The sequential events in this pathway leading from the undifferentiated stem cell to a mature chondrocyte were investigated by analysis of key matrix elements. Differentiation was rapidly induced in cells cultured in the presence of TGF-beta 3 or -beta 2 and was accompanied by the early expression of fibromodulin and cartilage oligomeric matrix protein. An increase in aggrecan and versican core protein synthesis defined an intermediate stage, which also involved the small leucine-rich proteoglycans decorin and biglycan. This was followed by the appearance of type II collagen and chondroadherin. The pathway was also characterized by the appearance of type X collagen, usually associated with hypertrophic cartilage. There was also a change in the pattern of sulfation of chondroitin sulfate, with a progressive increase in the proportion of 6-sulfated species. The major proportion of newly synthesized glycosaminoglycan was part of an aggregating proteoglycan network. These data allow us to define the phenotype of the differentiated cell and to understand in greater detail the sequential process of matrix assembly.

Lawrence D A. Mol Cell Biochem March 2001;219(1-2):163-70 Latent-TGF-beta: an overview.

The latency associated with the transforming growth factor-betas (TGF-betas) was discovered in 1984. Since the two publications on this subject in that year, there has been on average over sixty reports in which latency was the dominant theme for each of the past 10 years, proof enough of the interest in this field of growth factor research. As the mature 25 kD forms of the TGF-betas are required for them to exert their many, diverse biological effects, it was inevitable that an explanation of the structure and of the activation of the latent complexes be sought. This overview provides a description of these essential points. Now that it has been clearly shown that dysregulation of particular components of the TGF-beta signalling pathway is implicated in many human diseases, the activation of the latent TGF-beta complexes has taken on added importance. Technical improvements enable the distinction of active and latent TGF-beta proteins in vivo and have started to reveal anomalies in the control of activation in relation to various pathological situations.

Fagenholz P J, Warren S M, Greenwald J A, Bouletreau P J, Spector J A, Crisera F E, Longaker M T. J Craniofac Surg March 2001;12(2):183-90 Osteoblast gene expression is differentially regulated by TGF-beta isoforms.

The transforming growth factor beta (TGF-beta) superfamily encompasses a number of important growth factors including several TGF-beta isoforms, the bone morphogenetic proteins, activins, inhibins, and growth and differentiation factors. TGF-beta 1, -beta 2, and -beta 3 are three closely related isoforms that are widely expressed during skeletal morphogenesis and bone repair. Numerous studies suggest that each isoform has unique in vivo functions; however, the effects of these TGF-beta isoforms on osteoblast gene expression and maturation have never been directly compared. In the current study, we treated undifferentiated neonatal rat calvaria osteoblast-enriched cell cultures with 2.5 ng/ml of each TGF-beta isoform and analyzed gene expression at 0, 3, 6, and 24 hours. We demonstrated unique isoform-specific regulation of endogenous TGF-beta 1 and type I collagen mRNA transcription. To assess the effects of extended TGF-beta treatment on osteoblast maturation, we differentiated osteoblast cultures in the presence of 2.5 ng/ml of each TGF-beta isoform. Analysis of collagen I, alkaline phosphatase, and osteocalcin demonstrated that each TGF-beta isoform uniquely suppressed the transcription of these osteoblast differentiation markers. Interestingly, TGF-beta isoform treatment increased osteopontin expression in primary osteoblasts after 4 and 10 days of differentiation. To our knowledge, these data provide the first direct comparison of the effects of the TGF-beta isoforms on osteoblast gene expression in vitro. Furthermore, these data suggest that TGF-beta isoforms may exert their unique in vivo effects by differentially regulating osteoblast cytokine secretion, extracellular matrix production, and the rate of cellular maturation.

E. NOV7 (GMAP000808_A_da1)

Expression of gene GMAP000808_A_da1 was assessed using the primer-probe set Ag2496, described in Table 17EA. Results of the RTQ-PCR runs are shown in Tables 17EB, and 17EC.

TABLE 17EA

Probe Name Ag2496

| | | | |
|---|---|---|---|
| Primers | Sequences | Length | Start Position |

| | | | |
|---|---|---|---|
| Forward | 5′-gcaacagcatggtgatctg-3′ (SEQ ID NO: 137) | 19 | 133 |
| Probe | TET-5′-ctttcgaatgcacaggaaccccttct-3′-TAMRA (SEQ ID NO: 138) | 26 | 161 |
| Reverse | 5′-cgccaggttgaggatatagat-3′ (SEQ ID NO: 139) | 21 | 189 |

TABLE 17EB
Panel 1.3D

| | Rel. Exp. (%) Ag2496, | | Rel. Exp. (%) Ag2496, |
|---|---|---|---|
| Tissue Name | Run 165639540 | Tissue Name | Run 165639540 |
| Liver adenocarcinoma | 0.0 | Kidney (fetal) | 11.3 |
| Pancreas | 0.0 | Renal ca. 786-0 | 0.0 |
| Pancreatic ca. CAPAN 2 | 0.0 | Renal ca. A498 | 0.0 |
| Adrenal gland | 0.0 | Renal ca. RXF 393 | 0.0 |
| Thyroid | 22.7 | Renal ca. ACHN | 0.0 |
| Salivary gland | 0.0 | Renal ca. UO-31 | 0.0 |
| Pituitary gland | 10.5 | Renal ca. TK-10 | 0.0 |
| Brain (fetal) | 20.3 | Liver | 0.0 |
| Brain (whole) | 17.9 | Liver (fetal) | 0.0 |
| Brain (amygdala) | 8.6 | Liver ca. | 0.0 |
| | | (hepatoblast) HepG2 | |
| Brain (cerebellum) | 100.0 | Lung | 0.0 |
| Brain (hippocampus) | 22.4 | Lung (fetal) | 0.0 |
| Brain (substantia nigra) | 0.0 | Lung ca. (small cell) | 19.1 |
| | | LX-1 | |
| Brain (thalamus) | 0.0 | Lung ca. (small cell) | 0.0 |
| | | NCI-H69 | |
| Cerebral Cortex | 0.0 | Lung ca. (s.cell var.) | 10.2 |
| | | SHP-77 | |
| Spinal cord | 0.0 | Lung ca. (large | 45.7 |
| | | cell)NCI-H460 | |
| glio/astro U87-MG | 0.0 | Lung ca. (non-sm. | 13.3 |
| | | cell) A549 | |
| glio/astro U-118-MG | 0.0 | Lung ca. (non-s. cell) | 0.0 |
| | | NCI-1123 | |
| astrocytoma SW1783 | 0.0 | Lung ca. (non-s.cell) | 0.0 |
| | | HOP-62 | |
| neuro*; met SK-N-AS | 11.3 | Lung ca. (non-s.cl) | 0.0 |
| | | NCI-H522 | |
| astrocytoma SF-539 | 10.7 | Lung ca. (squam.) | 0.0 |
| | | SW 900 | |
| astrocytoma SNB-75 | 0.0 | Lung ca. (squam.) | 0.0 |
| | | NCI-H596 | |
| glioma SNB-19 | 0.0 | Mammary gland | 0.0 |
| glioma U251 | 0.0 | Breast ca.* (pl.ef) | 0.0 |
| | | MCF-7 | |
| glioma SF-295 | 0.0 | Breast ca.* (pl.ef) | 0.0 |
| | | MDA-MB-231 | |
| Heart (Fetal) | 0.0 | Breast ca.* (pl. ef) | 0.0 |
| | | T47D | |
| Heart | 10.1 | Breast ca. BT-549 | 0.0 |
| Skeletal muscle (Fetal) | 0.0 | Breast ca. MDA-N | 0.0 |
| Skeletal muscle | 0.0 | Ovary | 0.0 |
| Bone marrow | 0.0 | Ovarian ca. OVCAR-3 | 12.1 |
| Thymus | 13.3 | Ovarian ca. OVCAR-4 | 0.0 |
| Spleen | 0.0 | Ovarian ca. OVCAR-5 | 10.0 |
| Lymph node | 9.2 | Ovarian ca. OVCAR-8 | 0.0 |
| Colorectal | 18.8 | Ovarian ca. IGROV-1 | 0.0 |
| Stomach | 0.0 | Ovarian ca. (ascites) SK-OV-3 | 0.0 |
| Small intestine | 20.3 | Uterus | 51.8 |
| Colon ca. SW480 | 0.0 | Placenta | 0.0 |
| Colon ca.* SW620 | 11.0 | Prostate | 29.9 |
| (SW480 met) | | | |
| Colon ca. HT29 | 0.0 | Prostate ca.* (bone | 0.0 |
| | | met) PC-3 | |
| Colon ca. HCT-116 | 0.0 | Testis | 48.3 |
| Colon ca. CaCo-2 | 0.0 | Melanoma | 0.0 |
| | | Hs688(A).T | |
| CC Well to Mod Diff | 0.0 | Melanoma* (met) | 0.0 |
| (ODO3866) | | Hs688(B).T | |
| Colon ca. HCC-2998 | 0.0 | Melanoma UACC-62 | 0.0 |
| Gastric ca. (liver met) | 9.9 | Melanoma M14 | 0.0 |
| NCI-N87 | | | |
| Bladder | 11.3 | Melanoma LOX | 0.0 |
| | | IMVI | |
| Trachea | 0.0 | Melanoma* (met) | 0.0 |
| | | SK-MEL-5 | |
| Kidney | 0.0 | Adipose | 13.3 |

TABLE 17EC
Panel 4D

| | Rel. Exp.(%) | | Rel. Exp.(%) |
|---|---|---|---|
| | Ag2496, Run | | Ag2496, Run |
| Tissue Name | 158563772 | Tissue Name | 158563772 |
| Secondary Th1 act | 4.9 | HUVEC IL-1 beta | 0.0 |
| Secondary Th2 act | 7.1 | HUVEC IFN gamma | 2.8 |
| Secondary Tr1 act | 0.0 | HUVEC TNF alpha + IFN | 0.0 |
| | | gamma | |
| Secondary Th1 rest | 4.6 | HUVEC TNF alpha + IL4 | 0.0 |
| Secondary Th2 rest | 11.5 | HUVEC IL-11 | 0.0 |
| Secondary Tr1 rest | 15.2 | Lung Microvascular EC | 0.0 |
| | | none | |
| Primary Th1 act | 0.0 | Lung Microvascular EC | 0.0 |
| | | TNF alpha + IL-1 beta | |
| Primary Th2 act | 0.0 | Microvascular Dermal EC | 0.0 |
| | | none | |
| Primary Tr1 act | 0.0 | Microsvascular Dermal EC | 0.0 |
| | | TNF alpha + IL-1 beta | |
| Primary Th1 rest | 100.0 | Brochial epithelium | 0.0 |
| | | TNF alpha + IL1 beta | |
| Primary Th2 rest | 18.9 | Small airway epithelium | 0.0 |
| | | none | |
| Primary Tr1 rest | 0.0 | Small airway epithelium | 0.0 |
| | | TNF alpha + IL-1 beta | |
| CD45RA CD4 | 3.0 | Coronery artery SMC rest | 0.0 |
| lymphocyte act | | | |
| CD45RO CD4 | 7.7 | Coronery artery SMC | 0.0 |
| lymphocyte act | | TNF alpha + IL-1 beta | |
| CD8 lymphocyte act | 0.0 | Astrocytes rest | 0.0 |
| Secondary CD8 | 10.1 | Astrocytes TNF alpha + | 5.8 |
| lymphocyte rest | | IL-1 beta | |
| Secondary CD8 | 0.0 | KU-812 (Basophil) rest | 0.0 |
| lymphocyte act | | | |
| CD4 lymphocyte none | 28.3 | KU-812 (Basophil) | 1.8 |
| | | PMA/ionomycin | |
| 2ry Th1/Th2/Tr1_anti- | 2.5 | CCD1106 (Keratinocytes) | 0.0 |
| CD95 CH11 | | none | |
| LAK cells rest | 24.0 | CD1106 (Keratinocytes) | 0.0 |
| | | TNF alpha + IL-1 beta | |
| LAK cells IL-2 | 3.1 | Liver cirrhosis | 19.1 |
| LAK cells IL-2 + IL-12 | 7.0 | Lupus kidney | 0.0 |
| LAK cells IL-2 + IFN | 19.1 | NCI-H292 none | 2.7 |
| gamma | | | |
| LAK cells IL-2 + IL-18 | 12.8 | NCI-H292 IL-4 | 2.5 |
| LAK cells cells | 0.0 | NCI-H292 IL-9 | 2.6 |
| PMA/ionomycin | | | |
| NK Cells IL-2 rest | 0.0 | NCI-H292 IL-13 | 3.9 |
| Two Way MLR 3 day | 7.7 | NCI-H292 IFN gamma | 0.0 |
| Two Way MLR 5 day | 0.0 | HPAEC none | 0.0 |
| Two Way MLR 7 day | 0.0 | HPAEC TNF alpha + IL-1 | 0.0 |
| | | beta | |
| PBMC rest | 6.3 | Lung fibroblast none | 0.0 |
| PBMC PWM | 2.6 | Lung fibroblast TNF | 2.1 |
| | | alpha + IL-1 beta | |
| PBMC PHA-L | 5.8 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 2.8 | Lung fibroblast IFN | 0.0 |
| | | gamma | |
| B lymphocytes CD40L | 7.6 | Dermal fibroblast | 0.0 |
| and IL-4 | | CCD1070 rest | |
| EOL-1 dbcAMP | 0.0 | Dermal fibroblast | 9.7 |
| | | CCD1070 TNF alpha | |
| EOL-1 dbcAMP | 2.5 | Dermal fibroblast | 0.0 |
| PMA/ionomycin | | CCD1070 IL-1 beta | |
| Dendritic cells none | 0.0 | Dermal fibroblast IFN | 1.9 |
| | | gamma | |
| Dendritic cells LPS | 0.0 | Dermal fibroblast IL-4 | 0.0 |
| Dendritic cells anti- | 0.0 | IBD Colitis 2 | 0.0 |
| CD40 | | | |
| Monocytes rest | 4.6 | IBD Crohn's | 6.4 |
| Monocytes LPS | 0.0 | Colon | 13.2 |
| Macrophages rest | 0.0 | Lung | 18.6 |
| Macrophages LPS | 0.0 | Thymus | 9.2 |
| HUVEC none | 0.0 | Kidney | 24.0 |
| HUVEC starved | 0.0 | | |

CNS_neurodegeneration_v1.0 Summary: Ag2496 Expression is low/undetected in all samples in this panel (CT>35). (Data not shown.)

Panel 1.3D Summary: Ag2496 This gene appears to be specific to the cerebellum, and thus expression of this gene could be used to distinguish cerebellar tissue from other CNS tissue. Furthermore therapeutic modulation of the expression or function of this gene product may be of use in treating diseases which show a primary pathology in this region (spinocerebellar ataxia).

Panel 4D Summary: This transcript is most highly expressed (CT=31.5) in resting effector Th1 T cells and not in the corresponding activated cells. Thus, this gene may be a useful marker for Th1 cells. This gene is also expressed at a lower level in resting CD4 T cells and LAK cells. Therefore, small molecule antagonists that block the function of this encoded protein may be useful for treatment of Th1-mediated diseases such inflammatory bowel disease, rheumatoid arthritis, and other autoimmune diseases, such as delayed type hypersensitivity reactions. This transcript is also expressed at significant levels in kidney and thus could potentially serve as a marker for kidney tissue

F. NOV8 (AL163195_da2)

Expression of gene AL163195_da2_ was assessed using the primer-probe set Ag2477, described in Table 18FA. Results of the RTQ-PCR runs are shown in Tables 18FB and 18FC.

TABLE 18FA

Probe Name Ag2477

| | | | |
|---|---|---|---|
| Primers | Sequences | Length | Start Position |

| | | | |
|---|---|---|---|
| Forward | 5′-ctgcaaccacatgatcatacaa-3′ (SEQ ID NO: 140) | 22 | 158 |
| Probe | TET-5′-atcagggaacctgaccacacttgtaa-3′-TAMRA (SEQ ID NO: 141) | 26 | 186 |
| Reverse | 5′-atggatgaagacatgctcttt-3′ (SEQ ID NO: 142) | 22 | 212 |

TABLE 18FB
Panel 1.3D

| | Rel. Exp.(%) Ag2477, | | Rel. Exp.(%) Ag2477, |
|---|---|---|---|
| Tissue Name | Run 165639391 | Tissue Name | Run 165639391 |
| Liver adenocarcinoma | 0.0 | Kidney (fetal) | 0.0 |
| Pancreas | 0.0 | Renal ca. 786-0 | 0.0 |
| Pancreatic ca. CAPAN 2 | 0.0 | Renal ca. A498 | 0.0 |
| Adrenal gland | 0.0 | Renal ca. RXF 393 | 0.0 |
| Thyroid | 0.0 | Renal ca. ACHN | 0.0 |
| Salivary gland | 0.0 | Renal ca. UO-31 | 0.0 |
| Pituitary gland | 0.0 | Renal ca. TK-10 | 0.0 |
| Brain (fetal) | 0.0 | Liver | 0.0 |
| Brain (whole) | 0.0 | Liver (fetal) | 0.0 |
| Brain (amygdala) | 0.0 | Liver ca. | 0.0 |
| | | (hepatoblast) HepG2 | |
| Brain (cerebellum) | 0.0 | Lung | 0.0 |
| Brain (hippocampus) | 0.0 | Lung (fetal) | 0.0 |
| Brain (substantia nigra) | 0.0 | Lung ca. (small cell) | 0.0 |
| | | LX-1 | |
| Brain (thalamus) | 0.0 | Lung ca. (small cell) | 0.0 |
| | | NCI-H69 | |
| Cerebral Cortex | 0.0 | Lung ca. (s.cell var.) | 18.8 |
| | | SHP-77 | |
| Spinal cord | 0.0 | Lung ca. (large | 0.0 |
| | | cell)NCI-H460 | |
| glio/astro U87-MG | 0.0 | Lung ca. (non-sm. | 0.0 |
| | | cell) A549 | |
| glio/astro U-118-MG | 0.0 | Lung ca. (non-s. cell) | 0.0 |
| | | NCI-1123 | |
| astrocytoma SW1783 | 0.0 | Lung ca. (non-s.cell) | 0.0 |
| | | HOP-62 | |
| neuro*; met SK-N-AS | 0.0 | Lung ca. (non-s.cl) | 0.0 |
| | | NCI-H522 | |
| astrocytoma SF-539 | 0.0 | Lung ca. (squam.) | 0.0 |
| | | SW 900 | |
| astrocytoma SNB-75 | 0.0 | Lung ca. (squam.) | 0.0 |
| | | NCI-H596 | |
| glioma SNB-19 | 0.0 | Mammary gland | 0.0 |
| glioma U251 | 7.5 | Breast ca.* (pl.ef) | 0.0 |
| | | MCF-7 | |
| glioma SF-295 | 13.7 | Breast ca.* (pl.ef) | 0.0 |
| | | MDA-MB-231 | |
| Heart (Fetal) | 0.0 | Breast ca.* (pl. ef) | 0.0 |
| | | T47D | |
| Heart | 0.0 | Breast ca. BT-549 | 0.0 |
| Skeletal muscle (Fetal) | 0.0 | Breast ca. MDA-N | 0.0 |
| Skeletal muscle | 0.0 | Ovary | 0.0 |
| Bone marrow | 0.0 | Ovarian ca. OVCAR-3 | 0.0 |
| Thymus | 0.0 | Ovarian ca. OVCAR-4 | 0.0 |
| Spleen | 0.0 | Ovarian ca. OVCAR-5 | 0.0 |
| Lymph node | 0.0 | Ovarian ca. OVCAR-8 | 0.0 |
| Colorectal | 0.0 | Ovarian ca. IGROV-1 | 0.0 |
| Stomach | 0.0 | Ovarian ca. (ascites) SK-OV-3 | 0.0 |
| Small intestine | 0.0 | Uterus | 0.0 |
| Colon ca. SW480 | 0.0 | Placenta | 0.0 |
| Colon ca.* SW620 | 0.0 | Prostate | 0.0 |
| (SW480 met) | | | |
| Colon ca. HT29 | 0.0 | Prostate ca.* (bone | 0.0 |
| | | met) PC-3 | |
| Colon ca. HCT-116 | 0.0 | Testis | 100.0 |
| Colon ca. CaCo-2 | 0.0 | Melanoma | 0.0 |
| | | Hs688(A).T | |
| CC Well to Mod Diff | 0.0 | Melanoma* (met) | 0.0 |
| (ODO3866) | | Hs688(B).T | |
| Colon ca. HCC-2998 | 0.0 | Melanoma UACC-62 | 0.0 |
| Gastric ca. (liver met) | 0.0 | Melanoma M14 | 0.0 |
| NCI-N87 | | | |
| Bladder | 0.0 | Melanoma LOX | 0.0 |
| | | IMVI | |
| Trachea | 9.1 | Melanoma* (met) | 0.0 |
| | | SK-MEL-5 | |
| Kidney | 0.0 | Adipose | 0.0 |

TABLE 18FC
Panel 4D

| | Rel. Exp. (%) | | Rel. Exp. (%) |
|---|---|---|---|
| | Ag2477, Run | | Ag2477, Run |
| Tissue Name | 164391869 | Tissue Name | 164391869 |
| Secondary Th1 act | 0.0 | HUVEC IL-1 beta | 0.0 |
| secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 35.8 | Lung Microvascular EC | 0.0 |
| | | TNF alpha + IL-1 beta | |
| Primary Th2 act | 0.0 | Microvascular Dermal EC | 0.0 |
| | | none | |
| Primary Tr1 act | 0.0 | Microvascular Dermal EC | 0.0 |
| | | TNF alpha + IL-1 beta | |
| Primary Th1 rest | 0.0 | Bronchial epithelium | 0.0 |
| | | TNF alpha + IL-1 beta | |
| Primary Th2 rest | 0.0 | Small airway epithelium | 0.0 |
| | | none | |
| Primary Tr1 rest | 0.0 | Small airway epithelium | 0.0 |
| | | TNF alpha + IL-1 beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNF alpha + IL-1 beta | |
| CD8 lymphocyte act | 0.0 | Astrocytes rest | 0.0 |
| Secondary CD8 | 0.0 | Astrocytes TNF alpha + | 0.0 |
| lymphocyte rest | | 1 beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 0.0 | CCD1106 (Keratinocytes) | 0.0 |
| | | TNF alpha + IL-1 beta | |
| LAK cells IL-2 | 17.0 | Liver cirrhosis | 100.0 |
| LAK cells IL-2 + IL-12 | 0.0 | Lupus Kidney | 0.0 |
| LAK cells IL-2 + IFN | 0.0 | NCI-H292 none | 0.0 |
| gamma | | | |
| LAK cells IL-2 + IL-18 | 0.0 | NCI-H292 IL-4 | 0.0 |
| LAK cells | 0.0 | NCI-H292 IL-9 | 0.0 |
| PMA/ionomycin | | | |
| NK Cells IL-2 rest | 0.0 | NCI-H292 IL-13 | 0.0 |
| Two Way MLR 3 day | 0.0 | NCI-H292 IFN gamma | 0.0 |
| Two Way MLR 5 day | 0.0 | HPAEC none | 0.0 |
| Two Way MLR 7 day | 0.0 | HPAEC TNF alpha + IL-1 | 0.0 |
| | | beta | |
| PBMC rest | 0.0 | Lung fibroblast none | 0.0 |
| PBMC PWM | 0.0 | Lung fibroblast TNF | 0.0 |
| | | alpha + IL-1 beta | |
| PBMC PHA-L | 0.0 | Lung fibroblast IL-4 | 0.0 |
| Ramos (B cell) none | 0.0 | Lung fibroblast IL-9 | 0.0 |
| Ramos (B cell) | 0.0 | Lung fibroblast IL-13 | 0.0 |
| ionomycin | | | |
| B lymphocytes PWM | 0.0 | Lung fibroblast IFN | 0.0 |
| | | gamma | |
| B lymphocytes CD40L | 0.0 | Dermal fibroblast | 0.0 |
| and IL-4 | | CCD1070 rest | |
| EOL-1 dbcAMP | 0.0 | Dermal fibroblast | 0.0 |
| | | CCD1070 TNF alpha | |
| EOL-1 dbcAMP | 0.0 | Dermal fibroblast | 0.0 |
| PMA/ionomycin | | CCD1070 IL-1 beta | |
| Dendritic cells none | 0.0 | Dermal fibroblast IFN | 10.3 |
| | | gamma | |
| Dendritic cells LPS | 0.0 | Dermal fibroblast IL-4 | 0.0 |
| Dendritic cells anti- | 0.0 | IBD Colitis 2 | 22.8 |
| CD40 | | | |
| Monocytes rest | 0.0 | IBD Crohn's | 0.0 |
| Monocytes LPS | 0.0 | Colon | 0.0 |
| Macrophages rest | 0.0 | Lung | 0.0 |
| Macrophages LPS | 0.0 | Thymus | 8.0 |
| HUVEC none | 0.0 | Kidney | 0.0 |
| HUVEC starved | 0.0 | | |

Panel 1.3D Summary: Ag2477 Significant expression of this gene is restricted to the testis (CT=33.1). Thus, expression of this gene could be used to differentiate testis tissue from other tissues. Furthermore, the highly specific expression of this gene suggests that its protein product may be involved in the normal function of the testis. Thus, therapeutic modulation of the expression or function of this gene may be useful in the treatment of infertility and other disorders that involve the testis.

Panel 4D Summary: Ag 2477 This transcript is expressed almost exclusively in liver cirrhosis (CT=33.5) but not in normal liver. This suggests that the protein encoded by this transcript may be involved or associated with the pathology of the liver and may serve as a diagnostic marker for liver cirrhosis or other inflammatory liver diseases.

G. CG58610-01/SC87421058_A: AMINOTRANSFERASE

Expression of gene CG58610-01 was assessed using the primer-probe set Ag2267, described in Table 19GA.

TABLE 19GA

Probe Name Ag2267

| | | | |
|---|---|---|---|
| Primers | Sequences | Length | Start Position |

| | | | |
|---|---|---|---|
| Forward | 5′-caattttggttctggagaaaga-3′ (SEQ ID NO: 143) | 22 | 1218 |
| Probe | TET-5′-tctcagtgccgatggacctcatagaa-3′-TAMRA (SEQ ID NO: 144) | 26 | 1252 |
| Reverse | 5′-cagtgaagcacataggtggttt-3′ (SEQ ID NO: 145) | 22 | 1292 |

CNS_neurodegeneration_v1.0 Summary: Ag2267 Expression is low/undetectable in all samples in this panel (CTs>35). (Data not shown.)

Panel 1.3D Summary: Ag2267 Expression is low/undetectable in all samples in this panel (CTs>35). (Data not shown.)

Panel 2D Summary: Ag2267 Expression is low/undetectable in all samples in this panel (CTs>35). (Data not shown.)

Panel 4D Summary: Ag2267 Expression is low/undetectable in all samples in this panel (CTs>35). (Data not shown.)

H. NOV10a (CG50235-01)

Expression of gene CG50235-01 was assessed using the primer-probe set Ag4737, described in Table20HA. Results of the RTQ-PCR runs are shown in Tables 20HB, 20HC, and 20HD.

TABLE 20HA

Probe Name Ag4737

| | | | |
|---|---|---|---|
| Primers | Sequences | Length | Start Position |

| | | | |
|---|---|---|---|
| Forward | 5′-ctgggagagacatacgactttg-3′ (SEQ ID NO: 146) | 22 | 1229 |
| Probe | TET-5′-cccggaacaccttctcaagaggagt-3′-TAMRA (SEQ ID NO: 147) | 25 | 1269 |
| Reverse | 5′-gggaaggatggtgtctaagaaa-3′ (SEQ ID NO: 148) | 22 | 1294 |

TABLE 20HB
CNS_neurodegeneration_v1.0

| | Rel. ExP. (%) Ag4737, | | Rel. Exp. (%) Ag4737, |
|---|---|---|---|
| Tissue Name | Run 224721331 | Tissue Name | Run 224721331 |
| AD 1 Hippo | 23.3 | Control (path) 3 | 4.5 |
| | | Temporal Ctx | |
| AD 2 Hippo | 36.6 | Control (Path) 4 | 11.0 |
| | | Temporal Ctx | |
| AD 3 Hippo | 31.4 | AD 1 Occipital Ctx | 21.9 |
| AD 4 Hippo | 9.3 | AD 2 Occipital Ctx | 0.0 |
| | | (Missing) | |
| AD 5 Hippo | 38.4 | AD 3 Occipital Ctx | 25.7 |
| AD 6 Hippo | 15.2 | AD 4 Occipital Ctx | 38.7 |
| Control 2 Hippo | 39.8 | AD 5 Occipital Ctx | 63.3 |
| Control 4 Hippo | 30.1 | AD 5 Occipital Ctx | 23.0 |
| Control (Path) 3 | 20.3 | Control 1 Occipital | 4.7 |
| Hippo | | Ctx | |
| AD 1 Temporal Ctx | 38.7 | Control 2 Occipital | 41.5 |
| | | Ctx | |
| AD 2 Temporal Ctx | 74.2 | Control 3 Occipital | 20.4 |
| | | Ctx | |
| AD 3 Temporal Ctx | 8.1 | Control 4 Occipital | 18.2 |
| | | Ctx | |
| AD 4 Temporal Ctx | 32.8 | Control (Path) 1 | 84.1 |
| | | Occipital Ctx | |
| AD 5 Inf Temporal | 100.0 | Control (Path) 2 | 13.3 |
| Ctx | | Occipital Ctx | |
| AD 5 Sup Temporal | 48.6 | Control (Path) 3 | 4.6 |
| Ctx | | Occipital Ctx | |
| AD 6 Inf Temporal | 13.1 | Control (Path) 4 | 5.0 |
| Ctx | | Occipital Ctx | |
| AD 6 Sup Temporal | 22.7 | Control 1 Parietal | 14.1 |
| Ctx | | Ctx | |
| Control 1 Temporal | 5.1 | Control 2 Parietal | 38.7 |
| Ctx | | Ctx | |
| Control 2 Temporal | 62.0 | Control 3 Parietal | 36.9 |
| Ctx | | Ctx | |
| Control 3 Temporal | 39.8 | Control (Path) 1 | 38.7 |
| Ctx | | Parietal Ctx | |
| Control 3 Temporal | 31.4 | Control (Path) 2 | 55.5 |
| Ctx | | Parietal Ctx | |
| Control (Path) 1 | 43.8 | Control (Path) 3 | 0.0 |
| Temporal Ctx | | Parietal Ctx | |
| Control (Path) 2 | 14.1 | Control (path) 4 | 5.1 |
| Temporal Ctx | | Parietal Ctx | |

TABLE 20HC
General_screening_panel_v1.4

| | Rel. Exp. (%) Ag4737, | | Rel. Exp. (%) Ag4737, |
|---|---|---|---|
| Tissue Name | Run 222904895 | Tissue Name | Run 222904895 |
| Adipose | 0.0 | Renal ca. TK-10 | 1.1 |
| Melanoma* | 4.3 | Bladder | 6.8 |
| Hs688(A).T | | | |
| Melanoma* | 1.3 | Gastric ca. (liver met.) | 5.2 |
| Hs688(B).T | | NCI-N87 | |
| Melanoma* M14 | 0.0 | Gastric ca. KATO III | 2.0 |
| Melanoma* | 0.0 | Colon ca. SW-948 | 0.0 |
| LOXIMVI | | | |
| Melanoma* SK- | 1.0 | Colon ca. SW480 | 9.9 |
| MEL-5 | | | |
| Squamous cell | 7.3 | Colon ca.* (SW480 | 0.3 |
| carcinoma SCC-4 | | met) SW620 | |
| Testis Pool | 0.9 | Colon ca. HT29 | 0.0 |
| Prostate ca.* (bone | 0.5 | Colon ca. HCT-116 | 8.6 |
| met) PC-3 | | | |
| Prostate Pool | 5.6 | Colon ca. CaCo-2 | 0.5 |
| Placenta | 2.0 | Colon cancer tissue | 0.5 |
| Uterus Pool | 0.8 | Colon ca. SW1116 | 16.2 |
| Ovarian ca. | 9.1 | Colon ca. Colo-205 | 1.1 |
| OVCAR-3 | | | |
| Ovarian ca. SK-OV-3 | 67.8 | Colon ca. SW-48 | 0.0 |
| Ovarian ca. | 88.3 | Colon Pool | 0.0 |
| OVCAR-4 | | | |
| Ovarian ca. | 0.0 | Small Intestine Pool | 6.9 |
| OVCAR-5 | | | |
| Ovarian ca. IGROV-1 | 6.6 | Stomach Pool | 1.2 |
| Ovarian ca. | 8.7 | Bone Marrow Pool | 0.0 |
| OVCAR-8 | | | |
| Ovary | 3.3 | Fetal Heart | 26.1 |
| Breast ca. MCF-7 | 0.8 | Heart Pool | 21.9 |
| Breast ca. MDA- | 1.1 | Lymph Node Pool | 1.6 |
| MB-231 | | | |
| Breast ca. BT 549 | 0.0 | Fetal Skeletal Muscle | 2.7 |
| Breast ca. T47D | 3.1 | Skeletal Muscle Pool | 17.3 |
| Breast ca. MDA-N | 0.0 | Spleen Pool | 2.4 |
| Breast Pool | 0.3 | Thymus Pool | 1.4 |
| Trachea | 5.5 | CNS cancer (glio/astro) | 0.0 |
| | | U87-MG | |
| Lung | 0.9 | CNS cancer (glio/astro) | 0.0 |
| | | U-118-MG | |
| Fetal Lung | 0.5 | CNS cancer | 0.0 |
| | | (neuro;met) SK-N-AS | |
| Lung ca. NCI-N417 | 0.0 | CNS cancer (astro) SF- | 0.5 |
| | | 539 | |
| Lung ca. LX-1 | 0.5 | CNS cancer (astro) | 1.4 |
| | | SNB-75 | |
| Lung ca. NCI-H146 | 0.9 | CNS cancer (glio) | 5.3 |
| | | SNB-19 | |
| Lung ca. SHP-77 | 44.4 | CNS cancer (glio) SF- | 0.0 |
| | | 295 | |
| Lung ca. A549 | 0.9 | Brain (Amygdala) Pool | 7.8 |
| Lung ca. NCI-H526 | 54.7 | Brain (cerebellum) | 3.6 |
| Lung ca. NCI-H23 | 8.8 | Brain (fetal) | 1.1 |
| Lung ca. NCI-H460 | 0.3 | Brain (Hippocampus) | 7.2 |
| | | Pool | |
| Lung ca. HOP-62 | 0.0 | Cerebral Cortex Pool | 5.6 |
| Lung ca. NCI-H522 | 8.0 | Brain (Substantia nigra) | 19.2 |
| | | Pool | |
| Liver | 0.5 | Brain (Thalamus) Pool | 18.0 |
| Fetal Liver | 2.0 | Brain (whole) | 6.3 |
| Liver ca. HepG2 | 1.0 | Spinal Cord Pool | 42.0 |
| Kidney Pool | 1.4 | Adrenal Gland | 0.0 |
| Fetal Kidney | 1.2 | Pituitary gland Pool | 7.8 |
| Renal ca. 786-0 | 100.0 | Salivary Gland | 7.4 |
| Renal ca. A498 | 17.0 | Thyroid (female) | 1.8 |
| Renal ca. ACHN | 45.1 | Pancreatic ca. CAPAN2 | 0.0 |
| Renal ca. UO-31 | 82.9 | Pancreas Pool | 2.8 |

TABLE 20HD
Panel 4.1D

| | Rel. Exp. (%) | | Rel. Exp. (%) |
|---|---|---|---|
| | Ag4737, Run | | Ag4737, Run |
| Tissue Name | 204154022 | Tissue Name | 204154022 |
| Secondary Th1 act | 0.0 | HUVEC IL-1 beta | 0.0 |
| Secondary Th2 act | 0.0 | HUVEC IFN gamma | 3.3 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC | 0.0 |
| | | none | |
| Primary Th1 act | 0.0 | Lung Microvascular EC | 0.0 |
| | | TNF alpha + IL-1 beta | |
| Primary Th2 act | 2.5 | Microvascular Dermal EC | 0.0 |
| | | none | |
| Primary Tr1 act | 0.0 | Microvascular Dermal EC | 0.0 |
| | | TNF alpha + IL-1 beta | |
| Primary Th1 rest | 0.0 | Bronchial epithelium | 39.8 |
| | | TNF alpha + IL1 beta | |
| Primary Th2 rest | 0.0 | Small airway epithelium | 43.2 |
| | | none | |
| Primary Tr1 rest | 0.0 | Small airway epithelium | 12.3 |
| | | TNF alpha + IL-1 beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNF alpha + IL-1 beta | |
| CD8 lymphocyte act | 0.0 | Astrocytes rest | 73.7 |
| Secondary CD8 | 0.0 | Astrocytes TNF alpha + | 100.0 |
| lymphocyte rest | | IL-1 beta | |
| Secondary CD8 | 0.0 | KU-812 (Basophil) rest | 0.0 |
| lymphocyte act | | | |
| CD4 lymphocyte none | 0.0 | KU-812 (Basophil) | 2.7 |
| | | PMA/ionomycin | |
| 2ry Th1/Th2/Tr1_anti- | 0.0 | CCD1106 (Keratinocytes) | 29.5 |
| CD95 CH11 | | none | |
| LAK cells rest | 0.0 | CCD1106 (Keratinocytes) | 5.6 |
| | | TNF alpha + IL-1 beta | |
| LAK cells IL-2 | 0.0 | Liver cirrhosis | 0.0 |
| LAK cells IL-2 + IL-12 | 0.0 | NCI-H292 none | 12.5 |
| LAK cells IL-2 + IFN | 0.0 | NCI-H292 IL-4 | 0.0 |
| gamma | | | |
| LAK cells IL-2 + IL-18 | 0.0 | NCI-H292 IL-9 | 11.3 |
| LAK cells | 0.0 | NCI-H292 IL-13 | 0.0 |
| PMA/ionomycin | | | |
| NK Cells IL-2 rest | 0.0 | NCI-H292 IFN gamma | 0.0 |
| Two Way MLR 3 day | 0.0 | HPAEC none | 0.0 |
| Two Way MLR 5 day | 0.0 | HPAEC TNF alpha + IL-1 | 2.9 |
| | | beta | |
| Two Way MLR 7 day | 0.0 | Lung fibroblast none | 0.0 |
| PBMC rest | 0.0 | Lung fibroblast TNF alpha + | 0.0 |
| | | IL-1 beta | |
| PBMC PWM | 3.6 | Lung fibroblast IL-4 | 0.0 |
| PBMC PHA-L | 0.0 | Lung fibroblast IL-9 | 4.9 |
| Ramos (B cell) none | 3.0 | Lung fibroblast IL-13 | 0.0 |
| Ramos (B cell) | 2.4 | Lung fibroblast IFN | 3.4 |
| ionomycin | | gamma | |
| B lymphocytes PWM | 0.0 | Dermal fibroblast | 0.0 |
| | | CCD1070 rest | |
| B lymphocytes CD40L | 3.1 | Dermal fibroblast | 0.0 |
| and IL-4 | | CCD1070 TNF alpha | |
| EOL-1 dbcAMP | 0.0 | Dermal fibroblast | 0.0 |
| | | CCD1070 IL-1 beta | |
| EOL-1 dbcAMP | 0.0 | Dermal fibroblast IFN | 0.0 |
| PMA/ionomycin | | gamma | |
| Dendritic cells none | 2.7 | Dermal fibroblast IL-4 | 0.0 |
| Dendritic cells LPS | 0.0 | Dermal Fibroblasts rest | 0.0 |
| Dendritic cells anti- | 0.0 | Neutrophils TNFa + LPS | 0.0 |
| CD40 | | | |
| Monocytes rest | 0.0 | Neutrophils rest | 0.0 |
| Monocytes LPS | 0.0 | Colon | 4.1 |
| Macrophages rest | 0.0 | Lung | 0.0 |
| Macrophages LPS | 0.0 | Thymus | 2.5 |
| HUVEC none | 0.0 | Kidney | 2.7 |
| HUVEC starved | 0.0 | | |

CNS_neurodegeneration_v1.0 Summary: Ag4737 Panel CNS_Neurodegeneration does not show any difference in the expression of this gene between the postmortem brains of controls or Alzheimer's disease patients. This panel does, however, confirm the expression of this gene at low levels in the brains of an independent group of patients. See General_screening_panel_v1.4 for discussion of utility in the central nervous system.

General_screening_panel_v1.4 Summary: Ag4737 The expression of this gene appears to be highest in a sample derived from a renal cancer cell line (CT=29.9). Overall, there appears to be specific expression restricted to cell lines derived from renal cancer, ovarian cancer and lung cancer. Thus, the expression of this gene could be used to distinguish these cell lines from other samples in the panel. Moreover, therapeutic modulation of this gene, through the used of small molecule drugs, antibodies or protein therapeutics could be of benefit in the treatment of renal, ovarian or lung cancer.

This gene is also moderately expressed in several metabolic tissues including adult and fetal heart, pituitary, and skeletal muscle. Thus, this gene product may be important for the pathogenesis, diagnosis and/or treatment of metabolic diseases, including obesity. In addition, this gene appears to be differentially expressed in fetal (CT value=35) versus adult skeletal muscle (CT value=33), and may be useful for the differentiation of the adult vs fetal source of this tissue.

This gene is expressed at low levels in the CNS, except in the spinal cord where expression levels are moderate. Thus, this gene may be of use in treating conditions where the spinal cord is damaged such as spinal cord trauma or spinocerebellar ataxia.

Panel 4.1D Summary: Ag 4737 This transcript is most highly expressed in TNF-a and IL-1 b treated astrocytes (CT=31.9) and is expressed at a lower level in resting astrocytes (CT 32.3). This gene is also expressed at a low level in small airway epithelium and keratinocytes, with expression down regulated in both cell types upon treatment with the inflammatory cytokines TNF-a and IL-1b. This transcript encodes a tolloid like 2 protein, a BMP-1-related proteinase, which has been shown to play a role in extracellular matrix biosynthesis. Therefore, this gene product may be useful as a protein therapeutic to reduce or eliminate the symptoms of inflammatory reactions that occur in multiple sclerosis, chronic obstructive pulmonary disease, asthma, emphysema, and inflammatory skin diseases.

Reference:

Uzel M I, Scott I C, Babakhanlou-Chase H, Palamakumbura A H, Pappano W N, Hong H H, Greenspan D S, Trackman P C. J Biol Chem Jun. 22, 2001;276(25):22537-43 Multiple bone morphogenetic protein 1-related mammalian metalloproteinases process pro-lysyl oxidase at the correct physiological site and control lysyl oxidase activation in mouse embryo fibroblast cultures.

Lysyl oxidase catalyzes the final enzymatic step required for collagen and elastin cross-linking in extracellular matrix biosynthesis. Pro-lysyl oxidase is processed by procollagen C-proteinase activity, which also removes the C-propeptides of procollagens I-III. The Bmp1 gene encodes two procollagen C-proteinases: bone morphogenetic protein 1 (BMP-1) and mammalian Tolloid (mTLD). Mammalian Tolloid-like (mTLL)-1 and -2 are two genetically distinct BMP-1-related proteinases, and mTLL-1 has been shown to have procollagen C-proteinase activity. The present study is the first to directly compare pro-lysyl oxidase processing by these four related proteinases. In vitro assays with purified recombinant enzymes show that all four proteinases productively cleave pro-lysyl oxidase at the correct physiological site but that BMP-1 is 3-, 15-, and 20-fold more efficient than mTLL-1, mTLL-2, and mTLD, respectively. To more directly assess the roles of BMP-1 and mTLL-1 in lysyl oxidase activation by connective tissue cells, fibroblasts cultured from Bmp1-null, Tll1-null, and Bmp1/Tll1 double null mouse embryos, thus lacking BMP-1/mTLD, mTLL-1, or all three enzymes, respectively, were assayed for lysyl oxidase enzyme activity and for accumulation of pro-lysyl oxidase and mature approximately 30-kDa lysyl oxidase. Wild type cells or cells singly null for Bmp1 or Tll1 all produced both pro-lysyl oxidase and processed lysyl oxidase at similar levels, indicating apparently normal levels of processing, consistent with enzyme activity data. In contrast, double null Bmp1/Tll1 cells produced predominantly unprocessed 50-kDa pro-lysyl oxidase and had lysyl oxidase enzyme activity diminished by 70% compared with wild type, Bmp1-null, and Tll1-null cells. Thus, the combination of BMP-1/mTLD and mTLL-1 is shown to be responsible for the majority of processing leading to activation of lysyl oxidase by murine embryonic fibroblasts, whereas in vitro studies identify pro-lysyl oxidase as the first known substrate for mTLL-2.

Panel CNS_{—}1.1 Summary: Ag4737 Expression is low/undetected in all the samples on this panel (CTs>35). (Data not shown.)

I. NOV10b (CG50235-03)

Expression of gene CG50235-03 was assessed using the primer-probe set Ag5112, described in Table 21IA. Results of the RTQ-PCR runs are shown in Table 21IB.

TABLE 21IA

Probe Name Ag5112

| | | | |
|---|---|---|---|
| Primers | Sequences | Length | Start Position |

| | | | |
|---|---|---|---|
| Forward | 5′-tgtgcttttgttagccaga-3′ (SEQ ID NO: 149) | 19 | 1127 |
| Probe | TET-5′-catcaatctgcttgctacacttctcacca-3′-TAMRA (SEQ ID NO: 150) | 29 | 1149 |
| Reverse | 5′-ccaaagccctcggaac-3′ (SEQ ID NO: 151) | 16 | 1182 |

TABLE 21IB
Panel 4.1D

| | Rel. Exp. (%) | | Rel. Exp. (%) |
|---|---|---|---|
| | Ag5112, Run | | Ag5112, Run |
| Tissue Name | 225787031 | Tissue Name | 225787031 |
| Secondary Th1 act | 0.0 | HUVEC IL-1 beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.6 |
| Secondary Tr1 rest | 0.0 | Lung Microvascular EC | 0.0 |
| | | none | |
| Primary Th1 act | 0.0 | Lung Microvascular EC | 0.8 |
| | | TNF alpha + IL-1 beta | |
| Primary Th2 act | 0.0 | Microvascular Dermal EC | 0.0 |
| | | none | |
| Primary Tr1 act | 0.0 | Microsvasular Dermal EC | 0.0 |
| | | TNF alpha + IL-1 beta | |
| Primary Th1 rest | 0.0 | Bronchial epithelium | 1.2 |
| | | TNF alpha + IL1 beta | |
| Primary Th2 rest | 0.0 | Small airway epithelium | 0.0 |
| | | none | |
| Primary Tr1 rest | 0.0 | Small airway epithelium | 0.3 |
| | | TNF alpha + IL-1 beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNF alpha + IL-1 beta | |
| CD8 lymphocyte act | 0.0 | Astrocytes rest | 1.4 |
| Secondary CD8 | 0.0 | Astrocytes TNF alpha + | 0.4 |
| lymphocyte rest | | IL-1 beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 0.8 |
| CD95 CH11 | | none | |
| LAK cells rest | 0.4 | CCD1106 (Keratinocytes) | 0.3 |
| | | TNF alpha + IL-1 beta | |
| LAK cells IL-2 | 0.0 | Liver cirrhosis | 0.0 |
| LAK cells IL-2 + IL-12 | 0.0 | NCI-H292 none | 0.0 |
| LAK cells IL-2 + IFN | 0.0 | NCI-H292 IL-4 | 0.0 |
| gamma | | | |
| LAK cells IL-2 + IL-18 | 0.0 | NCI-H292 IL-9 | 0.0 |
| LAK cells | 0.0 | NCI-H292 IL-13 | 0.0 |
| PMA/ionomycin | | | |
| NK Cells IL-2 rest | 0.0 | NCI-H292 IFN gamma | 0.0 |
| Two Way MLR 3 day | 0.0 | HPAEC none | 0.3 |
| Two Way MLR 5 day | 0.0 | HPAEC TNF alpha + IL-1 | 0.0 |
| | | beta | |
| Two Way MLR 7 day | 0.0 | Lung fibroblast none | 0.0 |
| PBMC rest | 0.0 | Lung fibroblast | 0.0 |
| | | TNF alpha + IL-1 beta | |
| PBMC PWM | 0.0 | Lung fibroblast IL-4 | 0.0 |
| PBMC PHA-L | 0.0 | Lung fibroblast IL-9 | 0.0 |
| Ramos (B cell) none | 0.0 | Lung fibroblast IL-13 | 0.0 |
| Ramos (B cell) | 0.0 | Lung fibroblast IFN | 0.0 |
| ionomycin | | gamma | |
| B lymphocytes PWM | 0.0 | Dermal fibroblast | 0.0 |
| | | CCD1070 rest | |
| B lymphocytes CD40L | 0.0 | Dermal fibroblast | 0.0 |
| and IL-4 | | CCD1070 TNF alpha | |
| EOL-1 dbcAMP | 0.0 | Dermal fibroblast | 0.0 |
| | | CCD1070 IL-1 beta | |
| EOL-1 dbcAMP | 0.0 | Dermal fibroblast IFN | 0.0 |
| PMA/ionomycin | | gamma | |
| Dendritic cells none | 0.0 | Dermal fibroblast IL-4 | 0.0 |
| Dendritic cells LPS | 0.0 | Dermal Fibroblasts rest | 0.0 |
| Dendritic cells anti- | 0.0 | Neutrophils TNFa + LPS | 0.0 |
| CD40 | | | |
| Monocytes rest | 0.0 | Neutrophils rest | 0.6 |
| Monocytes LPS | 0.6 | Colon | 0.7 |
| Macrophages rest | 0.0 | Lung | 1.9 |
| Macrophages LPS | 0.0 | Thymus | 16.8 |
| HUVEC none | 0.0 | Kidney | 100.0 |
| HUVEC starved | 0.0 | | |

CNS_neurodegeneration_v1.0 Summary: Ag5112 Expression is low/undetectable in all the samples in this panel (CTs>35). (Data not shown.)

General_screening_panel_v1.5 Summary: Ag5112 Expression is low/undetectable in all the samples in this panel (CTs>35). (Data not shown.)

Panel 4.1D Summary: Ag 5112: This transcript is expressed almost exclusively in kidney (CT 31.5) and the thymus (CT 34). The transcript encoded by this transcript could be used for detection of kidney and kidney tissues. The putative protein encoded by this transcript may also play an important role in the normal homeostasis of these tissues. Therapeutics designed with the protein encoded for by this transcript could be important for maintaining or restoring normal function to these organs during inflammation

J. NOV11 (CG55748-01)

Expression of gene CG55748-01 was assessed using the primer-probe set Ag2230, described in Table 22JA.

TABLE 22JA

Probe Name Ag2230

| | | | |
|---|---|---|---|
| Primers | Sequences | Length | Start Position |

| | | | |
|---|---|---|---|
| Forward | 5′-tctcgggatgtcatacactaca-3′ (SEQ ID NO: 152) | 22 | 280 |
| Probe | TET-5′-tgtcaaaacagaccacccaagatttt-3′-TAMRA (SEQ ID NO: 153) | 26 | 303 |
| Reverse | 5′-atcaagtccagcatacaattgg-3′ (SEQ ID NO: 154) | 22 | 333 |

CNS_neurodegeneration_v1.0 Summary: Ag2230 Expression is low/undetectable in all samples in this panel (CTs>35). (Data not shown.)

Panel 1.3D Summary: Ag2230 Expression is low/undetectable in all samples in this panel (CTs>35). (Data not shown.)

Panel 2D Summary: Ag2230 Expression is low/undetectable in all samples in this panel (CTs>35). (Data not shown.)

Panel 4D Summary: Ag2230 Expression is low/undetectable in all samples in this panel (CTs>35). (Data not shown.)

### Example 3

### SNP Analysis of NOVX Clones

SeqCallingTM Technology: cDNA was derived from various human samples representing multiple tissue types, normal and diseased states, physiological states, and developmental states from different donors. Samples were obtained as whole tissue, cell lines, primary cells or tissue cultured primary cells and cell lines. Cells and cell lines may have been treated with biological or chemical agents that regulate gene expression for example, growth factors, chemokines, steroids. The cDNA thus derived was then sequenced using CuraGen's proprietary SeqCalling technology. Sequence traces were evaluated manually and edited for corrections if appropriate. cDNA sequences from all samples were assembled with themselves and with public ESTs using bioinformatics programs to generate CuraGen's human SeqCalling database of SeqCalling assemblies. Each assembly contains one or more overlapping cDNA sequences derived from one or more human samples. Fragments and ESTs were included as components for an assembly when the extent of identity with another component of the assembly was at least 95% over 50 bp. Each assembly can represent a gene and/or its variants such as splice forms and/or single nucleotide polymorphisms (SNPs) and their combinations.

Variant sequences are included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, however, in the case that a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern for example, alteration in temporal expression, physiological response regulation, cell type expression regulation, intensity of expression, stability of transcribed message.

Method of novel SNP Identification: SNPs are identified by analyzing sequence assemblies using CuraGen's proprietary SNPTool algorithm. SNPTool identifies variation in assemblies with the following criteria: SNPs are not analyzed within 10 base pairs on both ends of an alignment; Window size (number of bases in a view) is 10; The allowed number of mismatches in a window is 2; Minimum SNP base quality (PHRED score) is 23; Minimum number of changes to score an SNP is 2/assembly position. SNPTool analyzes the assembly and displays SNP positions, associated individual variant sequences in the assembly, the depth of the assembly at that given position, the putative assembly allele frequency, and the SNP sequence variation. Sequence traces are then selected and brought into view for manual validation. The consensus assembly sequence is imported into CuraTools along with variant sequence changes to identify potential amino acid changes resulting from the SNP sequence variation. Comprehensive SNP data analysis is then exported into the SNPCalling database.

Method of novel SNP Confirmation: SNPs are confirmed employing a validated method know as Pyrosequencing (Pyrosequencing, Westborough, Mass.). Detailed protocols for Pyrosequencing can be found in: Alderbom et al. Determination of Single Nucleotide Polymorphisms by Real-time Pyrophosphate DNA Sequencing. (2000). Genome Research. 10, Issue 8, August. 1249-1265. In brief, Pyrosequencing is a real time primer extension process of genotyping. This protocol takes double-stranded, biotinylated PCR products from genomic DNA samples and binds them to streptavidin beads. These beads are then denatured producing single stranded bound DNA. SNPs are characterized utilizing a technique based on an indirect bioluminometric assay of pyrophosphate (PPi) that is released from each dNTP upon DNA chain elongation. Following Klenow polymerase-mediated base incorporation, PPi is released and used as a substrate, together with adenosine 5′-phosphosulfate (APS), for ATP sulfurylase, which results in the formation of ATP. Subsequently, the ATP accomplishes the conversion of luciferin to its oxi-derivative by the action of luciferase. The ensuing light output becomes proportional to the number of added bases, up to about four bases. To allow processivity of the method dNTP excess is degraded by apyrase, which is also present in the starting reaction mixture, so that only dNTPs are added to the template during the sequencing. The process has been fully automated and adapted to a 96-well format, which allows rapid screening of large SNP panels. The DNA and protein sequences for the novel single nucleotide polymorphic variants are reported. Variants are reported individually but any combination of all or a select subset of variants are also included. In addition, the positions of the variant bases and the variant amino acid residues are underlined.

### Results

Variants are reported individually but any combination of all or a select subset of variants are also included as contemplated NOVX embodiments of the invention.

NOV1a SNP Data:

NOV1a has four SNP variants, whose variant positions for its nucleotide and amino acid sequences is numbered according to SEQ ID NOs: 1 and 2, respectively.

TABLE 23
cSNP and Coding Variants for NOV1

| NT Position | Wild Type | | Amino Acid | Amino Acid |
|---|---|---|---|---|
| of cSNP | NT | Variant NT | position | Change |
| 3082 | G | A | 1028 | Ala-Thr |
| 3120 | G | A | Silent | Silent |
| 3251 | T | C | 1084 | Val-Ala |
| 4085 | A | G | 1362 | Asp-Gly |

NOV4a SNP Data:

NOV4a has five SNP variants, whose variant positions for its nucleotide and amino acid sequences is numbered according to SEQ ID NOs:11 and 12, respectively.

TABLE 24
cSNP and Coding Variants for NOV4

| NT Position | Wild Type | | Amino Acid | Amino Acid |
|---|---|---|---|---|
| of cSNP | NT | Variant NT | position | Change |
| 321 | C | A | Silent | Silent |
| 566 | A | C | 174 | Asn-Thr |
| 751 | T | C | 236 | Phe-Leu |
| 770 | T | C | 242 | Val-Ala |
| 1702 | A | G | Silent | silent |

NOV6a SNP Data:

NOV6a has nine SNP variants, whose variant positions for its nucleotide and amino acid sequences is numbered according to SEQ ID NOs:27 and 28, respectively.

TABLE 25
cSNP and Coding Variants for NOV6a

| NT Position | Wild Type | | Amino Acid | Amino Acid |
|---|---|---|---|---|
| of cSNP | NT | Variant NT | position | Change |
| 3093 | C | T | 986 | Thr-Met |
| 3128 | C | T | 998 | Pro-Ser |
| Insertion before | — | G | 1003-end | Discordant |
| 3145 | | | | |
| Insertion before | — | C | 1020-end | Discordant |
| 3195 | | | | |
| Insertion before | — | C | 1062-end | Discordant |
| 3322 | | | | |
| 3376 | — | C | 1080-end | Discordant |
| 3378 | — | C | 1081-end | Discordant |
| Insertion before | — | C | 1095-end | Discordant |
| 3419 | | | | |
| 3559 | C | T | Silent | Silent |

NOV7 SNP Data:

NOV7 has two SNP variants, whose variant positions for its nucleotide and amino acid sequences is numbered according to SEQ ID NOs:35 and 36, respectively.

TABLE 26
cSNP and Coding Variants for NOV7

| NT Position | Wild Type | | Amino Acid | Amino Acid |
|---|---|---|---|---|
| of cSNP | NT | Variant NT | position | Change |
| 455 | G | A | Silent | Silent |
| 624 | A | G | 208 | Ser-Gly |

NOV8 SNP Data:

NOV8 has two SNP variants, whose variant positions for its nucleotide and amino acid sequences is numbered according to SEQ ID NOs:37 and 38, respectively.

TABLE 27
cSNP and Coding Variants for NOV8

| NT Position | Wild Type | | Amino Acid | Amino Acid |
|---|---|---|---|---|
| of cSNP | NT | Variant NT | position | Change |
| 149 | T | C | Silent | Silent |
| 237 | A | G | 72 | Arg-Gly |

NOV10a SNP Data:

NOV10a has two SNP variants, whose variant positions for its nucleotide and amino acid sequences is numbered according to SEQ ID NOs:41 and 42, respectively.

TABLE 28
cSNP and Coding Variants for NOV8

| NT Position | Wild Type | | Amino Acid | Amino Acid |
|---|---|---|---|---|
| of cSNP | NT | Variant NT | position | Change |
| 1677 | A | G | 438 | Asp-Gly |
| 1771 | C | G | 469 | Asn-Lys |

### Example 4

### PCR Cloning of NOV6B

The cDNA coding for a domain of CG50215-03 from residue 436 to 975 was targeted for “in-frame” cloning by PCR. The PCR template is based on human cDNA(s).

The following oligonucleotide primers were used to clone the target cDNA sequence:

| | |
|---|---|
| F3 | (SEQ ID NO: 155) |

5′-AAGGTT TGTCAGCGCAACCCCCAGGTGTGCGGCCCAGG-3

| | |
|---|---|
| F5 | (SEQ ID NO: 156) |

5′-CTCGAG ACAGCGTCCAGTCATGGGGTCAAACTCTTCC-3′

For downstream cloning purposes, the forward primer includes an in-frame HindIII restriction site and the reverse primer contains an in-frame XhoI restriction site.

Two parallel PCR reactions were set up using a total of 0.5-1.0 ng human pooled cDNAs as template for each reaction. The pool is composed of 5 micrograms of each of the following human tissue cDNAs: adrenal gland, whole brain, amygdala, cerebellum, thalamus, bone marrow, fetal brain, fetal kidney, fetal liver, fetal lung, heart, kidney, liver, lymphoma, Burkitt's Raji cell line, mammary gland, pancreas, pituitary gland, placenta, prostate, salivary gland, skeletal muscle, small Intestine, spleen, stomach, thyroid, trachea, uterus.

When the tissue of expression is known and available, the second PCR was performed using the above primers and 0.5 ng-1.0 ng of one of the following human tissue cDNAs:

skeleton muscle, testis, mammary gland, adrenal gland, ovary, colon, normal cerebellum, normal adipose, normal skin, bone marrow, brain amygdala, brain hippocampus, brain substantia nigra, brain thalamus, thyroid, fetal lung, fetal liver, fetal brain, kidney, heart, spleen, uterus, pituitary gland, lymph node, salivary gland, small intestine, prostate, placenta, spinal cord, peripheral blood, trachea, stomach, pancreas, hypothalamus.

The reaction mixtures contained 2 microliters of each of the primers (original concentration: 5 pmol/ul), 1 microliter of 10 mM dNTP (Clontech Laboratories, Palo Alto Calif.) and 1 microliter of 50×Advantage-HF 2 polymerase (Clontech Laboratories) in 50 microliter-reaction volume. The following reaction conditions were used:

PCR condition 1:

a) 96° C. 3 minutes

b) 96° C. 30 seconds denaturation

c) 60° C. 30 seconds, primer annealing

d) 72° C. 6 minutes extension

Repeat steps b-d 15 times

e) 96° C. 15 seconds denaturation

f) 60° C. 30 seconds, primer annealing

g) 72° C. 6 minutes extension

Repeat steps e-g 29 times

e) 72° C. 10 minutes final extension

PCR condition 2:

a) 96° C. 3 minutes

b) 96° C. 15 seconds denaturation

c) 76° C. 30 seconds, primer annealing, reducing the temperature by 1° C. per cycle

d) 72° C. 4 minutes extension

Repeat steps b-d 34 times

e) 72° C. 10 minutes final extension

An amplified product was detected by agarose gel electrophoresis. The fragment was gel-purified and ligated into the pCR2.1 vector (Invitrogen, Carlsbad, Calif.) following the manufacturer's recommendation. Twelve clones per PCR reaction were picked and sequenced. The inserts were sequenced using vector-specific M13 Forward and M13 Reverse primers and the following gene-specific primers:

| | | |
|---|---|---|
| SF1: | GAGAACACGCCAGGCAGCTT | (SEQ ID NO: 157) |
| SF2: | CTCCTTTCACTGTGCCTGCCC | (SEQ ID NO: 158) |
| SF3: | TGTCCTTCTGGCCACCACC | (SEQ ID NO: 159) |
| SF4: | GAGCCTCTTGCCTCGACGTTGACGAGT | (SEQ ID NO: 160) |
| SF5: | GTGTCCGGGACTGCGATCCT | (SEQ ID NO: 161) |
| SR1: | CGGTGGCACTCGTCCACAT | (SEQ ID NO: 162) |
| SR2: | CTGCCGTGTTGTCACAGCG | (SEQ ID NO: 163) |
| SR3: | AGGCCCTGCACTGGAAGGA | (SEQ ID NO: 164) |
| SR4: | GTCGGTAGCCAGGGGCACAAGTA | (SEQ ID NO: 165) |
| SR5: | AGTCCCGGACACAGCGGTA | (SEQ ID NO: 166) |

The insert assembly 197188002 was found to encode an open reading frame between residues 436 and 975 of the target sequence CG50215-03. 197188002 differs from the original sequence at 2 nucleotide positions and 2 amino acid positions.

The cDNA coding for a domain of CG50215-03 from residue 40 to 345 was targeted for “in-frame” cloning by PCR. The PCR template is based on human cDNA(s).

The following oligonucleotide primers were used to clone the target cDNA sequence:

(SEQ ID NO:167)

F2 5′-AAGCTT TGTCCCTTGATCTGTCACAATGGCGGTGTGTGC-3′

(SEQ ID NO: 168)

R2 5′-CTCGAG GATCTCCCGGAAACCCTCTGAGCCGAAGGG-3′

For downstream cloning purposes, the forward primer includes an in-frame HindIII restriction site and the reverse primer contains an in-frame XhoI restriction site.

Two parallel PCR reactions were set up using a total of 0.5-1.0 ng human pooled cDNAs as template for each reaction. The pool is composed of 5 micrograms of each of the following human tissue cDNAs: adrenal gland, whole brain, amygdala, cerebellum, thalamus, bone marrow, fetal brain, fetal kidney, fetal liver, fetal lung, heart, kidney, liver, lymphoma, Burkitt's Raji cell line, mammary gland, pancreas, pituitary gland, placenta, prostate, salivary gland, skeletal muscle, small Intestine, spleen, stomach, thyroid, trachea, uterus.

When the tissue of expression is known and available, the second PCR was performed using the above primers and 0.5 ng-1.0 ng of one of the following human tissue cDNAs:

skeleton muscle, testis, mammary gland, adrenal gland, ovary, colon, normal cerebellum, normal adipose, normal skin, bone marrow, brain amygdala, brain hippocampus, brain substantia nigra, brain thalamus, thyroid, fetal lung, fetal liver, fetal brain, kidney, heart, spleen, uterus, pituitary gland, lymph node, salivary gland, small intestine, prostate, placenta, spinal cord, peripheral blood, trachea, stomach, pancreas, hypothalamus.

The reaction mixtures contained 2 microliters of each of the primers (original concentration: 5 pmol/ul), 1 microliter of 10 mM dNTP (Clontech Laboratories, Palo Alto Calif.) and 1 microliter of 50×Advantage-HF 2 polymerase (Clontech Laboratories) in 50 microliter-reaction volume. The following reaction conditions were used:

PCR condition 1:

a) 96° C. 3 minutes

b) 96° C. 30 seconds denaturation

c) 60° C. 30 seconds, primer annealing

d) 72° C. 6 minutes extension

Repeat steps b-d 15 times

e) 96° C. 15 seconds denaturation

f) 60° C. 30 seconds, primer annealing

g) 72° C. 6 minutes extension

Repeat steps e-g 29 times

e) 72° C. 10 minutes final extension

PCR condition 2:

a) 96° C. 3 minutes

b) 96° C. 15 seconds denaturation

c) 76° C. 30 seconds, primer annealing, reducing the temperature by 1° C. per cycle

d) 72° C. 4 minutes extension

Repeat steps b-d 34 times

e) 72° C. 10 minutes final extension

An amplified product was detected by agarose gel electrophoresis. The fragment was gel-purified and ligated into the pCR2.1 vector (Invitrogen, Carlsbad, Calif.) following the manufacturer's recommendation. Twelve clones per PCR reaction were picked and sequenced. The inserts were sequenced using vector-specific M13 Forward and M13 Reverse primers and the following gene-specific primers:

| | | |
|---|---|---|
| SF1: | GGCAGCGCCCTACACGGT | (SEQ ID NO: 169) |
| SF2: | GATGAGTGCGCGACTGGC | (SEQ ID NO: 170) |
| SR1: | CCTCAGCGTCCGCCTCCT | (SEQ ID NO: 171) |
| SR2: | CGCACTCATCCACATCTTCGC | (SEQ ID NO: 172) |

The insert assemblies 197187970, 197187982, and 197187990 were all found to encode an open reading frame between residues 40 and 345 of the target sequence CG50215-03. The cloned insert of assembly 197187982 is 100% identical to the original sequence. 197187970 differs from the original sequence at 2 nucleotide positions and 1 amino acid position. 197187990 differs from the original sequence at 1 nucleotide position and one amino acid position.

### OTHER EMBODIMENTS

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims.

What is claimed is:

1. An isolated polypeptide comprising an amino acid sequence selected from the group consisting of:
(a) a mature form of an amino acid sequence selected from the group consisting of SEQ ID NOS:2, 4, 6, 8, 10, 12, 14, 16, 18, 20, 22, 24, 26, 28, 30, 32, 34, 26, 28, 40, 42 and 44;
(b) a variant of a mature form of an amino acid sequence selected from the group consisting of SEQ ID NOS:2, 4, 6, 8, 10, 12, 14, 16, 18, 20, 22, 24, 26, 28, 30, 32, 34, 26, 28, 40, 42 and 44, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting SEQ ID NOS:2, 4, 6, 8, 10, 12, 14, 16, 18, 20, 22, 24, 26, 28, 30, 32, 34, 26, 28, 40, 42 and 44; and
(d) a variant of an amino acid sequence selected from the group consisting of SEQ ID NOS:2, 4, 6, 8, 10, 12, 14, 16, 18, 20, 22, 24, 26, 28, 30, 32, 34, 26, 28, 40, 42 and 44, wherein one or more amino acid residues in said variant differs from the amino acid sequence of said mature form, provided that said variant differs in no more than 15% of amino acid residues from said amino acid sequence.

2. The polypeptide of claim 1, wherein said polypeptide comprises the amino acid sequence of a naturally-occurring allelic variant of an amino acid sequence selected from the group consisting SEQ ID NOS:2, 4, 6, 8, 10, 12, 14, 16, 18, 20, 22, 24, 26, 28, 30, 32, 34, 26, 28, 40, 42 and 44.

3. The polypeptide of claim 2, wherein said allelic variant comprises an amino acid sequence that is the translation of a nucleic acid sequence differing by a single nucleotide from a nucleic acid sequence selected from the group consisting of SEQ ID NOS:1, 3, 5, 7, 9, 11, 13, 15, 17, 19, 21, 23, 25, 27, 29, 31, 33, 35, 37, 39, 41 and 43.

4. The polypeptide of claim 1, wherein the amino acid sequence of said variant comprises a conservative amino acid substitution.

5. An isolated nucleic acid molecule comprising a nucleic acid sequence encoding a polypeptide comprising an amino acid sequence selected from the group consisting of:
(a) a mature form of an amino acid sequence selected from the group consisting of SEQ ID NOS:2, 4, 6, 8, 10, 12, 14, 16, 18, 20, 22, 24, 26, 28, 30, 32, 34, 26, 28, 40, 42 and 44;
(b) a variant of a mature form of an amino acid sequence selected from the group consisting of SEQ ID NOS:2, 4, 6, 8, 10, 12, 14, 16, 18, 20, 22, 24, 26, 28, 30, 32, 34, 26, 28, 40, 42 and 44, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS:2, 4, 6, 8, 10, 12, 14, 16, 18, 20, 22, 24, 26, 28, 30, 32, 34, 26, 28, 40, 42 and 44;
(d) a variant of an amino acid sequence selected from the group consisting SEQ ID NOS:2, 4, 6, 8, 10, 12, 14, 16, 18, 20, 22, 24, 26, 28, 30, 32, 34, 26, 28, 40, 42 and 44, wherein one or more amino acid residues in said variant differs from the amino acid sequence of said mature form, provided that said variant differs in no more than 15% of amino acid residues from said amino acid sequence;
(e) a nucleic acid fragment encoding at least a portion of a polypeptide comprising an amino acid sequence chosen from the group consisting of SEQ ID NOS:2, 4, 6, 8, 10, 12, 14, 16, 18, 20, 22, 24, 26, 28, 30, 32, 34, 26, 28, 40, 42 and 44, or a variant of said polypeptide, wherein one or more amino acid residues in said variant differs from the amino acid sequence of said mature form, provided that said variant differs in no more than 15% of amino acid residues from said amino acid sequence; and
(f) a nucleic acid molecule comprising the complement of (a), (b), (c), (d) or (e).

6. The nucleic acid molecule of claim 5, wherein the nucleic acid molecule comprises the nucleotide sequence of a naturally-occurring allelic nucleic acid variant.

7. The nucleic acid molecule of claim 5, wherein the nucleic acid molecule encodes a polypeptide comprising the amino acid sequence of a naturally-occurring polypeptide variant.

8. The nucleic acid molecule of claim 5, wherein the nucleic acid molecule differs by a single nucleotide from a nucleic acid sequence selected from the group consisting of SEQ ID NOS:1, 3, 5, 7, 9, 11, 13, 15, 17, 19, 21, 23, 25, 27, 29, 31, 33, 35, 37, 39, 41 and 43.

9. The nucleic acid molecule of claim 5, wherein said nucleic acid molecule comprises a nucleotide sequence selected from the group consisting of:
(a) a nucleotide sequence selected from the group consisting of SEQ ID NOS:1, 3, 5, 7, 9, 11, 13, 15, 17, 19, 21, 23, 25, 27, 29, 31, 33, 35, 37, 39, 41 and 43;
(b) a nucleotide sequence differing by one or more nucleotides from a nucleotide sequence selected from the group consisting of SEQ ID NOS:1, 3, 5, 7, 9, 11, 13, 15, 17, 19, 21, 23, 25, 27, 29, 31, 33, 35, 37, 39, 41 and 43, provided that no more than 20% of the nucleotides differ from said nucleotide sequence;
(c) a nucleic acid fragment of (a); and
(d) a nucleic acid fragment of (b).

10. The nucleic acid molecule of claim 5, wherein said nucleic acid molecule hybridizes under stringent conditions to a nucleotide sequence chosen from the group consisting SEQ ID NOS:1, 3, 5, 7, 9, 11, 13, 15, 17, 19, 21, 23, 25, 27, 29, 31, 33, 35, 37, 39, 41 and 43, or a complement of said nucleotide sequence.

11. The nucleic acid molecule of claim 5, wherein the nucleic acid molecule comprises a nucleotide sequence selected from the group consisting of:
(a) a first nucleotide sequence comprising a coding sequence differing by one or more nucleotide sequences from a coding sequence encoding said amino acid sequence, provided that no more than 20% of the nucleotides in the coding sequence in said first nucleotide sequence differ from said coding sequence;
(b) an isolated second polynucleotide that is a complement of the first polynucleotide; and
(c) a nucleic acid fragment of (a) or (b).

12. A vector comprising the nucleic acid molecule of claim 11.

13. The vector of claim 12, further comprising a promoter operably-linked to said nucleic acid molecule.

14. A cell comprising the vector of claim 12.

15. An antibody that binds immunospecifically to the polypeptide of claim 1.

16. The antibody of claim 15, wherein said antibody is a monoclonal antibody.

17. The antibody of claim 15, wherein the antibody is a humanized antibody.

18. A method for determining the presence or amount of the polypeptide of claim 1 in a sample, the method comprising:
(a) providing the sample;
(b) contacting the sample with an antibody that binds immunospecifically to the polypeptide; and
(c) determining the presence or amount of antibody bound to said polypeptide, thereby determining the presence or amount of polypeptide in said sample.

19. A method for determining the presence or amount of the nucleic acid molecule of claim 5 in a sample, the method comprising:
(a) providing the sample;
(b) contacting the sample with a probe that binds to said nucleic acid molecule; and
(c) determining the presence or amount of the probe bound to said nucleic acid molecule,
thereby determining the presence or amount of the nucleic acid molecule in said sample.

20. The method of claim 19 wherein presence or amount of the nucleic acid molecule is used as a marker for cell or tissue type.

21. The method of claim 20 wherein the cell or tissue type is cancerous.

22. A method of identifying an agent that binds to a polypeptide of claim 1, the method comprising:
(a) contacting said polypeptide with said agent; and
(b) determining whether said agent binds to said polypeptide.

23. The method of claim 22 wherein the agent is a cellular receptor or a downstream effector.

24. A method for identifying an agent that modulates the expression or activity of the polypeptide of claim 1, the method comprising:
(a) providing a cell expressing said polypeptide;
(b) contacting the cell with said agent, and
(c) determining whether the agent modulates expression or activity of said polypeptide,
whereby an alteration in expression or activity of said peptide indicates said agent modulates expression or activity of said polypeptide.

25. A method for modulating the activity of the polypeptide of claim 1, the method comprising contacting a cell sample expressing the polypeptide of said claim with a compound that binds to said polypeptide in an amount sufficient to modulate the activity of the polypeptide.

26. A method of treating or preventing a NOVX-associated disorder, said method comprising administering to a subject in which such treatment or prevention is desired the polypeptide of claim 1 in an amount sufficient to treat or prevent said NOVX-associated disorder in said subject.

27. The method of claim 26 wherein the disorder is selected from the group consisting of cardiomyopathy and atherosclerosis.

28. The method of claim 26 wherein the disorder is related to cell signal processing and metabolic pathway modulation.

29. The method of claim 26, wherein said subject is a human.

30. A method of treating or preventing a NOVX-associated disorder, said method comprising administering to a subject in which such treatment or prevention is desired the nucleic acid of claim 5 in an amount sufficient to treat or prevent said NOVX-associated disorder in said subject.

31. The method of claim 30 wherein the disorder is selected from the group consisting of cardiomyopathy and atherosclerosis.

32. The method of claim 30 wherein the disorder is related to cell signal processing and metabolic pathway modulation.

33. The method of claim 30, wherein said subject is a human.

34. A method of treating or preventing a NOVX-associated disorder, said method comprising administering to a subject in which such treatment or prevention is desired the antibody of claim 15 in an amount sufficient to treat or prevent said NOVX-associated disorder in said subject.

35. The method of claim 34 wherein the disorder is diabetes.

36. The method of claim 34 wherein the disorder is related to cell signal processing and metabolic pathway modulation.

37. The method of claim 34, wherein the subject is a human.

38. A pharmaceutical composition comprising the polypeptide of claim 1 and a pharmaceutically-acceptable carrier.

39. A pharmaceutical composition comprising the nucleic acid molecule of claim 5 and a pharmaceutically-acceptable carrier.

40. A pharmaceutical composition comprising the antibody of claim 15 and a pharmaceutically-acceptable carrier.

41. A kit comprising in one or more containers, the pharmaceutical composition of claim 38.

42. A kit comprising in one or more containers, the pharmaceutical composition of claim 39.

43. A kit comprising in one or more containers, the pharmaceutical composition of claim 40.

44. A method for determining the presence of or predisposition to a disease associated with altered levels of the polypeptide of claim 1 in a first mammalian subject, the method comprising:
(a) measuring the level of expression of the polypeptide in a sample from the first mammalian subject; and
(b) comparing the amount of said polypeptide in the sample of step (a) to the amount of the polypeptide present in a control sample from a second mammalian subject known not to have, or not to be predisposed to, said disease;
wherein an alteration in the expression level of the polypeptide in the first subject as compared to the control sample indicates the presence of or predisposition to said disease.

45. The method of claim 44 wherein the predisposition is to a cancer.

46. A method for determining the presence of or predisposition to a disease associated with altered levels of the nucleic acid molecule of claim 5 in a first mammalian subject, the method comprising:
(a) measuring the amount of the nucleic acid in a sample from the first mammalian subject; and
(b) comparing the amount of said nucleic acid in the sample of step (a) to the amount of the nucleic acid present in a control sample from a second mammalian subject known not to have or not be predisposed to, the disease;
wherein an alteration in the level of the nucleic acid in the first subject as compared to the control sample indicates the presence of or predisposition to the disease.

47. The method of claim 46 wherein the predisposition is to a cancer.

48. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising an amino acid sequence of at least one of SEQ ID NOS:2, 4, 6, 8, 10, 12, 14, 16, 18, 20, 22, 24, 26, 28, 30, 32, 34, 26, 28, 40, 42 and 44, or a biologically active fragment thereof.

49. A method of treating a pathological state in a mammal, the method comprising administering to the mammal the antibody of claim 15 in an amount sufficient to alleviate the pathological state.
